(19) United States
(12) Patent Application Publication
(10) Pub. No.: US 2025/0034648 A1
(43) Pub. Date: Jan. 30, 2025

(54) KITS AND METHODS USEFUL FOR PROGNOSING, DIAGNOSING, AND TREATING PROSTATE CANCER

(71) Applicant: The Regents of the University of Michigan, Ann Arbor, MI (US)

(72) Inventors: Arul M. Chinnaiyan, Ann Arbor, MI (US); Jeffrey J. Tosoian, Ann Arbor, MI (US); Yuping Zhang, Ann Arbor, MI (US); Lanbo Xiao, Ann Arbor, MI (US)

(21) Appl. No.: 18/425,867

(22) Filed: Jan. 29, 2024

Related U.S. Application Data
(60) Provisional application No. 63/446,596, filed on Feb. 17, 2023.
(60) Provisional application No. 63/442,045, filed on Jan. 30, 2023.

(51) Int. Cl. C12Q 1/6886

(52) U.S. Cl. CPC C12Q 1/6886; C12Q 2600/118; C12Q 2600/158

(57) ABSTRACT

Provided herein are kits and methods useful for cancer diagnosis, prognosis, research and therapy. In particular, provided herein are methods of diagnosing, prognosing, and/or treating prostate cancer based on expression levels of cancer markers.

### CROSS-REFERENCE TO RELATED APPLICATIONS

This application claims the benefit of and priority from U.S. Provisional Patent Application No. 63/442,045, filed Jan. 30, 2023, and U.S. Provisional Patent Application No. 63/446,596, filed Feb. 17, 2023, each of which is incorporated by reference herein in its entirety.

### STATEMENT REGARDING FEDERALLY SPONSORED RESEARCH OR DEVELOPMENT

This invention was made with government support under CA214170, CA186786, CA271854, and CA231996 awarded by the National Institutes of Health. The government has certain rights in the invention.

### REFERENCE TO AN ELECTRONIC SEQUENCE LISTING

The contents of the electronic sequence listing (LXDX-002-001WO.xml; Size 50,183 bytes; and Date of Creation: Jan. 28, 2024) are herein incorporated by reference in their entireties.

### FIELD OF THE DISCLOSURE

Provided herein are kits and methods useful for cancer diagnosis, prognosis, research and therapy. In particular, provided herein are methods of diagnosing, prognosing, and/or treating prostate cancer based on expression levels of cancer markers.

### BACKGROUND OF THE DISCLOSURE

Prostate cancer is the third most common urologic malignancy and can originate from the prostate parenchyma or urinary collecting system. Prostate cell carcinoma, arising from the prostate parenchyma, is the most common malignant prostate tumor associated with an incidence of 64,000 cases and approximately 14,000 deaths yearly in the United States. From the urinary collecting system, urothelial cell carcinoma is the most common malignancy representing approximately 10-15% of all prostate tumors. The overall incidence of malignant prostate tumors is increasing and currently is the third most common form of genitourinary cancer. Both malignant and benign prostate tumors are increasingly diagnosed in incidental fashion with the use of advanced cross-sectional imaging. Accurate diagnosis of benign versus malignant tumor types is lacking and accordingly patients might be subjected to unnecessary treatment or overtreatment. Furthermore, there are currently no diagnostic tests from needle biopsy, urine or blood that accurately characterize prostate tumors or identify patients at risk for prostate tumors. The diagnostic and therapeutic approach to prostate tumors is complicated by the presence of multiple benign prostate tumor types and the fact that many small malignant prostate parenchymal tumors can be observed rather than definitively treated.

Early detection and treatment of aggressive prostate cancers are critical to reducing its harms, but current diagnostic tests are unable to reliably identify clinically significant (e.g., classified as Grade Group [GG]≥2) prostate cancer. Poorly specific for cancer, the harms of serum prostate-specific antigen (PSA) as an isolated diagnostic tool are well-documented, and several cancer-specific biomarkers have been proposed to augment PSA. These tools have demonstrated incremental benefit, potentially avoiding 15-30% of biopsies performed due to PSA, at the cost of failing to diagnose 8-15% of GG≥2 prostate cancer. MRI has been similarly used in this role at several academic centers. In addition to mounting evidence that a proportion of GG≥2 cancers are MRI-invisible, MRI is costly, resource-intensive, and subjectively interpreted, making it less practical as a population-level diagnostic tool. Thus, there continues to be a critical need for a practical (affordable, reproducible, standardizable) non-invasive test to reliably detect aggressive prostate cancer in a localized, curable state.

While several molecular mechanisms yield aggressive prostate cancer biology, most patients harbor tumors reflecting a limited number of these mechanisms. At the present time, there are no accurate, user-friendly, and widely accessible screening tools at the tissue, blood or urinary level for ideal clinical management of prostate tumors.

### SUMMARY OF THE DISCLOSURE

Provided herein are methods of treating prostate cancer, comprising: a) assaying the level of expression of one or more genes selected from TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6 in a sample from a subject diagnosed with prostate cancer; and b) administering a prostate cancer treatment to a subject identified as having altered levels of expression of one or more of the genes relative to a subject without prostate cancer or a subject with low-grade prostate cancer.

Further provided are methods of characterizing, prognosing, or recommending a treatment for prostate cancer, comprising: a) assaying the level of expression of one or more genes selected from TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6 in a sample from a subject diagnosed with prostate cancer; and b) identifying said subject as having high-grade prostate cancer when the subject is identified as having altered levels of expression of the genes relative to a subject without prostate cancer or a subject with low-grade prostate cancer.

Further provided are methods for informing a prostate cancer survival outcome, comprising: (i) detecting an amount of expression of at least three genes selected from TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6, wherein the amount of expression is present in urine from a subject; (ii) determining a score based on the amount of expression, wherein the score correlates with or informs the subject's likelihood of having or developing Grade Group ≥2 prostate cancer; and (iii) generating a report comprising the score.

Further provided are methods for identifying a subject having a high likelihood of having or developing Grade Group ≥2 prostate cancer, comprising detecting an amount of expression of at least three genes selected from TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6, wherein the amount of expression is present in the subject's urine and indicates with a diagnostic accuracy (AUC) of ≥0.75 whether the subject has a high likelihood of having a Grade Group ≥2 prostate cancer.

Further provided are methods for identifying a likelihood of detecting Grade Group ≥2 prostate cancer from a prostate biopsy of a subject, the method comprising detecting an amount of expression of at least three genes selected from TMPRSS2-ERG, SCHLAP1, OR51 E2, APOC1, PCAT14, CAMKK2, PCA3, NKAIN1, B3GNT6, TFF3, SPON2, PCGEM1, TRGV9, TMSB15A, ERG, KLK4, and HOXC6, wherein the amount of expression is present in the subject's urine and indicates with a diagnostic accuracy (AUC) of ≥0.75 the likelihood that Grade Group ≥2 prostate cancer would be detected from the prostate biopsy of the subject.

Further provided are methods for screening for an amount of expression of at least three genes, comprising: (a) allowing a sample of urine from a human subject to react with a reagent for detecting an amount of expression of the at least three genes, wherein the at least three genes are selected from TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6; and (b) detecting the amount of expression of the at least three genes, wherein the amount of expression is present in the sample and the detecting comprises using an in vitro assay.

Further provided are methods for detecting an amount of mRNA expressed by at least three genes, comprising: (a) synthesizing cDNA from mRNA that is expressed by the at least three genes and present in a sample of urine from a human subject, wherein the at least three genes are selected from TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6; (b) amplifying the cDNA to provide amplified cDNA; and (c) detecting the amplified cDNA, wherein the amplified cDNA indicates the amount of mRNA expressed by the at least three genes.

Further provided are methods for detecting an amount of mRNA expressed by at least three genes, comprising: (a) isolating nucleic acid from a first composition comprising urine from a human subject to provide isolated nucleic acid; (b) allowing the isolated nucleic acid to react with a second composition comprising a reagent for detecting the amount of mRNA that is present in the first composition and expressed by the at least three genes, wherein the at least three genes are selected from TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6; and (c) detecting the amount of mRNA expressed by the at least three genes.

Further provided are kits comprising: a container, the container containing a reagent composition for detecting an amount of expression of at least three genes; and instructions for detecting the amount of expression, where the amount of expression is present in a subject's urine and the at least three genes are selected from TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6.

### DESCRIPTION OF THE FIGURES

FIGS. 1A and 1B. Flowcharts of biomarker discovery (FIG. 1A) and sample inclusion in a MyProstateScore 2.0 (MPS2) training cohort (from University of Michigan, UM) and validation cohort (from NCI-Early Detection Research Network, EDRN) (FIG. 1B).

FIGS. 2A-2C. Model development procedures. Redundant variables (highly correlated) were removed if variance inflation factor (VIF)>5 (FIG. 2A). To select a robust gene panel, elastic net regression models were trained on 40 subsampling data (FIG. 2B). Calibration Curves for Clinically Significant Prostate Cancer for MPS2 and MPS2+ in the External Validation Cohort (FIG. 2C).

FIGS. 3A-3E. Performance evaluation of MPS2 models on the training cohort. Receiver Operating Characteristic (ROCs) of original MyProstateScore (MPS) (TMPRSS2-ERG+PCA3) (FIG. 3A). ROCs of MPS2 gene panel (FIG. 3B). ROCs of MPS2 plus clinical variables (MPS2c) (FIG. 3C). ROCs of MPS2 plus clinical variables and prostate volume (MPS2cv) (FIG. 3D). Model calibration analysis after calibration by adjusting slope and intercept (FIG. 3E).

FIGS. 4A-4D. Performance evaluation of MPS2 models on the validation cohort. ROCs and area under the curves (AUCs) of MPS2 models (FIG. 4A). Calibration curves of calibrated risk probability (FIG. 4B). Decision curve analysis demonstrating net benefits of MPS2 models versus “Treat All” or “Treat None” across different probability thresholds (FIG. 4C). Interventions (biopsies) avoided across different probability thresholds (FIG. 4D).

FIGS. 5A-5G. Associations of selected genes with high-grade prostate cancer in the TCGA PRAD (The Cancer Genome Atlas prostate adenocarcinoma) cohort. The 17 genes used in the final MPS2 models are: TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6.

FIG. 6. Evaluation of feature selection methods. Boxplot showing areas under the curve (AUCs) of each feature selection method from repeated cross validation. RFE, recursive feature elimination.

FIG. 7. Calibration curves of MPS2 models on the validation cohort without calibration. Predicted risk is overestimated without correction for imbalanced classes in the validation cohort.

FIGS. 8A and 8B. MPS2 Values by Biopsy Pathology in the External Validation Cohort. Box and dot plots illustrating the distribution of MPS2 (FIG. 8A) and MPS2+ (FIG. 8B) values in men having a negative biopsy, GG1 cancer on biopsy, and GG≥2 cancer on biopsy in the external validation cohort. P-values were ≤0.001 for pairwise comparisons of GG≥2 cancer with negative biopsy and GG1 cancer for both MPS2 models.

FIG. 9. MPS2 and MPS2+ Area Under the ROC Curves for Clinically Significant Prostate Cancer in the External Validation Cohort. Receiver-operating characteristic curves and areas under the curve (AUC) for PSA (gray), PCPTrc (PCa Prevention Trial risk calculator, yellow), prostate health index (PHI, purple), dmx2 (derived multiplex 2-gene model (HOXC6, DLX1), pink), dmx3 (derived multiplex 3-gene model (PCA3, ERG, SPDEF), maroon), MPS (MyProstateScore, orange), MPS2 (green), and MPS2+ (blue) in the external validation cohort.

FIGS. 10A and 10B. Decision Curve Analysis for Clinically Significant Prostate Cancer in the External Validation Cohort. FIG. 10A shows decision curve analysis (DCA) plots for net clinical benefit of pre-biopsy testing with PSA (gray), PCPTrc (yellow), PHI (purple), dmx2 (pink), dmx3 (maroon), MPS (orange), MPS2 (green), and MPS2+ (blue) as compared to baseline approaches of “biopsy all” (black) or “biopsy none” (dark green). FIG. 10B shows DCA plots illustrating the net reduction in biopsies performed per 100 patients without missing a single diagnosis of GG≥2 cancer based on pre-biopsy testing with PSA (gray), PCPTrc (yellow), PHI (purple), dmx2 (pink), dmx3 (maroon), MPS (orange), MPS2 (green), and MPS2+ (blue) as compared to a baseline approach of biopsying all patients.

FIG. 11. Flow Diagram of the NCI-EDRN External Validation Cohort. Shown is the external validation cohort comprised of men undergoing prostate biopsy in the National Cancer Institute-Early Detection Research Network (NCI-EDRN) PCA3 Trial.

### DEFINITIONS

To facilitate an understanding of the present disclosure, a number of terms and phrases are defined below:

As used herein, the terms “detect”, “detecting” or “detection” may describe either the general act of discovering or discerning or the specific observation of a composition. Detecting a composition may comprise determining the presence or absence of a composition. Detecting may comprise quantifying a composition. For example, detecting comprises determining the expression level of a composition. The composition may comprise a nucleic acid molecule. For example, the composition may comprise at least a portion of the cancer markers disclosed herein. Alternatively, or additionally, the composition may be a detectably labeled composition.

As used herein, the term “subject” refers to any organisms that are screened using the diagnostic methods described herein. Such organisms preferably include, but are not limited to, mammals (e.g., murines, simians, equines, bovines, porcines, canines, felines, and the like), and most preferably includes humans. In some embodiments, the subject is a mammal having a prostate. In some embodiments, the subject is a human having a prostate.

The term “diagnosed,” as used herein, refers to the recognition of a disease by its signs and symptoms, or genetic analysis, pathological analysis, histological analysis, and the like.

As used herein, the language “characterizing cancer in a subject” refers to the identification of one or more properties of a cancer sample in a subject, including but not limited to, the presence of benign, pre-cancerous or cancerous tissue, the stage of the cancer, and the subject's prognosis. Cancers may be characterized by the identification of the expression of one or more cancer marker genes, including but not limited to, the cancer markers disclosed herein.

As used herein, the language “stage of cancer” refers to a qualitative or quantitative assessment of the level of advancement of a cancer. Criteria useful to determine the stage of a cancer include, but are not limited to, the size of the tumor and the extent of metastases (e.g., localized or distant).

As used herein, the term “high likelihood” when used, for example, in reference to the likelihood of having or developing prostate cancer (e.g., Grade Group ≥2 prostate cancer) refers to an increased likelihood of developing Grade Group ≥2 prostate cancer relative to a low-risk subject or a high absolute likelihood of developing Grade Group ≥2 prostate cancer. In some embodiments, a high likelihood of developing Grade Group ≥2 prostate cancer is determined based on the level of expression of 1 or more genes described herein. In some embodiments, a “high likelihood” is a likelihood that is increased by 50%, 100%, 200%, 500%, or more relative to a healthy subject or a subject that does not have altered expression of genes recited herein. In some embodiments, a high likelihood refers to the absolute likelihood of developing Grade Group ≥2 prostate cancer. In some embodiments, a “high likelihood” is a 50% or greater likelihood that a subject's prostate biopsy would detect Grade Group ≥2 prostate cancer in the subject. In some embodiments, a “high likelihood” is a 60% or greater likelihood that a subject's prostate biopsy would detect Grade Group ≥2 prostate cancer in the subject. In some embodiments, a “high likelihood” is a 70% or greater likelihood that a subject's prostate biopsy would detect Grade Group ≥2 prostate cancer in the subject. In some embodiments, a “high likelihood” is a 80% or greater likelihood that a subject's prostate biopsy would detect Grade Group ≥2 prostate cancer in the subject. In some embodiments, a “high likelihood” is a 90% or greater likelihood that a subject's prostate biopsy would detect Grade Group ≥2 prostate cancer in the subject. In some embodiments, a “high likelihood” is a 95% or greater likelihood that a subject's prostate biopsy would detect Grade Group ≥2 prostate cancer in the subject. In some embodiments, a “high likelihood” is a 96% or greater likelihood that a subject's prostate biopsy would detect Grade Group ≥2 prostate cancer in the subject. In some embodiments, a “high likelihood” is a 97% or greater likelihood that a subject's prostate biopsy would detect Grade Group ≥2 prostate cancer in the subject. In some embodiments, a “high likelihood” is a 98% or greater likelihood that a subject's prostate biopsy would detect Grade Group ≥2 prostate cancer in the subject. In some embodiments, a “high likelihood” is a 99% or greater likelihood that a subject's prostate biopsy would detect Grade Group ≥2 prostate cancer in the subject. In some embodiments, a “high likelihood” is a 100% likelihood that a subject's prostate biopsy would detect Grade Group ≥2 prostate cancer in the subject.

As used herein, the language “low likelihood” when used, for example, in reference to the likelihood of having or developing prostate cancer (e.g., Grade Group ≥2 prostate cancer) refers to a decreased likelihood of developing prostate cancer (e.g., Grade Group ≥2 prostate cancer) relative to an average-risk subject or a low absolute likelihood of developing prostate cancer (e.g., Grade Group ≥2 prostate cancer). In some embodiments, a low likelihood of developing Grade Group ≥2 prostate cancer is determined based on the level of expression of 1 or more genes described herein. In some embodiments, a “low likelihood” is a likelihood that is decreased by 50%, 100%, 200%, 500%, or more relative to a healthy subject or a subject that does not have altered expression of genes recited herein. In some embodiments, a low likelihood refers to the absolute likelihood of developing Grade Group ≥2 prostate cancer. In some embodiments, a “low likelihood” is a less than 50% likelihood that a subject's prostate biopsy would detect Grade Group ≥2 prostate cancer in the subject. In some embodiments, a “low likelihood” is a 40% or less likelihood that a subject's prostate biopsy would detect Grade Group ≥2 prostate cancer in the subject. In some embodiments, a “low likelihood” is a 30% or less likelihood that a subject's prostate biopsy would detect Grade Group ≥2 prostate cancer in the subject. In some embodiments, a “low likelihood” is an 20% or less likelihood that a subject's prostate biopsy would detect Grade Group ≥2 prostate cancer in the subject. In some embodiments, a “low likelihood” is a 10% or less likelihood that a subject's prostate biopsy would detect Grade Group ≥2 prostate cancer in the subject. In some embodiments, a “low likelihood” is a 5% or less likelihood that a subject's prostate biopsy would detect Grade Group ≥2 prostate cancer in the subject. In some embodiments, a “low likelihood” is a 4% or less likelihood that a subject's prostate biopsy would detect Grade Group ≥2 prostate cancer in the subject. In some embodiments, a “low likelihood” is a 3% or less likelihood that a subject's prostate biopsy would detect Grade Group ≥2 prostate cancer in the subject. In some embodiments, a “low likelihood” is a 2% or less likelihood that a subject's prostate biopsy would detect Grade Group ≥2 prostate cancer in the subject. In some embodiments, a “low likelihood” is a 1% or less likelihood that a subject's prostate biopsy would detect Grade Group ≥2 prostate cancer in the subject. In some embodiments, a “low likelihood” is a 0% likelihood that a subject's prostate biopsy would detect Grade Group ≥2 prostate cancer in the subject.

As used herein, the language “nucleic acid molecule” refers to any nucleic acid containing molecule, including but not limited to, DNA or RNA. The nucleic acid molecule may comprise one or more nucleotides. The language may include nucleotide polymers in which the nucleotides and the linkages between them include non-naturally occurring synthetic analogs, such as, for example and without limitation, phosphorothioates, phosphoramidates, methyl phosphonates, chiral-methyl phosphonates, 2-O-methyl ribonucleotides, peptide-nucleic acids (PNAs), and the like. The term further encompasses sequences that may include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine. It will be understood that when a nucleotide sequence is represented by a DNA sequence (i.e., A, T, G, C), the sequence also includes an RNA sequence (i.e., A, U, G, C) in which “U” replaces “T.”

The term “gene” refers to a nucleic acid (e.g., DNA) sequence that comprises coding sequences necessary for the production of a polypeptide, precursor, or RNA (e.g., rRNA, tRNA). The polypeptide can be encoded by a full-length coding sequence or by any portion of the coding sequence so long as the desired activity or functional properties (e.g., enzymatic activity, ligand binding, signal transduction, immunogenicity, etc.) of the full-length or fragments are retained. The term also encompasses the coding region of a structural gene and the sequences located adjacent to the coding region on both the 5′ and 3′ ends for a distance of about 1 kb or more on either end, such that the “gene” corresponds to the length of the full-length mRNA. Sequences located 5′ of the coding region and present on the mRNA are referred to as 5′ non-translated or untranslated sequences. Sequences located 3′ or downstream of the coding region and present on the mRNA are referred to as 3′ non-translated or untranslated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “oligonucleotide,” refers to a short length of single-stranded polynucleotide chain. Oligonucleotides are typically less than 200 nucleotide residues long (e.g., between 15 and 100), however, as used herein, the term is also intended to encompass longer polynucleotide chains. Oligonucleotides are often referred to by their length. For example, a 24-residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

The term “label” as used herein refers to any atom or molecule that can be used to provide a detectable (preferably quantifiable) effect, and that can be attached to a nucleic acid or protein. Labels include but are not limited to: dyes; radiolabels such as ³²P; binding moieties such as biotin; haptens such as digoxgenin; luminogenic, phosphorescent or fluorogenic moieties; and fluorescent dyes alone or in combination with moieties that can suppress or shift emission spectra by fluorescence resonance energy transfer (FRET). Labels may provide signals detectable by fluorescence, radioactivity, colorimetry, gravimetry, X-ray diffraction or absorption, magnetism, enzymatic activity, and the like. A label may be a charged moiety (e.g., a positive or negative charge) or alternatively, may be charge neutral. Labels can include or consist of nucleic acid or protein sequence, so long as the sequence comprising the label is detectable. In some embodiments, nucleic acids are detected directly without a label (e.g., directly reading a sequence).

As used herein, the term “sample” includes a specimen or culture obtained from any source, as well as biological and environmental samples. Biological samples may be obtained from animals (including humans) and encompass fluids (e.g., blood, urine), solids, tissues, and gases. Biological samples can include urine, urine supernatant, and urine cell pellet as well as blood products, such as plasma, serum and the like. Such examples are not however to be construed as limiting the sample types applicable to the present disclosure.

As used herein, “high-grade prostate cancer” means Grade Group ≥2 prostate cancer. In some embodiments, the high-grade prostate cancer is GG≥3 prostate cancer.

As used herein, “low-grade prostate cancer” means Grade Group <2 prostate cancer.

As used herein, “score” is a likelihood that a subject's prostate biopsy would detect Grade Group ≥2 prostate cancer in the subject, i.e., that a subject's prostate biopsy would be positive for a prostate cancer. The score is based on the level or amount of expression of one or more genes described herein present in a sample from a subject. In some embodiments, the score is a numerical value ranging from 0% to 100%. In some embodiments, the numerical value is expressed as a decimal number ranging from 0.0 to 100.0. In some embodiments, the score is a qualitative read-out of “low risk” or “elevated risk”.

As used herein, the term “altered,” for example in the context of “altered levels of expression of one or more of the genes,” refers to a level of gene expression that is different (e.g., increased or decreased) than the level of expression in, e.g., a subject without prostate cancer or a subject with low-grade prostate cancer.

As used herein, the term “variant,” e.g., a gene variant, refers to a sequence change that does not affect gene identity. Such sequence changes are readily appreciated by the skilled artisan. In some embodiments, a variant comprises a mutation, a substitution, and/or a deletion. In some embodiments, a variant comprises a polymorphism. In some embodiments, a variant comprises a splice variant.

As used herein, the term “about” means ±10% variation from nominal value unless otherwise indicated or inferred. When the term “about” is used before a number, the present disclosure also includes the specific number itself, unless specifically stated otherwise.

### DETAILED DESCRIPTION OF THE DISCLOSURE

Provided herein are kits and methods useful for cancer diagnosis, prognosis, research and therapy. In particular, provided herein are methods of diagnosing, prognosing, and/or treating prostate cancer based on expression levels of cancer markers.

The disclosure is based, at least in part, on the discovery of methods for determining a likelihood that a subject has Grade Group ≥2 prostate cancer based on an amount of expression of one or more genes described herein.

Described herein are methods and kits incorporating one or more of 17 markers useful for prognosing, diagnosing or treating prostate cancer. Importantly, detection of PSA (prostate specific antigen), the conventional method for prognosis and/or diagnosis of prostate cancer, is not a necessary step of the methods described herein. PSA elevation identified during PSA screening leads to a high rate of invasive and unnecessary biopsies in men without cancer and frequent overdiagnosis of low-grade, indolent cancers (grade group 1 (GG1)). The kits and methods of the present disclosure provide more precise prognosis or diagnosis of prostate cancer and help identify those subjects that can benefit from early, aggressive therapeutic interventions while sparing those subjects with indolent disease from an invasive procedure, such as a biopsy. The instant methods therefore provide a new and unconventional set of prostate cancer biomarkers, and particularly high-grade (e.g., GG≥2) prostate cancer biomarkers, independent of PSA.

Accordingly, provided herein are methods and kits useful for prognosing, diagnosing or treating subjects with prostate cancer, in some embodiments, Grade Group ≥2 prostate cancer. For example, in some embodiments, provided herein are methods of treating prostate cancer, comprising: a) assaying a level of expression of one or more (e.g., 1, 2, 3, 4, 5, 6, 7, 8, 9, 10, 11, 12, 13, 14, 15, 16, or all 17) genes selected from, for example, TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6 in a sample from a subject prognosed or diagnosed with prostate cancer; and b) administering a prostate cancer treatment to a subject identified as having altered levels of expression of one or more of the genes relative to a subject without prostate cancer or a subject with low-grade prostate cancer. In some embodiments, the subject has high-grade prostate cancer.

Further embodiments provide methods of characterizing, prognosing, or recommending a treatment for prostate cancer, comprising: a) assaying a level of expression of one or more (e.g., 1, 2, 3, 4, 5, 6, 7, 8, 9, 10, 11, 12, 13, 14, 15, 16, or all 17) genes selected from, for example, TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6 in a sample from a subject prognosed or diagnosed with prostate cancer; and b) identifying said subject as having high-grade prostate cancer when the subject is identified as having altered levels of expression of the genes relative to a subject without prostate cancer or a subject with low-grade prostate cancer. In some embodiments, the methods further comprise administering a prostate cancer treatment to the subject. In some embodiments, the methods further comprise administering a treatment for Grade Group ≥2 prostate cancer to the subject.

In some embodiments, the methods further comprise performing a prostate biopsy on the subject. In some embodiments, the methods further comprise recommending to the subject or the subject's health care provider that the subject undergo a prostate biopsy. In some embodiments, the prostate biopsy indicates the subject has Grade Group ≥2 prostate cancer. In some embodiments, the prostate biopsy indicates the subject does not have Grade Group ≥2 prostate cancer.

In some embodiments, the methods further comprise recommending to the subject or the subject's health care provider that the subject does not undergo a prostate biopsy.

In some embodiments, the methods do not comprise performing a prostate biopsy on the subject.

The methods described herein are useful to identify subjects with high-grade prostate cancer for treatment and allow those identified as not having high-grade prostate cancer to avoid a biopsy or treatment and, accordingly, its associated side effects. The methods as provided herein are useful to reduce the number of unnecessary prostate biopsies, sparing healthy subjects from a costly, invasive procedure.

### I. Methods of Assaying Marker Expression

As described herein, embodiments of the present disclosure provide methods for prognosis, diagnosis or treatment that utilize detection of an expression amount or level of one or more (e.g., 1, 2, 3, 4, 5, 6, 7, 8, 9, 10, 11, 12, 13, 14, 15, 16, or all 17) genes selected from, for example, TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6. Illustrative, non-limiting methods are described herein.

### Genes for Detecting

In some embodiments, the level or amount of expression of one or more genes is determined. In some embodiments, the level or amount of expression is the level or amount of mRNA or protein expressed by the genes.

In some embodiments, the one or more genes are selected from TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6.

In some embodiments, the methods and kits described herein are useful for detecting a level or an amount of expression of a TMPRSS2-ERG gene. A TMPRSS2-ERG gene fusion overexpresses the transcription factor ERG, which is present in both early- and late-stage prostate cancer. Numerous variations of TMPRSS2-ERG fusions have been identified, with the most common comprising exon 1 of TMPRSS2 and exons 4-11 of ERG. In some embodiments, a TMPRSS2-ERG gene fusion comprises a fusion of the nucleotide sequences of Ensembl gene identifiers ENSG00000184012 and ENSG00000157554. In some embodiments, a TMPRSS2-ERG gene fusion comprises the nucleotide sequence of SEQ ID NO:1 or a variant thereof.

In some embodiments, the methods and kits described herein are useful for detecting a level or an amount of expression of a SCHLAP1 gene. SCHLAP1 is a long noncoding RNA overexpressed in a subset of prostate cancers. SCHLAP1 antagonizes the genome-wide localization and regulatory functions of the SWI/SNF chromatin-modifying complex. In some embodiments, the SCHLAP1 gene comprises the nucleotide sequence provided by the HUGO Gene Nomenclature Committee (HGNC). In some embodiments, the HGNC identifier for SCHLAP1 is 48603. In some embodiments, the SCHLAP1 gene is located at chromosome position 2q31.3. In some embodiments, a SCHLAP1 gene comprises the nucleotide sequence of Ensembl gene identifier ENSG00000281131. In some embodiments, a SCHLAP1 gene comprises the nucleotide sequence of SEQ ID NO:2 or a variant thereof.

In some embodiments, the methods and kits described herein are useful for detecting a level or an amount of expression of a OR51E2 gene. OR51E2 is an odorant receptor (OR) which represent the largest G protein-coupled receptor (GPCR) family in the human genome. Activation of human ORs can influence cell proliferation. Specifically, OR51E2 has been identified as being involved in the regulation of cell growth, migration and the invasiveness of melanocytes, melanoma cells, and prostate cancer cells. In some embodiments, the OR51E2 gene comprises the nucleotide sequence provided by HGNC. In some embodiments, the HGNC identifier for OR51E2 is 15195. In some embodiments, the OR51E2 gene is located at chromosome position 11p15.4. In some embodiments, an OR51E2 gene comprises the nucleotide sequence of Ensembl gene identifier ENSG00000167332. In some embodiments, an OR51E2 gene comprises the nucleotide sequence of SEQ ID NO:3 or a variant thereof.

In some embodiments, the methods and kits described herein are useful for detecting a level or an amount of expression of an APOC1 gene. APOC1 is the smallest apolipoprotein and is a component of both triglyceride-rich lipoproteins and high-density lipoproteins. APOC1 is involved in various biological processes and is related to the progression of multiple diseases such as diabetic nephropathy, Alzheimer's disease, and glomerculosclerosis. Recent studies have shown APOC1 may be associated with the development of cancers, including breast cancer, pancreatic cancer, lung cancer, and prostate cancer. In some embodiments, the APOC1 gene comprises the nucleotide sequence provided by HGNC. In some embodiments, the HGNC identifier for APOC1 is 607. In some embodiments, the APOC1 gene is located at chromosome position 19q13.32. In some embodiments, an APOC1 gene comprises the nucleotide sequence of Ensembl gene identifier ENSG00000130208. In some embodiments, an APOC1 gene comprises the nucleotide sequence of SEQ ID NO:4 or a variant thereof.

In some embodiments, the methods and kits described herein are useful for detecting a level or an amount of expression of a PCAT14 gene. PCAT14 is a long non-coding RNA that exhibits both cancer and lineage specificity. PCAT14 is transcriptionally regulated by androgen receptor (AR) and endogenous PCAT14 overexpression suppresses cell invasion. In some embodiments, the PCAT14 gene comprises the nucleotide sequence provided by HGNC. In some embodiments, the HGNC identifier for PCAT14 is 48977. In some embodiments, the PCAT14 gene is located at chromosome position 22q11.23. In some embodiments, a PCAT14 gene comprises the nucleotide sequence of Ensembl gene identifier ENSG00000280623. In some embodiments, a PCAT14 gene comprises the nucleotide sequence of SEQ ID NO:5 or a variant thereof.

In some embodiments, the methods and kits described herein are useful for detecting a level or an amount of expression of a CAMKK2 gene. CAMKK2 is a direct target of the AR and regulation can vary across disease stages. CAMKK2 has been identified as a drive of prostate cancer progression. In some embodiments, the CAMKK2 gene comprises the nucleotide sequence provided by HGNC. In some embodiments, the HGNC identifier for CAMKK2 is 1470. In some embodiments, the CAMKK2 gene is located at chromosome position 12q24.31. In some embodiments, a CAMKK2 gene comprises the nucleotide sequence of Ensembl gene ENSG00000110931. In some embodiments, a CAMKK2 gene comprises the nucleotide sequence of SEQ ID NO:6 or a variant thereof.

In some embodiments, the methods and kits described herein are useful for detecting a level or an amount of expression of a PCA3 gene. PCA3 is a non-coding gene associated with prostate cancer. In some embodiments, the PCA3 gene comprises the nucleotide sequence provided by HGNC. In some embodiments, the HGNC identifier for PCA3 is 8637. In some embodiments, the PCA3 gene is located at chromosome position 9q21.2. In some embodiments, a PCA3 gene comprises the nucleotide sequence of Ensembl gene identifier ENSG00000225937. In some embodiments, a PCA3 gene comprises the nucleotide sequence of SEQ ID NO:7 or a variant thereof.

In some embodiments, the methods and kits described herein are useful for detecting a level or an amount of expression of an NKAIN1 gene. NKAIN1 is a sodium/potassium transporting ATPase. In some embodiments, the NKAIN1 gene comprises the nucleotide sequence provided by HGNC. In some embodiments, the HGNC identifier for NKAIN1 is 25743. In some embodiments, the NKAIN1 gene is located at chromosome position 1p35.2. In some embodiments, an NKAIN1 gene comprises the nucleotide sequence of Ensembl gene identifier ENSG00000084628. In some embodiments, an NKAIN1 gene comprises the nucleotide sequence of SEQ ID NO:8 or a variant thereof.

In some embodiments, the methods and kits described herein are useful for detecting a level or an amount of expression of a B3GNT6 gene. B3GNT6 is a member of the O-GlcNAc transferase (OGT) family and is responsible for the production of the core 3 structure of O-glycans. In some embodiments, the B3GNT6 gene comprises the nucleotide sequence provided by HGNC. In some embodiments, the HGNC identifier for B3GNT6 is 24141. In some embodiments, the B3GNT6 gene is located at chromosome position 11q13.5. In some embodiments, a B3GNT6 gene comprises the nucleotide sequence of Ensembl gene identifier ENSG00000198488. In some embodiments, a B3GNT6 gene comprises the nucleotide sequence of SEQ ID NO:9 or a variant thereof.

In some embodiments, the methods and kits described herein are useful for detecting a level or an amount of expression of a TFF3 gene. TFF3 is a trefoil factor, which are secreted peptides produced by normal intestinal mucosa. Members of the trefoil family are overexpressed in a variety of cancers and are associated with tumor invasion, resistance to apoptosis, and metastasis. In some embodiments, the TFF3 gene comprises the nucleotide sequence provided by HGNC. In some embodiments, the HGNC identifier for TFF3 is 11757. In some embodiments, the TFF3 gene is located at chromosome position 2122.3. In some embodiments, a TFF3 gene comprises the nucleotide sequence of Ensembl gene identifier ENSG00000160180. In some embodiments, a TFF3 gene comprises the nucleotide sequence of SEQ ID NO:10 or a variant thereof.

In some embodiments, the methods and kits described herein are useful for detecting a level or an amount of expression of a SPON2 gene. SPON2 belongs to the F-spondin family of secreted extracellular matrix proteins, and is deregulated in some tumors, including prostate cancer. In some embodiments, the SPON2 gene comprises the nucleotide sequence provided by HGNC. In some embodiments, the HGNC identifier for SPON2 is 11253. In some embodiments, the SPON2 gene is located at chromosome position 4p16.3. In some embodiments, a SPON2 gene comprises the nucleotide sequence of Ensembl gene identifier ENSG00000159674. In some embodiments, a SPON2 gene comprises the nucleotide sequence of SEQ ID NO:11 or a variant thereof.

In some embodiments, the methods and kits described herein are useful for detecting a level or an amount of expression of a PCGEM1 gene. PCGEM1 is a long non-coding RNA that is a prostate-specific transcript. In some embodiments, the PCGEM1 gene comprises the nucleotide sequence provided by HGNC. In some embodiments, the HGNC identifier for PCGEM1 is 30145. In some embodiments, the PCGEM1 gene is located at chromosome position 2q32.3. In some embodiments, a PCGEM1 gene comprises the nucleotide sequence of Ensembl gene identifier ENSG00000227418. In some embodiments, a PCGEM1 gene comprises the nucleotide sequence of SEQ ID NO:12 or a variant thereof.

In some embodiments, the methods and kits described herein are useful for detecting a level or an amount of expression of a TRGV9 gene. TRGV9 is encoded by the TRG locus that rearranges to encode a TCRγ chain containing 14 variable genes, of which only 6 are functional, including TRGV9. In some embodiments, the TRGV9 gene comprises the nucleotide sequence provided by HGNC. In some embodiments, the HGNC identifier for TRGV9 is 12295. In some embodiments, the TRGV9 gene is located at chromosome position 7p14.1. In some embodiments, a TRGV9 gene comprises the nucleotide sequence of Ensembl gene identifier ENSG00000211695. In some embodiments, a TRGV9 gene comprises the nucleotide sequence of SEQ ID NO:13 or a variant thereof.

In some embodiments, the methods and kits described herein are useful for detecting a level or an amount of expression of a TMSB15A gene. TMSB15A is an isoform of human thymosin beta 15 which is an actin-binding protein. TMSB15A is expressed in normal human prostate and prostate cancer tissue. In some embodiments, the TMSB15A gene comprises the nucleotide sequence provided by HGNC. In some embodiments, the HGNC identifier for TMSB15A is 30744. In some embodiments, the TMSB15A gene is located at chromosome position Xq22.1. In some embodiments, a TMSB15A gene comprises the nucleotide sequence of Ensembl gene identifier ENSG00000158164. In some embodiments, a TMSB15A gene comprises the nucleotide sequence of SEQ ID NO:14 or a variant thereof.

In some embodiments, the methods and kits described herein are useful for detecting a level or an amount of expression of an ERG gene. ERG is a transcriptional regulator overexpressed in prostate cancer. In some embodiments, the ERG gene comprises the nucleotide sequence provided by HGNC. In some embodiments, the HGNC identifier for ERG is 3446. In some embodiments, the ERG gene is located at chromosome position 21q22.2. In some embodiments, an ERG gene comprises the nucleotide sequence of Ensembl gene identifier ENSG00000157554. In some embodiments, an ERG gene comprises the nucleotide sequence of SEQ ID NO:15 or a variant thereof.

In some embodiments, the methods and kits described herein are useful for detecting a level or an amount of expression of a KLK4 gene. KLK4 is a member of the kallikrein (KLK) family of highly conserved serine proteases that play key roles in a variety of physiological and pathological processes. KLKs are secreted proteins that have extracellular substrates and function. KLK4 is overexpressed in prostate cancer. In some embodiments, the KLK4 gene comprises the nucleotide sequence provided by HGNC. In some embodiments, the HGNC identifier for KLK4 is 6365. In some embodiments, the KLK4 gene is located at chromosome position 19q13.41. In some embodiments, a KLK4 gene comprises the nucleotide sequence of Ensembl gene identifier ENSG00000167749. In some embodiments, a KLK4 gene comprises the nucleotide sequence of SEQ ID NO:16 or a variant thereof.

In some embodiments, the methods and kits described herein are useful for detecting a level or an amount of expression of a HOXC6 gene. HOXC6 is a homeobox (HOX) gene. HOX genes are involved in organ development and homeostasis and have been shown to be involved in normal prostate and prostate cancer development. HOXC6 is overexpressed in prostate cancer. In some embodiments, the HOXC6 gene comprises the nucleotide sequence provided by HGNC. In some embodiments, the HGNC identifier for HOXC6 is 5128. In some embodiments, the HOXC6 gene is located at chromosome position 12q13.13. In some embodiments, a HOXC6 gene comprises the nucleotide sequence of Ensembl gene identifier ENSG00000197757. In some embodiments, a HOXC6 gene comprises the nucleotide sequence of SEQ ID NO:17 or a variant thereof.

Illustrative nucleotide sequences of TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6 are provided in Table A.

TABLE A
Illustrative nucleotide sequences of genes of the disclosure.

| SEQ ID | | |
|---|---|---|
| NO | Gene | Sequence |
| 1 | TMPRSS2- | TAGGCGCGAG CTAAGCAGGA GGCGGAGGCG GAGGCGGAGG GCGAGGGGCG |
| | ERG | GGGAGCGCCG CCTGGAGCGC GGCAGGAAGC CTTATCAGTT GTGAGTGAGG |
| | | ACCAGTCGTT GTTTGAGTGT GCCTACGGAA CGCCACACCT GGCTAAGACA |
| | | GAGATGACCG CGTCCTCCTC CAGCGACTAT GGACAGACTT CCAAGATGAG |
| | | CCCACGCGTC CCTCAGCAGG ATTGGCTGTC TCAACCCCCA GCCAGGGTCA |
| | | CCATCAAAAT GGAATGTAAC CCTAGCCAGG TGAATGGCTC AAG |
| 2 | SCHLAP1 | GCTTTTATGA GCTGTAACAC TCACCGCGAA GGTCCGCAGC TTCACTCCTG |
| | | AAGCCAGCGA GACCACGAGC CTACTGGGAG GAACGAACAA CTCCCGACGC |
| | | GCCGCCTTAA GAGCTGTAAC ACTCACCGCG AAGGTCTGCA GCTTCACTCC |
| | | TGAGCCAGCG AGACCACGAA CCCACCAGAA GGAAAAAACT CCGAACACAT |
| | | CTGAACATCA GAAGCAACAA ACTCCGGACA CGCCGCCTTT AAGAACTGTA |
| | | ACACTCACTG CGAGGGTCCG CGGCTTCATT CTTGAAGTGA GTGAGACCAA |
| | | GAACCCACCA GTTCTGGACA CAATTTCAAG TCCTCAGGTG CCATCAATAT |
| | | TCTGAAAATG GCAGTGATTT TTATTCAACC TGTATAAGGC ACTTTCACCA |
| | | TGTACCTGGA AGCAACATCT ACATCTTTTT CAGTTTCTTC TACGCCAGGT |
| | | GTGTGCTTAG CTCCATGACA AAAGGTGACA GCTTATTCTG CAGCACACAC |
| | | ACATCATCAA AGTGGGAGGT GGTGAGACTG GCACACTGAC AGTCTGTCCT |
| | | AGCAGATTTC AGCTCACACT GCAATCTAGA TGCTGGGGAC ACAAGGTCCA |
| | | CCTTCCAGGA ATATGGCCAT GACACCAGAA ATCACAAACA TGATGAGAAT |
| | | GGAATGACTG GGGAAGAAGT GCCAGATGCT TCACTTGTAA ATGAAGACCC |
| | | AGCCTCTGGG GATGCAGATA CCACCTCCCT GAAGAAGCTG AATATCTGCA |
| | | GATAAGTGGA GTTCACCAAT GATGAGGAGC GGGATGGAGA AAGGAGGTAG |
| | | GGAGAGTCAT CCAAGGAACA TGAGCAACAT GTTAAAAGCC AAGTGGTTTA |
| | | ATTTCTGGAG ATGGTGAACC CAAGAGGCTC TGCTGGGAGA CAACAAAAAT |
| | | AATGAAGAAT TGAACCAGAG TCCGGTGAAT ATCAGCACTG GGACCAGTTA |
| | | GCAGAGGAAA AGGAAAGAAT AAAAGCGAAA AGAATGAAGA GTCATATGAT |
| | | TACCAACTTT TCCTTTTTCA TATAAATTGA GTGTATATGG GTCTGGAACA |
| | | ACCTGAATTT CCATCAAGTC CTGGCTAACC TCATTATGTC CTATGAATAT |
| | | TTTTGACTAA TCCCACTTTA CATTAATCTG TATTGTGAAT GTGGATATTG |
| | | AATTATATTT CTTTGTAATC CCATTATCCA AAATCCAGTT CAGAGACTAT |
| | | TAGTTACCAA TGTTCACTGT GAAGGAAAAA AAAAAAAAAA AAGCTCAGAG |
| | | GATAAACATG TGATATGGTT TGGCTGTGTC CCCACCCAAA TATCATCTTG |
| | | AATTGTAGCT CCCATAATTC CCACGTGTTG TGGGAGGGAC CCGGTGGGAG |
| | | ATAATTGTAT CATGGGGGTG GTTCCCCCAT ACTATTCTCA TAGTAGTGAA |
| | | TAAGTCTCAC AAAATCTGAT GGTTTTATGA GGGAAAACCC CTTTCACCTG |
| | | GTTCTCATTC TCTTCTCTGG TCTGTCGTCA TGTAAGACAT GCCTTTCACC |
| | | TTCTCCACCA TGACTGTGAG GCCTCCCCAG CCACGTGGAA CTGTGAGCCC |
| | | ATTAAACCTC TTTCACTTAT AAAT |
| 3 | OR51E2 | CTTCTGGGAA TCTCCACACC CTGAAGACAC AGTGAGTTAG CACCACCACC |
| | | AGGAATTGGC CTTTCAGCTC TGTGCCTGTC TCCAGTCAGG CTGGAATAAG |
| | | TCTCCTCATA TTTGCAAGCT CGGCCCTCCC CTGGAATCTA AAGCCTCCTC |
| | | AGCCTTCTGA GTCAGCCTGA AAGGAACAGG CCGAACTGCT GTATGGGCTC |
| | | TACTGCCAGT GTGACCTCAC CCTCTCCAGT CACCCCTCCT CAGTTCCAGC |
| | | TATGAGTTCC TGCAACTTCA CACATGCCAC CTTTGTGCTT ATTGGTATCC |
| | | CAGGATTAGA GAAAGCCCAT TTCTGGGTTG GCTTCCCCCT CCTTTCCATG |
| | | TATGTAGTGG CAATGTTTGG AAACTGCATC GTGGTCTTCA TCGTAAGGAC |
| | | GGAACGCAGC CTGCACGCTC CGATGTACCT CTTTCTCTGC ATGCTTGCAG |
| | | CCATTGACCT GGCCTTATCC ACATCCACCA TGCCTAAGAT CCTTGCCCTT |
| | | TTCTGGTTTG ATTCCCGAGA GATTAGCTTT GAGGCCTGTC TTACCCAGAT |
| | | GTTCTTTATT CATGCCCTCT CAGCCATTGA ATCCACCATC CTGCTGGCCA |
| | | TGGCCTTTGA CCGTTATGTG GCCATCTGCC ACCCACTGCG CCATGCTGCA |
| | | GTGCTCAACA ATACAGTAAC AGCCCAGATT GGCATCGTGG CTGTGGTCCG |
| | | CGGATCCCTC TTTTTTTTCC CACTGCCTCT GCTGATCAAG CGGCTGGCCT |
| | | TCTGCCACTC CAATGTCCTC TCGCACTCCT ATTGTGTCCA CCAGGATGTA |
| | | ATGAAGTTGG CCTATGCAGA CACTTTGCCC AATGTGGTAT ATGGTCTTAC |
| | | TGCCATTCTG CTGGTCATGG GCGTGGACGT AATGTTCATC TCCTTGTCCT |
| | | ATTTTCTGAT AATACGAACG GTTCTGCAAC TGCCTTCCAA GTCAGAGCGG |
| | | GCCAAGGCCT TTGGAACCTG TGTGTCACAC ATTGGTGTGG TACTCGCCTT |
| | | CTATGTGCCA CTTATTGGCC TCTCAGTGGT ACACCGCTTT GGAAACAGCC |
| | | TTCATCCCAT TGTGCGTGTT GTCATGGGTG ACATCTACCT GCTGCTGCCT |
| | | CCTGTCATCA ATCCCATCAT CTATGGTGCC AAAACCAAAC AGATCAGAAC |
| | | ACGGGTGCTG GCTATGTTCA AGATCAGCTG TGACAAGGAC TTGCAGGCTG |
| | | TGGGAGGCAA GTGACCCTTA ACACTACACT TCTCCTTATC TTTATTGGCT |
| | | TGATAAACAT AATTATTTCT AACACTAGCT TATTTCCAGT TGCCCATAAG |
| | | CACATCAGTA CTTTTCTCTG GCTGGAATAG TAAACTAAAG TATGGTACAT |
| | | CTACCTAAAG GACTATTATG TGGAATAATA CATACTAATG AAGTATTACA |
| | | TGATTTAAAG ACTACAATAA AACCAAACAT GCTTATAACA TTAAGAAAAA |
| | | CAATAAAGAT ACATGATTGA AACCAAGTTG AAAAATAGCA TATGCCTTGG |
| | | AGGAAATGTG CTCAAATTAC TAATGATTTA GTGTTGTCCC TACTTTCTCT |
| | | CTCTTTTTTC TTTCTTTTTT TTTTATTATG GTTAGCTGTC ACATACAACT |
| | | TTTTTTTTTT TTGAGATGGG GTCTCGCTCT GTCACCAGGC TGGAGTGCAG |
| | | TGGCGCGATC TCGGCTCACT GCAACCTCCA CATCCCATGT TGAAGTAATT |
| | | CTTCTGCCTC AGCCTCCCGA GTAGCTGGGA CTAGAGGAAC GTGCCACCAT |
| | | GACTGGCTAA TTTTCTGTAT TTTTTAGTAG AGACAGAGTT TCACCATGTT |
| | | GGCCAGGATG GTCTCGATCT CCTGACCTTG TGATCCACCC GCCTCAGCCT |
| | | CCCAAAGTGT TGGGATTACA GGTGTGAACC ACTGTGCCCG GCCTGTGTAC |
| | | AACTTTTTAA ATAGGGAATA TGATAGCTTC GCATGGTGGT GTGCACCTAT |
| | | AGCCCCCACT GCCTGGAAAG CTGAGGTGGG AGAATCGCTT GAGTCCAGGA |
| | | GTTTGAGGTT ACAGTGATCC ACGATCGTAC CACTACACTC CAGCCTGGGC |
| | | AACAGAGCAA GACCCTGTCT CAAAGCATAA AATGGAATAA CATATCAAAT |
| | | GAAACAGGGA AAATGAAGCT GACAATTTAT GGAAGCCAGG GCTTGTCACA |
| | | GTCTCTACTG TTATTATGCA TTACCTGGGA ATTTATATAA GCCCTTAATA |
| | | ATAATGCCAA TGAACATCTC ATGTGTGCTC ACAATGTTCT GGCACTATTA |
| | | TAAGTGCTTC ACAGGTTTTA TGTGTTCTTC GTAACTTTAT GGAGTAGGTA |
| | | CCATTTGTGT CTCTTTATTA TAAGTGAGAG AAATGAAGTT TATATTATCA |
| | | AGGGGACTAA AGTCACACGG CTTGTGGGCA CTGTGCCAAG ATTTAAAATT |
| | | AAATTTGATG GTTGAATACA GTTACTTAAT GACCATGTTA TATTGCTTCC |
| | | TGTGTAACAT CTGCCATTTA TTTCCTCAGC TGTACAAATC CTCTGTTTTC |
| | | TCTCTGTTAC ACACTAACAT CAATGGCTTT GTACTTGTGA TGAGAGATAA |
| | | CCTTGCCCTA GTTGTGGGCA ACACATGCAG AATAATCCTG TTTTACAGCT |
| | | GCCTTTCGTG ATCTTATTGC TTGCTTTTTT CCAGATTCAG GGAGAATGTT |
| | | GTTGTCTATT TGTCTCTTAC ATCTCCTTGA TCATGTCTTC ATTTTTTAAT |
| | | GTGCTCTGTA CCTGTCAAAA ATTTTGAATG TACACCACAT GCTATTGTCT |
| | | GAACTTGAGT ATAAGATAAA ATAAAATTTT ATTTTAAATT TT |
| 4 | APOC1 | AGGCGGTCAG GGGAAGGCTC AGGAGGAGGG AGATCAACAT CAACCTGCCC |
| | | CGCCCCCTCC CCAGCCTGAT AAAGGTCCTG CGGGCAGGAC AGGACCTCCC |
| | | AACCAAGCCC TCCAGCAAGG ATTCAGAGTG CCCCTCCGGC CTCGCCATGA |
| | | GGCTCTTCCT GTCGCTCCCG GTCCTGGTGG TGGTTCTGTC GATCGTCTTG |
| | | GAAGGCCCAG CCCCAGCCCA GGGGACCCCA GACGTCTCCA GTGCCTTGGA |
| | | TAAGCTGAAG GAGTTTGGAA ACACACTGGA GGACAAGGCT CGGGAACTCA |
| | | TCAGCCGCAT CAAACAGAGT GAACTTTCTG CCAAGATGCG GGAGTGGTTT |
| | | TCAGAGACAT TTCAGAAAGT GAAGGAGAAA CTCAAGATTG ACTCATGAGG |
| | | ACCTGAAGGG TGACATCCCA GGAGGGGCCT CTGAAATTTC CCACACCCCA |
| | | GCGCCTGTGC TGAGGACTCC CTCCATGTGG CCCCAGGTGC CACCAATAAA |
| | | AATCCTACAG AAAA |
| 5 | PCAT14 | GAGATACGGC CTCGTGGGAA GGGAAAGACC TGACCGTCCC CCAGCCCGAC |
| | | ACCCGTAAAG GGTCTGTGCT GAGGAGGATT AGTAAAAGGG GAAGGCCTCT |
| | | TGCAGTTGAG ATAAGAGGAA GGCCTCCGTC TCCTGCATGT CCTTGGGAAT |
| | | GGAATGTCTT GGTGTAAAAC CCGATAGTAC ATTCCTTCTA TTCTGAGAGA |
| | | AGAAAACCAC CCTGTGGCTG GAGGGTGAAG GTACTCTACA GTGTGGTCAT |
| | | TGAGGACAAG TTGACGAGAG AGTCCCAAGT ACGTCCACGG TCAGCCTTGC |
| | | GACATTTAAA GTTCTACAAT GAACTCACTG GAGATGCAAA GAAAAGTGTG |
| | | GAGATGGAGA CACCCCAATC GACTCGCCAG TCTACAGGTG TATCCAGCAG |
| | | CTCCAAAGAG ACAGCAACCA GCAAGAATGG GCCATAGTGA CGATGGTGGT |
| | | TTTGTCAAAA AGAAAAGGGG GGGATATGTA AGGAAAAGAG AGATCAGACT |
| | | TTCACTGTGT CTATGTAGAA AAGGAAGACA TAAGAAACTC CATTTTGATC |
| | | TGTACTAAGA AAAATTGTTT TGCCTTGAGA TGCTGTTAAT CTGTAACTTT |
| | | AGCCCCAACC CTGTGCTCAC GGAAACATGT GCTGTAAGGT TTAAGGGATC |
| | | TAGGGCTGTG CAGGATGTAC CTTGTTAACA ATATGTTTGC AGGCAGTATG |
| | | TTTGGTAAAA GTCATCGCCA TTCTCCATTC TCGATTAACC AGGGGCTCAA |
| | | TGCACTGTGG AAAGCCACAG GAACCTCTGC CCAAGAAAGC CTGGCTGTTG |
| | | TGGGAAGTCA GGGACCCCGA ATGGAGGGAC CAGCTGGTGC TGCATCAGGA |
| | | AACATAAATT GTGAAGATTT CTTGGACATT TATCAGTTTC CAAAATTAAT |
| | | ACTTTTATAA TTTCTTACAC CTGTCTTACT TTAATCTCTT AATCCTGTTA |
| | | TCTTTGTAAG CTGAGGATAT ACGTCACCTC AGGACCACTA TTGTACAAAT |
| | | TGATTGTAAA ACATGTTCAC ATGTGTTTGA ACAATATGAA ATCAGTGCAC |
| | | CTTGAAAATG AACAGAATAA CAGTGATTTT AGGGAACAAA GGAAGACAAC |
| | | CATAAGGTCT GACTGCCTGA GGGGTCGGGC AAAAAGCCAT ATTTTTCTTC |
| | | TTGCAGAGAG CCTATAAATG GACGTGCAAG TAGGAGAGAT ATTGCTAAAT |
| | | T |
| 6 | CAMKK2 | AGAGCAAGCT GAGCCGAGCC GAGCCGAGCT GGGGGCGCAG AGCGCGGGAG |
| | | GCGGCGGCGG CGCGGAGCCC AGGTGGCTCC GCTGCCGGAT GGGAGTGCCC |
| | | CAGTGTGCTG GATGAAGCTG GCGCATGCAC CATGTCATCA TGTGTCTCTA |
| | | GCCAGCCCAG CAGCAACCGG GCCGCCCCCC AGGATGAGCT GGGGGGCAGG |
| | | GGCAGCAGCA GCAGCGAAAG CCAGAAGCCC TGTGAGGCCC TGCGGGGCCT |
| | | CTCATCCTTG AGCATCCACC TGGGCATGGA GTCCTTCATT GTGGTCACCG |
| | | AGTGTGAGCC GGGCTGTGCT GTGGACCTCG GCTTGGCGCG GGACCGGCCC |
| | | CTGGAGGCCG ATGGCCAAGA GGTCCCCCTT GACACCTCCG GGTCCCAGGC |
| | | CCGGCCCCAC CTCTCCGGTC GCAAGCTGTC TCTGCAAGAG CGGTCCCAGG |
| | | GTGGGCTGGC AGCCGGTGGC AGCCTGGACA TGAACGGACG CTGCATCTGC |
| | | CCGTCCCTGC CCTACTCACC CGTCAGCTCC CCGCAGTCCT CGCCTCGGCT |
| | | GCCCCGGCGG CCGACAGTGG AGTCTCACCA CGTCTCCATC ACGGGTATGC |
| | | AGGACTGTGT GCAGCTGAAT CAGTATACCC TGAAGGATGA AATTGGAAAG |
| | | GGCTCCTATG GTGTCGTCAA GTTGGCCTAC AATGAAAATG ACAATACCTA |
| | | CTATGCAATG AAGGTGCTGT CCAAAAAGAA GCTGATCCGG CAGGCCGGCT |
| | | TTCCACGTCG CCCTCCACCC CGAGGCACCC GGCCAGCTCC TGGAGGCTGC |
| | | ATCCAGCCCA GGGGCCCCAT TGAGCAGGTG TACCAGGAAA TTGCCATCCT |
| | | CAAGAAGCTG GACCACCCCA ATGTGGTGAA GCTGGTGGAG GTCCTGGATG |
| | | ACCCCAATGA GGACCATCTG TACATGGTGT TCGAACTGGT CAACCAAGGG |
| | | CCCGTGATGG AAGTGCCCAC CCTCAAACCA CTCTCTGAAG ACCAGGCCCG |
| | | TTTCTACTTC CAGGATCTGA TCAAAGGCAT CGAGTACTTA CACTACCAGA |
| | | AGATCATCCA CCGTGACATC AAACCTTCCA ACCTCCTGGT CGGAGAAGAT |
| | | GGGCACATCA AGATCGCTGA CTTTGGTGTG AGCAATGAAT TCAAGGGCAG |
| | | TGACGCGCTC CTCTCCAACA CCGTGGGCAC GCCCGCCTTC ATGGCACCCG |
| | | AGTCGCTCTC TGAGACCCGC AAGATCTTCT CTGGGAAGGC CTTGGATGTT |
| | | TGGGCCATGG GTGTGACACT ATACTGCTTT GTCTTTGGCC AGTGCCCATT |
| | | CATGGACGAG CGGATCATGT GTTTACACAG TAAGATCAAG AGTCAGGCCC |
| | | TGGAATTTCC AGACCAGCCC GACATAGCTG AGGACTTGAA GGACCTGATC |
| | | ACCCGTATGC TGGACAAGAA CCCCGAGTCG AGGATCGTGG TGCCGGAAAT |
| | | CAAGCTGCAC CCCTGGGTCA CGAGGCATGG GGCGGAGCCG TTGCCGTCGG |
| | | AGGATGAGAA CTGCACGCTG GTCGAAGTGA CTGAAGAGGA GGTCGAGAAC |
| | | TCAGTCAAAC ACATTCCCAG CTTGGCAACC GTGATCCTGG TGAAGACCAT |
| | | GATACGTAAA CGCTCCTTTG GGAACCCATT CGAGGGCAGC CGGCGGGAGG |
| | | AACGCTCACT GTCAGCGCCT GGAAACTTGC TCACCAAAAA ACCAACCAGG |
| | | GAATGTGAGT CCCTGTCTGA GCTCAAGGAA GCAAGGCAGC GAAGACAACC |
| | | TCCAGGGCAC CGACCCGCCC CCCGTGGGGG AGGAGGAAGT GCTCTTGTGA |
| | | GAGGCAGTCC CTGCGTGGAA AGTTGCTGGG CCCCCGCCCC CGGCTCCCCC |
| | | GCACGCATGC ATCCACTGCG GCCGGAGGAG GCCATGGAGC CCGAGTAGCT |
| | | GCCTGGATCG CTCGACCTCG CATGCGCGCC GCGTCGCCTC TGGGGGGCTG |
| | | CTGCACCGCG TTTCCATAGC AGCATGTCCT ACGGAAACCC AGCACGTGTG |
| | | TAGAGCCTCG ATCGTCATCT CTGGTTATTT GTTTTTTCCT TTGTTGTTTT |
| | | AAAGGGGACA AAAAAAAAAA AAGGACTTGA CTCCATGACG TCGACCGTGG |
| | | CCGCTGGCTG GCTGGACAGG CGGGTGTGAG GAGTTGCAGA CCCAAACCCA |
| | | CGTGCATTTT GGGACAATTG CTTTTTAAAA CGTTTTTATG CCAAAAATCC |
| | | TTCATTGTGA TTTTCAGAAC CACGTCAGAT ATACCAAGTG ACTGTGTGTG |
| | | GGGTTTGACA ACTGTGGAAA GGCGAGCAGA AAACTCCGGC GGTCTGAGGC |
| | | CATGGAGGTG GTTGCTGCAT TTGAGAGGGA GTAGGGGGCT AGATGTGGCT |
| | | CCTAGTGCAA ACCGGAAACC ATGGCACCTT CCAGAGCCGT GGTCTCAAGG |
| | | AGTCAGAGCA GGGCTGGCCC TCAGTAGCTG CAGGGAGCTT TGATGCAACT |
| | | TATTTGTAAG AAGGATTTTT AAATTTTTTA TGGGTAGAAT TGTAGTCAGG |
| | | AAAACAGAAA GGGCTTGAAA TTTAATAAGT GCTGCTGGAA GGGGATTTTC |
| | | CAAGCCTGGA AGGGTATTCA GCAGCTGTGG TGGGGAAACA TTTCTCCTGA |
| | | AAGACTGAAC GTGTTTCTTC ATGACAGCTG CTCAAAGCAG GTTTCTGAGA |
| | | TAGCTGACCG AGCTCTGGTA AATCTCTTTG TCAAATTACG AAAACTTCAG |
| | | GGTGAAATCC TATGCTTCCA TGTACATTAC ATGGCTTAAG ATTAAACAAA |
| | | AACATTTTTC AAGTCTCTAA CTAGAGTGAA CTCTAGAGCA CAGTAGTTCA |
| | | GAAACTATTT AGAGCTTCCA GGATATATTT CACAGCTTCA GGCATGTGAT |
| | | CAGTTAGAGC CGATGAAACC TATGCCCGCC TGTATATATA TTAGCAGCTT |
| | | AGCTAGTTCA TAACCTGTAT ATTCTAAAGA CTGCTAAGGT TTTGTTTTCA |
| | | TTTTAAATCC TAGCTGATTG TTGTGGTCAA TGAAATACCC AGTTTCTGGA |
| | | GGGCCAGGTG GGAAATGCTT TCACTGGACC AACACACAAA TGATCATCCT |
| | | GAGGATCTGA GCTTCCCTAG ACTCCACACA ATAACCTTGG GGCACCCTTT |
| | | TAGAGAAGAC TGTTGAAACC CACAGCACTC GTTGGGGTAT GAGGAAACCA |
| | | GGGCTTGGCA CAGGAAGTTC CCCTTTGTAG CTAAAAGTCC AGAAAGAAAG |
| | | GGTTCATCTT TTTGACTTCC AACTGATATT GGGAAGTTTG GTTGAGGTTC |
| | | AAGTGTGACT CCTTCCAGAG CCACAGGTAG GGGAGTGTGA AGTTGAGGGG |
| | | GAGGAAAGCT GGAAGGACTC TGCCTTGGGA GATTCCCAGC TCTGCTTTCC |
| | | AGCGCTTGGT GGAATCTGGG CTGGGGAAAG ACGGCACCGG GAAACTCTGC |
| | | TTCCCCATTG TTTCCATCTG ATCAGCTGTG GTGTGAGGAC TTCTCAGACA |
| | | AAGGCAAGGC CTCGTGCCCC TGCCCAGCCC ATTCATGGAG CCCTGGGCCT |
| | | TCTTGGCTTC CATAGATCCT AAGCTCTTGA CTGTAGTTTA GCCAGACTTG |
| | | TTTTGCTATC TTATAAGCAG TTCAGAATTA GGGAATGCTG GTTTTGAAGA |
| | | GCAAAGGACA GGTAGTCTAG AGAGGGTCGT CTGGCCTGCT TGCTGGGTCT |
| | | TTGTAACCCA GCACTTCCTC TTGCCCTCCT GGCTTTATGT TTATGGGGAG |
| | | AGGACTCAAT AGCTCCACCC CTTCTGGCAC CAGATGGGGC TTGGTTAGTT |
| | | TGCAATAAGC ACCTTGCAGA GGTTAAAGCC AGCGGGTCCC TAGTCTTAGG |
| | | CCCAGCCTGC TTGTGTGGGC TCTGGCCTGG CCTGGTGGCT GGCCCAGGGG |
| | | GCAGCAGTGC TTAGAGCTTC TGCAGGGCTT CTCTTGTTTA CACAGCTGCA |
| | | TCAGACAATG CCATTTCTCC CCACCACGGA ACCTTCCATC TAAGATTTCT |
| | | TCCAGGGAAT GCCAGCAATC AGGCAGCACC CAGCTGTGGG GGCAGTGGGG |
| | | TGGGGGAGAC CCACATTGAT GACTTTTTTT TTTTCTTTTA ATGAAGAAAC |
| | | ACCAAAGAAA GCTGTGGAAA GGACCTGCCC CACATGAAAA GGATAAGCCA |
| | | AGATGGCTGT AAACACAGAG CATTTGAGCT GCCACTCTTG GAGCACATTG |
| | | ATTTTTCAAA AGCCAGCTCT GTCAGGAAAG GAGGTGCTGT TATGAGCAGC |
| | | TCTTCCAGTG GGCAAAGAGG ACGCCCATAA TTTCTTCCAT TGCTAGCTCA |
| | | TCTGTGGGAC CAATTTGGTG TAAGCAACCT GTGGCCTGCA CTTGTGGCCT |
| | | CGAAGGAAGC ACAAACCCTC CATCCACTTC CCATTTCCTC TGCCCTTTTC |
| | | CACCTCCCCC TTCCATCCCA CCAGCTGCCA GTGGCTCCCA GAAAGCCTTA |
| | | TTGAGCCCCT TGTTGACACT TGGGGCTGCG GAGGCCTCTC CCTACTGGTC |
| | | TGGCCTTTCC TGAGAGGCAG GTCTTCCGTC CTCAGAGCCT TTCTGGAACA |
| | | AGGAGAATGC CTGTGCAGGT GGACACACAG GCCTGGCCTG TCGCTCTCAC |
| | | TTGTCTTCCA GCGGGGAGCT TCACGTTGCC GAGTGGAAGA ACCATGACCT |
| | | CCACTTGCTT CCAAGGTGCT AGGGAAGTTT CAGGGTACGC TGGTTCCCCT |
| | | CTCCAGCTGG AGGCCGAGTT TCTGGGGACT GCAGATTTTT CTACTCTGTG |
| | | ATCGATTCAA TGCCCGATGC TTCTGTTTCA TTCCCGACCC TTTCTACTAT |
| | | GCATTTTCCT TTTATCAGGT GTATAAAGTT AAATACTGTG TATTTATCAC |
| | | TAAAAAGTAC ATGAACTTAA GAGACAACTA AGCCTTTCGT GTTTTTCCAC |
| | | AGGTGTTTAA GCTTCTCTGT ACAGTTGAAA TAAACAGACA GCAAAATGGT |
| | | GCCAA |
| 7 | PCA3 | ACAGAAGAAA TAGCAAGTGC CGAGAAGCTG GCATCAGAAA AACAGAGGGG |
| | | AGATTTGTGT GGCTGCAGCC GAGGGAGACC AGGAAGATCT GCATGGTGGG |
| | | AAGGACCTGA TGATACAGAG GTGAGAAATA AGAAAGGCTG CTGACTTTAC |
| | | CATCTGAGGC CACACATCTG CTGAAATGGA GATAATTAAC ATCACTAGAA |
| | | ACAGCAAGAT GACAATATAA TGTCTAAGTA GTGACATGTT TTTGCACATT |
| | | TCCAGCCCCT TTAAATATCC ACACACACAG GAAGCACAAA AGGAAGCACA |
| | | GAGATCCCTG GGAGAAATGC CCGGCCGCCA TCTTGGGTCA TCGATGAGCC |
| | | TCGCCCTGTG CCTGGTCCCG CTTGTGAGGG AAGGACATTA GAAAATGAAT |
| | | TGATGTGTTC CTTAAAGGAT GGGCAGGAAA ACAGATCCTG TTGTGGATAT |
| | | TTATTTGAAC GGGATTACAG ATTTGAAATG AAGTCACAAA GTGAGCATTA |
| | | CCAATGAGAG GAAAACAGAC GAGAAAATCT TGATGGCTTC ACAAGACATG |
| | | CAACAAACAA AATGGAATAC TGTGATGACA TGAGGCAGCC AAGCTGGGGA |
| | | GGAGATAACC ACGGGGCAGA GGGTCAGGAT TCTGGCCCTG CTGCCTAAAC |
| | | TGTGCGTTCA TAACCAAATC ATTTCATATT TCTAACCCTC AAAACAAAGC |
| | | TGTTGTAATA TCTGATCTCT ACGGTTCCTT CTGGGCCCAA CATTCTCCAT |
| | | ATATCCAGCC ACACTCATTT TTAATATTTA GTTCCCAGAT CTGTACTGTG |
| | | ACCTTTCTAC ACTGTAGAAT AACATTACTC ATTTTGTTCA AAGACCCTTC |
| | | GTGTTGCTGC CTAATATGTA GCTGACTGTT TTTCCTAAGG AGTGTTCTGG |
| | | CCCAGGGGAT CTGTGAACAG GCTGGGAAGC ATCTCAAGAT CTTTCCAGGG |
| | | TTATACTTAC TAGCACACAG CATGATCATT ACGGAGTGAA TTATCTAATC |
| | | AACATCATCC TCAGTGTCTT TGCCCATACT GAAATTCATT TCCCACTTTT |
| | | GTGCCCATTC TCAAGACCTC AAAATGTCAT TCCATTAATA TCACAGGATT |
| | | AACTTTTTTT TTTAACCTGG AAGAATTCAA TGTTACATGC AGCTATGGGA |
| | | ATTTAATTAC ATATTTTGTT TTCCAGTGCA AAGATGACTA AGTCCTTTAT |
| | | CCCTCCCCTT TGTTTGATTT TTTTTCCAGT ATAAAGTTAA AATGCTTAGC |
| | | CTTGTACTGA GGCTGTATAC AGCCACAGCC TCTCCCCATC CCTCCAGCCT |
| | | TATCTGTCAT CACCATCAAC CCCTCCCATG CACCTAAACA AAATCTAACT |
| | | TGTAATTCCT TGAACATGTC AGGCATACAT TATTCCTTCT GCCTGAGAAG |
| | | CTCTTCCTTG TCTCTTAAAT CTAGAATGAT GTAAAGTTTT GAATAAGTTG |
| | | ACTATCTTAC TTCATGCAAA GAAGGGACAC ATATGAGATT CATCATCACA |
| | | TGAGACAGCA AATACTAAAA GTGTAATTTG ATTATAAGAG TTTAGATAAA |
| | | TATATGAAAT GCAAGAGCCA CAGAGGGAAT GTTTATGGGG CACGTTTGTA |
| | | AGCCTGGGAT GTGAAGCAAA GGCAGGGAAC CTCATAGTAT CTTATATAAT |
| | | ATACTTCATT TCTCTATCTC TATCACAATA TCCAACAAGC TTTTCACAGA |
| | | ATTCATGCAG TGCAAATCCC CAAAGGTAAC CTTTATCCAT TTCATGGTGA |
| | | GTGCGCTTTA GAATTTTGGC AAATCATACT GGTCACTTAT CTCAACTTTG |
| | | AGATGTGTTT GTCCTTGTAG TTAATTGAAA GAAATAGGGC ACTCTTGTGA |
| | | GCCACTTTAG GGTTCACTCC TGGCAATAAA GAATTTACAA AGAGCTACTC |
| | | AGGACCAGTT GTTAAGAGCT CTGTGTGTGT GTGTGTGTGT GTGAGTGTAC |
| | | ATGCCAAAGT GTGCCTCTCT CTCTTTGACC CATTATTTCA GACTTAAAAA |
| | | CAAGCATGTT TTCAAATGGC ACTATGAGCT GCCAATGATG TATCACCACC |
| | | ATATCTCATT ATTCTCCAGT AAATGTGATA ATAATGTCAT CTGTTAACAT |
| | | AAAAAAAGTT TGACTTCACA AAAGCAGCTG GAAATGGACA ACCACAATAT |
| | | GCATAAATCT AACTCCTACC ATCAGCTACA CACTGCTTGA CATATATTGT |
| | | TAGAAGCACC TCGCATTTGT GGGTTCTCTT AAGCAAAATA CTTGCATTAG |
| | | GTCTCAGCTG GGGCTGTGCA TCAGGCGGTT TGAGAAATAT TCAATTCTCA |
| | | GCAGAAGCCA GAATTTGAAT TCCCTCATCT TTTAGGAATC ATTTACCAGG |
| | | TTTGGAGAGG ATTCAGACAG CTCAGGTGCT TTCACTAATG TCTCTGAACT |
| | | TCTGTCCCTC TTTGTGTTCA TGGATAGTCC AATAAATAAT GTTATCTTTG |
| | | AACTGATGCT CATAGGAGAG AATATAAGAA CTCTGAGTGA TATCAACATT |
| | | AGGGATTCAA AGAAATATTA GATTTAAGCT CACACTGGTC AAAAGGAACC |
| | | AAGATACAAA GAACTCTGAG CTGTCATCGT CCCCATCTCT GTGAGCCACA |
| | | ACCAACAGCA GGACCCAACG CATGTCTGAG ATCCTTAAAT CAAGGAAACC |
| | | AGTGTCATGA GTTGAATTCT CCTATTATGG ATGCTAGCTT CTGGCCATCT |
| | | CTGGCTCTCC TCTTGACACA TATTAGCTTC TAGCCTTTGC TTCCACGACT |
| | | TTTATCTTTT CTCCAACACA TCGCTTACCA ATCCTCTCTC TGCTCTGTTG |
| | | CTTTGGACTT CCCCACAAGA ATTTCAACGA CTCTCAAGTC TTTTCTTCCA |
| | | TCCCCACCAC TAACCTGAAT GCCTAGACCC TTATTTTTAT TAATTTCCAA |
| | | TAGATGCTGC CTATGGGCTA TATTGCTTTA GATGAACATT AGATATTTAA |
| | | AGCTCAAGAG GTTCAAAATC CAACTCATTA TCTTCTCTTT CTTTCACCTC |
| | | CCTGCTCCTC TCCCTATATT ACTGATTGCA CTGAACAGCA TGGTCCCCAA |
| | | TGTAGCCATG CAAATGAGAA ACCCAGTGGC TCCTTGTGGT ACATGCATGC |
| | | AAGACTGCTG AAGCCAGAAG GATGACTGAT TACGCCTCAT GGGTGGGAGG |
| | | GACCACTCCT GGGCCTTCGT GATTGTCAGG AGCAAGACCT GAGATGCTCC |
| | | CTGCCTTCAG TGTCCTCTGC ATCTCCCCTT TCTAATGAAG ATCCATAGAA |
| | | TTTGCTACAT TTGAGAATTC CAATTAGGAA CTCACATGTT TTATCTGCCC |
| | | TATCAATTTT TTAAACTTGC TGAAAATTAA GTTTTTTCAA AATCTGTCCT |
| | | TGTAAATTAC TTTTTCTTAC AGTGTCTTGG CATACTATAT CAACTTTGAT |
| | | TCTTTGTTAC AACTTTTCTT ACTCTTTTAT CACCAAAGTG GCTTTTATTC |
| | | TCTTTATTAT TATTATTTTC TTTTACTACT ATATTACGTT GTTATTATTT |
| | | TGTTCTCTAT AGTATCAATT TATTTGATTT AGTTTCAATT TATTTTTATT |
| | | GCTGACTTTT AAAATAAGTG ATTCGGGGGG TGGGAGAACA GGGGAGGGAG |
| | | AGCATTAGGA CAAATACCTA ATGCATGTGG GACTTAAAAC CTAGATGATG |
| | | GGTTGATAGG TGCAGCAAAC CACTATGGCA CACGTATACC TGTGTAACAA |
| | | ACCTACACAT TCTGCACATG TATCCCAGAA CGTAAAGTAA AATTTAAAAA |
| | | AAAGTGA |
| 8 | NKAIN1 | AGTGCTGCTC TGCGCTGCGC CGCGCTCGGG GCTCGCTCTC CTTGCTCCGC |
| | | GCTCCCCGCC AGCCGCCCCG GGGCAGGAGG CGCGCCTGAC GGACGGCCCG |
| | | CTAGACAAAG GAGGCGCGGC TCGGCGGGGC CAGCGCGCGG ACGGACGGAC |
| | | CATGGACTCG GAGCGCGGGC GGCCGGCCCC AGCCTTGGGG ACCGGACACT |
| | | CCCGGGCCCG GCCCTAGGCG CCCGGCCCCG CCGCCCGGCG CGCCCAGCGG |
| | | GGAGGACGTG GAGCCCGCGC GGCGCGAGCA GGCGGCGGCC GCGGAGCAAG |
| | | AAGGGCGCCG CGGCGTGCGG CCCGCGCAGC CCCCGGAGCC ATGGGCAAGT |
| | | GCAGCGGGCG CTGCACGCTG GTCGCCTTCT GCTGCCTGCA GCTGGTGGCT |
| | | GCGCTGGAGC GGCAGATCTT TGACTTCCTG GGCTACCAGT GGGCTCCCAT |
| | | CCTAGCCAAC TTCCTGCACA TCATGGCAGT CATCCTGGGC ATCTTTGGCA |
| | | CCGTGCAGTA CCGCTCCCGG TACCTCATCC TGTATGCAGC CTGGCTGGTG |
| | | CTCTGGGTTG GCTGGAATGC ATTTATCATC TGCTTCTACT TGGAGGTTGG |
| | | ACAGCTGTCC CAGGACCGGG ACTTCATCAT GACCTTCAAC ACATCCCTGC |
| | | ACCGCTCCTG GTGGATGGAG AATGGGCCAG GCTGCCTGGT GACACCTGTT |
| | | CTGAACTCCC GCCTGGCTCT GGAGGACCAC CATGTCATCT CTGTCACTGG |
| | | CTGCCTGCTT GACTACCCCT ACATTGAAGC CCTCAGCAGC GCCCTGCAGA |
| | | TCTTCCTGGC ACTGTTCGGC TTCGTGTTCG CCTGCTACGT GAGCAAAGTG |
| | | TTCCTGGAGG AGGAGGACAG CTTTGACTTC ATCGGCGGCT TTGACTCCTA |
| | | CGGATACCAG GCGCCCCAGA AGACGTCGCA TTTACAGCTG CAGCCTCTGT |
| | | ACACGTCGGG GTAGCCTCTG CCCCGCGCCC ACCCCGGCGC CTCGCCCTGG |
| | | GCTGACCGCA GCTGCCGCGA GCTCGGGCCA AGGCGCAGGC GTGTCCCCCT |
| | | GGTGGCCCGC GCGCTCACTG CAGCCTGTGC CCAACCCCGC GTCTGCATCT |
| | | GGAGATGCGG ACTTGGACGT GGACTTGGAC TTGGACTTGG ATTTGAGCTT |
| | | GGCTCTTCGC AGCCCGGACT TCGGAGGAGT GGGGGGGGGC GGGGGAGGGG |
| | | CACCACGGGT TTTTTGTTTT TTGTTTGTTT GTTTTTAATC TCAGCCTTGG |
| | | CGTGAGCTGG GGCCTTCCTC TCTTCTCCAG CCTCTCCCTT TCACTCTTCA |
| | | CCCAGCATCC TGCCCCCCTG TCCAAAAACA GCAGGACATC AGACCCATCC |
| | | CATCCCACCA CACTCACTCA CCAGCTCTGG GGAAAGCTAC TGTGAACTAG |
| | | GAGCAGGATT CCTGGGTTCT AATCGCAGGT CCATCACTGA CTGTGACGTC |
| | | TAGCAAAGCC CTTGCCCTCT CTGAGCCTCG GTTTCCGCAC CTCAAGTAAT |
| | | TAATCCCTTA GCAAATGGAC TCTTTTAGAC TTCTCATTTA ACTCAATTCC |
| | | CTGAGCTAGA CTGGGATTAA AATTCTCATT TTGCAGTACA TTAAAACTGA |
| | | GGCCCAGAGA TGTGATTTGC TTGAGGCCAC ACAGCTAGAT TTTTGGTGGA |
| | | AGTGGGCCTT GAACACAGTG TACTTTCTGC AGTTTCTGAC TGTAAAACCC |
| | | AGTGTCTGCT CTCTGAGTTC CATTTCCAAG CCCCCCTCCA TCTTGGACCT |
| | | ATGTGGTCTC CACCATATTC ACACACCACC ACCACCACTT GCCAATGCCT |
| | | CTCTTAAAGC AATATACCCA TTCGTTCTCT TATTGGGAAC TGGATGGATG |
| | | AAGCCCCAAA TTCAGCCCCA CCCACAGAGA AGCCTTCCTA CACTCAGCCT |
| | | CTGTCCACCC TTGGCAAATC TTTCAAGCTC TCTCCTCCAG GAAAGTGGGG |
| | | CCCCAACTCA GTCACTCCAC CCCCTTCCAG GTCCCTGAGG CTGGTTCTAC |
| | | TGTATCCCCA TCACCTCCAC AACTCCACTC ACCCCTGACG GCTCCATCCA |
| | | CCTCACCAGT TGGAAGGCTT GTGGTTTCAG AGAGGAGCAA TGCTGGTCAG |
| | | CGCTGCCCAG ACTCCAGTGT TTACAGATCA CCAGCATTTA CAACCAATCC |
| | | AATGGCCAGA AGCCTCCTCT AACAAGCCCA GAAGGAGTTC TGAAGGGGCA |
| | | GATGGGGGTG TGAGTAGTCG GGGAGTCGGG ATTGCCAGCA CCCTCACCCT |
| | | TCCTTGGGGG CAAGTAGAGG TGAGAACACT TTCCCCACCT CCCTCCACAG |
| | | ACACTCCTGA GGACGCTGCA TCCCACGCAC TGCCTGGTGC GTCCATAGAG |
| | | AGAGGATCAG GTCTCAGCAT TTCATCTGTG AAAGAGGCAT GGCCCTGGGT |
| | | TAGAAAGGAG GGCAGGAGAC ATGGAGGAAC TGGGGGGCAC CCAGATGGTG |
| | | CAGATGGTTT GCACACCTGA GCCTGTCTGT GGTGACCATT CCGCTCCTCT |
| | | CCCACTACCC TCCAATCTAT CATTCCCTAC TCTCTAAGGC CAAAATATCC |
| | | TGAGCAAGGC TGGCAACCCC ACCCCACCAT CCCAAATGCA AGCAGCCAGG |
| | | CCCAGGAGTT CCTCTGGCCC CCACAGGCAT GGAGCTCCCA GCTGGTGGGT |
| | | ACAGCTTGAG AGGGGGGCAG CTCCCTCAGG CTAAGCTACT GCCCTTCACT |
| | | GGGCCAGCCC TGCCTCCAGC CCTCACCTCT CTCACCCCAA CTCTCCCCCA |
| | | AGCCCCTTTC TACTCAACGG GTGTAGCCAC TGGTGCTTTG AAGCCTTTTG |
| | | TTTTTATAAG ATGGTTTTTG CAAGGGGACC AGGTTCTCTT TTCACTGGGA |
| | | TTTTCTTCTC AGGGGAGTGC TCTCCTGGTT TCTGTGCAGG CGGGTTGATT |
| | | AAAGATGGTG CCTTGCAAGG |
| | | TA |
| 9 | B3GNT6 | AGTGTGTGAA GTAAAGGGAT TAAAGGCTAG TCTCAGGCTG GGGATGGCTC |
| | | CTGTCTATTT CTTCTCTCTC AGAGACTGCA GATGGCTTTT CCCTGCCGCA |
| | | GGTCCCTGAC TGCCAAGACT CTGGCCTGCC TCCTGGTGGG CGTGAGTTTC |
| | | TTAGCACTGC AGCAGTGGTT CCTCCAGGCG CCAAGGTCCC CGCGGGAGGA |
| | | GAGGTCCCCG CAGGAGGAGA CGCCAGAGGG TCCCACCGAC GCTCCCGCGG |
| | | CTGACGAGCC GCCCTCGGAG CTCGTCCCCG GGCCCCCGTG CGTGGCGAAC |
| | | GCCTCGGCGA ACGCCACGGC CGACTTCGAG CAGCTGCCCG CGCGCATCCA |
| | | GGACTTCCTG CGGTACCGCC ACTGCCGCCA CTTCCCGCTG CTTTGGGACG |
| | | CACCGGCCAA GTGCGCCGGC GGCCGAGGCG TGTTCCTGCT CCTGGCGGTG |
| | | AAGTCGGCGC CTGAGCACTA CGAGCGACGC GAGCTCATCC GGCGCACGTG |
| | | GGGGCAAGAG CGCAGCTACG GCGGGCGGCC AGTGCGCCGC CTCTTTCTAT |
| | | TGGGCACCCC GGGCCCCGAG GACGAGGCGC GCGCGGAGCG GCTGGCGGAG |
| | | CTGGTGGCGC TGGAGGCGCG CGAGCACGGC GACGTGCTGC AGTGGGCCTT |
| | | CGCGGACACC TTCCTCAACC TCACGCTCAA GCACCTGCAC TTGCTCGACT |
| | | GGCTGGCTGC ACGCTGCCCG CACGCGCGCT TTCTGCTCAG CGGCGACGAC |
| | | GACGTGTTCG TGCACACCGC CAACGTAGTC CGCTTCCTGC AGGCGCAGCC |
| | | ACCCGGCCGC CACCTGTTCT CCGGCCAGCT CATGGAGGGC TCCGTGCCCA |
| | | TCCGCGACAG CTGGAGCAAG TACTTCGTGC CGCCGCAGCT CTTCCCCGGG |
| | | TCCGCTTACC CGGTGTACTG CAGCGGCGGC GGCTTCCTCC TGTCCGGCCC |
| | | CACGGCCCGG GCCCTGCGCG CGGCCGCCCG CCACACCCCG CTCTTCCCCA |
| | | TCGACGACGC CTACATGGGC ATGTGTCTGG AGCGCGCCGG CCTGGCGCCC |
| | | AGCGGCCACG AGGGCATCCG ACCCTTCGGC GTGCAGCTGC CTGGCGCACA |
| | | GCAGTCCTCC TTCGACCCCT GCATGTACCG CGAGTTGCTG CTAGTGCACC |
| | | GCTTCGCGCC CTACGAGATG CTGCTCATGT GGAAGGCGCT GCACAGCCCC |
| | | GCGCTCAGCT GTGACCGGGG ACACCGGGTC TCCTGAGGCC AGTTGGGCGG |
| | | CTTCAGCCCC GGGCCTCCAA CCATGTCCAT GCTGAGAAGG CAGCTTTCCC |
| | | GCTCTGGGTA CCTTACGTCC TGCCCAGCTC TGTGCACCTG AACCCCAGCT |
| | | GCGCACTGAA ATCAGCTGGG GTGGGGGGTG TGGAAAATGC CTACATCCTG |
| | | GCTCCATCTC CCGAAGTTTC GATTTGATTA GTCTGGGGTG GACCCAGACA |
| | | TGTTAAGTAT TTTTTAAGTT CCTCCAGTGA TGCGAATGTG CAGCTAGGCC |
| | | TGAGGACCAC TCGGCTAGAC TATCTCTTCA TCCTCGCAAA GCCAGCTCCA |
| | | CCGCCCTCTC TGCAAGAATT CCGGGCCCCT CGCTCCCACA CTCGGGTCCT |
| | | CTTGAGCAGT GGAGCAAGGG AGACCTGGGA GCGTGGGAGC CAGGATCAGC |
| | | GCCCCCTGCC ATGTGCCTAC AAATGTCAGT TGTGATTTCC ACTGTTTACA |
| | | AGTGAGTGGA GCTGGAGCTG GGCTGACAGT ATCAGGTGGA TCCCGCTTCC |
| | | CCCTCCCCCA AGAAGTCAGC CAACACGCAG CTGAGGCGCA TGTGGTGGCC |
| | | TTCTTCCCAC CACTACCCCA GTACACCGTG AGGTAGAAAT CTTCACCGTG |
| | | CAAAGTGGAA ACCAGAGGCC CGGTCAGACA GTGACTAATC CAGGGCCGTG |
| | | GCATTCCCAG ACAGCACACC ACTGTGGTCC CCTCCACACT CACCCCAACC |
| | | AAAGCTAATG GCCTAGTTGG GTCCTGCCCG CCAATAATCA CCCCCACGGG |
| | | TCAGAGACAG GCTCCTTGCC GGGGTCTGGG CCTCAGGCTC AGTGGGCCTT |
| | | GGACAACCCA GCAGGGAGTT CCGGGGAGTC CGAAGTGGAG AAAGGCTGGT |
| | | GGGAACATGG AGGCCAGTGT TGGGGAGCCT GTGGAGGCAG GTGTGTAGAA |
| | | TTGTGTTCGG GAGGTGGGGG ATCTGAGACC GAAGTGGACA GTGGTTAAGA |
| | | TTGTGGGGCC GGGCGAGGTG GCTCACGCCT GTAATCCCAG CACTTTGGGA |
| | | GGCTGAGGAG GTCGGATCAT GAGGTCAAGA GTTCGAGACC AGCCTGGCCA |
| | | ATATGGTGAA ACCCCGTCTC TATTGGGAGT ACAAAAATTA GCCGGCCATA |
| | | GTGGCTCGTG CCTGTAATCT CAGCTATTTG GGAGGCTGAG GCAGGAGAAT |
| | | CACTTGAACC TGGGAGGCGG AGGTTGCAGT GAGCCGAGAT CGTGCCACTG |
| | | CACTCCAGCC TGGGCGACAG AGCAAGACTG CATCTCAAAA AAAAAAAAAA |
| | | AAA |
| 10 | TFF3 | GAGTCCTGAG CTGCGTCCCG GAGCCCACGG TGGTCATGGC TGCCAGAGCG |
| | | CTCTGCATGC TGGGGCTGGT CCTGGCCTTG CTGTCCTCCA GCTCTGCTGA |
| | | GGAGTACGTG GGCCTGTCTG CAAACCAGTG TGCCGTGCCA GCCAAGGACA |
| | | GGGTGGACTG CGGCTACCCC CATGTCACCC CCAAGGAGTG CAACAACCGG |
| | | GGCTGCTGCT TTGACTCCAG GATCCCTGGA GTGCCTTGGT GTTTCAAGCC |
| | | CCTGCAGGAA GCAGAATGCA CCTTCTGAGG CACCTCCAGC TGCCCCCGGC |
| | | CGGGGGATGC GAGGCTCGGA GCACCCTTGC CCGGCTGTGA TTGCTGCCAG |
| | | GCACTGTTCA TCTCAGCTTT TCTGTCCCTT TGCTCCCGGC AAGCGCTTCT |
| | | GCTGAAAGTT CATATCTGGA GCCTGATGTC TTAACGAATA AAGGTCCCAT |
| | | GCTCCACCCG AGGACAGTTC TTCGTGCCTG AGACTTTCTG AGGTTGTGCT |
| | | TTATTTCTGC TGCGTCGTGG GAGAGGGCGG GAGGGTGTCA GGGGAGAGTC |
| | | TGCCCAGGCC TCAAGGGCAG GAAAAGACTC CCTAAGGAGC TGCAGTGCAT |
| | | GCAAGGATAT TTTGAATCCA GACTGGCACC CACGTCACAG GAAAGCCTAG |
| | | GAACACTGTA AGTGCCGCTT CCTCGGGAAA GCAGAAAAAA TACATTTCAG |
| | | GTAGAAGTTT TCAAAAATCA CAAGTCTTTC TTGGTGAAGA CAGCAAGCCA |
| | | ATAAAACTGT CTTCCAAAGT GGTCCTTTAT TTCACAACCA CTCTCGCTAC |
| | | TGTTCAATAC TTGTACTATT CCTGGGTTTT GTTTCTTTGT ACAGTAAACA |
| | | TTATGAACAA ACAGGCA |
| 11 | SPON2 | ACCCGACCGC TGCCGGCCGC GCTCCCGCTG CTCCTGCCGG GTGATGGAAA |
| | | ACCCCAGCCC GGCCGCCGCC CTGGGCAAGG CCCTCTGCGC TCTCCTCCTG |
| | | GCCACTCTCG GCGCCGCCGG CCAGCCTCTT GGGGGAGAGT CCATCTGTTC |
| | | CGCCAGAGCC CTGGCCAAAT ACAGCATCAC CTTCACGGGC AAGTGGAGCC |
| | | AGACGGCCTT CCCCAAGCAG TACCCCCTGT TCCGCCCCCC TGCGCAGTGG |
| | | TCTTCGCTGC TGGGGGCCGC GCATAGCTCC GACTACAGCA TGTGGAGGAA |
| | | GAACCAGTAC GTCAGTAACG GGCTGCGCGA CTTTGCGGAG CGCGGCGAGG |
| | | CCTGGGCGCT GATGAAGGAG ATCGAGGCGG CGGGGGAGGC GCTGCAGAGC |
| | | GTGCACGCGG TGTTTTCGGC GCCCGCCGTC CCCAGCGGCA CCGGGCAGAC |
| | | GTCGGCGGAG CTGGAGGTGC AGCGCAGGCA CTCGCTGGTC TCGTTTGTGG |
| | | TGCGCATCGT GCCCAGCCCC GACTGGTTCG TGGGCGTGGA CAGCCTGGAC |
| | | CTGTGCGACG GGGACCGTTG GCGGGAACAG GCGGCGCTGG ACCTGTACCC |
| | | CTACGACGCC GGGACGGACA GCGGCTTCAC CTTCTCCTCC CCCAACTTCG |
| | | CCACCATCCC GCAGGACACG GTGACCGAGA TAACGTCCTC CTCTCCCAGC |
| | | CACCCGGCCA ACTCCTTCTA CTACCCGCGG CTGAAGGCCC TGCCTCCCAT |
| | | CGCCAGGGTG ACACTGGTGC GGCTGCGACA GAGCCCCAGG GCCTTCATCC |
| | | CTCCCGCCCC AGTCCTGCCC AGCAGGGACA ATGAGATTGT AGACAGCGCC |
| | | TCAGTTCCAG AAACGCCGCT GGACTGCGAG GTCTCCCTGT GGTCGTCCTG |
| | | GGGACTGTGC GGAGGCCACT GTGGGAGGCT CGGGACCAAG AGCAGGACTC |
| | | GCTACGTCCG GGTCCAGCCC GCCAACAACG GGAGCCCCTG CCCCGAGCTC |
| | | GAAGAAGAGG CTGAGTGCGT CCCTGATAAC TGCGTCTAAG ACCAGAGCCC |
| | | CGCAGCCCCT GGGGCCCCCC GGAGCCATGG GGTGTCGGGG GCTCCTGTGC |
| | | AGGCTCATGC TGCAGGCGGC CGAGGGCACA GGGGGTTTCG CGCTGCTCCT |
| | | GACCGCGGTG AGGCCGCGCC GACCATCTCT GCACTGAAGG GCCCTCTGGT |
| | | GGCCGGCACG GGCATTGGGA AACAGCCTCC TCCTTTCCCA ACCTTGCTTC |
| | | TTAGGGGCCC CCGTGTCCCG TCTGCTCTCA GCCTCCTCCT CCTGCAGGAT |
| | | AAAGTCATCC CCAAGGCTCC AGCTACTCTA AATTATGTCT CCTTATAAGT |
| | | TATTGCTGCT CCAGGAGATT GTCCTTCATC GTCCAGGGGC CTGGCTCCCA |
| | | CGTGGTTGCA GATACCTCAG ACCTGGTGCT CTAGGCTGTG CTGAGCCCAC |
| | | TCTCCCGAGG GCGCATCCAA GCGGGGGCCA CTTGAGAAGT GAATAAATGG |
| | | GGCGGTTTCG GAAGCGTCAG TGTTTCCATG TTATGGATCT CTCTGCGTTT |
| | | GAATAAAGAC TATCTCTGTT GCTCACAAA |
| 12 | PCGEM1 | AAGGCACTCT GGCACCCAGT TTTGGAACTG CAGTTTTAAA AGTCATAAAT |
| | | TGAATGAAAA TGATAGCAAA GGTGGAGGTT TTTAAAGAGC TATTTATAGG |
| | | TCCCTGGACA GCATCTTTTT TCAATTAGGC AGCAACCTTT TTGCCCTATG |
| | | CCGTAACCTG TGTCTGCAAC TTCCTCTAAT TGGGAAATAG TTAAGCAGAT |
| | | TCATAGAGCT GAATGATAAA ATTGTACTAC GAGATGCACT GGGACTCAAC |
| | | GTGACCTTAT CAAGTGAGCA GGCTTGGTGC ATTTGACACT TCATGATATC |
| | | AGCCAAAGTG GAACTAAAAA CAGCTCCTGG AAGAGGACTA TGACATCATC |
| | | AGGTTGGGAG TCTCCAGGGA CAGCGGACCC TTTGGAAAAG GACTAGAAAG |
| | | TGTGAAATCT ATTAGTCTTC GATATGAAAT TCTCTGTCTC TGTAAAAGCA |
| | | TTTCATATTT ACAAGACACA GGCCTACTCC TAGGGCAGCA AAAAGTGGCA |
| | | ACAGGCAAGC AGAGGGAAAA GAGATCATGA GGCATTTCAG AGTGCACTGT |
| | | CTTTTCATAT ATTTCTCAAT GCCGTATGTT TGGTTTTATT TTGGCCAAGC |
| | | ATAACAATCT GCTCAAGAAA AAAAAATCTG GAGAAAACAA AGGTGCCTTT |
| | | GCCAATGTTA TGTTTCTTTT TGACAAGCCC TGAGATTTCT GAGGGGAATT |
| | | CACATAAATG GGATCAGGTC ATTCATTTAC GTTGTGTGCA AATATGATTT |
| | | AAAGATACAA CCTTTGCAGA GAGCATGCTT TCCTAAGGGT AGGCACGTGG |
| | | AGGACTAAGG GTAAAGCATT CTTCAAGATC AGTTAATCAA GAAAGGTGCT |
| | | CTTTGCATTC TGAAATGCCC TTGTTGCAAA TATTGGTTAT ATTGATTAAA |
| | | TTTACACTTA ATGGAAACAA CCTTTAACTT ACAGATGAAC AAACCCACAA |
| | | AAGCAAAAAA TCAAAAGCCC TACCTATGAT TTCATATTTT CTGTGTAACT |
| | | GGATTAAAGG ATTCCTGCTT GCTTTTGGGC ATAAATGATA ATGGAATATT |
| | | TCCAGGTATT GTTTAAAATG AGGGCCCATC TACAAATTCT TAGCAATACT |
| | | TTGGATAATT CTAAAATTCA GCTGGACATT GTCTAATTGT TTTTTATATA |
| | | CATCTTTGCT AGAATTTCAA ATTTTAAGTA TGTGAATTTA GTTAATTAGC |
| | | TGTGCTGATC AATTCAAAAA CATTACTTTC CTAAATTTTA GACTATGAAG |
| | | GTCATAAATT CAACAAATAT ATCTACACAT ACAATTATAG ATTGTTTTTC |
| | | ATTATAATGT CTTCATCTTA ACAGAATTGT CTTTGTGATT GTTTTTAGAA |
| | | AACTGAGAGT TTTAATTCAT AATTACTTGA TCAAAAAATT GTGGGAACAA |
| | | TCCAGCATTA ATTGTATGTG ATTGTTTTTA TGTACATAAG GAGTCTTAAG |
| | | CTTGGTGCCT TGAAGTCTTT TGTACTTAGT CCCATGTTTA AAATTACTAC |
| | | TTTATATCTA AAGCATTTAT GTTTTTCAAT TCAATTTACA TGATGCTAAT |
| | | TATGGCAATT ATAACAAATA TTAAAGATTT CGAAATAGAA AAAAAAAAAA |
| | | AAA |
| 13 | TRGV9 | GTGAGGACAC CGCTTTACAA CGATGCAGGG GGCCCCATGT CACCCTCACC |
| | | CATGGGAAGT TTGACTTGGT GGACTCAGCC AAGCCACAGA GGTCTAACGC |
| | | TTCTCTGCGG TGATTTCAGG CTGCCCTGGC AGAAAGCACA GTGCCTGCAG |
| | | ACATGCTGTC ACTGCTCCAC GCATCAACGC TGGCAGTCCT TGGGGCTCTG |
| | | TGTGTATATG GTGCAGGTCA CCTAGAGCAA CCTCAAATTT CCAGTACTAA |
| | | AACGCTGTCA AAAACAGCCC GCCTGGAATG TGTGGTGTCT GGAATAACAA |
| | | TTTCTGCAAC ATCTGTATAT TGGTATCGAG AGAGACCTGG TGAAGTCATA |
| | | CAGTTCCTGG TGTCCATTTC ATATGACGGC ACTGTCAGAA AGGAATCCGG |
| | | CATTCCGTCA GGCAAATTTG AGGTGGATAG GATACCTGAA ACGTCTACAT |
| | | CCACTCTCAC CATTCACAAT GTAGAGAAAC AGGACATAGC TACCTACTAC |
| | | TGTGCCTTGT TGGAGGGAAA TTATAAGAAA CTCTTTGGCA GTGGAACAAC |
| | | ACTTGTTGTC ACAGATAAAC AACTTGATGC AGATGTTTCC CCCAAGCCCA |
| | | CTATTTTTCT TCCTTCAATT GCTGAAACAA AGCTCCAGAA GGCTGGAACA |
| | | TACCTTTGTC TTCTTGAGAA ATTTTTCCCT GATGTTATTA AGATACATTG |
| | | GCAAGAAAAG AAGAGCAACA CGATTCTGGG ATCCCAGGAG GGGAACACCA |
| | | TGAAGACTAA CGACACATAC ATGAAATTTA GCTGGTTAAC GGTGCCAGAA |
| | | AAGTCACTGG ACAAAGAACA CAGATGTATC GTCAGACATG AGAATAATAA |
| | | AAACGGAGTT GATCAAGAAA TTATCTTTCC TCCAATAAAG ACAGATGTCA |
| | | TCACAATGGA TCCCAAAGAC AATTGTTCAA AAGATGCAAA TGATACACTA |
| | | CTGCTGCAGC TCACAAACAC CTCTGCATAT TACATGTACC TCCTCCTGCT |
| | | CCTCAAGAGT GTGGTCTATT TTGCCATCAT CACCTGCTGT CTGCTTAGAA |
| | | GAACGGCTTT CTGCTGCAAT GGAGAGAAAT CATAACAGAC GGTGGCACAA |
| | | GGAGGCCATC TTTTCCTCAT CGGTTATTGT CCCTAGAAGC GTCTTCTGAG |
| | | GATCTAGTTG GGCTTTCTTT CTGGGTTTGG GCCATTTCAG TTCTCATGTG |
| | | TGTACTATTC TATCATTATT GTATAACGGT TTTCAAACCA GTGGGCACAC |
| | | AGAGAACCTC ACTCTGTAAT AACAATGAGG AATAGCCACG GCGATCTCCA |
| | | GCACCAATCT CTCCATGTTT TCCACAGCTC CTCCAGCCAA CCCAAATAGC |
| | | GCCTGCTATA GTGTAGACAT CCTGCGGCTT CTAGCCTTGT CCCTCTCTTA |
| | | GTGTTCTTTA ATCAGATAAC TGCCTGGAAG CCTTTCATTT TACACGCCCT |
| | | GAAGCAGTCT TCTTTGCTAG TTGAATTATG TGGTGTGTTT TTCCGTAATA |
| | | AGCAAAATAA ATTTAAAAAA ATGAAAAGTT |
| 14 | TMSB15A | AACGCTAACC TGGTCCGGAG CGAGTCTGGG TCTCAGCCCC GCGAACAGCC |
| | | TTTCACGAGT CTTCAAGCTT TCAGGCTATC TTCTAGTCAA GATGAGTGAT |
| | | AAGCCAGACT TGTCGGAAGT GGAGAAGTTT GACAGGTCAA AACTGAAGAA |
| | | AACTAATACT GAAGAAAAAA ATACTCTTCC CTCAAAGGAA ACTATCCAGC |
| | | AAGAGAAAGA GTGTGTTCAA ACATCATAAA ATGGGGATCG CCTCCCAACA |
| | | GCAGATTTCG ACATTACCTG AGAGTCTTGA TTTTAGGCTT GTTTTTTGTA |
| | | AACCCATGTG TTTGTAGAGA TTTTAGGCGT CTTCGGATAT CTTCTCACCT |
| | | ATGTTCCCTG GCTAAGAAGT CAGAGGTAGC CAATGTTTCC TTAAATTCAT |
| | | TTTTAAACTT ACCATTGGTG CATATGTTCC AGATGGCAGA TGCTGTCAAT |
| | | AATCTCACCA TTGATGACCT TTGTGTATGT AGTTCTTGCA TCCTATACTG |
| | | GATAAGCCTG TTTTAACCTG CTATGATGGG TGCTTCCATT GCTTCATAAT |
| | | CTTCATGAAG TTGCATGCTT TTGCAGCTTT TCACAGTTTA TTTGCATTTC |
| | | TAATGTAGTA ATAAAGTAAC CAATATAATC ATTA |
| 15 | ERG | ATCCGCTCTA AACAACCTCA TCAAAACTAC TTTCTGGTCA GAGAGAAGCA |
| | | ATAATTATTA TTAACATTTA TTAACGATCA ATAAACTTGA TCGCATTATG |
| | | GCCAGCACTA TTAAGGAAGC CTTATCAGTT GTGAGTGAGG ACCAGTCGTT |
| | | GTTTGAGTGT GCCTACGGAA CGCCACACCT GGCTAAGACA GAGATGACCG |
| | | CGTCCTCCTC CAGCGACTAT GGACAGACTT CCAAGATGAG CCCACGCGTC |
| | | CCTCAGCAGG ATTGGCTGTC TCAACCCCCA GCCAGGGTCA CCATCAAAAT |
| | | GGAATGTAAC CCTAGCCAGG TGAATGGCTC AAGGAACTCT CCTGATGAAT |
| | | GCAGTGTGGC CAAAGGCGGG AAGATGGTGG GCAGCCCAGA CACCGTTGGG |
| | | ATGAACTACG GCAGCTACAT GGAGGAGAAG CACATGCCAC CCCCAAACAT |
| | | GACCACGAAC GAGCGCAGAG TTATCGTGCC AGCAGATCCT ACGCTATGGA |
| | | GTACAGACCA TGTGCGGCAG TGGCTGGAGT GGGCGGTGAA AGAATATGGC |
| | | CTTCCAGACG TCAACATCTT GTTATTCCAG AACATCGATG GGAAGGAACT |
| | | GTGCAAGATG ACCAAGGACG ACTTCCAGAG GCTCACCCCC AGCTACAACG |
| | | CCGACATCCT TCTCTCACAT CTCCACTACC TCAGAGAGAC TCCTCTTCCA |
| | | CATTTGACTT CAGATGATGT TGATAAAGCC TTACAAAACT CTCCACGGTT |
| | | AATGCATGCT AGAAACACAG GGGGTGCAGC TTTTATTTTC CCAAATACTT |
| | | CAGTATATCC TGAAGCTACG CAAAGAATTA CAACTAGGCC AGATTTACCA |
| | | TATGAGCCCC CCAGGAGATC AGCCTGGACC GGTCACGGCC ACCCCACGCC |
| | | CCAGTCGAAA GCTGCTCAAC CATCTCCTTC CACAGTGCCC AAAACTGAAG |
| | | ACCAGCGTCC TCAGTTAGAT CCTTATCAGA TTCTTGGACC AACAAGTAGC |
| | | CGCCTTGCAA ATCCAGGCAG TGGCCAGATC CAGCTTTGGC AGTTCCTCCT |
| | | GGAGCTCCTG TCGGACAGCT CCAACTCCAG CTGCATCACC TGGGAAGGCA |
| | | CCAACGGGGA GTTCAAGATG ACGGATCCCG ACGAGGTGGC CCGGCGCTGG |
| | | GGAGAGCGGA AGAGCAAACC CAACATGAAC TACGATAAGC TCAGCCGCGC |
| | | CCTCCGTTAC TACTATGACA AGAACATCAT GACCAAGGTC CATGGGAAGC |
| | | GCTACGCCTA CAAGTTCGAC TTCCACGGGA TCGCCCAGGC CCTCCAGCCC |
| | | CACCCCCCGG AGTCATCTCT GTACAAGTAC CCCTCAGACC TCCCGTACAT |
| | | GGGCTCCTAT CACGCCCACC CACAGAAGAT GAACTTTGTG GCGCCCCACC |
| | | CTCCAGCCCT CCCCGTGACA TCTTCCAGTT TTTTTGCTGC CCCAAACCCA |
| | | TACTGGAATT CACCAACTGG GGGTATATAC CCCAACACTA GGCTCCCCAC |
| | | CAGCCATATG CCTTCTCATC TGGGCACTTA CTACTAAAGA CCTGGCGGAG |
| | | GCTTTTCCCA TCAGCGTGCA TTCACCAGCC CATCGCCACA AACTCTATCG |
| | | GAGAACATGA ATCAAAAGTG CCTCAAGAGG AATGAAAAAA GCTTTACTGG |
| | | GGCTGGGGAA GGAAGCCGGG GAAGAGATCC AAAGACTCTT GGGAGGGAGT |
| | | TACTGAAGTC TTACTACAGA AATGAGGAGG ATGCTAAAAA TGTCACGAAT |
| | | ATGGACATAT CATCTGTGGA CTGACCTTGT AAAAGACAGT GTATGTAGAA |
| | | GCATGAAGTC TTAAGGACAA AGTGCCAAAG AAAGTGGTCT TAAGAAATGT |
| | | ATAAACTTTA GAGTAGAGTT TGGAATCCCA CTAATGCAAA CTGGGATGAA |
| | | ACTAAAGCAA TAGAAACAAC ACAGTTTTGA CCTAACATAC CGTTTATAAT |
| | | GCCATTTTAA GGAAAACTAC CTGTATTTAA AAATAGAAAC ATATCAAAAA |
| | | CAAGAGAAAA GACACGAGAG AGACTGTGGC CCATCAACAG ACGTTGATAT |
| | | GCAACTGCAT GGCATGTGCT GTTTTGGTTG AAATCAAATA CATTCCGTTT |
| | | GATGGACAGC TGTCAGCTTT CTCAAACTGT GAAGATGACC CAAAGTTTCC |
| | | AACTCCTTTA CAGTATTACC GGGACTATGA ACTAAAAGGT GGGACTGAGG |
| | | ATGTGTATAG AGTGAGCGTG TGATTGTAGA CAGAGGGGTG AAGAAGGAGG |
| | | AGGAAGAGGC AGAGAAGGAG GAGACCAGGG CTGGGAAAGA AACTTCTCAA |
| | | GCAATGAAGA CTGGACTCAG GACATTTGGG GACTGTGTAC AATGAGTTAT |
| | | GGAGACTCGA GGGTTCATGC AGTCAGTGTT ATACCAAACC CAGTGTTAGG |
| | | AGAAAGGACA CAGCGTAATG GAGAAAGGGG AAGTAGTAGA ATTCAGAAAC |
| | | AAAAATGCGC ATCTCTTTCT TTGTTTGTCA AATGAAAATT TTAACTGGAA |
| | | TTGTCTGATA TTTAAGAGAA ACATTCAGGA CCTCATCATT ATGTGGGGGC |
| | | TTTGTTCTCC ACAGGGTCAG GTAAGAGATG GCCTTCTTGG CTGCCACAAT |
| | | CAGAAATCAC GCAGGCATTT TGGGTAGGCG GCCTCCAGTT TTCCTTTGAG |
| | | TCGCGAACGC TGTGCGTTTG TCAGAATGAA GTATACAAGT CAATGTTTTT |
| | | CCCCCTTTTT ATATAATAAT TATATAACTT ATGCATTTAT ACACTACGAG |
| | | TTGATCTCGG CCAGCCAAAG ACACACGACA AAAGAGACAA TCGATATAAT |
| | | GTGGCCTTGA ATTTTAACTC TGTATGCTTA ATGTTTACAA TATGAAGTTA |
| | | TTAGTTCTTA GAATGCAGAA TGTATGTAAT AAAATAAGCT TGGCCTAGCA |
| | | TGGCAAATCA GATTTATACA GGAGTCTGCA TTTGCACTTT TTTTAGTGAC |
| | | TAAAGTTGCT TAATGAAAAC ATGTGCTGAA TGTTGTGGAT TTTGTGTTAT |
| | | AATTTACTTT GTCCAGGAAC TTGTGCAAGG GAGAGCCAAG GAAATAGGAT |
| | | GTTTGGCACC CAAATGGCGT CAGCCTCTCC AGGTCCTTCT TGCCTCCCCT |
| | | CCTGTCTTTT ATTTCTAGCC CCTTTTGGAA CAGAAGGACC CCGGGTTTCA |
| | | CATTGGAGCC TCCATATTTA TGCCTGGAAT GGAAAGAGGC CTATGAAGCT |
| | | GGGGTTGTCA TTGAGAAATT CTAGTTCAGC ACCTGGTCAC AAATCACCCT |
| | | TAATTCCTGC TATGATTAAA ATACATTTGT TGAACAGTGA ACAAGCTACC |
| | | ACTCGTAAGG CAAACTGTAT TATTACTGGC AAATAAAGCG TCATGGATAG |
| | | CTGCAATTTC TCACTTTACA GAAACAAGGG ATAACGTCTA GATTTGCTGC |
| | | GGGGTTTCTC TTTCAGGAGC TCTCACTAGG TAGACAGCTT TAGTCCTGCT |
| | | ACATCAGAGT TACCTGGGCA CTGTGGCTTG GGATTCACTA GCCCTGAGCC |
| | | TGATGTTGCT GGCTATCCCT TGAAGACAAT GTTTATTTCC ATAATCTAGA |
| | | GTCAGTTTCC CTGGGCATCT TTTCTTTGAA TCACAAATGC TGCCAACCTT |
| | | GGTCCAGGTG AAGGCAACTC AAAAGGTGAA AATACAAGGT GACCGTGCGA |
| | | AGGCGCTAGC CGAAACATCT TAGCTGAATA GGTTTCTGAA CTGGCCCTTT |
| | | TCATAGCTGT TTCAGGGCCT GTTTTTTTCA CGTTGCAGTC CTTTTGCTAT |
| | | GATTATGTGA AGTTGCCAAA CCTCTGTGCT GTGGATGTTT TGGCAGTGGG |
| | | CTTTGAAGTC GGCAGGACAC GATTACCAAT GCTCCTGACA CCCCGTGTCA |
| | | TTTGGATTAG ACGGAGCCCA ACCATCCATC ATTTTGCAGC AGCCTGGGAA |
| | | GGCCCACAAA GTGCCCGTAT CTCCTTAGGG AAAATAAATA AATACAATCA |
| | | TGAAAGCTGG CAGTTAGGCT GACCCAAACT GTGCTAATGG AAAAGATCAG |
| | | TCATTTTTAT TTTGGAATGC AAAGTCAAGA CACACCTACA TTCTTCATAG |
| | | AAATACACAT TTACTTGGAT AATCACTCAG TTCTCTCTTC AAGACTGTCT |
| | | CATGAGCAAG ATCATAAAAA CAAGACATGA TTATCATATT CAATTTTAAC |
| | | AGATGTTTTC CATTAGATCC CTCAACCCTC CACCCCCAGT CCAGGTTATT |
| | | AGCAAGTCTT ATGAGCAACT GGGATAATTT TGGATAACAT GATAATACTG |
| | | AGTTCCTTCA AATACATAAT TCTTAAATTG TTTCAAAATG GCATTAACTC |
| | | TCTGTTACTG TTGTAATCTA ATTCCAAAGC CCCCTCCAGG TCATATTCAT |
| | | AATTGCATGA ACCTTTTCTC TCTGTTTGTC CCTGTCTCTT GGCTTGCCCT |
| | | GATGTATACT CAGACTCCTG TACAATCTTA CTCCTGCTGG CAAGAGATTT |
| | | GTCTTCTTTT CTTGTCTTCA ATTGGCTTTC GGGCCTTGTA TGTGGTAAAA |
| | | TCACCAAATC ACAGTCAAGA CTGTGTTTTT GTTCCTAGTT TGATGCCCTT |
| | | ATGTCCCGGA GGGGTTCACA AAGTGCTTTG TCAGGACTGC TGCAGTTAGA |
| | | AGGCTCACTG CTTCTCCTAA GCCTTCTGCA CAGATGTGGC ACCTGCAACC |
| | | CAGGAGCAGG AGCCGGAGGA GCTGCCCTCT GACAGCAGGT GCAGCAGAGA |
| | | TGGCTACAGC TCAGGAGCTG GGAAGGTGAT GGGGCACAGG GAAAGCACAG |
| | | ATGTTCTGCA GCGCCCCAAA GTGACCCATT GCCTGGAGAA AGAGAAGAAA |
| | | ATATTTTTTA AAAAGCTAGT TTATTTAGCT TCTCATTAAT TCATTCAAAT |
| | | AAAGTCGTGA GGTGACTAAT TAGAGAATAA AAATTACTTT GGACTACTCA |
| | | AAAA |
| 16 | KLK4 | AGGCAGCAGG CTGGAGCTCA GCCCAGCAGT GGAATCCAGG AGCCCAGAGG |
| | | TGGCCGGGTG CTGACGTGAT GGCCACAGCA GGAAATCCCT GGGGCTGGTT |
| | | CCTGGGGTAC CTCATCCTTG GTGTCGCAGG ATCGCTCGTC TCTGGTAGCT |
| | | GCAGCCAAAT CATAAACGGC GAGGACTGCA GCCCGCACTC GCAGCCCTGG |
| | | CAGGCGGCAC TGGTCATGGA AAACGAATTG TTCTGCTCGG GCGTCCTGGT |
| | | GCATCCGCAG TGGGTGCTGT CAGCCGCACA CTGTTTCCAG AACTCCTACA |
| | | CCATCGGGCT GGGCCTGCAC AGTCTTGAGG CCGACCAAGA GCCAGGGAGC |
| | | CAGATGGTGG AGGCCAGCCT CTCCGTACGG CACCCAGAGT ACAACAGACC |
| | | CTTGCTCGCT AACGACCTCA TGCTCATCAA GTTGGACGAA TCCGTGTCCG |
| | | AGTCTGACAC CATCCGGAGC ATCAGCATTG CTTCGCAGTG CCCTACCGCG |
| | | GGGAACTCTT GCCTCGTTTC TGGCTGGGGT CTGCTGGCGA ACGGCAGAAT |
| | | GCCTACCGTG CTGCAGTGCG TGAACGTGTC GGTGGTGTCT GAGGAGGTCT |
| | | GCAGTAAGCT CTATGACCCG CTGTACCACC CCAGCATGTT CTGCGCCGGC |
| | | GGAGGGCAAG ACCAGAAGGA CTCCTGCAAC GGTGACTCTG GGGGGCCCCT |
| | | GATCTGCAAC GGGTACTTGC AGGGCCTTGT GTCTTTCGGA AAAGCCCCGT |
| | | GTGGCCAAGT TGGCGTGCCA GGTGTCTACA CCAACCTCTG CAAATTCACT |
| | | GAGTGGATAG AGAAAACCGT CCAGGCCAGT TAACTCTGGG GACTGGGAAC |
| | | CCATGAAATT GACCCCCAAA TACATCCTGC GGAAGGAATT CAGGAATATC |
| | | TGTTCCCAGC CCCTCCTCCC TCAGGCCCAG GAGTCCAGGC CCCCAGCCCC |
| | | TCCTCCCTCA AACCAAGGGT ACAGATCCCC AGCCCCTCCT CCCTCAGACC |
| | | CAGGAGTCCA GACCCCCCAG CCCCTCCTCC CTCAGACCCA GGAGTCCAGC |
| | | CCCTCCTCCC TCAGACCCAG GAGTCCAGAC CCCCCAGCCC CTCCTCCCTC |
| | | AGACCCAGGA GTCCAGCCCC TCCTCCCTCA GACCCAGGAG TCCAGACCCC |
| | | CCAGCCCCTC CTCCCTCAGA CCCAGGGGTC CAGGCCCCCA ACCCCTCCTC |
| | | CCTCAGACTC AGAGGTCCAG GCCCCCAACC CCTCCTTCCC CAGACCCAGA |
| | | GGTCCAGGTC CCAGCCCCTC CTCCCTCAGA CCCAGCGGTC CAATGCCACC |
| | | TAGACTCTCC CTGTACACAG TGCCCCCTTG TGGCACGTTG ACCCAACCTT |
| | | ACCAGTTGGT TTTTCATTTT TTGTCCCTTT CCCCTAGATC CAGAAATAAA |
| | | GTCTAAGAGA AGCGCA |
| 17 | HOXC6 | ATAACCATCT AGTTCCGAGT ACAAACTGGA GACAGAAATA AATATTAAAG |
| | | AAATCATAGA CCGACCAGGT AAAGGCAAAG GGATGAATTC CTACTTCACT |
| | | AACCCTTCCT TATCCTGCCA CCTCGCCGGG GGCCAGGACG TCCTCCCCAA |
| | | CGTCGCCCTC AATTCCACCG CCTATGATCC AGTGAGGCAT TTCTCGACCT |
| | | ATGGAGCGGC CGTTGCCCAG AACCGGATCT ACTCGACTCC CTTTTATTCG |
| | | CCACAGGAGA ATGTCGTGTT CAGTTCCAGC CGGGGGCCGT ATGACTATGG |
| | | ATCTAATTCC TTTTACCAGG AGAAAGACAT GCTCTCAAAC TGCAGACAAA |
| | | ACACCTTAGG ACATAACACA CAGACCTCAA TCGCTCAGGA TTTTAGTTCT |
| | | GAGCAGGGCA GGACTGCGCC CCAGGACCAG AAAGCCAGTA TCCAGATTTA |
| | | CCCCTGGATG CAGCGAATGA ATTCGCACAG TGGGGTCGGC TACGGAGCGG |
| | | ACCGGAGGCG CGGCCGCCAG ATCTACTCGC GGTACCAGAC CCTGGAACTG |
| | | GAGAAGGAAT TTCACTTCAA TCGCTACCTA ACGCGGCGCC GGCGCATCGA |
| | | GATCGCCAAC GCGCTTTGCC TGACCGAGCG ACAGATCAAA ATCTGGTTCC |
| | | AGAACCGCCG GATGAAGTGG AAAAAAGAAT CTAATCTCAC ATCCACTCTC |
| | | TCGGGGGGCG GCGGAGGGGC CACCGCCGAC AGCCTGGGCG GAAAAGAGGA |
| | | AAAGCGGGAA GAGACAGAAG AGGAGAAGCA GAAAGAGTGA CCAGGACTGT |
| | | CCCTGCCACC CCTCTCTCCC TTTCTCCCTC GCTCCCCACC AACTCTCCCC |
| | | TAATCACACA CTCTGTATTT ATCACTGGCA CAATTGATGT GTTTTGATTC |
| | | CCTAAAACAA AATTAGGGAG TCAAACGTGG ACCTGAAAGT CAGCTCTGGA |
| | | CCCCCTCCCT CACCGCACAA CTCTCTTTCA CCACGCGCCT CCTCCTCCTC |
| | | GCTCCCTTGC TAGCTCGTTC TCGGCTTGTC TACAGGCCCT TTTCCCCGTC |
| | | CAGGCCTTGG GGGCTCGGAC CCTGAACTCA GACTCTACAG ATTGCCCTCC |
| | | AAGTGAGGAC TTGGCTCCCC CACTCCTTCG ACGCCCCCAC CCCCGCCCCC |
| | | CGTGCAGAGA GCCGGCTCCT GGGCCTGCTG GGGCCTCTGC TCCAGGGCCT |
| | | CAGGGCCCGG CCTGGCAGCC GGGGAGGGCC GGAGGCCCAA GGAGGGCGCG |
| | | CCTTGGCCCC ACACCAACCC CCAGGGCCTC CCCGCAGTCC CTGCCTAGCC |
| | | CCTCTGCCCC AGCAAATGCC CAGCCCAGGC AAATTGTATT TAAAGAATCC |
| | | TGGGGGTCAT TATGGCATTT TACAAACTGT GACCGTTTCT GTGTGAAGAT |
| | | TTTTAGCTGT ATTTGTGGTC TCTGTATTTA TATTTATGTT TAGCACCGTC |
| | | AGTGTTCCTA TCCAATTTCA AAAAAGGAAA AAAAAGAGGG AAAATTACAA |
| | | AAAGAGAGAA AAAAAGTGAA TGACGTTTGT TTAGCCAGTA GGAGAAAATA |
| | | AATAAATAAA TAAATCCCTT CGTGTTACCC TCCTGTATAA ATCCAACCTC |
| | | TGGGTCCGTT CTCGAATATT TAATAAAACT GATATTATTT TTAAAACTTT |
| | | A |

In some embodiments, the methods and kits described herein are useful for detecting a level or an amount of expression of 1, 2, 3, 4, 5, 6, 7, 8, 9, 10, 11, 12, 13, 14, 15, 16 or 17 genes selected from TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6.

In some embodiments, the methods and kits described herein are useful for detecting a level or an amount of expression of at least three genes selected from TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6. In some embodiments, the at least three genes are TMPRSS2-ERG, PCA3 and PCAT14.

In some embodiments, the methods and kits described herein are useful for detecting a level or an amount of expression of at least four genes selected from TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6. In some embodiments, the at least four genes are TMPRSS2-ERG, PCA3, PCAT14, and OR51E2. In some embodiments, the at least four genes are TMPRSS2-ERG, PCA3, PCAT14, and TRGV9. In some embodiments, the at least four genes are TMPRSS2-ERG, PCA3, PCAT14, and ERG. In some embodiments, the at least four genes are TMPRSS2-ERG, PCA3, PCAT14, and TFF3. In some embodiments, the at least four genes are TMPRSS2-ERG, PCA3, PCAT14, and SCHLAP1. In some embodiments, the at least four genes are TMPRSS2-ERG, PCA3, PCAT14, and HOXC6. In some embodiments, the at least four genes are TMPRSS2-ERG, PCA3, PCAT14, and SPON2. In some embodiments, the at least four genes are TMPRSS2-ERG, PCA3, PCAT14, and TMSB15A. In some embodiments, the at least four genes are TMPRSS2-ERG, PCA3, PCAT14, and APOC1. In some embodiments, the at least four genes are TMPRSS2-ERG, PCA3, PCAT14, and B3GNT6. In some embodiments, the at least four genes are TMPRSS2-ERG, PCA3, PCAT14, and KLK4. In some embodiments, the at least four genes are TMPRSS2-ERG, PCA3, PCAT14, and CAMKK2. In some embodiments, the at least four genes are TMPRSS2-ERG, PCA3, PCAT14, and NKAIN1. In some embodiments, the at least four genes are TMPRSS2-ERG, PCA3, PCAT14, and PCGEM1.

In some embodiments, the methods and kits described herein are useful for detecting a level or an amount of expression of at least five genes selected from TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6. In some embodiments, the at least five genes are TMPRSS2-ERG, PCA3, PCAT14, OR51E2 and TRGV9. In some embodiments, the at least five genes are TMPRSS2-ERG, PCA3, PCAT14, ERG and OR51E2. In some embodiments, the at least five genes are TMPRSS2-ERG, PCA3, PCAT14, TFF3 and TRGV9. In some embodiments, the at least five genes are TMPRSS2-ERG, PCA3, PCAT14, OR51E2, and TFF3. In some embodiments, the at least five genes are TMPRSS2-ERG, PCA3, PCAT14, SCHLAP1, and TRGV9. In some embodiments, the at least five genes are TMPRSS2-ERG, PCA3, PCAT14, OR51E2 and SCHLAP1. In some embodiments, the at least five genes are TMPRSS2-ERG, PCA3, PCAT14, HOXC6 and TRGV9. In some embodiments, the at least five genes are TMPRSS2-ERG, PCA3, PCAT14, OR51E2 and HOXC6. In some embodiments, the at least five genes are TMPRSS2-ERG, PCA3, PCAT14, SPON2, and TRGV9. In some embodiments, the at least five genes are TMPRSS2-ERG, PCA3, PCAT14, ERG and SPON2. In some embodiments, the at least five genes are TMPRSS2-ERG, PCA3, PCAT14, TMPSB15A and TRGV9. In some embodiments, the at least five genes are TMPRSS2-ERG, PCA3, PCAT14, ERG and TMSB15A. In some embodiments, the at least five genes are TMPRSS2-ERG, PCA3, PCAT14, APOC1, and TRGV9. In some embodiments, the at least five genes are TMPRSS2-ERG, PCA3, PCAT14, ERG and APOC1. In some embodiments, the at least five genes are TMPRSS2-ERG, PCA3, PCAT14, B3GNT6 and TRGV9. In some embodiments, the at least five genes are TMPRSS2-ERG, PCA3, PCAT14, ERG and B3GNT6. In some embodiments, the at least five genes are TMPRSS2-ERG, PCA3, PCAT14, KLK4 and TRGV9. In some embodiments, the at least five genes are TMPRSS2-ERG, PCA3, PCAT14, ERG and KLK4. In some embodiments, the at least five genes are TMPRSS2-ERG, PCA3, PCAT14, CAMKK2 and TRGV9. In some embodiments, the at least five genes are TMPRSS2-ERG, PCA3, PCAT14, ERG and CAMKK2. In some embodiments, the at least five genes are TMPRSS2-ERG, PCA3, PCAT14, NKAIN1, and TRGV9. In some embodiments, the at least five genes are TMPRSS2-ERG, PCA3, PCAT14, ERG and NKAIN1. In some embodiments, the at least five genes are TMPRSS2-ERG, PCA3, PCAT14, PCGEM1 and TRGV9. In some embodiments, the at least five genes are TMPRSS2-ERG, PCA3, PCAT14, ERG and PCGEM1.

In some embodiments, the methods and kits described herein are useful for detecting a level or an amount of expression of at least six genes selected from TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6. In some embodiments, the at least six genes are TMPRSS2-ERG, PCA3, PCAT14, ERG, TRGV9 and OR51E2. In some embodiments, the at least six genes are TMPRSS2-ERG, PCA3, PCAT14, ERG, NKAIN1, and PCGEM1. In some embodiments, the at least six genes are TMPRSS2-ERG, PCA3, PCAT14, TRGV9 and PCGEM1. In some embodiments, the at least six genes are TMPRSS2-ERG, PCA3, PCAT14, ERG, CAMKK2 and PCGEM1. In some embodiments, the at least six genes are TMPRSS2-ERG, PCA3, PCAT14, ERG, TRGV9 and NKAIN1. In some embodiments, the at least six genes are TMPRSS2-ERG, PCA3, PCAT14, ERG, OR51E2 and TCGEM1. In some embodiments, the at least six genes are TMPRSS2-ERG, PCA3, PCAT14, ERG, TRGV9 and TFF3. In some embodiments, the at least six genes are TMPRSS2-ERG, PCA3, PCAT14, ERG, OR51E2 and SCHLAP1. In some embodiments, the at least six genes are TMPRSS2-ERG, PCA3, PCAT14, ERG, TFF3 and HOXC6. In some embodiments, the at least six genes are TMPRSS2-ERG, PCA3, PCAT14, ERG, SCHLAP1 and SPON2. In some embodiments, the at least six genes are TMPRSS2-ERG, PCA3, PCAT14, ERG, HOXC6 and TMSB15A. In some embodiments, the at least six genes are TMPRSS2-ERG, PCA3, PCAT14, ERG, SPON2 and APOC1. In some embodiments, the at least six genes are TMPRSS2-ERG, PCA3, PCAT14, ERG, TMSB15A and APOC1. In some embodiments, the at least six genes are TMPRSS2-ERG, PCA3, PCAT14, ERG, TMSB15A and B3GNT6. In some embodiments, the at least six genes are TMPRSS2-ERG, PCA3, PCAT14, ERG, APOC1 and KLK4. In some embodiments, the at least six genes are TMPRSS2-ERG, PCA3, PCAT14, ERG, B3GNT6 and CAMKK2. In some embodiments, the at least six genes are TMPRSS2-ERG, PCA3, PCAT14, ERG, KLK4 and NKAIN1. In some embodiments, the at least six genes are TMPRSS2-ERG, PCA3, PCAT14, ERG, CAMKK2 and PCGEM1. In some embodiments, the at least six genes are TMPRSS2-ERG, PCA3, PCAT14, ERG, KLK4 and PCGEM1. In some embodiments, the at least six genes are TMPRSS2-ERG, PCA3, PCAT14, ERG, TRGV9 and CAMKK12. In some embodiments, the at least six genes are TMPRSS2-ERG, PCA3, PCAT14, ERG, OR51E2 and NKAIN1. In some embodiments, the at least six genes are TMPRSS2-ERG, PCA3, PCAT14, ERG, TFF3 and PCGEM1. In some embodiments, the at least six genes are TMPRSS2-ERG, PCA3, PCAT14, ERG, TRGV9 and SCHLAP1. In some embodiments, the at least six genes are TMPRSS2-ERG, PCA3, PCAT14, ERG, OR51E2 and HOXC6. In some embodiments, the at least six genes are TMPRSS2-ERG, PCA3, PCAT14, ERG, TFF3 and SPON2. In some embodiments, the at least six genes are TMPRSS2-ERG, PCA3, PCAT14, ERG, SCHLAP1 and TMSB15A. In some embodiments, the at least six genes are TMPRSS2-ERG, PCA3, PCAT14, ERG, HOXC6 and APOC1. In some embodiments, the at least six genes are TMPRSS2-ERG, PCA3, PCAT14, ERG, SPON2 and B2GNT6. In some embodiments, the at least six genes are TMPRSS2-ERG, PCA3, PCAT14, ERG, TMSB15A and KLK4. In some embodiments, the at least six genes are TMPRSS2-ERG, PCA3, PCAT14, ERG, APOC1 and CAMKK2. In some embodiments, the at least six genes are TMPRSS2-ERG, PCA3, PCAT14, ERG, B3GNT6 and NKAIN1. In some embodiments, the at least six genes are TMPRSS2-ERG, PCA3, PCAT14, ERG, B3GNT6 and PCGEM1. In some embodiments, the at least six genes are TMPRSS2-ERG, PCA3, PCAT14, ERG, TRGV9 and KLK4. In some embodiments, the at least six genes are TMPRSS2-ERG, PCA3, PCAT14, ERG, OR51E2 and CAMKK2. In some embodiments, the at least six genes are TMPRSS2-ERG, PCA3, PCAT14, ERG, TFF3 and NKAIN1. In some embodiments, the at least six genes are TMPRSS2-ERG, PCA3, PCAT14, ERG, SCHLAP1 and PCGEM1. In some embodiments, the at least six genes are TMPRSS2-ERG, PCA3, PCAT14, ERG, TRGV9 and HOXC6. In some embodiments, the at least six genes are TMPRSS2-ERG, PCA3, PCAT14, ERG, OR51E2 and SPON2. In some embodiments, the at least six genes are TMPRSS2-ERG, PCA3, PCAT14, ERG, TFF3 and TMSB15A. In some embodiments, the at least six genes are TMPRSS2-ERG, PCA3, PCAT14, ERG, SCHLAP1 and APOC1. In some embodiments, the at least six genes are TMPRSS2-ERG, PCA3, PCAT14, ERG, HOXC6 and B3GNT6. In some embodiments, the at least six genes are TMPRSS2-ERG, PCA3, PCAT14, ERG, SPON2 and KLK4. In some embodiments, the at least six genes are TMPRSS2-ERG, PCA3, PCAT14, ERG, TMSB15A and CAMKK2. In some embodiments, the at least six genes are TMPRSS2-ERG, PCA3, PCAT14, ERG, APOC1 and NKAIN1. In some embodiments, the at least six genes are TMPRSS2-ERG, PCA3, PCAT14, ERG, APOC1 and PCGEM1. In some embodiments, the at least six genes are TMPRSS2-ERG, PCA3, PCAT14, ERG, TRGV9 and B3GNT6. In some embodiments, the at least six genes are TMPRSS2-ERG, PCA3, PCAT14, ERG, OR51E2 and KLK4. In some embodiments, the at least six genes are TMPRSS2-ERG, PCA3, PCAT14, ERG, TFF3 and CAMKK2. In some embodiments, the at least six genes are TMPRSS2-ERG, PCA3, PCAT14, ERG, SCHLAP1 and NKAIN1. In some embodiments, the at least six genes are TMPRSS2-ERG, PCA3, PCAT14, ERG, HOXC6 and PCGEM1. In some embodiments, the at least six genes are TMPRSS2-ERG, PCA3, PCAT14, ERG, HOXC6 and SPON2. In some embodiments, the at least six genes are TMPRSS2-ERG, PCA3, PCAT14, ERG, OR51E2 and TMSB15A. In some embodiments, the at least six genes are TMPRSS2-ERG, PCA3, PCAT14, ERG, TMSB15A and PCGEM1. In some embodiments, the at least six genes are TMPRSS2-ERG, PCA3, PCAT14, ERG, TRGV9 and APOC1. In some embodiments, the at least six genes are TMPRSS2-ERG, PCA3, PCAT14, ERG, OR51E2 and B3GNT6. In some embodiments, the at least six genes are TMPRSS2-ERG, PCA3, PCAT14, ERG, TFF3 and KLK4. In some embodiments, the at least six genes are TMPRSS2-ERG, PCA3, PCAT14, ERG, SCHLAP1 and CAMKK2. In some embodiments, the at least six genes are TMPRSS2-ERG, PCA3, PCAT14, ERG, HOXC6 and NKAIN1. In some embodiments, the at least six genes are TMPRSS2-ERG, PCA3, PCAT14, ERG, SPON2 and PCGEM1.

In some embodiments, the methods and kits described herein are useful for detecting a level or an amount of expression of at least seven genes selected from TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6. In some embodiments, the at least seven genes are TMPRSS2-ERG, PCA3, PCAT14, ERG, TRGV9, OR51E2 and TFF3. In some embodiments, the at least seven genes are TMPRSS2-ERG, PCA3, PCAT14, ERG, TRGV9, NKAIN1 and PCGEM1. In some embodiments, the at least seven genes are TMPRSS2-ERG, PCA3, PCAT14, ERG, TRGV9, OR51E2 and PCGEM1. In some embodiments, the at least seven genes are TMPRSS2-ERG, PCA3, PCAT14, ERG, TRGV9, CAMKK2, and PCGEM1. In some embodiments, the at least seven genes are TMPRSS2-ERG, PCA3, PCAT14, ERG, TRGV9, OR51E2 and NKAIN1. In some embodiments, the at least seven genes are TMPRSS2-ERG, PCA3, PCAT14, ERG, TRGV9, KLK4 and PCGEM1. In some embodiments, the at least seven genes are TMPRSS2-ERG, PCA3, PCAT14, ERG, TRGV9, OR51E2 and CAMKK2. In some embodiments, the at least seven genes are TMPRSS2-ERG, PCA3, PCAT14, ERG, TRGV9, SCHLAP1 and HOXC6. In some embodiments, the at least seven genes are TMPRSS2-ERG, PCA3, PCAT14, ERG, TRGV9, B3GNT6 and PCGEM1. In some embodiments, the at least seven genes are TMPRSS2-ERG, PCA3, PCAT14, ERG, TRGV9, OR51E2 and KLK4. In some embodiments, the at least seven genes are TMPRSS2-ERG, PCA3, PCAT14, ERG, TRGV9, HOXC6 and SPON2. In some embodiments, the at least seven genes are TMPRSS2-ERG, PCA3, PCAT14, ERG, TRGV9, APOC1 and PCGEM1. In some embodiments, the at least seven genes are TMPRSS2-ERG, PCA3, PCAT14, ERG, TRGV9, OR51E2 and B3GNT6. In some embodiments, the at least seven genes are TMPRSS2-ERG, PCA3, PCAT14, ERG, TRGV9, TMSB15A and PCGEM1. In some embodiments, the at least seven genes are TMPRSS2-ERG, PCA3, PCAT14, ERG, TRGV9, OR51E2 and APOC1. In some embodiments, the at least seven genes are TMPRSS2-ERG, PCA3, PCAT14, ERG, KLK4, NKAIN1 and PCGEM1. In some embodiments, the at least seven genes are TMPRSS2-ERG, PCA3, PCAT14, ERG, B3GNT6, NKAIN1, and PCGEM1. In some embodiments, the at least seven genes are TMPRSS2-ERG, PCA3, PCAT14, ERG, OR51E2, TFF3 and NKAIN1. In some embodiments, the at least seven genes are TMPRSS2-ERG, PCA3, PCAT14, ERG, OR51E2, HOXC6 and SPON2. In some embodiments, the at least seven genes are TMPRSS2-ERG, PCA3, PCAT14, ERG, OR51E2, TFF3 and CAMKK2. In some embodiments, the at least seven genes are TMPRSS2-ERG, PCA3, PCAT14, ERG, OR51E2, SPON2 and TMPSB15A. In some embodiments, the at least seven genes are TMPRSS2-ERG, PCA3, PCAT14, ERG, OR51E2, TFF3 and KLK4. In some embodiments, the at least seven genes are TMPRSS2-ERG, PCA3, PCAT14, ERG, TFF3, SCHLAP1 and PCGEM1. In some embodiments, the at least seven genes are TMPRSS2-ERG, PCA3, PCAT14, ERG, TFF3, SPON2 and TMSB15A. In some embodiments, the at least seven genes are TMPRSS2-ERG, PCA3, PCAT14, ERG, TFF3, SCHLAP1 and NKAIN1. In some embodiments, the at least seven genes are TMPRSS2-ERG, PCA3, PCAT14, ERG, TFF3, TMSB15A and APOC1. In some embodiments, the at least seven genes are TMPRSS2-ERG, PCA3, PCAT14, ERG, TFF3, SCHLAP1 and CAMKK2. In some embodiments, the at least seven genes are TMPRSS2-ERG, PCA3, PCAT14, ERG, SCHLAP1, TMSB15A and APOC1. In some embodiments, the at least seven genes are TMPRSS2-ERG, PCA3, PCAT14, ERG, SCHLAP1, HOXC6 and PCGEM1. In some embodiments, the at least seven genes are TMPRSS2-ERG, PCA3, PCAT14, ERG, SCHLAP1, APOC1 and B3GNT6. In some embodiments, the at least seven genes are TMPRSS2-ERG, PCA3, PCAT14, ERG, SCHLAP1, HOXC6 and NKAIN1. In some embodiments, the at least seven genes are TMPRSS2-ERG, PCA3, PCAT14, ERG, HOXC6, APOC1 and B3GNT6. In some embodiments, the at least seven genes are TMPRSS2-ERG, PCA3, PCAT14, ERG, HOXC6, B3GNT6 and KLK4. In some embodiments, the at least seven genes are TMPRSS2-ERG, PCA3, PCAT14, ERG, HOXC6, SPON2 and PCGEM1. In some embodiments, the at least seven genes are TMPRSS2-ERG, PCA3, PCAT14, ERG, SPON2, B3GNT6 and KLK4. In some embodiments, the at least seven genes are TMPRSS2-ERG, PCA3, PCAT14, ERG, SPON2, KLK4 and CAMKK2. In some embodiments, the at least seven genes are TMPRSS2-ERG, PCA3, PCAT14, ERG, SPON2, NKAIN1 and PCGEM1. In some embodiments, the at least seven genes are TMPRSS2-ERG, PCA3, PCAT14, ERG, TMSB15A, KLK4 and CAMKK2. In some embodiments, the at least seven genes are TMPRSS2-ERG, PCA3, PCAT14, ERG, TMSB15A, CAMKK2 and NKAIN1. In some embodiments, the at least seven genes are TMPRSS2-ERG, PCA3, PCAT14, ERG, TMSB15A, NKAIN1 and PCGEM1. In some embodiments, the at least seven genes are TMPRSS2-ERG, PCA3, PCAT14, ERG, APOC1, NKAIN1, and CAMKK2. In some embodiments, the at least seven genes are TMPRSS2-ERG, PCA3, PCAT14, ERG, APOC1, NKAIN1 and PCGEM1.

In some embodiments, the methods and kits described herein are useful for detecting a level or an amount of expression of at least eight genes selected from TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6. In some embodiments, the at least eight genes are TMPRSS2-ERG, PCA3, PCAT14, ERG, TRGV9, OR51E2, TFF3 and SCHLAP1. In some embodiments, the at least eight genes are TMPRSS2-ERG, PCA3, PCAT14, ERG, TRGV9, OR51E2, NKAIN1, and PCGEM1. In some embodiments, the at least eight genes are TMPRSS2-ERG, PCA3, PCAT14, ERG, TRGV9, OR51E2, TFF3 and PCGEM1. In some embodiments, the at least eight genes are TMPRSS2-ERG, PCA3, PCAT14, ERG, TRGV9, OR51E2, CAMKK2 and PCGEM1. In some embodiments, the at least eight genes are TMPRSS2-ERG, PCA3, PCAT14, ERG, TRGV9, OR51E2, TFF3 and NKAIN1. In some embodiments, the at least eight genes are TMPRSS2-ERG, PCA3, PCAT14, ERG, TRGV9, OR51E2, SCHLAP1 and HOXC6. In some embodiments, the at least eight genes are TMPRSS2-ERG, PCA3, PCAT14, ERG, TRGV9, OR51E2, KLK4 and PCGEM1. In some embodiments, the at least eight genes are TMPRSS2-ERG, PCA3, PCAT14, ERG, TRGV9, OR51E2, TFF3 and CAMKK2. In some embodiments, the at least eight genes are TMPRSS2-ERG, PCA3, PCAT14, ERG, TRGV9, OR51E2, HOXC6 and SPON2. In some embodiments, the at least eight genes are TMPRSS2-ERG, PCA3, PCAT14, ERG, TRGV9, OR51E2, B3GNT6 and PCGEM1. In some embodiments, the at least eight genes are TMPRSS2-ERG, PCA3, PCAT14, ERG, TRGV9, OR51E2, TFF3 and KLK4. In some embodiments, the at least eight genes are TMPRSS2-ERG, PCA3, PCAT14, ERG, TRGV9, OR51E2, SPON2 and PCGEM1. In some embodiments, the at least eight genes are TMPRSS2-ERG, PCA3, PCAT14, ERG, TRGV9, OR51E2, APOC1 and PCGEM1. In some embodiments, the at least eight genes are TMPRSS2-ERG, PCA3, PCAT14, ERG, TRGV9, OR51E2, TFF3 and B2GNT6. In some embodiments, the at least eight genes are TMPRSS2-ERG, PCA3, PCAT14, ERG, TRGV9, CAMKK2, NKAIN1 and PCGEM1. In some embodiments, the at least eight genes are TMPRSS2-ERG, PCA3, PCAT14, ERG, TRGV9, KLK4, NKAIN1 and PCGEM1. In some embodiments, the at least eight genes are TMPRSS2-ERG, PCA3, PCAT14, ERG, TRGV9, TFF3, SCHLAP1 and PCGEM1. In some embodiments, the at least eight genes are TMPRSS2-ERG, PCA3, PCAT14, ERG, TRGV9, TFF3, HOXC6 and SPON2. In some embodiments, the at least eight genes are TMPRSS2-ERG, PCA3, PCAT14, ERG, TRGV9, TFF3, SCHLAP1 and NKAIN1. In some embodiments, the at least eight genes are TMPRSS2-ERG, PCA3, PCAT14, ERG, TRGV9, TFF3, SPON2 and TMSB15A. In some embodiments, the at least eight genes are TMPRSS2-ERG, PCA3, PCAT14, ERG, TRGV9, TFF3, SCHLAP1 and CAMKK2. In some embodiments, the at least eight genes are TMPRSS2-ERG, PCA3, PCAT14, ERG, TRGV9, TFF3, SCHLAP1 and KLK4. In some embodiments, the at least eight genes are TMPRSS2-ERG, PCA3, PCAT14, ERG, TRGV9, SCHLAP1, SPON2 and TMSB15A. In some embodiments, the at least eight genes are TMPRSS2-ERG, PCA3, PCAT14, ERG, TRGV9, SCHLAP1, HOXC6 and PCGEM1. In some embodiments, the at least eight genes are TMPRSS2-ERG, PCA3, PCAT14, ERG, TRGV9, SCHLAP1, TMSB15A and APOC1. In some embodiments, the at least eight genes are TMPRSS2-ERG, PCA3, PCAT14, ERG, TRGV9, SCHLAP1, HOXC6 and NKAIN1. In some embodiments, the at least eight genes are TMPRSS2-ERG, PCA3, PCAT14, ERG, TRGV9, SCHLAP1, HOXC6 and CAMKK2. In some embodiments, the at least eight genes are TMPRSS2-ERG, PCA3, PCAT14, ERG, TRGV9, HOXC6, TMSB15A and APOC1. In some embodiments, the at least eight genes are TMPRSS2-ERG, PCA3, PCAT14, ERG, TRGV9, HOXC6, APOC1 and B3GNT6. In some embodiments, the at least eight genes are TMPRSS2-ERG, PCA3, PCAT14, ERG, TRGV9, HOXC6, SPON2 and NKAIN1. In some embodiments, the at least eight genes are TMPRSS2-ERG, PCA3, PCAT14, ERG, TRGV9, SPON2, APOC1 and B3GNT6. In some embodiments, the at least eight genes are TMPRSS2-ERG, PCA3, PCAT14, ERG, TRGV9, SPON2, B3GNT6 and KLK4. In some embodiments, the at least eight genes are TMPRSS2-ERG, PCA3, PCAT14, ERG, TRGV9, SPON2, TMSB15A and PCGEM1. In some embodiments, the at least eight genes are TMPRSS2-ERG, PCA3, PCAT14, ERG, TRGV9, TMSB15A, B3GNT6 and KLK4. In some embodiments, the at least eight genes are TMPRSS2-ERG, PCA3, PCAT14, ERG, TRGV9, TMSB15A, KLK4 and CAMKK2. In some embodiments, the at least eight genes are TMPRSS2-ERG, PCA3, PCAT14, ERG, TRGV9, TMSB15A, NKAIN1 and PCGEM1. In some embodiments, the at least eight genes are TMPRSS2-ERG, PCA3, PCAT14, ERG, TRGV9, APOC1, KLK4 and CAMKK2. In some embodiments, the at least eight genes are TMPRSS2-ERG, PCA3, PCAT14, ERG, TRGV9, APOC1, CAMKK2 and NKAIN1. In some embodiments, the at least eight genes are TMPRSS2-ERG, PCA3, PCAT14, ERG, TRGV9, APOC1, NKAIN1 and PCGEM1. In some embodiments, the at least eight genes are TMPRSS2-ERG, PCA3, PCAT14, ERG, TRGV9, B3GNT6, CAMKK2 and NKAIN1. In some embodiments, the at least eight genes are TMPRSS2-ERG, PCA3, PCAT14, ERG, TRGV9, B3GNT6, NKAIN1 and PCGEM1.

In some embodiments, the methods and kits described herein are useful for detecting a level or an amount of expression of at least nine genes selected from TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6. In some embodiments, the at least nine genes are TMPRSS2-ERG, PCA3, PCAT14, ERG, TRGV9, OR51E2, TFF3, SCHLAP1 and HOXC6. In some embodiments, the at least nine genes are TMPRSS2-ERG, PCA3, PCAT14, ERG, TRGV9, OR51E2, TFF3, SCHLAP1 and pCGEM1. In some embodiments, the at least nine genes are TMPRSS2-ERG, PCA3, PCAT14, ERG, TRGV9, OR51E2, TFF3, SCHLAP1 and NKAIN1. In some embodiments, the at least nine genes are TMPRSS2-ERG, PCA3, PCAT14, ERG, TRGV9, OR51E2, TFF3, SCHLAP1 and SPON2. In some embodiments, the at least nine genes are TMPRSS2-ERG, PCA3, PCAT14, ERG, TRGV9, OR51E2, TFF3, SCHLAP1 and CAMKK2. In some embodiments, the at least nine genes are TMPRSS2-ERG, PCA3, PCAT14, ERG, TRGV9, OR51E2, TFF3, SCHLAP1 and HOXC6. In some embodiments, the at least nine genes are TMPRSS2-ERG, PCA3, PCAT14, ERG, TRGV9, OR51E2, TFF3, SCHLAP1 and TMSB15A. In some embodiments, the at least nine genes are TMPRSS2-ERG, PCA3, PCAT14, ERG, TRGV9, OR51E2, TFF3, SCHLAP1 and KLK4. In some embodiments, the at least nine genes are TMPRSS2-ERG, PCA3, PCAT14, ERG, TRGV9, OR51E2, TFF3, SCHLAP1 and B3GNT6. In some embodiments, the at least nine genes are TMPRSS2-ERG, PCA3, PCAT14, ERG, TRGV9, OR51E2, TFF3, NKAIN1 and PCGEM1. In some embodiments, the at least nine genes are TMPRSS2-ERG, PCA3, PCAT14, ERG, TRGV9, OR51E2, TFF3, NKAIN1 and KLK4. In some embodiments, the at least nine genes are TMPRSS2-ERG, PCA3, PCAT14, ERG, TRGV9, OR51E2, TFF3, NKAIN1 and B3GNT6. In some embodiments, the at least nine genes are TMPRSS2-ERG, PCA3, PCAT14, ERG, TRGV9, OR51E2, TFF3, NKAIN1 and SPON2. In some embodiments, the at least nine genes are TMPRSS2-ERG, PCA3, PCAT14, ERG, TRGV9, OR51E2, TFF3, NKAIN1 and TMSB15A. In some embodiments, the at least nine genes are TMPRSS2-ERG, PCA3, PCAT14, ERG, TRGV9, OR51E2, TFF3, CAMKK2 and PCGEM1. In some embodiments, the at least nine genes are TMPRSS2-ERG, PCA3, PCAT14, ERG, TRGV9, OR51E2, TFF3, CAMKK2 and B3GNT6. In some embodiments, the at least nine genes are TMPRSS2-ERG, PCA3, PCAT14, ERG, TRGV9, OR51E2, TFF3, CAMKK2 and APOC1. In some embodiments, the at least nine genes are TMPRSS2-ERG, PCA3, PCAT14, ERG, TRGV9, OR51E2, TFF3, CAMKK2 and HOXC6. In some embodiments, the at least nine genes are TMPRSS2-ERG, PCA3, PCAT14, ERG, TRGV9, OR51E2, TFF3, CAMKK2 and SPON2. In some embodiments, the at least nine genes are TMPRSS2-ERG, PCA3, PCAT14, ERG, TRGV9, OR51E2, TFF3, HOXC6 and PCGEM1. In some embodiments, the at least nine genes are TMPRSS2-ERG, PCA3, PCAT14, ERG, TRGV9, OR51E2, TFF3, HOXC6 and TMSB15A. In some embodiments, the at least nine genes are TMPRSS2-ERG, PCA3, PCAT14, ERG, TRGV9, OR51E2, TFF3, HOXC6 and APOC1. In some embodiments, the at least nine genes are TMPRSS2-ERG, PCA3, PCAT14, ERG, TRGV9, OR51E2, TFF3, HOXC6 and KLK4. In some embodiments, the at least nine genes are TMPRSS2-ERG, PCA3, PCAT14, ERG, TRGV9, OR51E2, TFF3, SPON2 and APOC1. In some embodiments, the at least nine genes are TMPRSS2-ERG, PCA3, PCAT14, ERG, TRGV9, OR51E2, TFF3, SPON2 and PCGEM1. In some embodiments, the at least nine genes are TMPRSS2-ERG, PCA3, PCAT14, ERG, TRGV9, OR51E2, TFF3, SPON2 and B3GNT6. In some embodiments, the at least nine genes are TMPRSS2-ERG, PCA3, PCAT14, ERG, TRGV9, OR51E2, TFF3, TMSB15A and B3GNT6. In some embodiments, the at least nine genes are TMPRSS2-ERG, PCA3, PCAT14, ERG, TRGV9, OR51E2, TFF3, TMSB15A and KLK4. In some embodiments, the at least nine genes are TMPRSS2-ERG, PCA3, PCAT14, ERG, TRGV9, OR51E2, TFF3, TMSB15A and PCGEM1. In some embodiments, the at least nine genes are TMPRSS2-ERG, PCA3, PCAT14, ERG, TRGV9, OR51E2, TFF3, TMSB15A and APOC1. In some embodiments, the at least nine genes are TMPRSS2-ERG, PCA3, PCAT14, ERG, TRGV9, OR51E2, TFF3, APOC1 and KLK4. In some embodiments, the at least nine genes are TMPRSS2-ERG, PCA3, PCAT14, ERG, TRGV9, OR51E2, TFF3, APOC1 and PCGEM1. In some embodiments, the at least nine genes are TMPRSS2-ERG, PCA3, PCAT14, ERG, TRGV9, OR51E2, TFF3, KLK4 and PCGEM1. In some embodiments, the at least nine genes are TMPRSS2-ERG, PCA3, PCAT14, ERG, TRGV9, OR51E2, TFF3, B3GNT6 and PCGEM1.

In some embodiments, the methods and kits described herein are useful for detecting a level or an amount of expression of at least ten genes selected from TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6. In some embodiments, the at least ten genes are TMPRSS2-ERG, PCA3, PCAT14, ERG, TRGV9, OR51E2, TFF3, SCHLAP1, HOXC6 and SPON2. In some embodiments, the at least ten genes are TMPRSS2-ERG, PCA3, PCAT14, ERG, TRGV9, OR51E2, TFF3, SCHLAP1, HOXC6 and PCGEM1. In some embodiments, the at least ten genes are TMPRSS2-ERG, PCA3, PCAT14, ERG, TRGV9, OR51E2, TFF3, SCHLAP1, NKAIN1 and pCGEM1. In some embodiments, the at least ten genes are TMPRSS2-ERG, PCA3, PCAT14, ERG, TRGV9, OR51E2, TFF3, SCHLAP1, CAMKK2 and PCGEM1. In some embodiments, the at least ten genes are TMPRSS2-ERG, PCA3, PCAT14, ERG, TRGV9, OR51E2, TFF3, SCHLAP1, HOXC6 and NKAIN1. In some embodiments, the at least ten genes are TMPRSS2-ERG, PCA3, PCAT14, ERG, TRGV9, OR51E2, TFF3, SCHLAP1, SPON2 and TMSB15A. In some embodiments, the at least ten genes are TMPRSS2-ERG, PCA3, PCAT14, ERG, TRGV9, OR51E2, TFF3, SCHLAP1, KLK4 and PCGEM1. In some embodiments, the at least ten genes are TMPRSS2-ERG, PCA3, PCAT14, ERG, TRGV9, OR51E2, TFF3, SCHLAP1, HOXC6 and CAMKK2. In some embodiments, the at least ten genes are TMPRSS2-ERG, PCA3, PCAT14, ERG, TRGV9, OR51E2, TFF3, SCHLAP1, TMBS15A and APOC1. In some embodiments, the at least ten genes are TMPRSS2-ERG, PCA3, PCAT14, ERG, TRGV9, OR51E2, TFF3, SCHLAP1, B3GNT6 and PCGEM1. In some embodiments, the at least ten genes are TMPRSS2-ERG, PCA3, PCAT14, ERG, TRGV9, OR51E2, TFF3, SCHLAP1, HOXC6 and KLK4. In some embodiments, the at least ten genes are TMPRSS2-ERG, PCA3, PCAT14, ERG, TRGV9, OR51E2, TFF3, SCHLAP1, APOC1 and B3GNT6. In some embodiments, the at least ten genes are TMPRSS2-ERG, PCA3, PCAT14, ERG, TRGV9, OR51E2, TFF3, CAMKK2, NKAIN1 and PCGEM1. In some embodiments, the at least ten genes are TMPRSS2-ERG, PCA3, PCAT14, ERG, TRGV9, OR51E2, TFF3, CAMKK2, TMSB15A and KLK4. In some embodiments, the at least ten genes are TMPRSS2-ERG, PCA3, PCAT14, ERG, TRGV9, OR51E2, TFF3, CAMKK2, APOC1 and NKAIN1. In some embodiments, the at least ten genes are TMPRSS2-ERG, PCA3, PCAT14, ERG, TRGV9, OR51E2, TFF3, CAMKK2, HOXC6 and SPON2. In some embodiments, the at least ten genes are TMPRSS2-ERG, PCA3, PCAT14, ERG, TRGV9, OR51E2, TFF3, KLK4, NKAIN1 and PCGEM1. In some embodiments, the at least ten genes are TMPRSS2-ERG, PCA3, PCAT14, ERG, TRGV9, OR51E2, TFF3, KLK4, SPON2 and B3GNT6. In some embodiments, the at least ten genes are TMPRSS2-ERG, PCA3, PCAT14, ERG, TRGV9, OR51E2, TFF3, HOXC6, SPON2 and PCGEM1. In some embodiments, the at least ten genes are TMPRSS2-ERG, PCA3, PCAT14, ERG, TRGV9, OR51E2, TFF3, HOXC6, TMSB15A and APCOC1. In some embodiments, the at least ten genes are TMPRSS2-ERG, PCA3, PCAT14, ERG, TRGV9, OR51E2, TFF3, HOXC6, SPON2 and NKAIN1. In some embodiments, the at least ten genes are TMPRSS2-ERG, PCA3, PCAT14, ERG, TRGV9, OR51E2, TFF3, HOXC6, APOC1 and B3GNT6. In some embodiments, the at least ten genes are TMPRSS2-ERG, PCA3, PCAT14, ERG, TRGV9, OR51E2, TFF3, SPON2, APOC1 and B3GNT6. In some embodiments, the at least ten genes are TMPRSS2-ERG, PCA3, PCAT14, ERG, TRGV9, OR51E2, TFF3, SPON2, TMSB15A and PCGEM1. In some embodiments, the at least ten genes are TMPRSS2-ERG, PCA3, PCAT14, ERG, TRGV9, OR51E2, TFF3, SPON2, TMSB15A and NKAIN1. In some embodiments, the at least ten genes are TMPRSS2-ERG, PCA3, PCAT14, ERG, TRGV9, OR51E2, TFF3, B3GNT6, NKAIN1 and PCGEM1. In some embodiments, the at least ten genes are TMPRSS2-ERG, PCA3, PCAT14, ERG, TRGV9, OR51E2, TFF3, B3GNT6, APOC1 and TMSB15A. In some embodiments, the at least ten genes are TMPRSS2-ERG, PCA3, PCAT14, ERG, TRGV9, OR51E2, TFF3, APOC1, NKAIN1 and PCGEM1. In some embodiments, the at least ten genes are TMPRSS2-ERG, PCA3, PCAT14, ERG, TRGV9, OR51E2, TFF3, APOC1, TMSB15A and PCGEM1. In some embodiments, the at least ten genes are TMPRSS2-ERG, PCA3, PCAT14, ERG, TRGV9, OR51E2, TFF3, TMSB15A, NKAIN1 and PCGEM1.

In some embodiments, the methods and kits described herein are useful for detecting a level or an amount of expression of at least eleven genes selected from TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6. In some embodiments, the at least eleven genes are TMPRSS2-ERG, PCA3, PCAT14, ERG, TRGV9, OR51E2, TFF3, SCHLAP1, HOXC6, SPON2, and TMSB15A. In some embodiments, the at least eleven genes are TMPRSS2-ERG, PCA3, PCAT14, ERG, TRGV9, OR51E2, TFF3, SCHLAP1, HOXC6, SPON2 and PCGEM1. In some embodiments, the at least eleven genes are TMPRSS2-ERG, PCA3, PCAT14, ERG, TRGV9, OR51E2, TFF3, SCHLAP1, HOXC6, NKAIN1 and PCGEM1. In some embodiments, the at least eleven genes are TMPRSS2-ERG, PCA3, PCAT14, ERG, TRGV9, OR51E2, TFF3, SCHLAP1, HOXC6, B3GNT6 and PCGEM1. In some embodiments, the at least eleven genes are TMPRSS2-ERG, PCA3, PCAT14, ERG, TRGV9, OR51E2, TFF3, SCHLAP1, HOXC6, SPON2 and KLK4. In some embodiments, the at least eleven genes are TMPRSS2-ERG, PCA3, PCAT14, ERG, TRGV9, OR51E2, TFF3, SCHLAP1, CAMKK2, NKAIN1 and PCGEM1. In some embodiments, the at least eleven genes are TMPRSS2-ERG, PCA3, PCAT14, ERG, TRGV9, OR51E2, TFF3, SCHLAP1, CAMKK2, KLK4 and NKAIN1. In some embodiments, the at least eleven genes are TMPRSS2-ERG, PCA3, PCAT14, ERG, TRGV9, OR51E2, TFF3, SCHLAP1, CAMKK2, B3GNT6 and KLK4. In some embodiments, the at least eleven genes are TMPRSS2-ERG, PCA3, PCAT14, ERG, TRGV9, OR51E2, TFF3, SCHLAP1, CAMKK2, SPON2 and TMSB15A. In some embodiments, the at least eleven genes are TMPRSS2-ERG, PCA3, PCAT14, ERG, TRGV9, OR51E2, TFF3, SCHLAP1, APOC1, B3GNT6 and KLK4. In some embodiments, the at least eleven genes are TMPRSS2-ERG, PCA3, PCAT14, ERG, TRGV9, OR51E2, TFF3, SCHLAP1, APOC1, NKAIN1 and PCGEM1. In some embodiments, the at least eleven genes are TMPRSS2-ERG, PCA3, PCAT14, ERG, TRGV9, OR51E2, TFF3, SCHLAP1, APOC1, TMSB15A and B3GNT6. In some embodiments, the at least eleven genes are TMPRSS2-ERG, PCA3, PCAT14, ERG, TRGV9, OR51E2, TFF3, SCHLAP1, APOC1, SPON2 and TMSB15A. In some embodiments, the at least eleven genes are TMPRSS2-ERG, PCA3, PCAT14, ERG, TRGV9, OR51E2, TFF3, SCHLAP1, APOC1, TMSB15A and NKAIN1. In some embodiments, the at least eleven genes are TMPRSS2-ERG, PCA3, PCAT14, ERG, TRGV9, OR51E2, TFF3, SCHLAP1, APOC1, B3GNT6 and PCGEM1. In some embodiments, the at least eleven genes are TMPRSS2-ERG, PCA3, PCAT14, ERG, TRGV9, OR51E2, TFF3, SCHLAP1, TMSB15A, KLK4 and PCGEM1.

In some embodiments, the methods and kits described herein are useful for detecting a level or an amount of expression of at least twelve genes selected from TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6. In some embodiments, the at least twelve genes are TMPRSS2-ERG, PCA3, PCAT14, ERG, TRGV9, OR51E2, TFF3, SCHLAP1, HOXC6, CAMKK2, NKAIN1 and PCGEM1. In some embodiments, the at least twelve genes are TMPRSS2-ERG, PCA3, PCAT14, ERG, TRGV9, OR51E2, TFF3, SCHLAP1, HOXC6, CAMKK2, B3GNT6 and KLK4. In some embodiments, the at least twelve genes are TMPRSS2-ERG, PCA3, PCAT14, ERG, TRGV9, OR51E2, TFF3, SCHLAP1, HOXC6, CAMKK2, KLK$ and NKAIN1. In some embodiments, the at least twelve genes are TMPRSS2-ERG, PCA3, PCAT14, ERG, TRGV9, OR51E2, TFF3, SCHLAP1, HOXC6, CAMKK2, SPON2 and PCGEM1. In some embodiments, the at least twelve genes are TMPRSS2-ERG, PCA3, PCAT14, ERG, TRGV9, OR51E2, TFF3, SCHLAP1, HOXC6, CAMKK2, SPON2 and TMSB15A. In some embodiments, the at least twelve genes are TMPRSS2-ERG, PCA3, PCAT14, ERG, TRGV9, OR51E2, TFF3, SCHLAP1, HOXC6, SPON2, NKAIN1 and PCGEM1. In some embodiments, the at least twelve genes are TMPRSS2-ERG, PCA3, PCAT14, ERG, TRGV9, OR51E2, TFF3, SCHLAP1, HOXC6, SPON2, TMSB15A and PCGEM1. In some embodiments, the at least twelve genes are TMPRSS2-ERG, PCA3, PCAT14, ERG, TRGV9, OR51E2, TFF3, SCHLAP1, HOXC6, SPON2, TMSB15A and APOC1. In some embodiments, the at least twelve genes are TMPRSS2-ERG, PCA3, PCAT14, ERG, TRGV9, OR51E2, TFF3, SCHLAP1, HOXC6, SPON2, TMSB15A and NKAIN1. In some embodiments, the at least twelve genes are TMPRSS2-ERG, PCA3, PCAT14, ERG, TRGV9, OR51E2, TFF3, SCHLAP1, HOXC6, SPON2, APOC1 and B3GNT6. In some embodiments, the at least twelve genes are TMPRSS2-ERG, PCA3, PCAT14, ERG, TRGV9, OR51E2, TFF3, SCHLAP1, HOXC6, SPON2, KLK4 and PCGEM1. In some embodiments, the at least twelve genes are TMPRSS2-ERG, PCA3, PCAT14, ERG, TRGV9, OR51E2, TFF3, SCHLAP1, HOXC6, TMSB15A, APOC1 and B3GNT6. In some embodiments, the at least twelve genes are TMPRSS2-ERG, PCA3, PCAT14, ERG, TRGV9, OR51E2, TFF3, SCHLAP1, HOXC6, TMSB15A, APOC1 and PCGEM1. In some embodiments, the at least twelve genes are TMPRSS2-ERG, PCA3, PCAT14, ERG, TRGV9, OR51E2, TFF3, SCHLAP1, HOXC6, TMSB15A, B3GNT6 and KLK4. In some embodiments, the at least twelve genes are TMPRSS2-ERG, PCA3, PCAT14, ERG, TRGV9, OR51E2, TFF3, SCHLAP1, HOXC6, TMSB15A, APOC1 and NKAIN1. In some embodiments, the at least twelve genes are TMPRSS2-ERG, PCA3, PCAT14, ERG, TRGV9, OR51E2, TFF3, SCHLAP1, HOXC6, APOC1, B3GNT6 and KLK4. In some embodiments, the at least twelve genes are TMPRSS2-ERG, PCA3, PCAT14, ERG, TRGV9, OR51E2, TFF3, SCHLAP1, HOXC6, KLK4, NKAIN1 and PCGEM1. In some embodiments, the at least twelve genes are TMPRSS2-ERG, PCA3, PCAT14, ERG, TRGV9, OR51E2, TFF3, SCHLAP1, HOXC6, B3GNT6, NKAIN1 and PCGEM1. In some embodiments, the at least twelve genes are TMPRSS2-ERG, PCA3, PCAT14, ERG, TRGV9, OR51E2, TFF3, SCHLAP1, HOXC6, APOC1, B3GNT6 and PCGEM1.

In some embodiments, the methods and kits described herein are useful for detecting a level or an amount of expression of at least thirteen genes selected from TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6. In some embodiments, the at least thirteen genes are TMPRSS2-ERG, PCA3, PCAT14, ERG, TRGV9, OR51E2, TFF3, SCHLAP1, HOXC6, SPON2, TMSB15A, APOC1 and B3GNT6. In some embodiments, the at least thirteen genes are TMPRSS2-ERG, PCA3, PCAT14, ERG, TRGV9, OR51E2, TFF3, SCHLAP1, HOXC6, SPON2, TMSB15A, NKAIN1 and PCGEM1. In some embodiments, the at least thirteen genes are TMPRSS2-ERG, PCA3, PCAT14, ERG, TRGV9, OR51E2, TFF3, SCHLAP1, HOXC6, SPON2, TMSB15A, KLK4 and PCGEM1. In some embodiments, the at least thirteen genes are TMPRSS2-ERG, PCA3, PCAT14, ERG, TRGV9, OR51E2, TFF3, SCHLAP1, HOXC6, SPON2, TMSB15A, APOC1 and CAMKK2. In some embodiments, the at least thirteen genes are TMPRSS2-ERG, PCA3, PCAT14, ERG, TRGV9, OR51E2, TFF3, SCHLAP1, HOXC6, SPON2, TMSB15A, KLK4 and CAMKK2. In some embodiments, the at least thirteen genes are TMPRSS2-ERG, PCA3, PCAT14, ERG, TRGV9, OR51E2, TFF3, SCHLAP1, HOXC6, TMSB15A, APOC1, B3GNT6 and KLK4. In some embodiments, the at least thirteen genes are TMPRSS2-ERG, PCA3, PCAT14, ERG, TRGV9, OR51E2, TFF3, SCHLAP1, HOXC6, TMSB15A, APOC1, B3GNT6 and NKAIN1. In some embodiments, the at least thirteen genes are TMPRSS2-ERG, PCA3, PCAT14, ERG, TRGV9, OR51E2, TFF3, SCHLAP1, HOXC6, TMSB15A, APOC1, CAMKK2 and NKAIN1. In some embodiments, the at least thirteen genes are TMPRSS2-ERG, PCA3, PCAT14, ERG, TRGV9, OR51E2, TFF3, SCHLAP1, HOXC6, APOC1, B3GNT6, KLK4 and CAMKK2. In some embodiments, the at least thirteen genes are TMPRSS2-ERG, PCA3, PCAT14, ERG, TRGV9, OR51E2, TFF3, SCHLAP1, HOXC6, APOC1, B3GNT6, KLK4 and PGEM1. In some embodiments, the at least thirteen genes are TMPRSS2-ERG, PCA3, PCAT14, ERG, TRGV9, OR51E2, TFF3, SCHLAP1, HOXC6, APOC1, B3GNT6, NKAIN1 and PCGEM1. In some embodiments, the at least thirteen genes are TMPRSS2-ERG, PCA3, PCAT14, ERG, TRGV9, OR51E2, TFF3, SCHLAP1, HOXC6, B3GNT6, KLK4, NKAIN1 and CAMKK2. In some embodiments, the at least thirteen genes are TMPRSS2-ERG, PCA3, PCAT14, ERG, TRGV9, OR51E2, TFF3, SCHLAP1, HOXC6, KLK4, CAMKK2, NKAIN1 and PCGEM1. In some embodiments, the at least thirteen genes are TMPRSS2-ERG, PCA3, PCAT14, ERG, TRGV9, OR51E2, TFF3, SCHLAP1, HOXC6, SPON2, CAMKK2, NKAIN1 and PCGEM1. In some embodiments, the at least thirteen genes are TMPRSS2-ERG, PCA3, PCAT14, ERG, TRGV9, OR51E2, TFF3, SCHLAP1, HOXC6, APOC1, CAMKK2, NKAIN1, and PCGEM1. In some embodiments, the at least thirteen genes are TMPRSS2-ERG, PCA3, PCAT14, ERG, TRGV9, OR51E2, TFF3, SCHLAP1, HOXC6, SPON2, B3GNT6, NKAIN1, and PCGEM1. In some embodiments, the at least thirteen genes are TMPRSS2-ERG, PCA3, PCAT14, ERG, TRGV9, OR51E2, TFF3, SCHLAP1, HOXC6, SPON2, B3GNT6, KLK4 and CAMKK2. In some embodiments, the at least thirteen genes are TMPRSS2-ERG, PCA3, PCAT14, ERG, TRGV9, OR51E2, TFF3, SCHLAP1, HOXC6, SPON2, B3GNT6, KLK$ and PCGEM1. In some embodiments, the at least thirteen genes are TMPRSS2-ERG, PCA3, PCAT14, ERG, TRGV9, OR51E2, TFF3, SCHLAP1, HOXC6, SPON2, APOC1, KLK4 and NKAIN1. In some embodiments, the at least thirteen genes are TMPRSS2-ERG, PCA3, PCAT14, ERG, TRGV9, OR51E2, TFF3, SCHLAP1, HOXC6, TMSB15A, B3GNT6, CAMKK2 and PCGEM1.

In some embodiments, the methods and kits described herein are useful for detecting a level or an amount of expression of at least fourteen genes selected from TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6. In some embodiments, the at least fourteen genes are TMPRSS2-ERG, PCA3, PCAT14, ERG, TRGV9, OR51E2, TFF3, SCHLAP1, HOXC6, PCGEM1, B3GNT6, KLK4, CAMKK2, and NKAIN1. In some embodiments, the at least fourteen genes are TMPRSS2-ERG, PCA3, PCAT14, ERG, TRGV9, OR51E2, TFF3, SCHLAP1, HOXC6, PCGEM1, SPON2, KLK4, CAMKK2 and NKAIN1. In some embodiments, the at least fourteen genes are TMPRSS2-ERG, PCA3, PCAT14, ERG, TRGV9, OR51E2, TFF3, SCHLAP1, HOXC6, PCGEM1, SPON2, TMSB15A, CAMKK2 and NKAIN1. In some embodiments, the at least fourteen genes are TMPRSS2-ERG, PCA3, PCAT14, ERG, TRGV9, OR51E2, TFF3, SCHLAP1, HOXC6, PCGEM1, SPON2, TMSB15A, APOC1 and NKAIN1. In some embodiments, the at least fourteen genes are TMPRSS2-ERG, PCA3, PCAT14, ERG, TRGV9, OR51E2, TFF3, SCHLAP1, HOXC6, PCGEM1, SPON2, TMSB15A, APOC1, and B3GNT6. In some embodiments, the at least fourteen genes are TMPRSS2-ERG, PCA3, PCAT14, ERG, TRGV9, OR51E2, TFF3, SCHLAP1, HOXC6, PCGEM1, NKAIN1, CAMKK2, KLK4 and APOC1. In some embodiments, the at least fourteen genes are TMPRSS2-ERG, PCA3, PCAT14, ERG, TRGV9, OR51E2, TFF3, SCHLAP1, HOXC6, PCGEM1, NKAIN1, CAMKK2, B3GNT6 and SPON2. In some embodiments, the at least fourteen genes are TMPRSS2-ERG, PCA3, PCAT14, ERG, TRGV9, OR51E2, TFF3, SCHLAP1, HOXC6, PCGEM1, NKAIN1, KLK4, TMSB15A and SPON2. In some embodiments, the at least fourteen genes are TMPRSS2-ERG, PCA3, PCAT14, ERG, TRGV9, OR51E2, TFF3, SCHLAP1, HOXC6, PCGEM1, CAMKK2, APOC1, TMSB15A and SPON2. In some embodiments, the at least fourteen genes are TMPRSS2-ERG, PCA3, PCAT14, ERG, TRGV9, OR51E2, TFF3, SCHLAP1, HOXC6, PCGEM1, NKAIN1, CAMKK2, B3GNT6 and APOC1. In some embodiments, the at least fourteen genes are TMPRSS2-ERG, PCA3, PCAT14, ERG, TRGV9, OR51E2, TFF3, SCHLAP1, HOXC6, PCGEM1, NKAIN1, KLK4, B3GNT6 and SPON2. In some embodiments, the at least fourteen genes are TMPRSS2-ERG, PCA3, PCAT14, ERG, TRGV9, OR51E2, TFF3, SCHLAP1, HOXC6, PCGEM1, CAMKK2, KLK4, TMSB15A and SPON1. In some embodiments, the at least fourteen genes are TMPRSS2-ERG, PCA3, PCAT14, ERG, TRGV9, OR51E2, TFF3, SCHLAP1, HOXC6, PCGEM1, NKAIN1, B3GNT6, APOC1 and TMSB15A. In some embodiments, the at least fourteen genes are TMPRSS2-ERG, PCA3, PCAT14, ERG, TRGV9, OR51E2, TFF3, SCHLAP1, HOXC6, PCGEM1, KLK4, B3GNT6, APOC1 and SPON2. In some embodiments, the at least fourteen genes are TMPRSS2-ERG, PCA3, PCAT14, ERG, TRGV9, OR51E2, TFF3, SCHLAP1, HOXC6, PCGEM1, NKAIN1, KLK4, B3GNT6 and APOC1. In some embodiments, the at least fourteen genes are TMPRSS2-ERG, PCA3, PCAT14, ERG, TRGV9, OR51E2, TFF3, SCHLAP1, HOXC6, PCGEM1, CAMKK2, KLK4, B3GNT6, and SPON2. In some embodiments, the at least fourteen genes are TMPRSS2-ERG, PCA3, PCAT14, ERG, TRGV9, OR51E2, TFF3, SCHLAP1, HOXC6, PCGEM1, NKAIN1, CAMKK2, APOC1 and TMSB15A. In some embodiments, the at least fourteen genes are TMPRSS2-ERG, PCA3, PCAT14, ERG, TRGV9, OR51E2, TFF3, SCHLAP1, HOXC6, PCGEM1, NKIAN1, B3GNT6, APOC1 and SPON2. In some embodiments, the at least fourteen genes are TMPRSS2-ERG, PCA3, PCAT14, ERG, TRGV9, OR51E2, TFF3, SCHLAP1, HOXC6, PCGEM1, KLK4, B3GNT6, TMSB15A and SPON2. In some embodiments, the at least fourteen genes are TMPRSS2-ERG, PCA3, PCAT14, ERG, TRGV9, OR51E2, TFF3, SCHLAP1, HOXC6, PCGEM1, CAMKK2, KLK4, B3GNT6 and APOC1. In some embodiments, the at least fourteen genes are TMPRSS2-ERG, PCA3, PCAT14, ERG, TRGV9, OR51E2, TFF3, SCHLAP1, HOXC6, PCGEM1, NKAIN1, CAMKK2, KLK4 and TMSB15A. In some embodiments, the at least fourteen genes are TMPRSS2-ERG, PCA3, PCAT14, ERG, TRGV9, OR51E2, TFF3, SCHLAP1, HOXC6, PCGEM1, NKAIN1, CAMKK2, APOC1 and SPON2. In some embodiments, the at least fourteen genes are TMPRSS2-ERG, PCA3, PCAT14, ERG, TRGV9, OR51E2, TFF3, SCHLAP1, HOXC6, PCGEM1, NKAIN1, B3GNT6, TMSB15A and SPON2. In some embodiments, the at least fourteen genes are TMPRSS2-ERG, PCA3, PCAT14, ERG, TRGV9, OR51E2, TFF3, SCHLAP1, HOXC6, PCGEM1, NKAIN1, CAMKK2, B3GNT6, and TMSB15A. In some embodiments, the at least fourteen genes are TMPRSS2-ERG, PCA3, PCAT14, ERG, TRGV9, OR51E2, TFF3, SCHLAP1, HOXC6, PCGEM1, NKAIN1, KLK4, APOC1 and SPON2. In some embodiments, the at least fourteen genes are TMPRSS2-ERG, PCA3, PCAT14, ERG, TRGV9, OR51E2, TFF3, SCHLAP1, HOXC6, PCGEM1, CAMKK2, B3GNT6, TMSB15A and SPON2. In some embodiments, the at least fourteen genes are TMPRSS2-ERG, PCA3, PCAT14, ERG, TRGV9, OR51E2, TFF3, SCHLAP1, HOXC6, PCGEM1, CAMKK2, B3GNT6, APOC1 and TMSB15A. In some embodiments, the at least fourteen genes are TMPRSS2-ERG, PCA3, PCAT14, ERG, TRGV9, OR51E2, TFF3, SCHLAP1, HOXC6, PCGEM1, CAMKK2, KLK4, B3GNT6 and TMSB15A. In some embodiments, the at least fourteen genes are TMPRSS2-ERG, PCA3, PCAT14, ERG, TRGV9, OR51E2, TFF3, SCHLAP1, HOXC6, PCGEM1, KLK4, APOC1, TMSB15A and SPON2. In some embodiments, the at least fourteen genes are TMPRSS2-ERG, PCA3, PCAT14, ERG, TRGV9, OR51E2, TFF3, SCHLAP1, HOXC6, NKAIN1, B3GNT6, APOC1, TMSB15A and SPON2. In some embodiments, the at least fourteen genes are TMPRSS2-ERG, PCA3, PCAT14, ERG, TRGV9, OR51E2, TFF3, SCHLAP1, HOXC6, NKAIN1, CAMKK2, APOC1, TMSB15A and SPON2. In some embodiments, the at least fourteen genes are TMPRSS2-ERG, PCA3, PCAT14, ERG, TRGV9, OR51E2, TFF3, SCHLAP1, HOXC6, NKAIN1, CAMKK2, KLK4, TMSB15A and SPON2. In some embodiments, the at least fourteen genes are TMPRSS2-ERG, PCA3, PCAT14, ERG, TRGV9, OR51E2, TFF3, SCHLAP1, HOXC6, NKAIN1, CAMKK2, KLK4, B3GNT6 and SPON2. In some embodiments, the at least fourteen genes are TMPRSS2-ERG, PCA3, PCAT14, ERG, TRGV9, OR51E2, TFF3, SCHLAP1, HOXC6, NKAIN1, CAMKK2, KLK4, APOC1 and SPON2. In some embodiments, the at least fourteen genes are TMPRSS2-ERG, PCA3, PCAT14, ERG, TRGV9, OR51E2, TFF3, SCHLAP1, HOXC6, NKAIN1, KLK4, B3GNT6, APOC1 and SPON2. In some embodiments, the at least fourteen genes are TMPRSS2-ERG, PCA3, PCAT14, ERG, TRGV9, OR51E2, TFF3, SCHLAP1, HOXC6, NKAIN1, KLK4, APOC1, TMSB15A and SPON2. In some embodiments, the at least fourteen genes are TMPRSS2-ERG, PCA3, PCAT14, ERG, TRGV9, OR51E2, TFF3, SCHLAP1, HOXC6, KLK4, B3GNT6, APOC1, TMSB15A and SPON2.

In some embodiments, the methods and kits described herein are useful for detecting a level or an amount of expression of at least fifteen genes selected from TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6. In some embodiments, the at least fifteen genes are TMPRSS2-ERG, PCA3, PCAT14, ERG, TRGV9, OR51E2, TFF3, SCHLAP1, HOXC6, SPON2, B3GNT6, KLK4, CAMKK2, NKAIN1 and PCGEM1. In some embodiments, the at least fifteen genes are TMPRSS2-ERG, PCA3, PCAT14, ERG, TRGV9, OR51E2, TFF3, SCHLAP1, HOXC6, SPON2, TMSB15A, KLK4, CAMKK2, NKAIN1 and PCGEM1. In some embodiments, the at least fifteen genes are TMPRSS2-ERG, PCA3, PCAT14, ERG, TRGV9, OR51E2, TFF3, SCHLAP1, HOXC6, SPON2, TMSB15A, APOC1, CAMKK2, NKAIN1 and PCGEM1. In some embodiments, the at least fifteen genes are TMPRSS2-ERG, PCA3, PCAT14, ERG, TRGV9, OR51E2, TFF3, SCHLAP1, HOXC6, SPON2, TMSB15A, APOC1, B3GNT6, NKAIN1 and PCGEM1. In some embodiments, the at least fifteen genes are TMPRSS2-ERG, PCA3, PCAT14, ERG, TRGV9, OR51E2, TFF3, SCHLAP1, HOXC6, SPON2, TMSB15A, APOC1, B3GNT6, KLK4 and PCEM1. In some embodiments, the at least fifteen genes are TMPRSS2-ERG, PCA3, PCAT14, ERG, TRGV9, OR51E2, TFF3, SCHLAP1, HOXC6, SPON2, TMSB15A, APOC1, B3GNT6, KLK4 and CAMKK2. In some embodiments, the at least fifteen genes are TMPRSS2-ERG, PCA3, PCAT14, ERG, TRGV9, OR51E2, TFF3, SCHLAP1, HOXC6, SPON2, TMSB15A, B3GNT6, CAMKK2, NKAIN1 and PCGEM1. In some embodiments, the at least fifteen genes are TMPRSS2-ERG, PCA3, PCAT14, ERG, TRGV9, OR51E2, TFF3, SCHLAP1, HOXC6, SPON2, TMSB15A, APOC1, KLK4, NKAIN1 and PCGEM1. In some embodiments, the at least fifteen genes are TMPRSS2-ERG, PCA3, PCAT14, ERG, TRGV9, OR51E2, TFF3, SCHLAP1, HOXC6, SPON2, TMSB15A, APOC1, B3GNT6, CAMKK2 and PCGEM1. In some embodiments, the at least fifteen genes are TMPRSS2-ERG, PCA3, PCAT14, ERG, TRGV9, OR51E2, TFF3, SCHLAP1, HOXC6, SPON2, TMSB15A, APOC1, B3GNT6, KLK4, and NKAIN1. In some embodiments, the at least fifteen genes are TMPRSS2-ERG, PCA3, PCAT14, ERG, TRGV9, OR51E2, TFF3, SCHLAP1, HOXC6, SPON2, APOC1, KLK4, CAMKK2, NKAIN1 and PCGEM1. In some embodiments, the at least fifteen genes are TMPRSS2-ERG, PCA3, PCAT14, ERG, TRGV9, OR51E2, TFF3, SCHLAP1, HOXC6, SPON2, APOC1, B3GNT6, CAMKK2, NKAIN1 and PCGEM1. In some embodiments, the at least fifteen genes are TMPRSS2-ERG, PCA3, PCAT14, ERG, TRGV9, OR51E2, TFF3, SCHLAP1, HOXC6, SPON2, TMSB15A, B3GNT6, KLK4, NKAIN1 and PCGEM1. In some embodiments, the at least fifteen genes are TMPRSS2-ERG, PCA3, PCAT14, ERG, TRGV9, OR51E2, TFF3, SCHLAP1, HOXC6, SPON2, TMSB15A, APOC1, KLK4, CAMKK2 and PCGEM1. In some embodiments, the at least fifteen genes are TMPRSS2-ERG, PCA3, PCAT14, ERG, TRGV9, OR51E2, TFF3, SCHLAP1, HOXC6, SPON2, TMSB15A, APOC1, B3GNT6, CAMKK2 and NKAIN1. In some embodiments, the at least fifteen genes are TMPRSS2-ERG, PCA3, PCAT14, ERG, TRGV9, OR51E2, TFF3, SCHLAP1, HOXC6, SPON2, TMSB15A, B3GNT6, KLK4, CAMKK2 and PCGEM1. In some embodiments, the at least fifteen genes are TMPRSS2-ERG, PCA3, PCAT14, ERG, TRGV9, OR51E2, TFF3, SCHLAP1, HOXC6, SPON2, TMSB15A, APOC1, KLK4, CAMKK2 and NKAIN1. In some embodiments, the at least fifteen genes are TMPRSS2-ERG, PCA3, PCAT14, ERG, TRGV9, OR51E2, TFF3, SCHLAP1, HOXC6, SPON2, TMSB15A, B3GNT6, KLK4, CAMKK2 and NKAIN1. In some embodiments, the at least fifteen genes are TMPRSS2-ERG, PCA3, PCAT14, ERG, TRGV9, OR51E2, TFF3, SCHLAP1, TMSB15A, APOC1, B3GNT6, KLK4, CAMKK2, NKAIN1 and PCGEM1. In some embodiments, the at least fifteen genes are TMPRSS2-ERG, PCA3, PCAT14, ERG, TRGV9, OR51E2, TFF3, SCHLAP1, HOXC6, APOC1, B3GNT6, KLK4, CAMKK2, NKAIN1 and PCGEM1. In some embodiments, the at least fifteen genes are TMPRSS2-ERG, PCA3, PCAT14, ERG, TRGV9, OR51E2, TFF3, SCHLAP1, SPON2, APOC1, B3GNT6, KLK4, CAMKK2, NKAIN1 an PCGEM1. In some embodiments, the at least fifteen genes are TMPRSS2-ERG, PCA3, PCAT14, ERG, TRGV9, OR51E2, TFF3, SCHLAP1, HOXC6, TMSB15A, B3GNT6, KLK4, CAMKK2, NKAIN1 and PCGEM1. In some embodiments, the at least fifteen genes are TMPRSS2-ERG, PCA3, PCAT14, ERG, TRGV9, OR51E2, TFF3, SCHLAP1, SPON2, TMSB15A, APOC1, B3GNT6, KLK4, CAMKK2 and NKAIN1. In some embodiments, the at least fifteen genes are TMPRSS2-ERG, PCA3, PCAT14, ERG, TRGV9, OR51E2, TFF3, SCHLAP1, SPON2, TMSB15A, B3GNT6, KLK4, CAMKK2, NKAIN1 and PCGEM1. In some embodiments, the at least fifteen genes are TMPRSS2-ERG, PCA3, PCAT14, ERG, TRGV9, OR51E2, TFF3, SCHLAP1, SPON2, TMSB15A, APOC1, B3GNT6, KLK4, NKAIN1 and PCGEM1. In some embodiments, the at least fifteen genes are TMPRSS2-ERG, PCA3, PCAT14, ERG, TRGV9, OR51E2, TFF3, SCHLAP1, SPON2, TMSB15A, APOC1, KLK4, CAMKK2, NKAIN1 and PCGEM1. In some embodiments, the at least fifteen genes are TMPRSS2-ERG, PCA3, PCAT14, ERG, TRGV9, OR51E2, TFF3, SCHLAP1, HOXC6, TMSB15A, APOC1, KLK4, CAMKK2, NKAIN1 and PCGEM1. In some embodiments, the at least fifteen genes are TMPRSS2-ERG, PCA3, PCAT14, ERG, TRGV9, OR51E2, TFF3, SCHLAP1, HOXC6, TMSB15A, APOC1, B3GNT6, KLK4, CAMKK2 and PCGEM1. In some embodiments, the at least fifteen genes are TMPRSS2-ERG, PCA3, PCAT14, ERG, TRGV9, OR51E2, TFF3, SCHLAP1, HOXC6, TMSB15A, APOC1, B3GNT6, CAMKK2, NKAIN1 and PCGEM1. In some embodiments, the at least fifteen genes are TMPRSS2-ERG, PCA3, PCAT14, ERG, TRGV9, OR51E2, TFF3, SCHLAP1, HOXC6, SPON2, APOC1, B3GNT6, KLK4, CAMKK2 and NKAIN1. In some embodiments, the at least fifteen genes are TMPRSS2-ERG, PCA3, PCAT14, ERG, TRGV9, OR51E2, TFF3, SCHLAP1, HOXC6, SPON2, APOC1, B3GNT6, KLK4, NKAIN1 and PCGEM1. In some embodiments, the at least fifteen genes are TMPRSS2-ERG, PCA3, PCAT14, ERG, TRGV9, OR51E2, TFF3, SCHLAP1, HOXC6, SPON2, APOC1, B3GNT6, KLK4, CAMKK2 and PCGEM1. In some embodiments, the at least fifteen genes are TMPRSS2-ERG, PCA3, PCAT14, ERG, TRGV9, OR51E2, TFF3, SCHLAP1, HOXC6, TMSB15A, APOC1, B3GNT6, KLK4, NKAIN1 and PCGEM1. In some embodiments, the at least fifteen genes are TMPRSS2-ERG, PCA3, PCAT14, ERG, TRGV9, OR51E2, TFF3, SCHLAP1, HOXC6, TMSB15A, APOC1, B3GNT6, KLK4, NKAIN1 and PCGEM1. In some embodiments, the at least fifteen genes are TMPRSS2-ERG, PCA3, PCAT14, ERG, TRGV9, OR51E2, TFF3, SCHLAP1, HOXC6, TMSB15A, APOC1, B3GNT6, CAMKK2, NKAIN1 and PCGEM1. In some embodiments, the at least fifteen genes are TMPRSS2-ERG, PCA3, PCAT14, ERG, TRGV9, OR51E2, TFF3, SCHLAP1, HOXC6, TMSB15A, APOC1, B3GNT6,KLK$, CAMKK2 and PCGEM1. In some embodiments, the at least fifteen genes are TMPRSS2-ERG, PCA3, PCAT14, ERG, TRGV9, OR51E2, TFF3, SCHLAP1, HOXC6, TMSB15A, APOC1, KLK4, CAMKK2, NKAIN1 and PCGEM1. In some embodiments, the at least fifteen genes are TMPRSS2-ERG, PCA3, PCAT14, ERG, TRGV9, OR51E2, TFF3, SCHLAP1, HOXC6, TMSB15A, APOC1, B3GNT6, KLK4, CAMKK2 and NKAIN1. In some embodiments, the at least fifteen genes are TMPRSS2-ERG, PCA3, PCAT14, ERG, TRGV9, OR51E2, TFF3, SCHLAP1, HOXC6, SPON2, APOC1, B3GNT6, KLK4, CAMKK2 and PCGEM1. In some embodiments, the at least fifteen genes are TMPRSS2-ERG, PCA3, PCAT14, ERG, TRGV9, OR51E2, TFF3, SCHLAP1, HOXC6, SPON2, APOC1, B3GNT6, KLK4, NKAIN1 and PCGEM1. In some embodiments, the at least fifteen genes are TMPRSS2-ERG, PCA3, PCAT14, ERG, TRGV9, OR51E2, TFF3, SCHLAP1, HOXC6, SPON2, TMSB15A, B3GNT6, KLK4, CAMKK2 and PCGEM1. In some embodiments, the at least fifteen genes are TMPRSS2-ERG, PCA3, PCAT14, ERG, TRGV9, OR51E2, TFF3, SCHLAP1, HOXC6, SPON2, APOC1, B3GNT6, CAMKK2, NKAIN1 and PCGEM1. In some embodiments, the at least fifteen genes are TMPRSS2-ERG, PCA3, PCAT14, ERG, TRGV9, OR51E2, TFF3, SCHLAP1, HOXC6, SPON2, TMSB15A, B3GNT6, KLK4, NKAIN1 and PCGEM1. In some embodiments, the at least fifteen genes are TMPRSS2-ERG, PCA3, PCAT14, ERG, TRGV9, OR51E2, TFF3, SCHLAP1, HOXC6, SPON2, TMSB15A, APOC1, KLK4, CAMKK2 and PCGEM1. In some embodiments, the at least fifteen genes are TMPRSS2-ERG, PCA3, PCAT14, ERG, TRGV9, OR51E2, TFF3, SCHLAP1, HOXC6, SPON2, TMSB15A, APOC1, B3GNT6, CAMKK2 and NKAIN1. In some embodiments, the at least fifteen genes are TMPRSS2-ERG, PCA3, PCAT14, ERG, TRGV9, OR51E2, TFF3, SCHLAP1, SPON2, TMSB15A, APOC1, B3GNT6, CAMKK2, NKAIN1 and PCGEM1. In some embodiments, the at least fifteen genes are TMPRSS2-ERG, PCA3, PCAT14, ERG, TRGV9, OR51E2, TFF3, SCHLAP1, SPON2, TMSB15A, APOC1, KLK4, CAMKK2, NKAIN1 and PCGEM1. In some embodiments, the at least fifteen genes are TMPRSS2-ERG, PCA3, PCAT14, ERG, TRGV9, OR51E2, TFF3, SCHLAP1, SPON2, TMSB15A, APOC1, B3NGTZ6, KLK4, NKAIN1 and PCGEM1. In some embodiments, the at least fifteen genes are TMPRSS2-ERG, PCA3, PCAT14, ERG, TRGV9, OR51E2, TFF3, SCHLAP1, SPON2, TMSB15A, APOC1, B3GNT6, KLK4, CAMKK2 and PCGEM1. In some embodiments, the at least fifteen genes are TMPRSS2-ERG, PCA3, PCAT14, ERG, TRGV9, OR51E2, TFF3, SCHLAP1, SPON2, TMSB15A, B3GNT6, KLK4, CAMKK2, NKAIN1 and PCGEM1.

In some embodiments, the methods and kits described herein are useful for detecting a level or an amount of expression of at least sixteen genes selected from TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6. In some embodiments, the at least sixteen genes are TMPRSS2-ERG, PCA3, PCAT14, ERG, TRGV9, OR51E2, TFF3, SCHLAP1, HOXC6, SPON2, TMSB15A, APOC1, B3GNT6, KLK4, CAMKK2 and NKAIN1. In some embodiments, the at least sixteen genes are TMPRSS2-ERG, PCA3, PCAT14, ERG, TRGV9, OR51E2, TFF3, SCHLAP1, HOXC6, SPON2, TMSB15A, APOC1, B3GNT6, KLK4, CAMKK2 and PCGEM1. In some embodiments, the at least sixteen genes are TMPRSS2-ERG, PCA3, PCAT14, ERG, TRGV9, OR51E2, TFF3, SCHLAP1, HOXC6, SPON2, TMSB15A, APOC1, B3GNT6, KLK4, NKAIN1 and PCGEM1. In some embodiments, the at least sixteen genes are TMPRSS2-ERG, PCA3, PCAT14, ERG, TRGV9, OR51E2, TFF3, SCHLAP1, HOXC6, SPON2, TMSB15A, APOC1, B3GNT6, CAMKK2, NKAIN1 and PCGEM1. In some embodiments, the at least sixteen genes are TMPRSS2-ERG, PCA3, PCAT14, ERG, TRGV9, OR51E2, TFF3, SCHLAP1, HOXC6, SPON2, TMSB15A, APOC1, KLK4, CAMKK2, NKAIN1 and PCGEM1. In some embodiments, the at least sixteen genes are TMPRSS2-ERG, PCA3, PCAT14, ERG, TRGV9, OR51E2, TFF3, SCHLAP1, HOXC6, SPON2, TMSB15A, B3NGT6, KLK4, CAMKK2, NKAIN1 and PCGEM1. In some embodiments, the at least sixteen genes are TMPRSS2-ERG, PCA3, PCAT14, ERG, TRGV9, OR51E2, TFF3, SCHLAP1, HOXC6, SPON2, APOC1, B3GNT6, KLK4, CAMKK2, NKAIN1 and PCGEM1. In some embodiments, the at least sixteen genes are TMPRSS2-ERG, PCA3, PCAT14, ERG, TRGV9, OR51E2, TFF3, SCHLAP1, HOXC6, TMSB15A, APOC1, B3GNT6, KLK4, CAMKK2, NKAIN1 and PCGEM1. In some embodiments, the at least sixteen genes are TMPRSS2-ERG, PCA3, PCAT14, ERG, TRGV9, OR51E2, TFF3, SCHLAP1, SPON2, TMSB15A, APOC1, B3GNT6, KLK4, CAMKK2, NKAIN1 and PCGEM1. In some embodiments, the at least sixteen genes are TMPRSS2-ERG, PCA3, PCAT14, ERG, TRGV9, OR51E2, TFF3, HOXC6, SPON2, TMSB15A, APOC1, B3GNT6, KLK4, CAMKK2, NKAIN1 and PCGEM1. In some embodiments, the at least sixteen genes are TMPRSS2-ERG, PCA3, PCAT14, ERG, TRGV9, OR51E2, SCHLAP1, HOXC6, SPON2, TMSB15A, APOC1, B3GNT6, KLK4, CAMKK2, NKAIN1 and PCGEM1. In some embodiments, the at least sixteen genes are TMPRSS2-ERG, PCA3, PCAT14, ERG, TRGV9, TFF3, SCHLAP1, HOXC6, SPON2, TMSB15A, APOC1, B3GNT6, KLK4, CAMKK2, NKAIN1 and PCGEM1. In some embodiments, the at least sixteen genes are TMPRSS2-ERG, PCA3, PCAT14, ERG, OR51E2, TFF3, SCHLAP1, HOXC6, SPON2, TMSB15A, APOC1, B3GNT6, KLK4, CAMKK2, NKAIN1 and PCGEM1. In some embodiments, the at least sixteen genes are TMPRSS2-ERG, PCA3, PCAT14, TRGV9, OR51E2, TFF3, SCHLAP1, HOXC6, SPON2, TMSB15A, APOC1, B3GNT6, KLK4, CAMKK2, NKAIN1 and PCGEM1. In some embodiments, the at least sixteen genes are TMPRSS2-ERG, PCA3, ERG, TRGV9, OR51E2, TFF3, SCHLAP1, HOXC6, SPON2, TMSB15A, APOC1, B3GNT6, KLK4, CAMKK2, NKAIN1 and PCGEM1.

In some embodiments, the methods and kits described herein are useful for detecting a level or an amount of expression of TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6.

In some embodiments, the level or amount of expression of the at least three, four, five, six, seven, eight, nine, ten, eleven, twelve, thirteen, fourteen, fifteen, sixteen or seventeen genes described herein is higher in a subject having or at risk of developing Grade Group ≥2 prostate cancer relative to subject having or at risk of developing Grade Group <2 prostate cancer, or in a subject having no prostate cancer. In some embodiments, the level or amount of expression of at least one of TMPRSS2-ERG, SCHLAP1, OR51E2, PCAT14, PCA3, B3GNT6, TFF3, SPON2, TRGV9, TMSB15A, ERG, KLK4, and HOXC6 is higher in a subject at risk for having Grade Group ≥2 prostate cancer than in a subject at risk for having or developing a Grade Group <2 prostate cancer or in a subject having no prostate cancer. In some embodiments, the level or amount of expression of each of TMPRSS2-ERG, SCHLAP1, OR51E2, PCAT14, PCA3, B3GNT6, TFF3, SPON2, TRGV9, TMSB15A, ERG, KLK4, and HOXC6 is higher in a subject at risk for having Grade Group ≥2 prostate cancer than in a subject at risk for having or developing a Grade Group <2 prostate cancer or in a subject having no prostate cancer. In some embodiments, the total level or amount of expression of TMPRSS2-ERG, SCHLAP1, OR51E2, PCAT14, PCA3, B3GNT6, TFF3, SPON2, TRGV9, TMSB15A, ERG, KLK4, and HOXC6 is higher in a subject at risk for having Grade Group ≥2 prostate cancer than in a subject at risk for having or developing a Grade Group <2 prostate cancer or in a subject having no prostate cancer.

In some embodiments, the level or amount of expression of the at least three, four, five, six, seven, eight, nine, ten, eleven, twelve, thirteen, fourteen, fifteen, sixteen or seventeen genes described herein is lower in a subject having or at risk of developing Grade Group ≥2 prostate cancer relative to subject having or at risk of developing Grade Group <2 prostate cancer, or in a subject having no prostate cancer. In some embodiments, the level or amount of expression of at least one of APOC1, CAMKK2, NKAIN1 and PCGEM1 is lower in a subject at risk for having Grade Group ≥2 prostate cancer than in a subject at risk for having or developing a Grade Group <2 prostate cancer or in a subject having no prostate cancer. In some embodiments, the level or amount of expression of each of APOC1, CAMKK2, NKAIN1 and PCGEM1 is lower in a subject at risk for having Grade Group ≥2 prostate cancer than in a subject at risk for having or developing a Grade Group <2 prostate cancer or in a subject having no prostate cancer. In some embodiments, the total level or amount of expression of APOC1, CAMKK2, NKAIN1 and PCGEM1 is lower in a subject at risk for having Grade Group ≥2 prostate cancer than in a subject at risk for having or developing a Grade Group <2 prostate cancer or in a subject having no prostate cancer.

### Methods for Detecting Expression of Genes

The level or amount of expression of one or more genes of the present disclosure can be detected using any of a variety of nucleic acid techniques, including but not limited to: nucleic acid sequencing; nucleic acid hybridization; and nucleic acid amplification.

In some embodiments, nucleic acid sequencing methods are utilized (e.g., for detection of amplified nucleic acids). In some embodiments, the technology provided herein finds use in a Second Generation (i.e., Next Generation or Next-Gen), Third Generation (i.e., Next-Next-Gen), or Fourth Generation (i.e., N3-Gen) sequencing technology including, but not limited to, pyrosequencing, sequencing-by-ligation, single molecule sequencing, sequence-by-synthesis (SBS), semiconductor sequencing, massive parallel clonal, massive parallel single molecule SBS, massive parallel single molecule real-time, massive parallel single molecule real-time nanopore technology, etc. Morozova and Marra provide a review of some such technologies in Genomics, 92: 255 (2008), herein incorporated by reference in its entirety. Those of skill in the art will recognize that because RNA is less stable in the cell and more prone to nuclease attack experimentally RNA can be reverse transcribed to DNA before sequencing.

A number of DNA sequencing techniques are suitable for use with the instant methods, including fluorescence-based sequencing methodologies (See, e.g., Birren et al., Genome Analysis: Analyzing DNA, 1, Cold Spring Harbor, N.Y.; herein incorporated by reference in its entirety). In some embodiments, the sequencing is automated sequencing techniques understood in the art. In some embodiments, the sequencing is parallel sequencing of partitioned amplicons (PCT Publication No: WO2006084132 to Kevin McKernan et al., herein incorporated by reference in its entirety). In some embodiments, the sequencing is DNA sequencing by parallel oligonucleotide extension (See, e.g., U.S. Pat. No. 5,750,341 to Macevicz et al., and U.S. Pat. No. 6,306,597 to Macevicz et al., both of which are herein incorporated by reference in their entireties). Additional examples of sequencing techniques include the Church polony technology (Mitra et al., 2003, Analytical Biochemistry 320, 55-65; Shendure et al., 2005 Science 309, 1728-1732; U.S. Pat. Nos. 6,432,360, 6,485,944, 6,511,803; herein incorporated by reference in their entireties), the 454 picotiter pyrosequencing technology (Margulies et al., 2005 Nature 437, 376-380; US 20050130173; herein incorporated by reference in their entireties), the Solexa single base addition technology (Bennett et al., 2005, Pharmacogenomics, 6, 373-382; U.S. Pat. Nos. 6,787,308; 6,833,246; herein incorporated by reference in their entireties), the Lynx massively parallel signature sequencing technology (Brenner et al. (2000). Nat. Biotechnol. 18:630-634; U.S. Pat. Nos. 5,695,934; 5,714,330; herein incorporated by reference in their entireties), and the Adessi PCR colony technology (Adessi et al. (2000). Nucleic Acid Res. 28, E87; WO 00018957; herein incorporated by reference in its entirety).

Illustrative non-limiting examples of nucleic acid hybridization techniques include, but are not limited to, in situ hybridization (ISH), microarray, and Southern or Northern blot. In situ hybridization (ISH) is a type of hybridization that uses a labeled complementary DNA or RNA strand as a probe to localize a specific DNA or RNA sequence in a portion or section of tissue (in situ), or, if the tissue is small enough, the entire tissue (whole mount ISH). DNA ISH can be used to determine the structure of chromosomes. RNA ISH can be used to measure and localize mRNAs and other transcripts (e.g., cancer markers) within tissue sections or whole mounts. Sample cells and tissues can be treated to fix the target transcripts in place and to increase access of the probe. The probe hybridizes to the target sequence at elevated temperature, and then the excess probe is washed away. The probe that was labeled with either radio-, fluorescent- or antigen-labeled bases is localized and quantitated in the tissue using either autoradiography, fluorescence microscopy or immunohistochemistry, respectively. ISH can also use two or more probes, labeled with radioactivity or the other non-radioactive labels, to simultaneously detect two or more transcripts.

The one or more cancer markers in the methods described herein can be detected by conducting one or more hybridization reactions. The one or more hybridization reactions may comprise one or more hybridization arrays, hybridization reactions, hybridization chain reactions, isothermal hybridization reactions, nucleic acid hybridization reactions, or a combination thereof. The one or more hybridization arrays may comprise hybridization array genotyping, hybridization array proportional sensing, DNA hybridization arrays, macroarrays, microarrays, high-density oligonucleotide arrays, genomic hybridization arrays, comparative hybridization arrays, or a combination thereof.

Different kinds of biological assays are called microarrays including, but not limited to: DNA microarrays (e.g., cDNA microarrays and oligonucleotide microarrays); protein microarrays; tissue microarrays; transfection or cell microarrays; chemical compound microarrays; and antibody microarrays. A DNA microarray, commonly known as gene chip, DNA chip, or biochip, is a collection of microscopic DNA spots attached to a solid surface (e.g., glass, plastic or silicon chip) forming an array for the purpose of expression profiling or monitoring expression levels for thousands of genes simultaneously. The affixed DNA segments are known as probes, thousands of which can be used in a single DNA microarray. Microarrays can be used to identify disease genes or transcripts (e.g., cancer markers) by comparing gene expression in disease and normal cells. Microarrays can be fabricated using a variety of technologies, including but not limited to: printing with fine-pointed pins onto glass slides; photolithography using pre-made masks; photolithography using dynamic micromirror devices; ink-jet printing; or, electrochemistry on microelectrode arrays.

The methods disclosed herein can comprise conducting one or more amplification reactions. Nucleic acids (e.g., cancer markers) may be amplified prior to or simultaneous with detection. Conducting one or more amplification reactions may comprise one or more PCR-based amplifications, non-PCR based amplifications, or a combination thereof. Illustrative non-limiting examples of nucleic acid amplification techniques include, but are not limited to, polymerase chain reaction (PCR), reverse transcription polymerase chain reaction (RT-PCR), nested PCR, linear amplification, multiple displacement amplification (MDA), real-time SDA, rolling circle amplification, circle-to-circle amplification transcription-mediated amplification (TMA), ligase chain reaction (LCR), strand displacement amplification (SDA), and nucleic acid sequence based amplification (NASBA). Those of ordinary skill in the art will recognize that certain amplification techniques (e.g., PCR) require that RNA be reversed transcribed to DNA prior to amplification (e.g., RT-PCR), whereas other amplification techniques directly amplify RNA (e.g., TMA and NASBA).

The polymerase chain reaction (U.S. Pat. Nos. 4,683,195, 4,683,202, 4,800,159 and 4,965,188, each of which is herein incorporated by reference in its entirety), commonly referred to as PCR, uses multiple cycles of denaturation, annealing of primer pairs to opposite strands, and primer extension to exponentially increase copy numbers of a target nucleic acid sequence. In a variation called RT-PCR, reverse transcriptase (RT) is used to make a complementary DNA (cDNA) from mRNA, and the cDNA is then amplified by PCR to produce multiple copies of DNA. For other various permutations of PCR see, e.g., U.S. Pat. Nos. 4,683,195, 4,683,202 and 4,800,159; Mullis et al., Meth. Enzymol. 155: 335 (1987); and Murakawa et al., DNA 7: 287 (1988), each of which is herein incorporated by reference in its entirety.

Transcription mediated amplification (U.S. Pat. Nos. 5,480,784 and 5,399,491, each of which is herein incorporated by reference in its entirety), commonly referred to as TMA, synthesizes multiple copies of a target nucleic acid sequence autocatalytically under conditions of substantially constant temperature, ionic strength, and pH in which multiple RNA copies of the target sequence autocatalytically generate additional copies. See, e.g., U.S. Pat. Nos. 5,399,491 and 5,824,518, each of which is herein incorporated by reference in its entirety. In a variation described in U.S. Publ. No. 20060046265 (herein incorporated by reference in its entirety), TMA optionally incorporates the use of blocking moieties, terminating moieties, and other modifying moieties to improve TMA process sensitivity and accuracy.

The ligase chain reaction (Weiss, R., Science 254: 1292 (1991), herein incorporated by reference in its entirety), commonly referred to as LCR, uses two sets of complementary DNA oligonucleotides that hybridize to adjacent regions of the target nucleic acid. The DNA oligonucleotides are covalently linked by a DNA ligase in repeated cycles of thermal denaturation, hybridization and ligation to produce a detectable double-stranded ligated oligonucleotide product.

Strand displacement amplification (Walker, G. et al., Proc. Natl. Acad. Sci. USA 89: 392-396 (1992); U.S. Pat. Nos. 5,270,184 and 5,455,166, each of which is herein incorporated by reference in its entirety), commonly referred to as SDA, uses cycles of annealing pairs of primer sequences to opposite strands of a target sequence, primer extension in the presence of a dNTPαS to produce a duplex hemiphosphorothioated primer extension product, endonuclease-mediated nicking of a hemimodified restriction endonuclease recognition site, and polymerase-mediated primer extension from the 3′ end of the nick to displace an existing strand and produce a strand for the next round of primer annealing, nicking and strand displacement, resulting in geometric amplification of product. Thermophilic SDA (tSDA) uses thermophilic endonucleases and polymerases at higher temperatures in essentially the same method (EP Pat. No. 0 684 315).

Other amplification methods include, for example: nucleic acid sequence-based amplification (U.S. Pat. No. 5,130,238, herein incorporated by reference in its entirety), commonly referred to as NASBA; one that uses an RNA replicase to amplify the probe molecule itself (Lizardi et al., BioTechnol. 6: 1197 (1988), herein incorporated by reference in its entirety), commonly referred to as Q3 replicase; a transcription-based amplification method (Kwoh et al., Proc. Natl. Acad. Sci. USA 86:1173 (1989)); and, self-sustained sequence replication (Guatelli et al., Proc. Natl. Acad. Sci. USA 87: 1874 (1990), each of which is herein incorporated by reference in its entirety). For further discussion of known amplification methods see Persing, David H., “In Vitro Nucleic Acid Amplification Techniques” in Diagnostic Medical Microbiology: Principles and Applications (Persing et al., Eds.), pp. 51-87 (American Society for Microbiology, Washington, DC (1993)).

In some embodiments, amplification methods are real time quantitative PCR methods (QPCR). A real-time polymerase chain reaction (real-time PCR, or qPCR) is a laboratory technique of molecular biology based on the polymerase chain reaction (PCR). It monitors the amplification of a targeted DNA molecule during the PCR (i.e., in real time), not at its end, as in conventional PCR. Real-time PCR can be used quantitatively (quantitative real-time PCR) and semi-quantitatively (i.e., above/below a certain amount of DNA molecules) (semi-quantitative real-time PCR). Two common methods for the detection of PCR products in real-time PCR are (1) non-specific fluorescent dyes that intercalate with any double-stranded DNA and (2) sequence-specific DNA probes consisting of oligonucleotides that are labelled with a fluorescent reporter, which permits detection only after hybridization of the probe with its complementary sequence.

Illustrative non-limiting examples of immunoassays include, but are not limited to: immunoprecipitation; Western blot; ELISA; immunohistochemistry; immunocytochemistry; flow cytometry; and, immuno-PCR. Polyclonal or monoclonal antibodies detectably labeled using various techniques known to those of ordinary skill in the art (e.g., colorimetric, fluorescent, chemiluminescent or radioactive) are suitable for use in the immunoassays.

Immunoprecipitation is the technique of precipitating an antigen out of solution using an antibody specific to that antigen. The process can be used to identify protein complexes present in cell extracts by targeting a protein believed to be in the complex. The complexes are brought out of solution by insoluble antibody-binding proteins isolated initially from bacteria, such as Protein A and Protein G. The antibodies can also be coupled to sepharose beads that can easily be isolated out of solution. After washing, the precipitate can be analyzed using mass spectrometry, Western blotting, or any number of other methods for identifying constituents in the complex.

A Western blot, or immunoblot, is a method to detect protein in a given sample of tissue homogenate or extract. It uses gel electrophoresis to separate denatured proteins by mass. The proteins are then transferred out of the gel and onto a membrane, typically polyvinyldiflroride or nitrocellulose, where they are probed using antibodies specific to the protein of interest. As a result, researchers can examine the amount of protein in a given sample and compare levels between several groups.

An ELISA, short for Enzyme-Linked ImmunoSorbent Assay, is a biochemical technique to detect the presence of an antibody or an antigen in a sample. It utilizes a minimum of two antibodies, one of which is specific to the antigen and the other of which is coupled to an enzyme. The second antibody will cause a chromogenic or fluorogenic substrate to produce a signal. Variations of ELISA include sandwich ELISA, competitive ELISA, and ELISPOT. Because the ELISA can be performed to evaluate either the presence of antigen or the presence of antibody in a sample, it is a useful tool both for determining serum antibody concentrations and also for detecting the presence of antigen.

Immunohistochemistry and immunocytochemistry refer to the process of localizing proteins in a tissue section or cell, respectively, via the principle of antigens in tissue or cells binding to their respective antibodies. Visualization is enabled by tagging the antibody with color producing or fluorescent tags. Typical examples of color tags include, but are not limited to, horseradish peroxidase and alkaline phosphatase. Typical examples of fluorophore tags include, but are not limited to, fluorescein isothiocyanate (FITC) or phycoerythrin (PE).

Immuno-polymerase chain reaction (IPCR) utilizes nucleic acid amplification techniques to increase signal generation in antibody-based immunoassays. Because no protein equivalence of PCR exists, that is, proteins cannot be replicated in the same manner that nucleic acid is replicated during PCR, the only way to increase detection sensitivity is by signal amplification. The target proteins are bound to antibodies which are directly or indirectly conjugated to oligonucleotides. Unbound antibodies are washed away and the remaining bound antibodies have their oligonucleotides amplified. Protein detection occurs via detection of amplified oligonucleotides using standard nucleic acid detection methods, including real-time methods.

In some embodiments, the level or amount of mRNA is detected using RT-qPCR analysis which provides Ct (cycle threshold values) for each mRNA detected. In a real-time PCR assay a positive reaction is detected by accumulation of a fluorescent signal. The Ct value is defined as the number of cycles required for the fluorescent signal to cross the threshold (i.e., exceeds the background level). Ct levels are inversely proportional to the amount of target nucleic acid in the sample (i.e., the lower the Ct value the greater the amount of mRNA in the sample).

In some embodiments, the level or amount of expression of any one of the genes described herein is normalized to a level or an amount of expression of a reference gene. In some embodiments, the amount of expression of mRNA is normalized to the level or amount of expression of mRNA of a reference gene. Reference genes suitable for normalization are known to those of skill in the art and include, but are not limited to, KLK3, CYPB561A3, EEF1A2, GAPDH, HPN, KLK2, KLK4, LBH, NUDT8, SPDEF, or TRGV. In some embodiments, the reference gene is KLK3.

Compositions for use in the methods described herein, such as reagent compositions, include, but are not limited to, antibodies, probes, amplification oligonucleotides, and the like.

The compositions and kits can comprise 1 or more, 2 or more, 3 or more, or 4 or more antibodies, probes, pairs of probes, pairs of amplification oligonucleotide, or sequencing primers.

The probes or primers can hybridize to 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 20 or more, or 21 or more target molecules. The target molecules may be RNA, DNA, cDNA, mRNA, a portion or fragment thereof or a combination thereof. In some instances, at least a portion of the target molecules are cancer markers. The probes may hybridize to 1 or more, or 2 or more cancer markers disclosed herein.

Typically, the probes or primers comprise a target specific sequence. The target specific sequence may be complementary to at least a portion of the target molecule. The target specific sequence may be at least about 50% or more, 55% or more, 60% or more, 65% or more, 70% or more, 75% or more, 80% or more, 85% or more, 90% or more, 95% or more, 97% or more, 98% or more, or 100% complementary to at least a portion of the target molecule.

The target specific sequence can be at least about 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more nucleotides in length. In some instances, the target specific sequence is between about 8 to about 20 nucleotides, 10 to about 18 nucleotides, or 12 to about 16 nucleotides in length.

The compositions and kits can comprise a plurality of probes or primers, wherein the two or more probes of the plurality of probes comprise identical target specific sequences. The compositions and kits may comprise a plurality of probes, wherein the two or more probes of the plurality of probes comprise different target specific sequences.

The probes can further comprise a unique sequence. The unique sequence is noncomplementary to the cancer marker. The unique sequence may comprise a label, barcode, or unique identifier. The unique sequence may comprise a random sequence, nonrandom sequence, or a combination thereof. The unique sequence may be at least about 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 22 or more, 24 or more, 26 or more, 28 or more, 30 or more nucleotides in length. In some instances, the unique sequence is between about 8 to about 20 nucleotides, 10 to about 18 nucleotides, or 12 to about 16 nucleotides in length.

The probes can further comprise a universal sequence. The universal sequence may comprise a primer binding site. The universal sequence may enable detection of the target sequence. The universal sequence may enable amplification of the target sequence. The universal sequence may enable transcription or reverse transcription of the target sequence. The universal sequence may enable sequencing of the target sequence.

The probe or primer compositions of the present disclosure can be provided on a solid support. The solid support can comprise one or more beads, plates, solid surfaces, wells, chips, or a combination thereof. The beads can be magnetic, antibody coated, protein A crosslinked, protein G crosslinked, streptavidin coated, oligonucleotide conjugated, silica coated, or a combination thereof. Examples of beads include, but are not limited to, Ampure beads, AMPure XP beads, streptavidin beads, agarose beads, magnetic beads, Dynabeads®, MACS® microbeads, antibody conjugated beads (e.g., anti-immunoglobulin microbead), protein A conjugated beads, protein G conjugated beads, protein A/G conjugated beads, protein L conjugated beads, oligo-dT conjugated beads, silica beads, silica-like beads, anti-biotin microbead, anti-fluorochrome microbead, and BcMag™ Carboxy-Terminated Magnetic Beads.

The compositions and kits can comprise primers and primer pairs capable of amplifying target molecules, or fragments or subsequences or complements thereof. The nucleotide sequences of the target molecules may be provided in computer-readable media for in silico applications and as a basis for the design of appropriate primers for amplification of one or more target molecules.

Primers based on the nucleotide sequences of target molecules can be designed for use in amplification of the target molecules. For use in amplification reactions such as PCR, a pair of primers can be used. The exact composition of the primer sequences is not critical to the disclosure, but for most applications the primers may hybridize to specific sequences of the target molecules or the universal sequence of the probe under stringent conditions, particularly under conditions of high stringency, as known in the art. The pairs of primers are usually chosen so as to generate an amplification product of at least about 15 or more, 20 or more, 30 or more, 40 or more, 50 or more, 60 or more, 70 or more, 80 or more, 90 or more, 100 or more, 125 or more, 150 or more, 175 or more, 200 or more, 250 or more, 300 or more, 350 or more, 400 or more, 450 or more, 500 or more, 600 or more, 700 or more, 800 or more, 900 or more, or 1000 or more nucleotides. Algorithms for the selection of primer sequences are generally known, and are available in commercial software packages. These primers may be used in standard quantitative or qualitative PCR-based assays to assess transcript expression levels of target molecules. Alternatively, these primers may be used in combination with probes, such as molecular beacons in amplifications using real-time PCR.

The nucleotide sequence of the entire length of the primer does not need to be derived from the target sequence. Thus, for example, the primer may comprise nucleotide sequences at the 5′ and/or 3′ termini that are not derived from the target molecule. Nucleotide sequences which are not derived from the nucleotide sequence of the target molecule may provide additional functionality to the primer. For example, they may provide a restriction enzyme recognition sequence or a “tag” that facilitates detection, isolation, purification or immobilization onto a solid support. Alternatively, the additional nucleotides may provide a self-complementary sequence that allows the primer to adopt a hairpin configuration. Such configurations may be necessary for certain primers, for example, molecular beacon and Scorpion primers, which can be used in solution hybridization techniques.

The probes or primers can incorporate moieties useful in detection, isolation, purification, or immobilization, if desired. Such moieties are well-known in the art (see, for example, Ausubel et al., (1997 & updates) Current Protocols in Molecular Biology, Wiley & Sons, New York) and are chosen such that the ability of the probe to hybridize with its target molecule is not affected.

Examples of suitable moieties are detectable labels, such as radioisotopes, fluorophores, chemiluminophores, enzymes, colloidal particles, and fluorescent microparticles, as well as antigens, antibodies, haptens, avidin/streptavidin, biotin, haptens, enzyme cofactors/substrates, enzymes, and the like.

A label can optionally be attached to or incorporated into a probe or primer to allow detection and/or quantitation of a target polynucleotide representing the target molecule of interest. The target polynucleotide may be the expressed target molecule RNA itself, a cDNA copy thereof, or an amplification product derived therefrom, and may be the positive or negative strand, so long as it can be specifically detected in the assay being used. Similarly, an antibody may be labeled.

In certain multiplex formats, labels used for detecting different target molecules may be distinguishable. The label can be attached directly (e.g., via covalent linkage) or indirectly, e.g., via a bridging molecule or series of molecules (e.g., a molecule or complex that can bind to an assay component, or via members of a binding pair that can be incorporated into assay components, e.g., biotin-avidin or streptavidin). Many labels are commercially available in activated forms which can readily be used for such conjugation (for example through amine acylation), or labels may be attached through known or determinable conjugation schemes, many of which are known in the art.

Labels useful in the disclosure described herein include any substance which can be detected when bound to or incorporated into the target molecule. Any effective detection method can be used, including optical, spectroscopic, electrical, piezoelectrical, magnetic, Raman scattering, surface plasmon resonance, colorimetric, calorimetric, etc. A label is typically selected from a chromophore, a lumiphore, a fluorophore, one member of a quenching system, a chromogen, a hapten, an antigen, a magnetic particle, a material exhibiting nonlinear optics, a semiconductor nanocrystal, a metal nanoparticle, an enzyme, an antibody or binding portion or equivalent thereof, an aptamer, and one member of a binding pair, and combinations thereof. Quenching schemes may be used, wherein a quencher and a fluorophore as members of a quenching pair may be used on a probe, such that a change in optical parameters occurs upon binding to the target introduce or quench the signal from the fluorophore. One example of such a system is a molecular beacon. Suitable quencher/fluorophore systems are known in the art. The label may be bound through a variety of intermediate linkages. For example, a target polynucleotide may comprise a biotin-binding species, and an optically detectable label may be conjugated to biotin and then bound to the labeled target polynucleotide. Similarly, a polynucleotide sensor may comprise an immunological species such as an antibody or fragment, and a secondary antibody containing an optically detectable label may be added.

Chromophores useful in the methods described herein include any substance which can absorb energy and emit light. For multiplexed assays, a plurality of different signaling chromophores can be used with detectably different emission spectra. The chromophore can be a lumophore or a fluorophore. Typical fluorophores include fluorescent dyes, semiconductor nanocrystals, lanthanide chelates, polynucleotide-specific dyes and green fluorescent protein.

Coding schemes may optionally be used, comprising encoded particles and/or encoded tags associated with different polynucleotides of the disclosure. A variety of different coding schemes are known in the art, including fluorophores, including SCNCs, deposited metals, and RF tags.

### Subjects and Samples

The methods and kits described herein are suitable for detecting a level or an amount of expression of one or more of the genes described herein in a sample from a subject. In some embodiments, a subject from whom a sample is obtained can be selected by the skilled practitioner. In some embodiments, selection of the subject is based upon consideration or analysis of one or more factors. Such factors for consideration include, but are not limited to, family history of a specific disease, genetic predisposition for the disease, increased risk for the disease, physical symptoms which indicate the disease, or environmental reasons. Environmental reasons can include, but are not limited to, lifestyle or exposure to agents which cause or contribute to the specific disease. In some embodiments, selection of a subject is based on the subject's previous history with the disease, positive diagnosis prior to therapy or after therapy, treatment for the disease, or remission or recovery from the disease.

In some embodiments, samples for use with the kits and in the methods of the present disclosure comprise nucleic acids suitable for providing RNA expression information. In principle, the biological sample from which the expressed RNA is obtained and analyzed for target molecule expression can be any material suspected of comprising cancer tissue or cells. The sample can be a biological sample used directly in a method of the disclosure. Alternatively, the sample can be a sample prepared from a biological sample.

In some embodiments, the sample or portion of the sample comprising or suspected of comprising cancer tissue or cells can be any source of biological material, including cells, tissue, secretions, or fluid, including bodily fluids. Non-limiting examples of the source of the sample include an aspirate, a needle biopsy, a cytology pellet, a bulk tissue preparation or a section thereof obtained for example by surgery or autopsy, lymph fluid, blood, plasma, serum, tumors, and organs. Alternatively, or additionally, the source of the sample can be urine, bile, excrement, sweat, tears, spinal fluid, and stool. In some embodiments, the sources of the sample are secretions. In some embodiments, the secretions are exosomes. In some embodiments, the sample is a urine sample. In some embodiments, the urine sample is obtained after a subject's digital rectal examination (DRE). In some embodiments, the urine sample is obtained within 30 minutes after a subject's DRE. In some embodiments, the urine sample is obtained from 30 minutes to 60 minutes after a subject's DRE. In some embodiments, the urine sample is obtained from 30 minutes to 180 minutes after a subject's DRE. In some embodiments, the urine sample is obtained within one hour after a subject's DRE. In some embodiments, the urine sample is obtained within two hours after a subject's DRE. In some embodiments, the urine sample is obtained within three hours after a subject's DRE. In some embodiments, a urine sample is obtained from a subject who has not had a DRE.

Without wishing to be bound by theory, it is believed that the DRE increases the sample's, e.g., urine sample's, concentration of the mRNA or protein expressed by one or more of TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6. This increased concentration facilitates detection of mRNA or protein expressed by the one or more genes.

In some embodiments, a sample is combined with a buffer, e.g., for processing. In some embodiments, an amount of expression of one or more genes described herein is determined from a composition, e.g., a solution or suspension, comprising the sample and a buffer. Buffers suitable for samples are known to those of skill in the art and can be determined based on the type of sample being collected. In some embodiments, the composition further comprises a preservative for adequate stability of the sample. In some embodiments, the buffer to sample ratio is 2:5. In some embodiments, the buffer to sample ratio is 1:5, 2:5, 3:5 or 4:5.

The samples may be archival samples, having a known and documented medical outcome, or may be samples from current patients whose ultimate medical outcome is not yet known.

In some embodiments, the sample may be dissected prior to molecular analysis. The sample may be prepared via macrodissection of a bulk tumor specimen or portion thereof, or may be treated via microdissection, for example via Laser Capture Microdissection (LCM).

The sample may initially be provided in a variety of states, as fresh tissue, fresh frozen tissue, fine needle aspirates, and may be fixed or unfixed. Frequently, medical laboratories routinely prepare medical samples in a fixed state, which facilitates tissue storage. A variety of fixatives can be used to fix tissue to stabilize the morphology of cells, and may be used alone or in combination with other agents. Exemplary fixatives include crosslinking agents, alcohols, acetone, Bouin's solution, Zenker solution, Helv solution, osmic acid solution and Carnoy solution.

Crosslinking fixatives can comprise any agent suitable for forming two or more covalent bonds, for example, an aldehyde. Sources of aldehydes typically used for fixation include formaldehyde, paraformaldehyde, glutaraldehyde or formalin. Preferably, the crosslinking agent comprises formaldehyde, which may be included in its native form or in the form of paraformaldehyde or formalin. One of skill in the art would appreciate that for samples in which crosslinking fixatives have been used special preparatory steps may be necessary including for example heating steps and proteinase-k digestion.

One or more alcohols may be used to fix tissue, alone or in combination with other fixatives. Exemplary alcohols used for fixation include methanol, ethanol and isopropanol.

Formalin fixation is frequently used in medical laboratories. Formalin comprises both an alcohol, typically methanol, and formaldehyde, both of which can act to fix a biological sample.

Whether fixed or unfixed, the biological sample may optionally be embedded in an embedding medium. Exemplary embedding media used in histology including paraffin, Tissue-Tek® V.I.P.™, Paramat, Paramat Extra, Paraplast, Paraplast X-tra, Paraplast Plus, Peel Away Paraffin Embedding Wax, Polyester Wax, Carbowax Polyethylene Glycol, Polyfin™, Tissue Freezing Medium TFMFM, Cryo-Gef™, and OCT Compound (Electron Microscopy Sciences, Hatfield, PA). Prior to molecular analysis, the embedding material may be removed via any suitable techniques, as known in the art. For example, where the sample is embedded in wax, the embedding material may be removed by extraction with organic solvent(s), for example, xylenes. Kits are commercially available for removing embedding media from tissues. Samples or sections thereof may be subjected to further processing steps as needed, for example serial hydration or dehydration steps.

In some embodiments, the sample is a fixed, wax-embedded biological sample. Frequently, samples from medical laboratories are provided as fixed, wax-embedded samples, most commonly as formalin-fixed, paraffin embedded (FFPE) tissues.

In some embodiments, a subject is prostate biopsy naïve, i.e., the subject has not had a prostate biopsy. In some embodiments, a subject has had a prior negative prostate biopsy result. In some embodiments, the prostate biopsy result is negative for Grade Group ≥2 prostate cancer. In some embodiments, one or more additional clinical variables are associated with the subject. In some embodiments, the methods comprise assaying one or more additional clinical variables (e.g., including but not limited to, the subject's prostate volume, PSA level or amount, PSA density, biopsy Gleason score, race, family history of prostate cancer, previous negative prostate biopsy, or abnormal DRE. In some embodiments, one or more additional clinical variables are associated with a subject that had a prior negative prostate biopsy result.

### Determining Likelihood of Having or Developing Grade Group ≥2 Prostate Cancer

In some embodiments, the level or amount of expression of one or more genes described herein determines the likelihood of detecting prostate cancer in a subject. In some embodiments, the likelihood of detecting prostate cancer in a subject is based on a prostate biopsy of the subject. In some embodiments, the level or amount of expression of one or more genes described herein determines the likelihood of detecting Grade Group ≥2 prostate cancer in a subject. In some embodiments, the likelihood is presented as a score based on the amount or level of expression of one or more genes described herein present in a sample from a subject. In some embodiments, the likelihood of detecting Grade Group ≥2 prostate cancer is provided as a score ranging from 0% to 100%. In some embodiments, the likelihood of detecting Grade Group ≥2 prostate cancer is provided as a score ranging from 0.0 to 100.0. In some embodiments, a biopsy naïve subject receiving a score of 0-7.5% means that there is a low risk or low likelihood that Grade Group ≥2 prostate cancer would be detected from a prostate biopsy of the subject. In some embodiments, a subject with a prior negative prostate biopsy result receiving a score of 0-5.4% means that there is a low risk or low likelihood that Grade Group ≥2 prostate cancer would be detected from a prostate biopsy of the subject. In some embodiments, a biopsy naïve subject receiving a score of ≥7.6% means that there is a high risk or high likelihood that Grade Group ≥2 prostate cancer would be detected from a prostate biopsy of the subject. In some embodiments, a subject with a prior negative prostate biopsy result receiving a score of ≥5.5% has a high risk or high likelihood that Grade Group ≥2 prostate cancer would be detected from a prostate biopsy of the subject.

In some embodiments, a computer-based analysis program is used to translate the raw data generated by a detection assay (e.g., the presence, absence, or amount of a given marker or markers) into data of predictive value for a clinician, subject or subject's healthcare provider. The clinician, subject or subject's healthcare provider can access the raw data using any suitable means. Thus, in some embodiments, the present disclosure provides the further benefit that the clinician, subject or subject's healthcare provider, who might not be trained in genetics or molecular biology, need not understand the raw data. The data can be presented directly to the clinician subject or subject's healthcare provider in its most useful form. This enables the clinician or healthcare provider to immediately utilize the information in order to optimize the care of the subject.

The information can be received, processed or transmitted to or from one or more laboratories conducting the assays, information providers, medical personnel, or subjects using any suitable method. For example, in some embodiments of the present disclosure, a sample (e.g., a biopsy or a serum or urine sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject can visit a medical center to have the sample obtained and sent to the profiling center, or the subject itself can collect the sample (e.g., a urine sample) and directly send it to a profiling center. Where the sample comprises previously determined biological information, the information can be directly sent to the profiling service by the subject (e.g., an information card containing the information may be scanned by a computer and the data transmitted to a computer of the profiling center using an electronic communication systems). Once received by the profiling service, the sample can be processed and a profile can be produced (i.e., expression data), useful for the diagnostic or prognostic information desired for the subject.

The profile data is then prepared in a format suitable for interpretation by one or more medical personnel (e.g., a treating clinician, physician assistant, nurse, or pharmacist). For example, rather than providing raw expression data, the prepared format may represent a diagnosis or risk assessment (e.g., levels of the cancer markers described herein) for the subject, along with recommendations for particular treatment options. The data may be displayed to the medical personnel by any suitable method. For example, in some embodiments, the profiling service generates a report that can be printed for the medical personnel (e.g., at the point of care) or displayed to the medical personnel on a computer monitor.

In some embodiments, the information is first analyzed at the point of care or at a regional facility. The raw data is then sent to a central processing facility for further analysis and/or to convert the raw data to information useful for medical personnel or subject. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the medical personnel, the subject, or researchers.

In some embodiments, the subject or the subject's healthcare provider is able to directly access the data using the electronic communication system. The subject may choose further intervention or counseling based on the results.

In some embodiments, the data is used for research use. For example, the data may be used to further optimize the inclusion or elimination of markers as useful indicators of a particular condition or stage of disease or as a companion diagnostic to determine a treatment course of action.

In some embodiments, the level or amount of expression of one or more genes described herein is used for determining a score. In some embodiments, determining the score comprises performing an algorithm that generates the score. In some embodiments, the score correlates with or informs the subject's likelihood of having or developing Grade Group ≥2 prostate cancer. In some embodiments, the score indicates a likelihood that Grade Group ≥2 prostate cancer would be detected from a prostate biopsy of the subject. An algorithm to determine the score with an acceptable diagnostic accuracy may be derived based on, for example and without limitation, logistic regression with stepwise feature selection, logistic regression with recursive feature elimination, and regularized logistic regression with elastic net. In some embodiments, performing the algorithm comprises using a processor.

In some embodiments, the algorithm is Equation 1, 2, 3 or 4, below.

In some embodiments, the subject has had a prior negative prostate biopsy result, and determining the score comprises performing Equation 1:

$$MPS 2 = \frac{{e}^{x}}{{e}^{x} + 1} Where x = Intercept + Slope ( ( - 1 ) ( ( a ) ( CRTmeanAPOC 1 - CRTmeanReference ) + ( b ) ( CRTmeanB 3 GNT 6 - CRT meanReference ) + ( c ) ( CRTmeanCAMKK 2 - CRTmeanReference ) + ( d ) ( CRTmeanERG - CRTmeanReference ) + ( e ) ( CRTmeanHOXC 6 - CRTmeanReference ) + ( f ) ( CRTmeanKLK 4 - CRTmeanReference ) + ( g ) ( CRTmeanNKAIN 1 - CRTmeanReference ) + ( h ) ( CRTmeanOR 51 E 2 - CRTmeanReference ) + ( i ) ( CRTmeanPCA 3 - CRTmeanReference ) + ( j ) ( CRTmeanPCAT 14 - CRTmeanReference ) + ( k ) ( CRTmeanPCGEM 1 - CRTmeanReference ) + ( l ) ( CRTmeanSCHLAP 1 - CRTmeanReference ) + ( m ) ( CRTmeanSPON 2 - CRTmeanReference ) + ( n ) ( CRTmeanTFF 3 - CRTmeanReference ) + ( o ) ( CRTmeanT 2 : ERG - CRTmeanReference ) + ( p ) ( CRTmeanTMSB 15 A - CRTmeanReference ) + ( q ) ( CRTmeanTRGV 9 - CRTmeanReference ) ) + ( ( r ) ( Age ) + ( s ) ( FamilyHx ) + ( t ) ( AbnormalDRE ) + ( u ) ( BxPriorNeg ) + ( v ) ( PSA ) + ( w ) ( ProstateVolume ) ) \qquad Equation 1$$

In some embodiments, the subject is prostate biopsy naïve (i.e., has not had a prior prostate biopsy), and determining the score comprises performing Equation 2:

$$MPS 2 = \frac{{e}^{x}}{{e}^{x} + 1} Where x = Intercept + Slope ( ( - 1 ) ( ( a ) ( CRTmeanAPOC 1 - CRTmeanReference ) + ( b ) ( CRTmeanB 3 GNT 6 - CRT meanReference ) + ( c ) ( CRTmeanCAMKK 2 - CRTmeanReference ) + ( d ) ( CRTmeanERG - CRTmeanReference ) + ( e ) ( CRTmeanHOXC 6 - CRTmeanReference ) + ( f ) ( CRTmeanKLK 4 - CRTmeanReference ) + ( g ) ( CRTmeanNKAIN 1 - CRTmeanReference ) + ( h ) ( CRTmeanOR 51 E 2 - CRTmeanReference ) + ( i ) ( CRTmeanPCA 3 - CRTmeanReference ) + ( j ) ( CRTmeanPCAT 14 - CRTmeanReference ) + ( k ) ( CRTmeanPCGEM 1 - CRTmeanReference ) + ( l ) ( CRTmeanSCHLAP 1 - CRTmeanReference ) + ( m ) ( CRTmeanSPON 2 - CRTmeanReference ) + ( n ) ( CRTmeanTFF 3 - CRTmeanReference ) + ( o ) ( CRTmeanT 2 : ERG - CRTmeanReference ) + ( p ) ( CRTmeanTMSB 15 A - CRTmeanReference ) + ( q ) ( CRTmeanTRGV 9 - CRTmeanReference ) ) ) \qquad Equation 2$$

In Equations 1 and 2, “Reference” refers to a reference gene described herein (e.g., KLK3).

In some embodiments, the subject has had a prior negative prostate biopsy result, and determining the score comprises performing Equation 3:

$$MPS 2 = \frac{{e}^{x}}{{e}^{x} + 1} Where x = Intercept + Slope ( ( - 1 ) ( ( a ) ( CRTmeanAPOC 1 - CRTmeanKLK 3 ) + ( b ) ( CRTmeanB 3 GNT 6 - CRTmeanKLK 3 ) + ( c ) ( CRTmeanCAMKK 2 - CRTmeanKLK 3 ) + ( d ) ( CRTmeanERG - CRTmeanKLK 3 ) + ( e ) ( CRTmeanHOXC 6 - CRTmeanKLK 3 ) + ( f ) ( CRTmeanKLK 4 - CRTmeanKLK 3 ) + ( g ) ( CRTmeanNKAIN 1 - CRTmeanKLK 3 ) + ( h ) ( CRTmeanOR 51 E 2 - CRTmeanKLK 3 ) + ( i ) ( CRTmeanPCA 3 - CRTmeanKLK 3 ) + ( j ) ( CRTmeanPCAT 14 - CRTmeanKLK 3 ) + ( k ) ( CRTmeanPCGEM 1 - CRTmeanKLK 3 ) + ( l ) ( CRTmeanSCHLAP 1 - CRTmeanKLK 3 ) + ( m ) ( CRTmeanSPON 2 - CRTmeanKLK 3 ) + ( n ) ( CRTmeanTFF 3 - CRTmeanKLK 3 ) + ( o ) ( CRTmeanT 2 : ERG - CRTmeanKLK 3 ) + ( p ) ( CRTmeanTMSB 15 A - CRTmeanKLK 3 ) + ( q ) ( CRTmeanTRGV 9 - CRTmeanKLK 3 ) ) ) \qquad Equation 3$$

In some embodiments, the subject is prostate biopsy naïve (i.e., has not had a prior prostate biopsy), and determining the score comprises performing Equation 4:

$$MPS 2 = \frac{{e}^{x}}{{e}^{x} + 1} Where x = Intercept + Slope ( ( - 1 ) ( ( a ) ( CRTmeanAPOC 1 - CRTmeanKLK 3 ) + ( b ) ( CRTmeanB 3 GNT 6 - CRTmeanKLK 3 ) + ( c ) ( CRTmeanCAMKK 2 - CRTmeanKLK 3 ) + ( d ) ( CRTmeanERG - CRTmeanKLK 3 ) + ( e ) ( CRTmeanHOXC 6 - CRTmeanKLK 3 ) + ( f ) ( CRTmeanKLK 4 - CRTmeanKLK 3 ) + ( g ) ( CRTmeanNKAIN 1 - CRTmeanKLK 3 ) + ( h ) ( CRTmeanOR 51 E 2 - CRTmeanKLK 3 ) + ( i ) ( CRTmeanPCA 3 - CRTmeanKLK 3 ) + ( j ) ( CRTmeanPCAT 14 - CRTmeanKLK 3 ) + ( k ) ( CRTmeanPCGEM 1 - CRTmeanKLK 3 ) + ( l ) ( CRTmeanSCHLAP 1 - CRTmeanKLK 3 ) + ( m ) ( CRTmeanSPON 2 - CRTmeanKLK 3 ) + ( n ) ( CRTmeanTFF 3 - CRTmeanKLK 3 ) + ( o ) ( CRTmeanT 2 : ERG - CRTmeanKLK 3 ) + ( p ) ( CRTmeanTMSB 15 A - CRTmeanKLK 3 ) + ( q ) ( CRTmeanTRGV 9 - CRTmeanKLK 3 ) ) + ( ( r ) ( Age ) + ( s ) ( FamilyHx ) + ( t ) ( AbnormalDRE ) + ( u ) ( BxPriorNeg ) + ( v ) ( PSA ) + ( w ) ( ProstateVolume ) ) ) \qquad Equation 4$$

In each of Equations 1-4: (i) “CRT” refers to the cycle threshold value identified by a method described herein for determining the amount of expression of a gene, (ii) “e” is Euler's number, and (iii) “MPS2” is the score indicating the likelihood that Grade Group ≥2 prostate cancer would be detected from a prostate biopsy of a subject. In Equations 1 and 3, “FamilyHx”, “AbnormalDRE” and “BxPriorNeg” are binary values of 1 or 0 where 1=yes and 0=no. Specifically, if a subject has a family history of prostate cancer, the value is 1; if a subject had an abnormal DRE, the value is 1; and if the subject had a prior negative prostate biopsy, the value is 1. In each of Equations 1-4, (a)-(w) are independent coefficients based on the model selected, e.g., logistic regression with stepwise feature selection, logistic regression with recursive feature elimination, or regularized logistic regression with elastic net. Illustrative coefficients are provided in Table B.

TABLE B
Illustrative coefficients for use in Equations 1-4

| | Coefficient | | |
|---|---|---|---|
| | in | | |
| | Equations | Biopsy Naïve | Biopsy Negative |
| Gene Name | 1-4 | Coefficient | Coefficient |
| APOC1 | (a) | −0.06 | −0.0889639 |
| B3GNT6 | (b) | 0.05 | 0.07252488 |
| CAMKK2 | (c) | −0.35 | −0.2779419 |
| ERG | (d) | 0.02 | 0.03008525 |
| HOXC6 | (e) | 0.01 | 0 |
| KLK3 | — | N/A | N/A |
| KLK4 | (f) | 0 | 0.21460919 |
| NKAIN1 | (g) | −0.08 | −0.0937911 |
| OR51E2 | (h) | 0.25 | 0.22879188 |
| PCA3.1 | (i) | 0.13 | 0.07420989 |
| PCAT14 | (j) | 0.13 | 0.16009867 |
| PCGEM1 | (k) | −0.14 | −0.1490843 |
| SCHLAP1 | (l) | 0.22 | 0.20526883 |
| SPON2 | (m) | 0.11 | 0.1740959 |
| TFF3 | (n) | 0.17 | 0.2128103 |
| T2ERG | (o) | 0.14 | 0.14858463 |
| (TMPRSS2-ERG) | | | |
| TMSB15A | (p) | 0.17 | 0.21487077 |
| TRGV9 | (q) | 0.13 | 0.17797231 |
| Age | (r) | — | 0.02144648 |
| aa | — | — | 1.23249323 |
| Family history | (s) | — | 0.29275737 |
| DRE abnormal | (t) | — | 1.09443244 |
| Biopsy prior neg | (u) | — | −0.6169421 |
| PSA | (v) | — | 0.09255434 |
| Prostate Volume | (w) | — | −0.0240516 |
| Intercept | — | −1.406563436 | −1.412069526 |
| Slope | — | 1.128771142 | 1.07706078 |

N/A = not applicable.
— = not present in any of Equations 1-4.

The methods disclosed herein can also comprise transmitting the data/information. For example, data/information derived from the detection and/or quantification of the target may be transmitted to another device and/or instrument. In some instances, the information obtained from an algorithm may also be transmitted to another device and/or instrument. Transmission of the data/information may comprise the transfer of data/information from a first source to a second source. The first and second sources may be in the same approximate location (e.g., within the same room, building, block, campus). Alternatively, first and second sources may be in multiple locations (e.g., multiple cities, states, countries, continents, etc.).

Transmission of the data/information can comprise digital transmission or analog transmission. Digital transmission may comprise the physical transfer of data (a digital bit stream) over a point-to-point or point-to-multipoint communication channel. Examples of such channels are copper wires, optical fibers, wireless communication channels, and storage media. The data may be represented as an electromagnetic signal, such as an electrical voltage, radiowave, microwave, or infrared signal.

Analog transmission may comprise the transfer of a continuously varying analog signal. The messages can either be represented by a sequence of pulses by means of a line code (baseband transmission), or by a limited set of continuously varying wave forms (passband transmission), using a digital modulation method. The passband modulation and corresponding demodulation (also known as detection) can be carried out by modem equipment. According to the most common definition of digital signal, both baseband and passband signals representing bit-streams are considered as digital transmission, while an alternative definition only considers the baseband signal as digital, and passband transmission of digital data as a form of digital-to-analog conversion.

In some embodiments, a report is generated comprising a score. In some embodiments, the score indicates a likelihood that Grade Group ≥2 prostate cancer would be detected from a prostate biopsy of the subject. In some embodiments, the report is accessible by or provided to the subject's healthcare provider. In some embodiments, the report is accessible or provided as a digital or paper copy. In some embodiments, the report is delivered to the subject's healthcare provider by a digital format as described herein (e.g., via electronic mail), or via a courier if the report is in paper copy.

In some embodiments, the report comprises a treatment option. In some embodiments, the report comprises treatment options for Grade Group ≥2 prostate cancer.

### Diagnostic Accuracy

Diagnostic accuracy of the methods or kits described herein can be determined by analyzing the Area Under the Curve (AUC) derived from Receiver Operator Characteristic (ROC) curves. ROC curves are graphical plots that illustrate the ability of a binary classifier system as its discrimination threshold is varied. ROC curves are plotted with true positive rate against the false positive rate, with true positive rate on the y-axis and false positive rate on the x-axis. The true positive rate, also referred to as the sensitivity, is calculated by dividing the number of true positives by the sum of true positives and false negatives. The false positive rate is calculated by either (1) dividing the number of false positives by the sum of true negatives and false positives, or (2) subtracting the specificity from one, wherein specificity is calculated by dividing the number of true negatives by the sum of true negatives and false positives. In some embodiments, ROC curves are generated based on individual amounts of expression of each gene. In some embodiments, ROC curves are generated based on a combination of amounts of expression of each gene.

In some embodiments, the AUC value of the methods or kits described herein is greater than 0.50. In some embodiments, the AUC value of the methods or kits described herein is at least 0.60. In some embodiments, the AUC value of the methods or kits described herein is at least 0.70. In some embodiments, the AUC value of the methods or kits described herein is at least 0.71. In some embodiments, the AUC value of the methods or kits described herein is at least 0.72. In some embodiments, the AUC value of the methods or kits described herein is at least 0.73. In some embodiments, the AUC value the methods or kits described herein is at least 0.74. In some embodiments, the AUC value of the methods or kits described herein is at least 0.75. In some embodiments, the AUC value of the methods or kits described herein is at least 0.76. In some embodiments, the AUC value of the methods or kits described herein is at least 0.77. In some embodiments, the AUC value of the methods or kits described herein is at least 0.78. In some embodiments, the AUC value of the methods or kits described herein is at least 0.79. In some embodiments, the AUC value of the methods or kits described herein is at least 0.80. In some embodiments, the AUC value of the methods or kits described herein is at least 0.81. In some embodiments, the AUC value of the methods or kits described herein is at least 0.82. In some embodiments, the AUC value of the methods or kits described herein is at least 0.83. In some embodiments, the AUC value of the methods or kits described herein is at least 0.84. In some embodiments, the AUC value of the methods or kits described herein is at least 0.85. In some embodiments, the AUC value of the methods or kits described herein is at least 0.86. In some embodiments, the AUC value of the methods or kits described herein is at least 0.87. In some embodiments, the AUC value of the methods or kits described herein is at least 0.88. In some embodiments, the AUC value of the methods or kits described herein is at least 0.89. In some embodiments, the AUC value of the methods or kits described herein is at least 0.90.

Diagnostic accuracy of the amount of expression of an individual gene or combination of amounts of expression of specific genes can be maximized by implementing a cut-off analysis that takes into account the sensitivity, specificity, negative predictive value (NPV), positive predictive value (PPV), positive likelihood ratio (PLR) and negative likelihood ratio (NLR) necessary for clinical utility. Results of amounts of expression are analyzed in any of a variety of ways. In some embodiments, the results are analyzed using a univariate, or single-variable analysis (SV), In some embodiments, the results are analyzed using multivariate analysis (MV).

The generation of ROC curves and analysis of a population of samples can be used to establish the cutoff value used to distinguish between different subject sub-groups. For example, the cutoff value can be used to distinguish between a high likelihood of detecting Grade Group ≥2 prostate cancer from a subject's prostate biopsy and a low likelihood of detecting Grade Group ≥2 prostate cancer from a subject's prostate biopsy. In some embodiments, the cutoff value can distinguish between these subjects. In some embodiments, the cutoff value may distinguish between subjects with a non-aggressive cancer from an aggressive cancer.

In some embodiments, the methods or kits described herein provide a score indicating with a diagnostic accuracy of at least 0.70 the likelihood that. Grade Group ≥2 prostate cancer would be detected from a subject's prostate biopsy. In some embodiments, the methods or kits described herein provide a score indicating with a diagnostic accuracy of at least 0.75 the likelihood that Grade Group ≥2 prostate cancer would be detected from a subject's prostate biopsy. In some embodiments, the methods or kits described herein provide a score indicating with a diagnostic accuracy of at least 0.80 the likelihood that Grade Group ≥2 prostate cancer would be detected from a subject's prostate biopsy.

In some embodiments, the methods or kits described herein provide a score indicating with a diagnostic accuracy of at least 0.70 the likelihood that Grade Group ≥2 prostate cancer would be detected from a prostate biopsy naïve subject's prostate biopsy. In some embodiments, the methods or kits described herein provide a score indicating with a diagnostic accuracy of at least 0.75 the likelihood that Grade Group ≥2 prostate cancer would be detected from a prostate biopsy naïve subject's prostate biopsy. In some embodiments, the methods or kits described herein provide a score indicating with a diagnostic accuracy of at least 0.80 the likelihood that Grade Group ≥2 prostate cancer would be detected from a prostate biopsy naïve subject's prostate biopsy.

In some embodiments, the methods or kits described herein provide a score indicating with a diagnostic accuracy of at least 0.70 the likelihood that Grade Group ≥2 prostate cancer would be detected from a prostate biopsy of a subject having a previous negative prostate biopsy. In some embodiments, the methods or kits described herein provide a score indicating with a diagnostic accuracy of at least 0.75 the likelihood that Grade Group ≥2 prostate cancer would be detected from a prostate biopsy of a subject having a previous negative prostate biopsy. In some embodiments, the methods or kits described herein provide a score indicating with a diagnostic accuracy of at least 0.80 the likelihood that Grade Group ≥2 prostate cancer would be detected from a prostate biopsy of a subject having a previous negative prostate biopsy. In some embodiments, the methods or kits described herein provide a score indicating with a diagnostic accuracy of at least 0.81 the likelihood that Grade Group ≥2 prostate cancer would be detected from a prostate biopsy of a subject having a previous negative prostate biopsy. In some embodiments, the methods or kits described herein provide a score indicating with a diagnostic accuracy of at least 0.82 the likelihood that Grade Group ≥2 prostate cancer would be detected from a prostate biopsy of a subject having a previous negative prostate biopsy.

In some embodiments, each referenced diagnostic accuracy is achievable where the urine sample is obtained within one hour after a subject's digital rectal examination (DR E), In some embodiments, each referenced diagnostic accuracy is achievable where the urine sample is obtained from 30 minutes to 60 minutes after a subject's DRE. In some embodiments, the urine sample is obtained from 30 minutes to 180 minutes after a subject's DRE. In some embodiments, the urine sample is obtained within one hour after a subject's DRE. In some embodiments, the urine sample is obtained within two hours after a subject's DRE. In some embodiments, the urine sample is obtained within three hours after a subject's DRE. In some embodiments, a urine sample is obtained from a subject who has not had a DRE,

### Kits and Devices

In some embodiments, the disclosure provides kits for analyzing a cancer, comprising (a) a probe set comprising a plurality of probes comprising target specific sequences complementary to one or more target molecules, wherein the one or more target molecules comprise one or more cancer markers; and (b) a computer model or algorithm for analyzing an expression level and/or expression profile of the one or more target molecules in a sample. The target molecules may comprise one or more of those described herein or a combination thereof.

In some embodiments, the disclosure provides kits for analyzing a cancer, comprising (a) a probe set comprising a plurality of probes comprising target specific sequences complementary to one or more target molecules of a biomarker library; and (b) a computer model or algorithm for analyzing an expression level and/or expression profile of the one or more target molecules in a sample. Control samples and/or nucleic acids may optionally be provided in the kit. Control samples may include tissue and/or nucleic acids obtained from or representative of tumor samples from a healthy subject, as well as tissue and/or nucleic acids obtained from or representative of tumor samples from subjects diagnosed with a cancer.

Instructions for using the kits to perform one or more methods of the disclosure can be provided, and can be provided in any fixed medium. The instructions may be located inside or outside a container or housing, and/or may be printed on the interior or exterior of any surface thereof. A kit may be in multiplex form for concurrently detecting and/or quantitating one or more different target polynucleotides representing the expressed target molecules.

In some embodiments, the disclosure provides kits comprising a container comprising a reagent composition for detecting an amount of expression of at least three genes described herein; and instructions for detecting the amount of expression. In some embodiments, the reagent composition comprises a polynucleotide reagent for detecting the amount of mRNA expressed by the at least three genes. In some embodiments, the reagent composition comprises a polynucleotide reagent for detecting an amount of expression of a reference gene, and the instructions are additionally for normalizing the amount of expression of the at least three genes to the amount of expression of the reference gene. In some embodiments, the instructions are additionally for generating a report comprising a score determined by the amount of expression of the at least three genes, wherein the score indicates the likelihood that Grade Group ≥2 prostate cancer would be detected from a subject's prostate biopsy.

Devices useful for performing methods of the disclosure are also provided. The devices can comprise means for characterizing the expression level of a target molecule of the disclosure, for example components for performing one or more methods of nucleic acid extraction, amplification, and/or detection. Such components may include one or more of an amplification chamber (for example, a thermal cycler), a plate reader, a spectrophotometer, capillary electrophoresis apparatus, a chip reader, and or robotic sample handling components. These components ultimately can obtain data that reflects the expression level of the target molecules used in the assay being employed.

The devices can include an excitation and/or a detection means. Any instrument that provides a wavelength that can excite a species of interest and is shorter than the emission wavelength(s) to be detected can be used for excitation. Commercially available devices can provide suitable excitation wavelengths as well as suitable detection component.

Illustrative excitation sources include a broadband UV light source such as a deuterium lamp with an appropriate filter, the output of a white light source such as a xenon lamp or a deuterium lamp after passing through a monochromator to extract out the desired wavelength(s), a continuous wave (cw) gas laser, a solid-state diode laser, or any of the pulsed lasers. Emitted light can be detected through any suitable device or technique; many suitable approaches are known in the art. For example, a fluorimeter or spectrophotometer may be used to detect whether the test sample emits light of a wavelength characteristic of a label used in an assay.

The devices can comprise a means for identifying a given sample, and of linking the results obtained to that sample. Such means can include manual labels, barcodes, and other indicators which can be linked to a sample vessel, and/or may optionally be included in the sample itself, for example where an encoded particle is added to the sample. The results may be linked to the sample, for example in a computer memory that contains a sample designation and a record of expression levels obtained from the sample. Linkage of the results to the sample can also include a linkage to a particular sample receptacle in the device, which is also linked to the sample identity.

The devices can also comprise a means for correlating the expression levels of the target molecules being studied with a prognosis of disease outcome. Such means may comprise one or more of a variety of correlative techniques, including lookup tables, algorithms, multivariate models, and linear or nonlinear combinations of expression models or algorithms. The expression levels may be converted to one or more likelihood scores, reflecting a likelihood that the subject providing the sample may exhibit a particular disease outcome. The models and/or algorithms can be provided in machine readable format and can optionally further designate a treatment modality for a subject or class of subjects.

The devices can also comprise output means for outputting the disease status, prognosis and/or a treatment modality. Such output means can take any form which transmits the results to a subject and/or a healthcare provider, and may include a monitor, a printed format, or both. The device may use a computer system for performing one or more of the steps provided.

### II. Prognosis, Diagnosis or Treatment

The methods, compositions, and kits disclosed herein are useful for the prognosis, diagnosis, predication, monitoring and/or treatment of cancer (e.g., prostate cancer, and in some embodiments Grade Group ≥2 prostate cancer) in a subject. In some embodiments, the predicting, and/or monitoring the status or outcome of a cancer includes assessing the presence or risk of high-grade prostate cancer (i.e., Grade Group ≥2 prostate cancer). In some embodiments, predicting, and/or monitoring the status or outcome of a cancer comprises determining the efficacy of treatment. In some embodiments, methods and kits disclosed herein are useful for indicating the likelihood that Grade Group ≥2 prostate cancer would be detected from a subject's prostate biopsy.

In some embodiments, the methods comprise determining, recommending or administering a therapeutic regimen. In some embodiments, the therapeutic regimen is an anti-cancer therapy. In some embodiments, the methods comprise modifying a therapeutic regimen. Modifying a therapeutic regimen can comprise increasing a therapeutic dosage, decreasing a therapeutic dosage, or terminating a therapeutic regimen.

For example, in some embodiments, the methods described herein are useful to identify subjects with high-grade prostate cancer. In some embodiments, the methods described herein are useful to identify subjects with a high likelihood of having high-grade prostate cancer detectable from a prostate biopsy. Such subjects can be administered prostate cancer therapy (e.g., one or more of surgery, radiation therapy, hormonal therapy, targeted therapy, chemotherapy, immunotherapy, radiopharmaceuticals, or bone-modifying drugs).

Conversely, in some embodiments, subjects identified as having a low-grade prostate cancer, or having a low likelihood of having high-grade prostate cancer, e.g., based on the levels of expression of the described markers, can be given an option to avoid a biopsy or treatment and opt for watchful waiting or minimal treatments.

In some embodiments, the prostate cancer therapy comprises administering a chemotherapeutic agent. Examples of chemotherapeutic agents include alkylating agents, anti-metabolites, plant alkaloids and terpenoids, vinca alkaloids, podophyllotoxin, taxanes, topoisomerase inhibitors, and cytotoxic antibiotics. Cisplatin, carboplatin, and oxaliplatin are examples of alkylating agents. Other alkylating agents include mechlorethamine, cyclophosphamide, chlorambucil, ifosfamide. Alkylating agents may impair cell function by forming covalent bonds with the amino, carboxyl, sulfhydryl, and phosphate groups in biologically important molecules. Alternatively, alkylating agents may chemically modify a cell's DNA.

Biological therapy (sometimes called immunotherapy, biotherapy, or biological response modifier (BRM) therapy) uses the body's immune system, either directly or indirectly, to fight cancer or to lessen the side effects that may be caused by some cancer treatments. Biological therapies include interferons, interleukins, colony-stimulating factors, monoclonal antibodies, vaccines, gene therapy, and nonspecific immunomodulating agents.

In some embodiments, the biological therapy is immune checkpoint therapy. Immune checkpoint inhibitors target CTLA-4, PD-1, or PD-L1. Examples include but are not limited to, ipilimumab, nivolumab, cemiplimab, avelumab, durvalumab, tremelimumab, dostarlimab, pembrolizumab, spartalizumab, and atezolizumab.

In some embodiments, the prostate cancer therapy is FDA-approved for treating prostate cancer. In some embodiments, the prostate cancer therapy is: abiraterone acetate, apulutamide, bicalutamide, cabazitaxel, casodex, darolutamide, degarelix, docetaxel, eligard, enzalutamide, erleada, firmagon, flutamide, goserelin acetate, jevtana, leuprolide acetate, Lupron depot, lutetium lu 177 vipivotide tetraxetan, Lynparza, mitoxantrone hydrochloride, nilandron, nilutamide, nubeqa, Olaparib, orgovyx, pluvicto, provenge, radium 223 dichloride, relugolix, rubraca, rucaparib camsylate, sipuleucel-t, taxotere, xofigo, xtandi, yonsa, zoladex, xytiga, or any combination thereof.

### EXPERIMENTAL

The following Examples are provided in order to demonstrate and further illustrate certain embodiments and aspects of the present disclosure and are not to be construed as limiting the scope thereof.

### Example 1

### Methods

### Initial Gene Screening

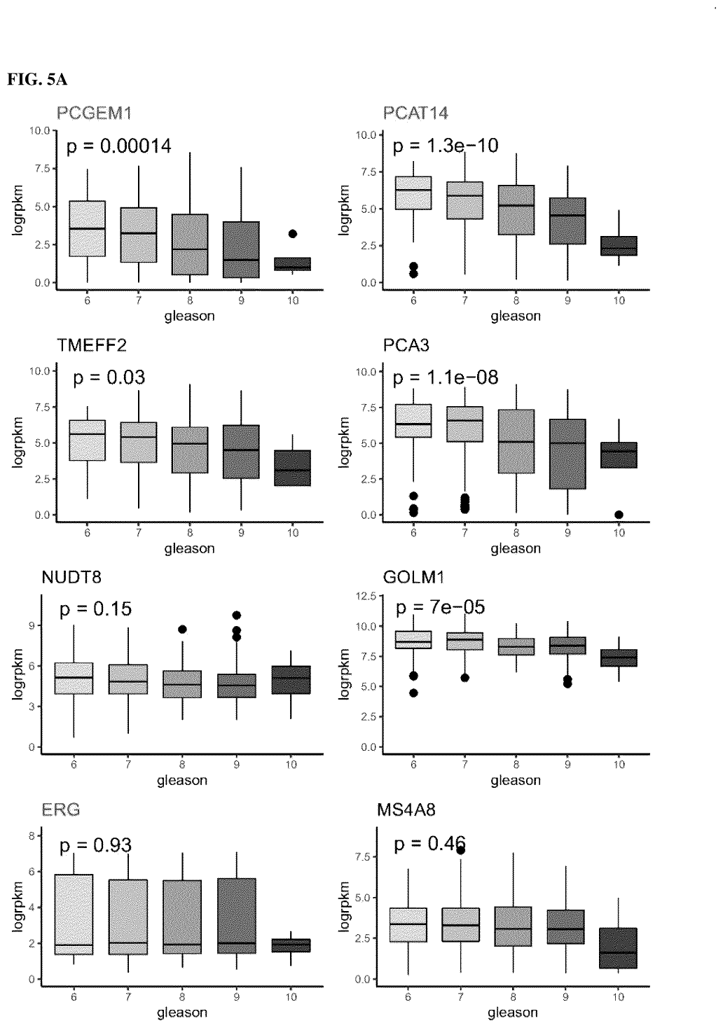

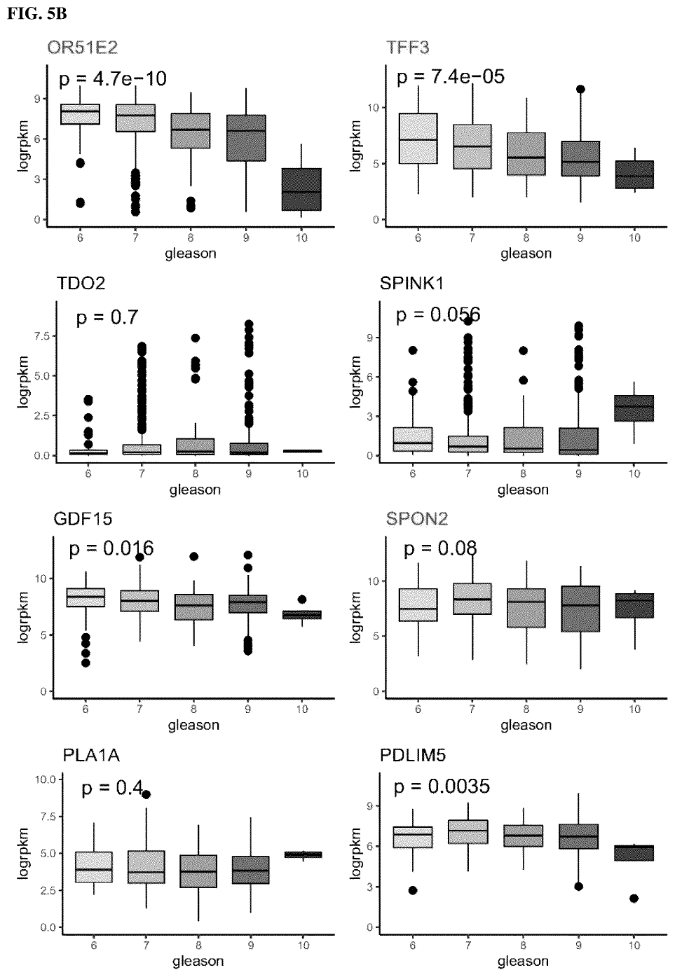

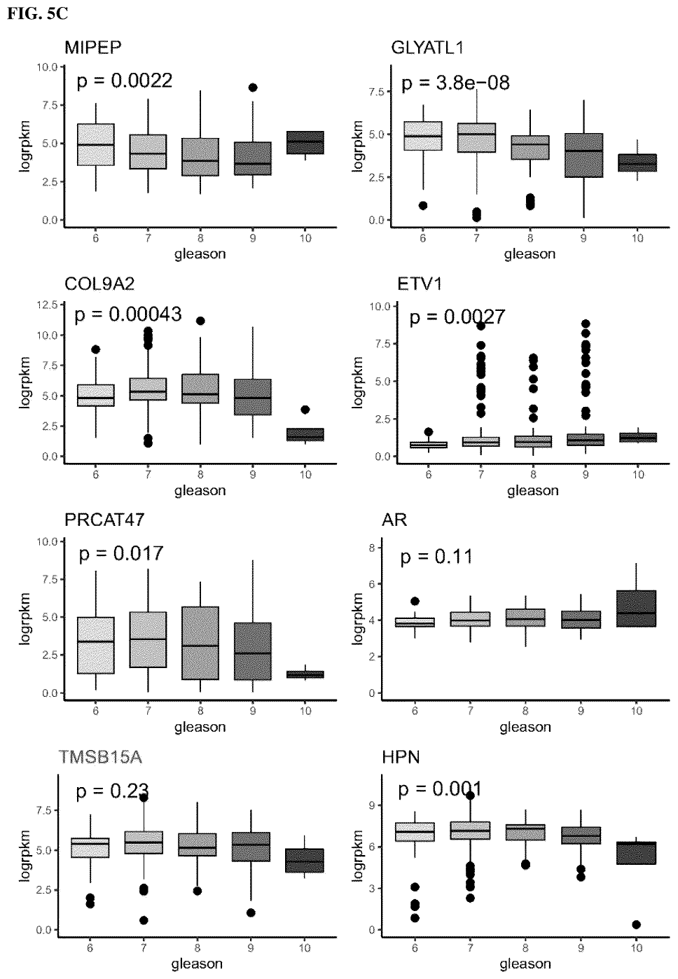

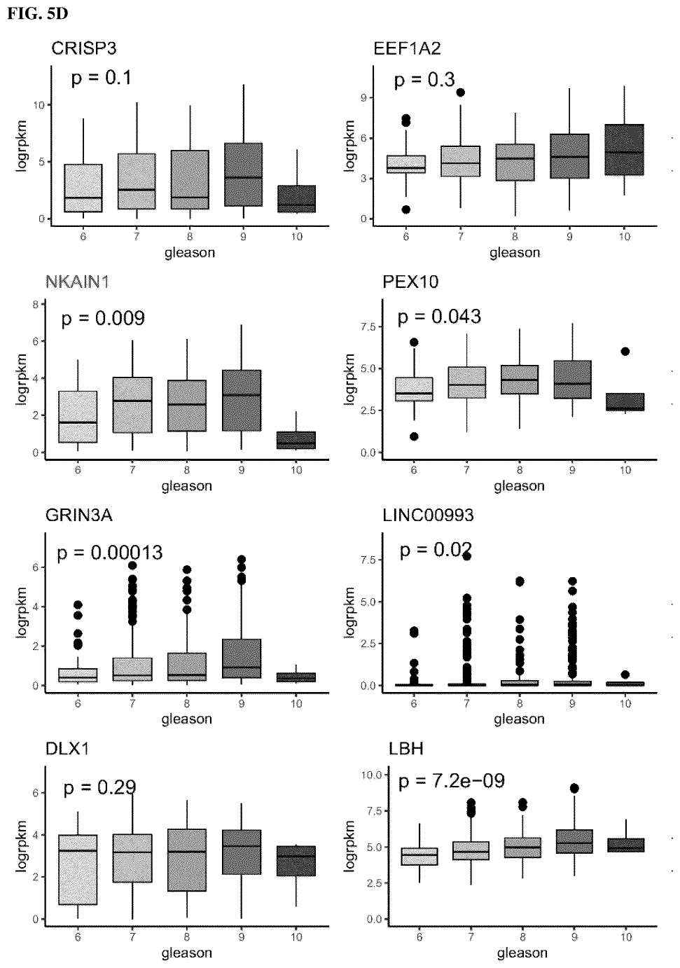

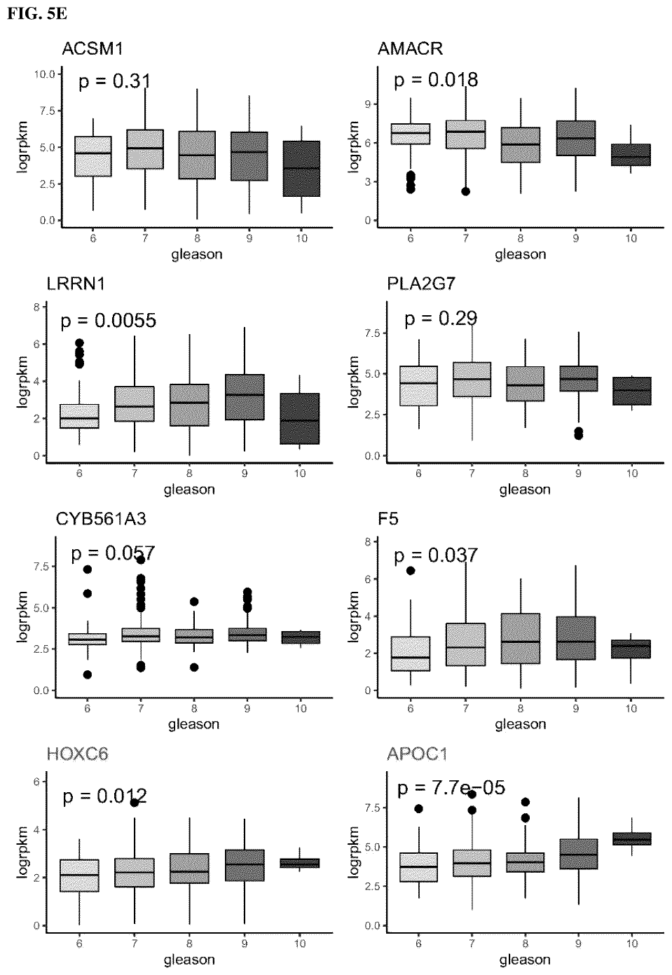

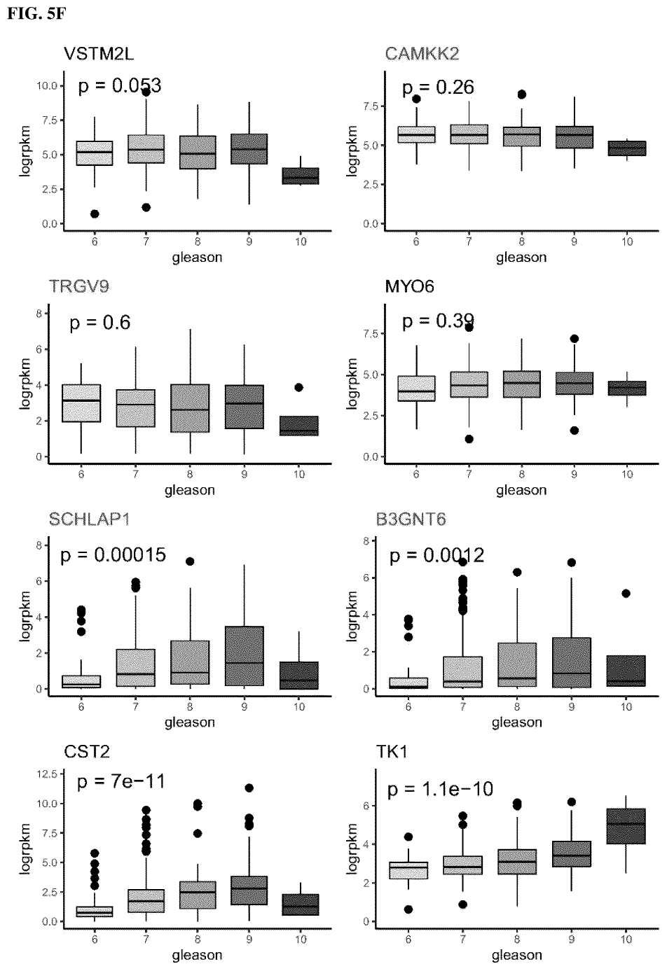

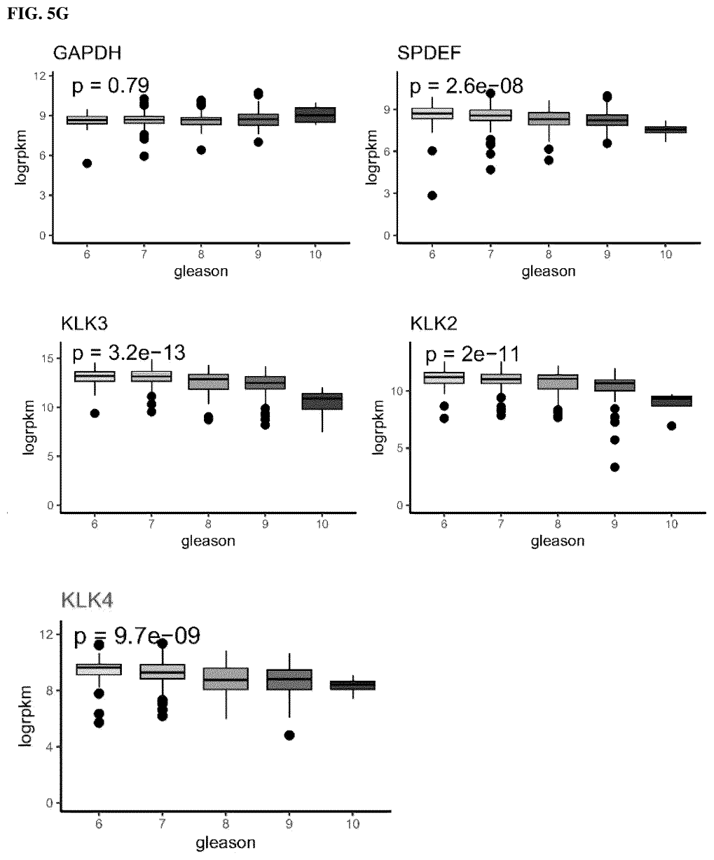

RNA-seq data from The Cancer Genome Atlas (TCGA) prostate adenocarcinoma (PRAD) cohort was used to select potential grade-associated genes (The Cancer Genome Atlas Research Network. The Molecular Taxonomy of Primary Prostate Cancer. Cell. 2015; 163(4):1011-25). Differential analyses between high- (Gleason >6) and low-grade (Gleason=6), and between high-grade and benign, were performed following the limma+voom procedure (Law C W, et al., voom: precision weights unlock linear model analysis tools for RNA-seq read counts. Genome Biol. 2014; 15(2):R29). Candidate genes were manually picked by evaluating log FCs and p values of both comparisons. Several genes known to be prostate cancer biomarkers were also included (Table 5). A total of 53 genes and 1 gene fusion (TMPRSS2-ERG) were selected (FIG. 5).

### Patient Cohorts

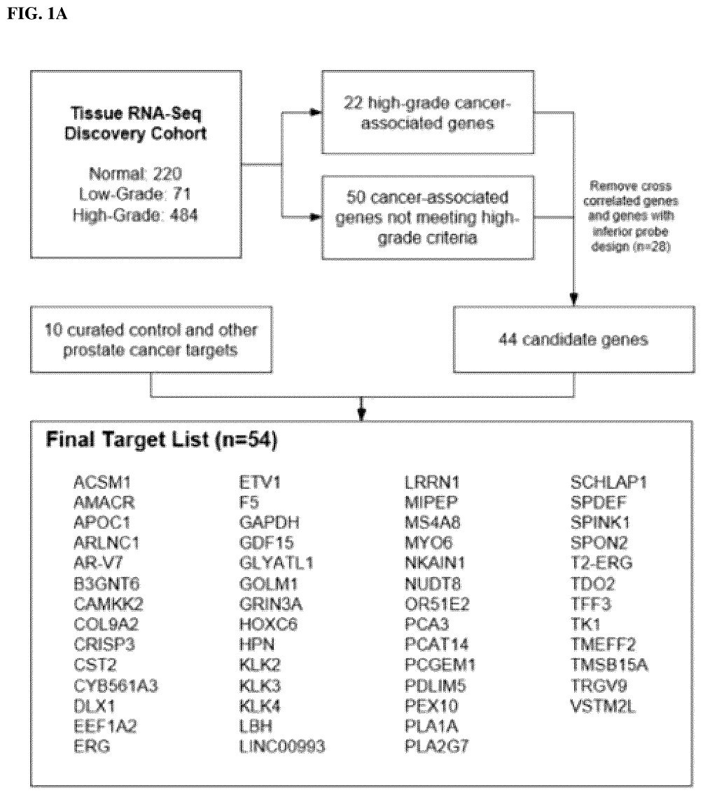

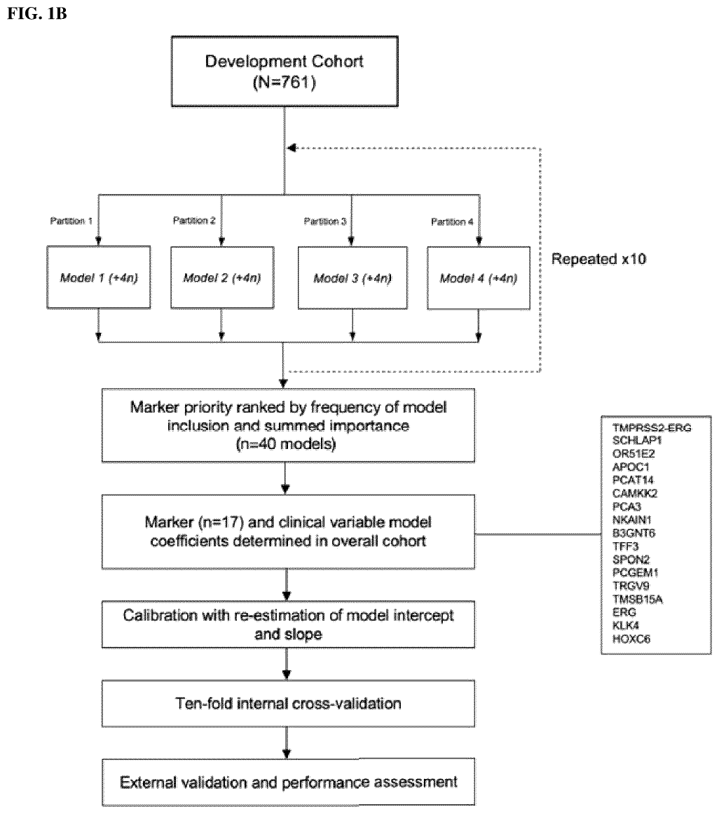

Information on the training (University of Michigan) and validation (NCI-EDRN) cohorts is given in FIG. 1B and Table 1.

Among 815 participants, qPCR yielded valid results in 761 (93%). Median age was 63 years (IQR 58-68), median PSA was 5.6 ng/mL (IQR 4.6-7.2), and 163 patients (21%) had a prior negative biopsy result (Table 1). On study biopsy, 293 men (39%) had GG≥2 cancer. The accuracy of each candidate gene was quantified across elastic net mathematical models (Table 4).

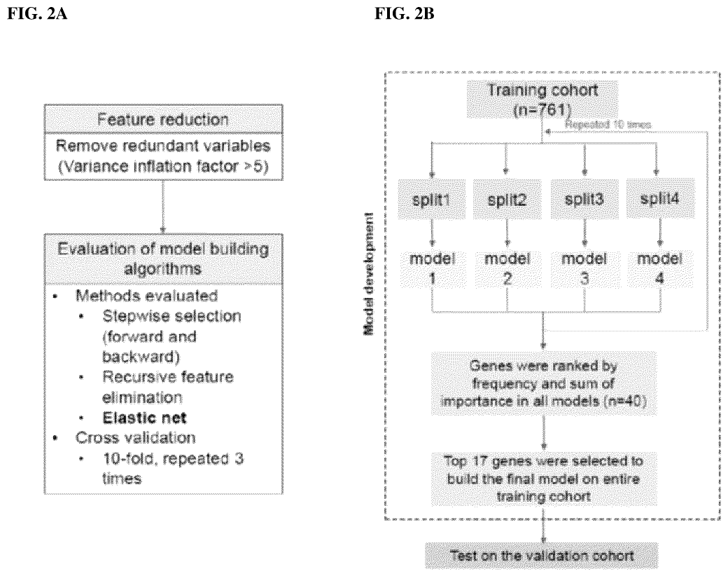

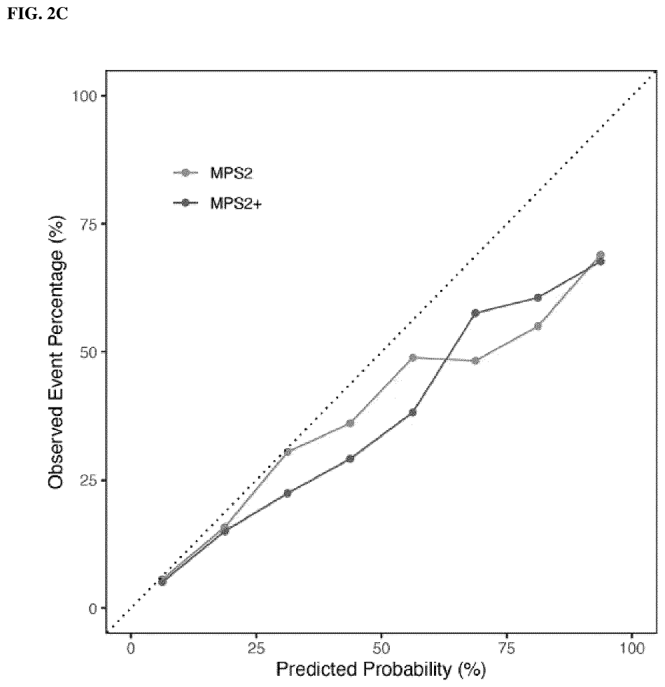

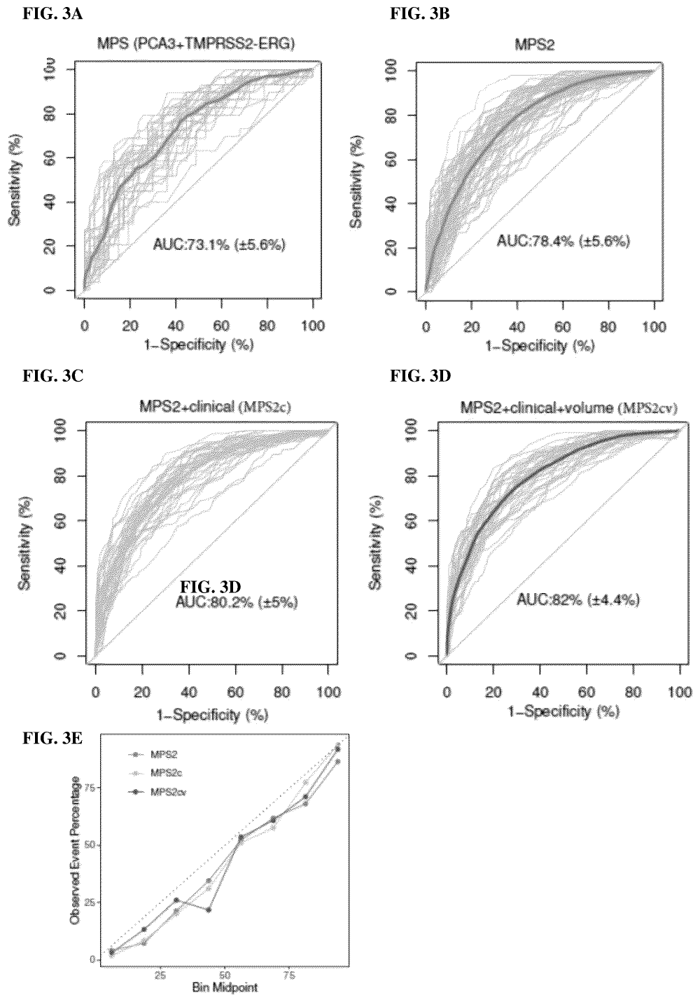

The final MPS2 model included standard clinical variables and the 17 most informative markers, including 13 from the discovery analysis (four high-grade-specific [APOC1, B3GNT6, NKAIN1, SCHLAP1] and nine prostate cancer-specific [PCGEM1, SPON2, TRGV9, PCA3, OR51 E2, CAMKK2, TFF3, PCAT14, TMSB15A]), four curated markers (HOXC6, ERG, TMPRSS2:ERG, KLK4), and the reference gene KLK3. Model coefficients were determined in the overall cohort 10 (Table 6). Calibration and internal cross-validation were performed (FIG. 2C and FIG. 3C-3D), and the MPS2 models were locked for external validation.

Urinary RNA Extraction and cDNA Synthesis

RNA isolation for MPS2 analysis was performed using the MagMAX® mirVana Total RNA Isolation Kit (ThermoFisher Scientific®). Briefly, 500 μL of urine/Hologic urine transport media 1:1 mixture was mixed using the Lysis Binding Mix (a component of MagMAX® mirVana Total RNA Isolation Kit from ThermoFisher Scientific®). Binding Beads Mix (a component of MagMAX® mirVana Total RNA Isolation Kit from ThermoFisher Scientific®) was then added to enrich nucleic acids from urine samples followed by TURBO DNase® digestion and washing. Finally, RNA was eluted. For high-throughput urine RNA extraction, urine samples were processed through the semi-automatic KingFisher Flex System® (ThermoFisher Scientific®). After RNA extraction, 16 μL of RNA was used to synthesize cDNA by using SuperScript IV VILO® Master Mix (ThermoFisher Scientific®) followed by pre-amplification by using TaqMan® PreAmp® Master Mix (ThermoFisher Scientific®).

### OpenArray® Profiling

OpenArray® Technology (ThermoFisher Scientific®) is a high-throughput real-time PCR genotyping method that allows for rapid screening of several TaqMan® assays in several samples. This real-time method involves the use of an array composed of 3072 through-holes running on the QuantStudio® 12K Flex Real Time PCR System with an OpenArray® block.

For each sample, 2.5 μL of pre-amplified cDNA and 2.5 μL of 2× TaqMan® OpenArray® Master Mix were manually mixed and loaded into 384 well-plates according to the manufacturer's instructions (ThermoFisher Scientific®). The QuantStudio® 12K Flex OpenArray® AccuFill® System transferred the previously generated mix to the TaqMan® OpenArray plate. The amplification was performed using the QuantStudio® 12K Flex Real Time PCR System (ThermoFisher Scientific®) instrument, and the ΔΔCt method was used to analyze expression with the QuantStudio 12K Flex Software (ThermoFisher Scientific®).

### Data Preprocessing

To remove obvious outliers from 3 technical replicates of OpenArray® QPCR and address undetected data points, the following data preprocessing steps were established: 1) if Ct=“Undetermined” or Amp.Status=“Inconclusive/No Amp”, Ct was set to 35; 2) standard deviation (SD) for 3 replicates were calculated; 3) if SD >=1, the replicate with the largest difference from mean was removed; if SD<1, all 3 replicates were kept; 4) Ct mean was calculated from the remaining 2 or 3 replicates. All the Ct mean were normalized by KLK3 using the formula −[Ct mean of gene X−Ct mean of KLK3]. The normalized Ct was used for downstream model building. Since exponentiation was not applied, the normalized data was in logarithmic scale. Reasoning that samples with low KLK3 might indicate ineffective DRE and might not be reliable, a cutoff of 95th percentile was set to remove samples with high KLK3 Ct means.

Mathematical Model building

To avoid multicollinearity in regression models, highly correlated variables were identified and removed with a stepwise procedure. Specifically, variance inflation factor (VIF) was calculated for all variables (gene expression from 54 probes+clinical variables including PSA density and prostate volume), and the variable with the highest VIF was removed; VIF was re-calculated with remaining variables, and this step was repeated until no more variables had VIFs>5. Nine probes (including probes targeting COL9A2, PLA2G7, HPN, CYB561A3, PDLIM5, MYO6, GAPDH, GDF15, and one of the two probes targeting PCA3) and PSA density were removed in the prefiltering step. To select important genes, 3 model building strategies, including logistic regression with stepwise feature selection (James D A, et al., Modern applied statistics with S-PLUS. Technometrics. 1996; 38(1):77), logistic regression with recursive feature elimination, and regularized logistic regression with elastic net (Friedman J, et al., Regularization Paths for Generalized Linear Models via Coordinate Descent. J Stat Softw. 2010; 33(1):1-22), were evaluated. The model building step was implemented with glmStepAIC (in both forward and reverse direction), rfe and glmnet functions from the R package “caret” (Kuhn M, et al., caret: Classification and Regression Training. R package version 6.0-86. Astrophysics Source Code). Elastic-net is considered a mathematical model with built-in feature selection as nonimportant variables are given zero importance. Repeated cross validation (10-fold repeated 3 times) was used to evaluate mathematical model performance. The minor class (high-grade, 39%, Table 1) was up sampled to create balanced classes during training. For stepwise and RFE, feature selection was considered part of mathematical model building and was encapsulated inside each fold (FIG. 2A).

Elastic-net was chosen for final mathematical model building as it showed best performance in terms of median AUC in a total of 30 resampling. To select a robust gene panel, the final mathematical model was developed in an ensemble approach by integrating information of multiple elastic-net regression models built from resampling (FIG. 2B). Specifically, the training data was firstly randomly split into 4 partitions and the step was repeated 4 times to generate a total of 40 resampling. The elastic-net regression model was fit to the 40 subsampling of the training data; the frequency and importance of each gene in all resampling was then summed together. Genes were subsequently ranked by selection frequency and summed importance, and the top 17 genes were selected to include in the final model. The number of genes was determined based on preliminary analysis of optimal feature size using RFE as well as optimal design of the OpenArray® plate. Coefficients for each of the 17 genes were estimated by fitting the entire training data with an elastic-net regression model (referred to as “MPS2”). Additionally, an enhanced model was built by incorporating the 17 genes and clinical variables (age, race, family history, abnormal DRE, and prior negative biopsy) (referred to as “MPS2c”). Besides the above variables, prostate volume was added to build a third model which can be used when this information is available (referred to as “MPS2cv” or “MPS2+”).

### Mathematical Model Calibration

Calibration curves were used to evaluate the concordance between predicted probability and observed prevalence in each bin. Logistic regression is considered a well calibrated classifier. However, calibration is necessary when there is a distributional shift between training and validation population. In this study, classes were balanced during training while the validation cohort was a consecutive cohort, and its 20% high-grade prevalence reflected the true distribution which was imbalanced. Without calibration, calibration curves (FIG. 7) showed overall overestimation of risk. Therefore, it was important to calibrate the models to make predictions for each individual patient reflect the real risk in a true population. Two calibration techniques were tested—recalibration in the large (re-estimation of model intercept) and recalibration (re-estimation of intercept and slope) (Vergouwe Y, et al., A closed testing procedure to select an appropriate method for updating prediction models. Stat Med. 2017; 36(28):4529-39) in a resampled UM cohort to match 20% high-grade in the validation cohort. Calibration curves were generated with original and calibrated probabilities. Recalibration performed better at high probability bins, and all following calibration was performed with this approach.

### Model Validation

Raw data from the validation cohort was preprocessed in the same manner as the training cohort. Using normalized Ct values of the validation cohort, predictions from the three models (MPS2, MPS2c, and MPS2cv) were made with the “predict” function from caret (Kuhn M, et al., caret: Classification and Regression Training. R package version 6.0-86. Astrophysics Source Code.). Calibration on the model prediction was then conducted using the intercept and slope for each model separately, estimated as described in the “Model calibration” section above.

### Statistical Analysis

Statistical analyses were performed using R version 4.1 (R Core Team. R: A language and environment for statistical computing. Vienna, Austria: R Foundation for Statistical Computing; 2013 2013). Kruskal-Wallis test was used for group comparison, and p values <0.05 were considered statistically significant. Regularized logistic regression with elastic-net was used to build models to predict high-grade prostate cancer, implemented with wrapper function of “glmnet” provided by caret (Kuhn M, et al., caret: Classification and Regression Training. R package version 6.0-86. Astrophysics Source Code.). Diagnostic potential was visualized by Receiver Operating Characteristic (ROC) curves and quantified by Area Under Curve (AUC), using R package pROC (Robin X, et al., pROC: an open-source package for R and S+ to analyze and compare ROC curves. BMC Bioinformatics. 2011; 12:77). Calibration analysis was performed with calibration function from caret by setting cuts=8 (number of bins). Decision Curve Analysis (DCA) was performed using dca function from dcurves (Sjoberg D D. Dcurves: decision curve analysis for model evaluation, 2021).

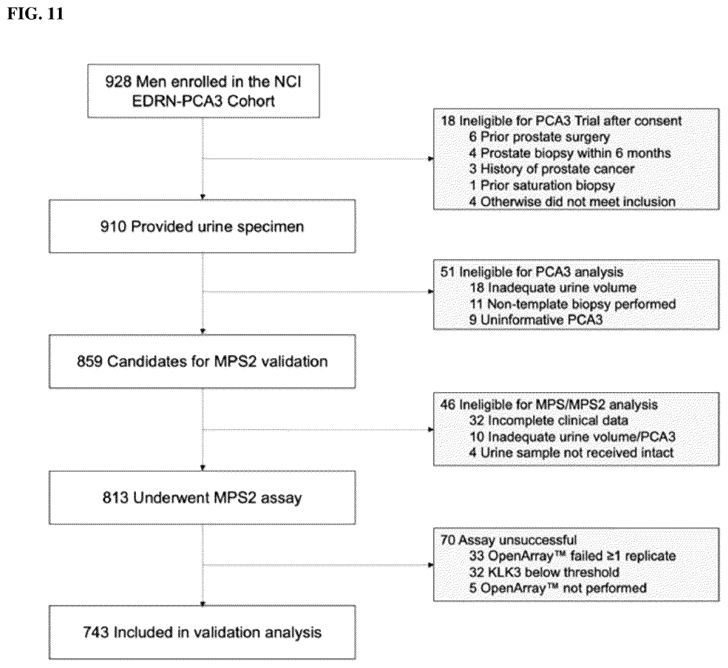

Across the clinically pertinent threshold values spanning about 4% to 20%, MPS2 models provided the highest net clinical benefit across all tests (FIG. 10A). The threshold probability (x-axis) reflects how the patient and clinician value potential clinical outcomes. For example, a threshold probability of about 4% applies to patients that would choose to pursue biopsy if their risk of clinically significant prostate cancer is about 4% or higher. For clinically significant prostate cancer, a about 4% threshold probability represents a risk-averse population, such as younger men with a long life-expectancy. At a practice level, this implies that the clinician would be willing to perform as many as 20 biopsies to detect an additional clinically significant prostate cancer. At the other end of the spectrum, a threshold probability of 20% applies to patients that would choose to pursue biopsy only if their risk of clinically significant prostate cancer was ≥20%. Such a population strongly values avoiding biopsy and is willing to accept a higher risk of delayed detection of clinically significant prostate cancer. The unit of net benefit (y-axis) is true positives. A net benefit of 0.15 is equivalent to an approach in which 15 patients per 100 are directed to biopsy based on use of the test, and all 15 patients are found to have clinically significant prostate cancer. Plots were made with ggplot2 (Wickham H. Springer; New York: 2009. Ggplot2: elegant graphics for data analysis).

### Results

The results include urinary transcript markers of high-grade prostate cancer.

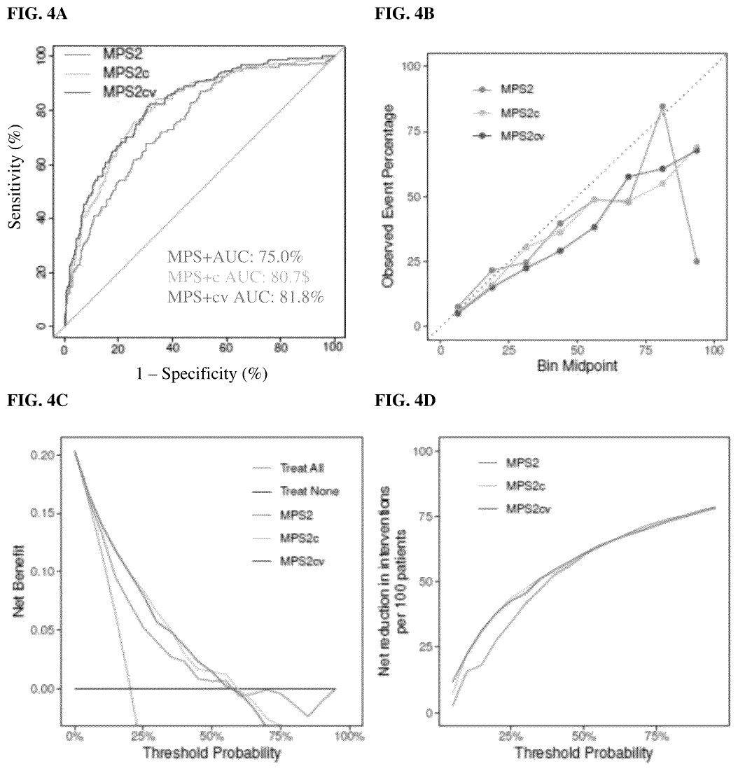

Additional cancer- and high-grade prostate cancer-specific transcripts were identified to be added to the MPS test (Tomlins S A, et al., Urine TMPRSS2:ERG fusion transcript stratifies prostate cancer risk in men with elevated serum PSA. Sci Transl Med. 2011; 3(94):94ra72; Tomlins S A, et al., Urine TMPRSS2:ERG Plus PCA3 for Individualized Prostate Cancer Risk Assessment. Eur Urol. 2016; 70(1):45-53). For transcript 4 nomination, RNA-seq data from The Cancer Genome Atlas (TCGA) prostate adenocarcinoma (PRAD) cohort were analyzed (see Methods above). Briefly, biomarker discovery was performed using RNA sequencing (RNAseq) data from 220 benign prostates, 71 GG1 and 484 GG≥2 cancers available through The Cancer Genome Atlas (TCGA), the Genotype-Tissue Expression (GTEx) portal, and the University of Michigan (U-M). Forty-four transcripts meeting predefined nomination criteria were supplemented with 10 curated cancer-associated genes. This analysis resulted in a 54-marker panel, which included two reference genes as well as PCA3 and the T2:ERG gene fusion from the original MPS assay (Table 5). Expressions of each of these genes in relation to increasing Gleason score are shown in FIGS. 4A-4D.

A custom ThermoFisher® OpenArray® QPCR-based platform (see Methods) was then developed for detection of these transcripts in urine samples collected from patients immediately following a digital rectal exam (DRE). The training cohort of patients (FIG. 1B, Table 1) consisted of men undergoing prostate biopsy at the University of Michigan (U-M). Of the initial 921 patients included in the UM training cohort, 761 men had Prostate Cancer Prevention Trial (PCPT) clinical variables available, a PSA <10 ng/mL, prostate volume available, and a threshold cycle (Ct)<27 for the KLK (PSA) transcript in their urine biospecimen (FIG. 1B). In these 761 patients, 293 (38.5%) were found to have GG≥2 prostate cancer on biopsy (Table 1).

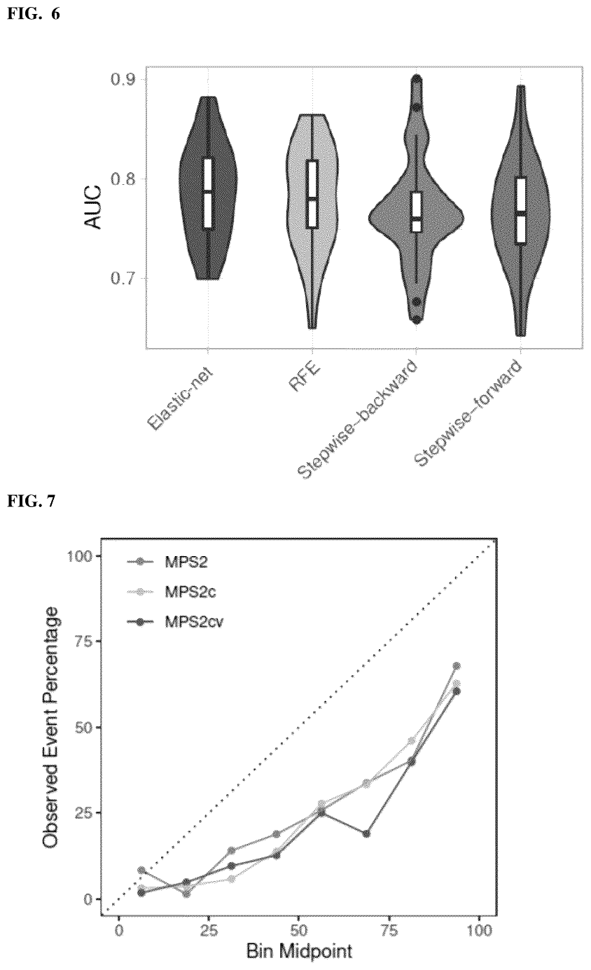

Following QPCR analysis of the 54 markers in urine samples from the training cohort, MPS2 model development was undertaken (FIG. 2A). One important aspect in model development is dimension reduction to reduce the model complexity and avoid overfitting. After prefiltering with a variance inflation factor (VIF)>5 to remove redundant variables, 46 genes remained. Three different model building algorithms with encapsulated feature selection were firstly evaluated using repeated cross validation (CV) on the training cohort, including logistic regression with stepwise feature selection (forward and backward direction), logistic regression with recursive feature selection (RFE), and regularized logistic regression with elastic net. Elastic net has built-in feature selection as it provides feature importance, which can be used for this purpose. The median area under the curve (AUC) of the elastic net regression approach was highest (FIG. 6); therefore, it was selected to build the final MPS2 mathematical models. Using an ensemble approach, which integrates data from multiple mathematical models over resamplings, the development set was randomly divided into four partitions, and the model yielding the highest AUC was identified for each partition. This approach was repeated ten times with different random seeds, yielding 40 elastic net models in total. The frequency of model inclusion and importance to clinically significant prostate cancer detection was tabulated across models. Based on analysis of optimal feature size and technical features of the OpenArray™ platform, the 17 biomarkers providing optimal discriminative accuracy for GG≥2 prostate cancer were included with standard clinical variables and the normalization gene KLK3 in the MPS2 and MPS2+ (plus prostate volume) models. Models were calibrated and internally cross-validated prior to external validation (see FIG. 1B).The performances of models, starting with the 54 MPS2 genes, as well as MPS2 genes plus clinical variables (with and without prostate volume), were evaluated using repeated CV. Prediction of high-grade prostate cancer was improved by including more genes compared to PCA3 and T2:ERG only in terms of AUC (0.784 vs 0.731) (FIGS. 3A and 3B). Incorporating clinical variables (not including prostate volume) increased the AUC to 0.802 (FIG. 3C), and including prostate volume brought the AUC up to 0.820 (FIG. 3D).

It was observed that certain genes were frequently selected in different CV folds, while selection of others was more random. To select a robust gene set, the training data with all genes was partitioned into four subsamples, and the data partition was repeated 10 times using different random seeds, resulting in a total of 40 subsamples (FIG. 2B). Each subsample was trained with elastic net, and the top 17 genes were selected for the final mathematical model based on importance and frequency (Table 3). The final MPS2 model was built using the 17 genes from the entire training cohort, and the MPS2c and MPS2cv models were built by adding clinical variables without and with prostate volume, respectively. Calibration curves after applying class imbalance correction showed that predicted risks lined up with observed risks (FIG. 3E). Performance of the locked 17-transcript MPS2 models was next tested on a validation cohort, which consisted of a blinded, multi-institutional National Cancer Institute-Early Detection Research Network (NCI-EDRN) prostate biopsy cohort (FIG. 1B, Table 1). Of the 743 final patients included in the validation cohort, 20.3% had GG≥2 prostate cancer on biopsy (Table 1). For the MPS2 models, although logistic regression is considered a well calibrated classifier, calibration is necessary when there is a distributional shift between training and validation population. Classes in this study were balanced during training while the validation cohort was a consecutive cohort, and its 20% high-grade prevalence reflected the true distribution which was imbalanced. Without calibration, MPS2 calibration curves (FIG. 7) showed an overall overestimation of risk. Therefore, it was important to calibrate the models to make predictions for each individual patient reflect the real risk in a true population, as detailed in the Methods section above (see also Tables 6, 7, and 8).

As shown in FIG. 4A, the final MPS2 models outperformed the original MPS model, with values similar to those obtained in the training cohort. MPS2, MPS2c, and MPS2cv had AUC values of 0.750, 0.807, and 0.818, respectively, compared to 0.730 for the original MPS assay. Final calibration curves of each model showed that predicted risks lined up with observed risks in the validation cohort (FIG. 4B). Decision curve analysis also demonstrated net benefits of the MPS2 models versus “Treat All” or “Treat None” across different probability thresholds (FIG. 4C), and interventions (biopsies) avoided across different probability thresholds with each model were also calculated (FIG. 4D, Table 4).

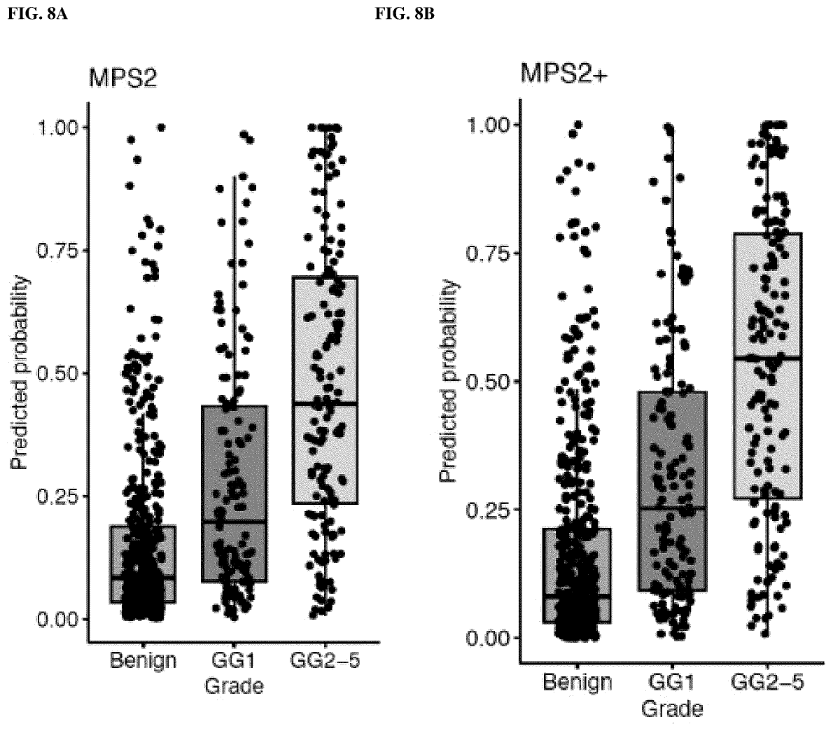

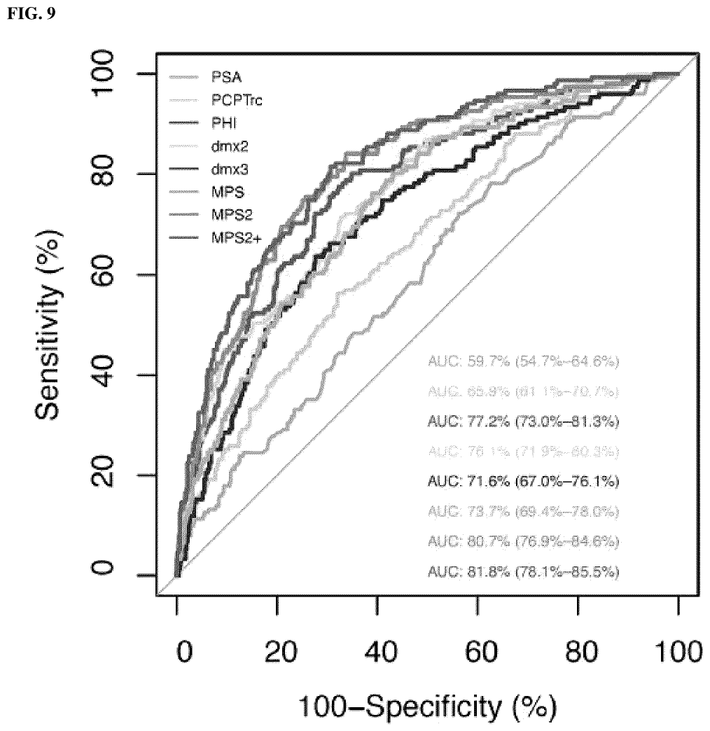

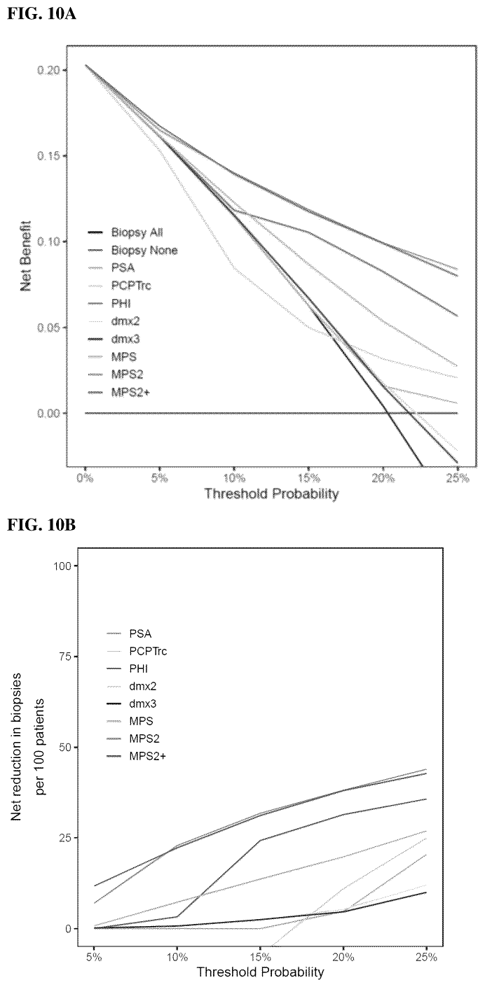

Of 859 men participating in the PCA3 trial, 46 (5.4%) were ineligible for the current analysis due to inadequate urine volume or unavailable clinical data. Of 813 validation patients (FIG. 11), qPCR was successful in 743 (91%). Median PSA was 5.6 ng/mL (IQR 4.1-8.0), and 247 men (33%) had a previous negative biopsy (Table 1). On study biopsy, 151 men (20%) had GG≥2 PCa (prostate cancer). Median MPS2 values were significantly higher in men with GG≥2 prostate cancer than in men with negative biopsies and men with GG1 prostate cancer (0.44 vs. 0.08 and 0.20, respectively; both p<0.001) (Table 1, FIG. 8A). Similarly, median MPS2+ was significantly higher in men with GG≥2 cancer relative to negative or GG1 biopsies (0.54 vs. 0.08 and 0.25, respectively; p<0.001, FIG. 8B). The AUC for GG≥2 cancer was 0.60 for PSA, 0.66 for PCPTrc, 0.77 for PHI, 0.76 for dmx2, 0.72 for dmx3, and 0.74 for MPS, as compared to 0.81 for MPS2 and 0.82 for MPS2+ (FIG. 9). The observed prevalence of GG≥2 cancer closely approximated the MPS2 and MPS2+ predicted probabilities (FIG. 2C), reflecting good calibration. Critically, the MPS2 models were particularly well-calibrated for predicted probabilities <30%.

Using testing thresholds detecting 95% of GG≥2 prostate cancers (i.e., 95% sensitivity), the proportions of unnecessary biopsies that would have been avoided using each test were 11% for PSA, 20% for PCPTrc, 26% for PHI, 27% for dmx2, 17% for dmx3, and 23% for MPS, as compared to 37% for MPS2 and 41% for MPS2+. Full performance measures and unnecessary biopsies avoided are listed in Table 2. Critically, MPS2 and MPS2+ provided 99% sensitivity and 99% NPV for GG≥3 prostate cancer.

The initial biopsy population included 496 patients with median PSA 5.0 ng/mL (IQR 3.8-6.6) (Table 7). On study biopsy, 133 patients (27%) had GG≥2 cancer. Using a 95% sensitivity threshold, the proportions of unnecessary biopsies avoided were 15% for PSA, 27% for PCPT, 30% for PHI, 30% for dmx2, 17% for dmx3, and 27% for MPS, as compared to 35% for MPS2 (Table 2). Although initial biopsy patients often may not have prostate volume available, use of MPS2+ would have avoided 42% of unnecessary biopsies.

The repeat biopsy population included 247 men with median PSA 7.2 ng/mL (IQR 5.5-9.8), of which 18 (7.3%) were found to have GG≥2 prostate cancer (Table 7). At 95% sensitivity for GG≥2 cancer, the proportions of unnecessary biopsies avoided were 15% for PSA, 8.7% for PHI, 14% for dmx2, 16% for dmx3, and 15% for MPS, as compared to 46% for MPS2 and 51% for MPS2+ (Table 2). Accordingly, MPS2 testing would have avoided approximately one-half of unnecessary biopsies while maintaining detection of 95% of GG≥2 prostate cancers. Performance of MPS2 models with and without clinical factors are provided by subpopulation (Tables 8-9). Across the clinically pertinent threshold values spanning 5% to 20%, MPS2 models provided the highest net clinical benefit across all tests (FIG. 10A). Expressing benefit as net reduction in unnecessary biopsies, MPS2 provided the greatest net reduction in unnecessary biopsies without failing to biopsy a single patient with GG≥2 prostate cancer (FIG. 10B).

Translating sequencing-based discovery to an expandable qPCR platform, provided herein is a test incorporating 17 markers of Pca, and markers uniquely overexpressed by high-grade cancers. Three MPS2 models, which incorporated 17 biomarkers alone or with clinical data (MPS2c) and prostate volume (MPS2cv), were developed. Validation of the MPS2 models in a blinded, external cohort showed that the models improved upon diagnostic accuracy of the original MPS model as well as increased specificity. MPS2 testing with 95% sensitivity for GG≥2 cancer provided 95-99% NPV and 35-51% specificity across subgroups. For individual patients, NPVs approaching 100% provide clear guidance for confident decision-making. For clinicians, uniform use of MPS2 could avoid up to one-half of unnecessary biopsies, while preserving immediate detection of 95% of GG≥2 cancers diagnosed under the “biopsy all” approach. Critically, MPS2 provided 99% sensitivity and 99% NPV for GG≥3 cancers, meaning the rare false-negative MPS2 results were almost uniformly more favorable GG2 cancers least likely to metastasize.

Altogether, the findings indicate that the MPS2 test can improve detection of clinically significant prostate cancer and may be useful in identifying prostate cancer patients which will most benefit from more aggressive treatments.

TABLE 1
Characteristics of the development and validation populations overall and stratified by
pathologic findings on prostate biopsy.

| | Development Cohort | | | | | External Validation Cohort | | | | |
|---|---|---|---|---|---|---|---|---|---|---|
| | | | | Negative/ | | | | | Negative/ | |
| | Total | Negative | GG1 | GG1 | GG 2 to 5 | Total | Negative | GG1 | GG1 | GG 2 to 5 |
| N | 761 | 362 | 105 | 4 [symbol] 8 (61%) | 293 (39%) | 743 | 452 (61%) | 140 (19%) | 582 ( [symbol] 0%) | 151 (20%) |
| Median | 63 (58-88) | 62 (57-87) | 64 (57-88) | 63 (57-68) | 64 (58-89) | 62 (57-88) | 62 (57-67) | 63 (57-67) | 62 (57-68) | 64 (59-70) |
| age (IQR);- | | | | | | | | | | |
| years | | | | | | | | | | |
| African- | 33 (4.3%) | 12(3.3%) | 4 (3.8%) | 16 (3.4%) | 17 (5.8%) | 85 (13%) | 51 (11%) | 19 (14%) | 70 (12%) | 25 (17%) |
| American- | | | | | | | | | | |
| No. (%) | | | | | | | | | | |
| Postive | 206 (27%) | 88 (24.3%) | 23 (26.4%) | 116 (25%) | 90 (31%) | 212 (29%} | 118 (26%) | 46 (33%) | 164 (28%) | 48 (32%) |
| family | | | | | | | | | | |
| history- | | | | | | | | | | |
| No. (%) | | | | | | | | | | |
| Previous | 163 (21%) | 105 (28%) | 22 (20.8%) | 127 (27%) | 36 (12%) | 247 (33%) | 198 (23%) | 33(24%) | 220 (39%) | 18(12%) |
| negative | | | | | | | | | | |
| [symbol] -No. (%) | | | | | | | | | | |
| Abnormal | 104 (14%) | 34 (8.4%) | 4 (3.8%) | 38 (8.1%) | 66 (23%) | 139 (19%) | 72 (16%) | 18 (11%) | 88 (15%) | 51 (34%) |
| DRE- | | | | | | | | | | |
| No. (%) | | | | | | | | | | |
| Median | 48 (36-86) | 56 (42-76) | 47 (37-81) | 53 (40-72) | 41 (30-54) | 43 (32-80) | 48 (35-69) | 40 (29-52) | 46 (34-65) | 38 (28-47) |
| prostate | | | | | | | | | | |
| volume [symbol] - | | | | | | | | | | |
| mL | | | | | | | | | | |
| Median | 5.6 | 5.6 | [symbol] | 5.6 | 5.6 | 5.6 | 5.5 | 5.3 | 5.4 | 5.2 |
| PSA (IQR)- | (4.6-7.2) | (4.6-6.8) | (4.6-7.0) | (4.6-6.8) | (4.7-7.5) | (4.1-8.0) | (4.0-8.0) | (4.3-7.0) | (4.8-7.7) | (4.7-8.9) |
| ng/mL | | | | | | | | | | |
| Median | 0.12 | 0.10 | 0.12 | 0.10 | 0.15 | 0.12 | 0.11 | 0.12 | 0.11 | 0.17 |
| PSA | (0.08-0.18) | (0.07-0.14) | (0.09-0.18) | (0.07-0.14) | (0.10-0.20) | (0.06-0.18) | (0.07-0.17) | (0.09-0.18) | (0.08-0.17) | (0.12-0.31) |
| density [symbol] | | | | | | | | | | |
| (IQR)- | | | | | | | | | | |
| ng/mL³ | | | | | | | | | | |
| Median | NA | NA | NA | NA | NA | 40.5 | 35.5 | 40.8 | 37.4 | 57.5 |
| PHI (IQR) | | | | | | (30.0-55.0) | (27.7-47.5) | (32.2-50.7) | (28.4-49.5) | (44.9-86.8) |
| Median | NA | NA | NA | NA | NA | 0.48 | 0.40 | 0.45 | 0.42 | 0.70 |
| mxSMDx | | | | | | (0.33-0.67) | (0.08-0.59) | (0.36-0.64) | (0.01-0.8) | (0.49-0.90) |
| Median | NA | NA | NA | NA | NA | 0.45 | 0.38 | 0.50 | 0.41 | 0.59 |
| mxEPI | | | | | | (0.31-0.60) | (0.28-0.51) | (0.39-0.66) | (0.290.55) | (0.46-0.70) |
| Median | 37 (20-58) | 26 (14-42) | 42 (24-63) | 29 (16-46) | 51 (33-72) | 35 (17-56) | [symbol] (12-44) | 42 (26-65) | 30 (15-49) | 55 (37-72) |
| MPS (IQR) | | | | | | | | | | |
| Median | 0.18 | 0.07 | 0.15 | 0.07 | 0.40 | 0.13 | 0 08 | 0.20 | 0.10 | 0.44 |
| MPS2 [symbol] | (0.05-(0.08) | (0.03-0.16) | (0.06-0.33) | (0.03-0.19) | (0.20-0.61) | (0.05-0.37) | (0.03-0.19) | (0.08-0.43) | (0.04-0.24) | (0.23-0.89) |
| Median | 0.14 | 0 06 | 0.124 | 0.07 | 0 44 | 0.15 | 0 08 | 0.25 | 0.11 | 0.54 |
| MPS2+ [symbol] | (0.05-0.42) | (0.02-0.14) | (0.06-0.32) | (0.03-0.17) | (0.22-0.68) | (0.05-0.43) | (0.03-0.21) | (0.09-0.48) | (0.044-0.30) | (0.27-0.79) |
| (IQR) | | | | | | | | | | |

Abbreviations:
DRE, digital rectal examination;
GG, grade group;
IGR, interquartile range;
MPS, MyProstateScore;
MPS2, MyProstateScore 2.0;
MPS2+, MyPrestateScore 2.0 plus;
mxSMDx, multiplex SelectMDx, mxEPI, multiplex ExoDx Prostate Intelliscore;
PHI, prostate health index;
PSA, prostate-specific antigen [symbol]
Measured by transrectal ultrasound. [symbol]
PSA density equals semm PSA divided by prostate volume. [symbol]
MPS2 and MPS2+ values are reported on a continuous scale as the likelihood of [symbol]
indicates data missing or illegible when filed

TABLE 2
Performance of PSA, PCPTrc, PHI, dmx2, dmx3, MPS, MPS2, and
MPS2+ in the EDRN Validation Cohort: Overall (N = 743), Initial
Biopsy (N = 496), and Repeat Biopsy (N = 247) Subpopulations.

| | | | | | No. Estimated |
|---|---|---|---|---|---|
| | | | | | Unnecessary Biopsies |
| | | | | | Avoided per 1000 |
| | Sensitivity | Specificity | NPV | PPV | Patients |
| Overall | | | | | |
| PSA | 95% | 11% | 90% | 21% | 110 |
| PCPTrc | 95% | 20% | 94% | 23% | 201 |
| PHI | 95% | 26% | 96% | 22% | 260 |
| dmx2 | 95% | 27% | 96% | 23% | 271 |
| dmx3 | 95% | 17% | 94% | 23% | 371 |
| MPS | 95% | 23% | 94% | 24% | 230 |
| MPS2 | 95% | 37% | 97% | 28% | 370 |
| MPS2+ | 95% | 41% | 97% | 29% | 410 |
| Initial | | | | | |
| PSA | 95% | 15% | 89% | 29% | 149 |
| PCPTrc | 95% | 27% | 94% | 32% | 270 |
| PHI | 95% | 30% | 95% | 33% | 300 |
| dmx2 | 95% | 30% | 95% | 33% | 300 |
| dmx3 | 95% | 17% | 91% | 30% | 169 |
| MPS | 95% | 27% | 93% | 32% | 270 |
| MPS2 | 95% | 35% | 95% | 35% | 351 |
| MPS2+ | 95% | 42% | 96% | 37% | 419 |
| Repeat | | | | | |
| PSA | 94.4% | 15% | 97% | 8.0% | 150 |
| PCPTrc | 94.4% | 21% | 98% | 8.6% | 211 |
| PHI | 94.4% | 8.7% | 95% | 7.5% | 87 |
| dmx2 | 94.4% | 14% | 97% | 8.0% | 142 |
| dmx3 | 94.4% | 16% | 97% | 8.1% | 162 |
| MPS | 94.4% | 15% | 97% | 8.0% | 150 |
| MPS2 | 94.4% | 46% | 99% | 12% | 462 |
| MPS2+ | 94.4% | 51% | 99% | 13% | 510 |

Abbreviations: MPS2, MyProstateScore 2.0; MPS2+, MyProstateScore 2.0 plus; dmx2, derived multiplex 2-gene model (HOXC6, DLX1); dmx3, derived multiplex 3-gene model (PCA3, ERG, SPDEF); PHI, prostate health index; PSA, prostate-specific antigen.

TABLE 3
Genes selected for the MPS2 prediction model of high-grade
prostate cancer. Frequency of Inclusion and Cumulative
Importance of the 17 Most Informative Markers Across 40
Elastic Net Models Assessed in Development.

| | | Chro- | | | |
|---|---|---|---|---|---|
| | Gene | mo- | | Fre- | Cumulative |
| No. | Name | some | Gene ID | quency | Importance ^{a} |
| 1 | TMPRSS2- | 21-21 | ENSG00000184012, | 40 | 1265 |
| | ERG | | ENSG00000157554 | | |
| 2 | SCHLAP1 | 2 | ENSG00000281131 | 35 | 1582 |
| 3 | OR51E2 | 11 | ENSG00000167332 | 33 | 2006 |
| 4 | APOC1 | 19 | ENSG00000130208 | 31 | 456 |
| 5 | PCAT14 | 22 | ENSG00000280623 | 30 | 841 |
| 6 | CAMKK2 | 12 | ENSG00000110931 | 29 | 1604 |
| 7 | PCA3 | 9 | ENSG00000225937 | 28 | 1015 |
| 8 | NKAIN1 | 1 | ENSG00000084628 | 28 | 456 |
| 9 | B3GNT6 | 11 | ENSG00000198488 | 28 | 211 |
| 10 | TFF3 | 21 | ENSG00000160180 | 26 | 1329 |
| 11 | SPON2 | 4 | ENSG00000159674 | 26 | 1080 |
| 12 | PCGEM1 | 2 | ENSG00000227418 | 26 | 725 |
| 13 | TRGV9 | 7 | ENSG00000211685 | 24 | 955 |
| 14 | TMSB15A | X | ENSG00000158164 | 22 | 548 |
| 15 | ERG | 21 | ENSG00000157554 | 21 | 221 |
| 16 | KLK4 | 19 | ENSG00000167749 | 20 | 1094 |
| 17 | HOXC6 | 12 | ENSG00000197757 | 20 | 354 |

^{a} Cumulative importance indicates the relative weight of marker importance summed across repeat samplings as derived by elastic net modeling.

TABLE 4
Performance metrics at various thresholds on the validation cohort.
PPV, positive predictive value; NPV, negative predictive value.

| Model | Threshold | Sensitivity | Specificity | PPV | NPV |
|---|---|---|---|---|---|
| MPS2 | 0.026 | 0.974 | 0.093 | 0.215 | 0.932 |
| MPS2 | 0.064 | 0.960 | 0.328 | 0.267 | 0.970 |
| MPS2 | 0.071 | 0.954 | 0.340 | 0.269 | 0.966 |
| MPS2 | 0.081 | 0.940 | 0.390 | 0.282 | 0.963 |
| MPS2 | 0.081 | 0.934 | 0.399 | 0.284 | 0.959 |
| MPS2 | 0.087 | 0.921 | 0.416 | 0.287 | 0.953 |
| MPS2 | 0.087 | 0.914 | 0.416 | 0.285 | 0.950 |
| MPS2 | 0.095 | 0.901 | 0.446 | 0.293 | 0.946 |
| MPS2 | 0.117 | 0.854 | 0.500 | 0.304 | 0.931 |
| MPS2 | 0.134 | 0.801 | 0.541 | 0.308 | 0.914 |
| MPS2 | 0.173 | 0.702 | 0.650 | 0.339 | 0.895 |
| MPS2c | 0.036 | 0.974 | 0.226 | 0.243 | 0.971 |
| MPS2c | 0.044 | 0.960 | 0.284 | 0.255 | 0.966 |
| MPS2c | 0.060 | 0.954 | 0.351 | 0.273 | 0.967 |
| MPS2c | 0.065 | 0.940 | 0.382 | 0.28 | 0.962 |
| MPS2c | 0.068 | 0.934 | 0.392 | 0.281 | 0.959 |
| MPS2c | 0.079 | 0.921 | 0.436 | 0.294 | 0.956 |
| MPS2c | 0.087 | 0.914 | 0.458 | 0.301 | 0.954 |
| MPS2c | 0.114 | 0.901 | 0.535 | 0.331 | 0.955 |
| MPS2c | 0.132 | 0.854 | 0.598 | 0.351 | 0.941 |
| MPS2c | 0.178 | 0.801 | 0.679 | 0.389 | 0.931 |
| MPS2c | 0.270 | 0.702 | 0.779 | 0.447 | 0.911 |
| MPS2cv | 0.043 | 0.974 | 0.264 | 0.252 | 0.975 |
| MPS2cv | 0.061 | 0.960 | 0.360 | 0.277 | 0.973 |
| MPS2cv | 0.066 | 0.954 | 0.380 | 0.282 | 0.970 |
| MPS2cv | 0.079 | 0.940 | 0.421 | 0.293 | 0.965 |
| MPS2cv | 0.082 | 0.934 | 0.431 | 0.295 | 0.862 |
| MPS2cv | 0.086 | 0.921 | 0.443 | 0.296 | 0.956 |
| MPS2cv | 0.102 | 0.914 | 0.478 | 0.309 | 0.956 |
| MPS2cv | 0.113 | 0.901 | 0.514 | 0.321 | 0.953 |
| MPS2cv | 0.170 | 0.854 | 0.620 | 0.364 | 0.943 |
| MPS2cv | 0.227 | 0.801 | 0.696 | 0.402 | 0.932 |
| MPS2cv | 0.322 | 0.702 | 0.769 | 0.436 | 0.910 |

TABLE 5
Information for the 54 genes included in the OpenArray assay.

| | | | | | | | | High. vs Low-Grade | | High-Grade vs [symbol] | | | |
|---|---|---|---|---|---|---|---|---|---|---|---|---|---|
| gene_id | sequence | start | end | strand | gene_name | gene_type | [symbol] | logFC | adj.P.Vol | logFC | adj.P.Vol | A [symbol] C | Note |
| [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | | | | | | | | | | | | | |
| [symbol] | | | | | | | | | | | | | |
| [symbol] | | | | | | | | | | | | | |
| [symbol] | | | | | | | | | | | | | |
| [symbol] | | | | | | | | | | | | | |
| [symbol] | | | | | | | | | | | | | |
| [symbol] | | | | | | | | | | | | | |
| [symbol] | | | | | | | | | | | | | |
| [symbol] | | | | | | | | | | | | | |
| [symbol] | | | | | | | | | | | | | |
| [symbol] | | | | | | | | | | | | | |
| [symbol] | | | | | | | | | | | | | |
| [symbol] | | | | | | | | | | | | | |
| [symbol] | | | | | | | | | | | | | |
| [symbol] | | | | | | | | | | | | | |
| [symbol] | | | | | | | | | | | | | |
| [symbol] | | | | | | | | | | | | | |
| [symbol] | | | | | | | | | | | | | |
| [symbol] | | | | | | | | | | | | | |
| [symbol] | | | | | | | | | | | | | |
| [symbol] | | | | | | | | | | | | | |
| [symbol] | | | | | | | | | | | | | |
| [symbol] | | | | | | | | | | | | | |
| [symbol] | | | | | | | | | | | | | |
| [symbol] | | | | | | | | | | | | | |
| [symbol] | | | | | | | | | | | | | |
| [symbol] | | | | | | | | | | | | | |
| [symbol] | | | | | | | | | | | | | |
| [symbol] | | | | | | | | | | | | | |
| [symbol] | | | | | | | | | | | | | |
| [symbol] | | | | | | | | | | | | | |
| [symbol] | | | | | | | | | | | | | |
| [symbol] | | | | | | | | | | | | | |
| [symbol] | | | | | | | | | | | | | |
| [symbol] | | | | | | | | | | | | | |
| [symbol] | | | | | | | | | | | | | |
| [symbol] | | | | | | | | | | | | | |
| [symbol] | | | | | | | | | | | | | |
| [symbol] | | | | | | | | | | | | | |
| [symbol] | | | | | | | | | | | | | |
| [symbol] | | | | | | | | | | | | | |
| [symbol] | | | | | | | | | | | | | |
| [symbol] | | | | | | | | | | | | | |
| [symbol] | | | | | | | | | | | | | |
| [symbol] | | | | | | | | | | | | | |
| [symbol] | | | | | | | | | | | | | |
| [symbol] | | | | | | | | | | | | | |
| [symbol] | | | | | | | | | | | | | |
| [symbol] | | | | | | | | | | | | | |
| [symbol] | | | | | | | | | | | | | |
| [symbol] | | | | | | | | | | | | | |
| [symbol] | | | | | | | | | | | | | |
| [symbol] | | | | | | | | | | | | | |

[symbol]

indicates data missing or illegible when filed

TABLE 6
Model Coefficients for the MPS2 and MPS2+ Models.

| Covariate | MPS2 ^{a} | MPS2+ ^{b} |
|---|---|---|
| (Intercept) | 5.902430658 | 6.676363594 |
| T2ERG | 0.111906862 | 0.148584627 |
| SCHLAP1 | 0.17335791 | 0.205268829 |
| OR51E2 | 0.200676934 | 0.228791882 |
| APOC1 | −0.07916931 | −0.08896388 |
| PCAT14 | 0.14420976 | 0.16009867 |
| CAMKK2 | −0.26364401 | −0.277941927 |
| PCA3.1 | 0.080881661 | 0.074209893 |
| NKAIN1 | −0.06946207 | −0.093791082 |
| B3GNT6 | 0.047475092 | 0.072524885 |
| TFF3 | 0.186395669 | 0.2128103 |
| SPON2 | 0.156664808 | 0.1740959 |
| PCGEM1 | −0.16940833 | −0.149084289 |
| TRGV9 | 0.096184103 | 0.177972309 |
| TMSB15A | 0.151071453 | 0.214870771 |
| ERG | 0.023544761 | 0.030085251 |
| KLK4 | 0.149451849 | 0.214609188 |
| HOXC6 | 0.05612131 | 0 |
| Age | 0.000134221 | 0.021446485 |
| African American | 0.828856591 | 1.232493234 |
| Family History | 0.148709502 | 0.292757369 |
| Abnormal DRE | 0.888379309 | 1.094432439 |
| Previous Biopsy | −0.8505938 | −0.61694213 |
| PSA | 0.073709982 | 0.092554335 |
| Prostate Volume | N/A | −0.024051593 |

^{a} MPS2: Calibrated logit = −1.453526 + logit*1.302089
^{b} MPS2+: Calibrated logit = −1.41207 + logit*1.077061

TABLE 7
Characteristics of the NCI-EDRN External Validation Population
Stratified by Previous Biopsy Status.

| | Initial Biopsy | | Repeat Biopsy | |
|---|---|---|---|---|
| Characteristic | (N = 496) | | (N = 247) | |
| Median age (IQR) - years | 62 | (56-67) | 63 | (59-68) |
| African-American - No. (%) | 70 | (14%) | 25 | (10%) |
| Positive family history - | 134 | (27%) | 78 | (32%) |
| No. (%) | | | | |
| Previous negative biopsy - | 0 | (0%) | 247 | (100%) |
| No. (%) | | | | |
| Abnormal DRE - No. (%) | 111 | (22%) | 28 | (11%) |
| Median prostate volume ^{a} - | 40 | (29-51) | 56 | (39-81) |
| mL | | | | |
| Median PSA (IQR) - ng/mL | 5.0 | (3.8-6.6) | 7.2 | (5.5-9.8) |
| Median PSA density ^{b} | 0.12 | (0.08-0.19) | 0.12 | (0.09-0.20) |
| (IQR) - ng/mL² | | | | |
| Median PHI (IQR) | 40.8 | (30.2-55.1) | 39.3 | (29.6-54.7) |
| Median PCA3 (IQR) | 27.4 | (12.4-61.1) | 21.2 | (10.0-45.1) |
| Median MPS (IQR) | 34 | (16-57) | 35 | (17-55) |
| Median MPS2 ^{c} (IQR) | 0.20 | (0.08-0.46) | 0.06 | (0.02-0.14) |
| Median MPS2+ ^{c} (IQR) | 0.22 | (0.08-0.53) | 0.06 | (0.02, 0.21) |
| Biopsy GG ≥2 - No. (%) | 133 | (27%) | 18 | (7.3%) |

Abbreviations: DRE, digital rectal examination; GG, denotes grade group; IQR, interquartile range; MPS, MyProstateScore; MPS2, MyProstateScore 2.0, MPS2+, MyProstateScore 2.0 plus, PCA3, prostate cancer antigen 3; PHI, prostate health index; PSA, prostate-specific antigen
^{a} Measured by transrectal ultrasound.
^{b} PSA density equals serum PSA divided by prostate volume.
^{c} MPS2 and MPS2+ values are reported on a continuous scale as the likelihood of detecting clinically significant prostate cancer on biopsy

TABLE 8
Clinical Performance of High Sensitivity MPS2 Threshold Values in
the Initial Biopsy Subpopulation of the External Validation
Cohort (N = 496).

| Threshold | Sensitivity | Specificity | NPV | PPV |
|---|---|---|---|---|
| MPS2+ | | | | |
| 0.05 | 97% | 21% | 95% | 31% |
| 0.06 | 97% | 25% | 96% | 32% |
| 0.07 | 96% | 29% | 95% | 33% |
| 0.075 | 96% | 31% | 96% | 34% |
| 0.08 | 95% | 32% | 95% | 34% |
| 0.09 | 95% | 35% | 95% | 35% |
| 0.10 | 95% | 38% | 95% | 36% |
| 0.11 ^{a} | 95% | 42% | 96% | 37% |
| 0.12 | 92% | 44% | 94% | 38% |
| 0.13 | 92% | 48% | 95% | 39% |
| 0.14 | 92% | 51% | 94% | 41% |
| 0.15 | 90% | 53% | 94% | 41% |
| MPS2 | | | | |
| 0.05 | 96% | 21% | 94% | 31% |
| 0.06 | 96% | 25% | 95% | 32% |
| 0.07 | 96% | 28% | 95% | 33% |
| 0.075 | 96% | 31% | 96% | 34% |
| 0.08 | 95% | 33% | 94% | 34% |
| 0.087 ^{a} | 95% | 35% | 95% | 35% |
| 0.09 | 94% | 36% | 94% | 35% |
| 0.10 | 94% | 39% | 95% | 36% |
| 0.11 | 93% | 41% | 94% | 37% |
| 0.12 | 92% | 46% | 94% | 38% |
| 0.13 | 91% | 49% | 94% | 39% |
| 0.14 | 89% | 52% | 93% | 41% |
| 0.15 | 89% | 54% | 93% | 41% |
| Markers Only | | | | |
| 0.05 | 95% | 20% | 92% | 30% |
| 0.06 | 95% | 28% | 94% | 33% |
| 0.07 | 95% | 33% | 94% | 34% |
| 0.075 | 95% | 35% | 95% | 35% |
| 0.077 ^{a} | 95% | 35% | 95% | 35% |
| 0.08 | 94% | 37% | 94% | 35% |
| 0.09 | 92% | 41% | 93% | 36% |
| 0.10 | 90% | 44% | 92% | 37% |

^{a} Optimal threshold value providing 95% sensitivity for GG2 or higher cancer.

TABLE 9
Clinical Performance of High Sensitivity MPS2 Threshold Values
in the Repeat Biopsy Subpopulation of the External Validation
Cohort (N = 247).

| Threshold | Sensitivity | Specificity | NPV | PPV |
|---|---|---|---|---|
| MPS2+ | | | | |
| 0.04 | 100% | 40% | 100% | 12% |
| 0.05 | 94% | 47% | 99% | 12% |
| 0.054 | 94% | 49% | 99% | 13% |
| 0.058 ^{a} | 94% | 51% | 99% | 13% |
| 0.06 | 88% | 52% | 98% | 13% |
| MPS2 | | | | |
| 0.038 | 100% | 41% | 100% | 12% |
| 0.04 | 94% | 42% | 99% | 11% |
| 0.044 ^{a} | 94% | 46% | 99% | 12% |
| 0.05 | 89% | 48% | 98% | 12% |
| 0.054 | 89% | 49% | 98% | 12% |
| 0.06 | 89% | 52% | 98% | 13% |
| Markers Only | | | | |
| 0.04 | 100% | 22% | 100% | 9.1% |
| 0.05 | 100% | 26% | 100% | 9.6% |
| 0.054 | 100% | 29% | 100% | 10% |
| 0.06 | 100% | 32% | 100% | 10% |
| 0.07 | 100% | 35% | 100% | 11% |
| 0.08 ^{a} | 94% | 42% | 99% | 11% |

^{a} Optimal threshold value providing 94.4% sensitivity for GG2 or higher cancer.

TABLE 10
Model Coefficients from MPS2 Re-Development Using Maximum
Grade Group from All Biopsy and Surgical Specimens.

| | | RP-Derived | | RP-Derived |
|---|---|---|---|---|
| Covariate | MPS2 | MPS2^{a} | MPS2+ | MPS2+^{a} |
| (Intercept) | 5.902430658 | 5.36963703 | 6.676363594 | 5.66713268 |
| TZERG | 0.111906862 | 0.12028164 | 0.148584627 | 0.15333868 |
| SCHLAP1 | 0.17335791 | 0.15765111 | 0.205268829 | 0.14709972 |
| OR51E2 | 0.200676934 | 0.17998234 | 0.228791882 | 0.15904006 |
| APOC1 | −0.07916931 | −0.0798262 | −0.08896388 | −0.0924547 |
| PCAT14 | 0.14420976 | 0.05653337 | 0.16009867 | 0.08193915 |
| CAMKK2 | −0.26364401 | −0.2662075 | −0.277941927 | −0.2934813 |
| PCA3.1 | 0.080881661 | 0.12143166 | 0.074209893 | 0.09433227 |
| NKAIN1 | −0.06946207 | N/A | −0.093791082 | N/A |
| B3GNT6 | 0.047475092 | 0.04017777 | 0.072524885 | 0.05564982 |
| TFF3 | 0.186395669 | 0.20852164 | 0.2128103 | 0.24750486 |
| SPON2 | 0.156664808 | 0.08542159 | 0.1740959 | 0.03934708 |
| PCGEM1 | −0.16940833 | −0.1154365 | −0.149084289 | −0.1389629 |
| TRGV9 | 0.096184103 | 0.2130775 | 0.177972309 | 0.24262976 |
| TMSB15A | 0.151071453 | N/A | 0.214870771 | N/A |
| ERG | 0.023544761 | N/A | 0.030085251 | N/A |
| KLK4 | 0.149451849 | N/A | 0.214609188 | N/A |
| HOXC6 | 0.05612131 | 0.07807035 | 0 | 0.06422307 |
| ACSM1 | N/A | 0 | N/A | −5.73E−05 |
| LRRN1 | N/A | −0.0212629 | N/A | |
| MS4A8 | N/A | 0.00093909 | N/A | 0.02808881 |
| GRIN3A | N/A | 0 | N/A | −0.0040861 |
| Age | 0.000134221 | 0.00168473 | 0.021446485 | 0.0210196 |
| African | 0.828856591 | 0.47328332 | 1.232493234 | 0.56924532 |
| American | | | | |
| Family | 0.148709502 | 0.00459593 | 0.292757369 | 0.0820011 |
| History | | | | |
| Abnormal | 0.888379309 | 0.68733551 | 1.094432439 | 0.86237867 |
| DRE | | | | |
| Previous | −0.8505938 | −0.6063101 | −0.61694213 | −0.4762385 |
| Biopsy | | | | |
| PSA | 0.073709982 | 0.03565447 | 0.092554335 | 0.10335382 |
| Prostate | N/A | N/A | −0.024051593 | −0.0255555 |
| Volume | | | | |

Abbreviations: DRE, digital rectal exam; MPS2, MyProstateScore2.0; MPS2+ MyProstateScore 2.0 plus; PSA, prostate-specific antigen; RP radical prostatectomy and/or repeat biopsy.
^{a} Considering potential misclassification of clinically significant prostate cancer due to biopsy undersampling, we evaluated MPS2 models including pathologic data obtained subsequent to study urine collection (e.g., repeated biopsy, radical prostatectomy). Of 761 patients in the development set, 382 (50%) underwent additional biopsy (N = 201) and/or radical prostatectomy (N-217), and 71 (11%) were upgraded to clinically significant prostate cancer. The table includes parameters from models derived based on the highest cancer grade detected (i.e., RP-derived models). Of the 17 informative markers in the MPS2 model, 13 were retained in the RP-derived model. The direction of biomarker association with the outcome was unchanged for all markers. The AUC of the cross validated models differed by 1% for MPS2 (0.802 vs. 0.792) and 0.1% for MPS2+ (0.821 vs. 0.822).

### Example 2

Sample Collection and mRNA Detection

A urine sample is collected from a subject suspected of having or at risk of developing prostate cancer to determine the likelihood that Grade Group ≥2 prostate cancer would be detected from a prostate biopsy of the subject. A subject is given a digital rectal examination (DRE), and a urine sample is collected within approximately 1 hour after the DRE. The urine is placed into a collection tube having a stabilization buffer at a ratio of about 2:5 of buffer:sample by volume.

Positive and negative controls are prepared. Negative controls are samples from previously reported subjects in the “low risk category”. Positive controls are samples from previously reported subjects in the “elevated risk category”.

RNA is isolated from the test sample, negative control and positive control using a commercially available RNA isolation kit. The extracted RNA is subjected to RT-PCR to generate cDNA, pre-amplification, and qPCR to determine the amount of mRNA expressed from each of the following genes: TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6. The amount of mRNA expressed from a reference gene, such as KLK3, is also determined using RT-PCR, pre-amplification, and qPCR. Target-specific primers are used to amplify the cDNA, and gene-specific probes that release fluorophores are used to accurately and quantitatively measure the expression levels of the target genes listed above.

### Analysis and MPS2 Score Generation

Based on the qPCR performed above, average Crt values (cycle threshold values) are determined for each target gene and normalized to the average Crt value of the reference gene (e.g., KLK3) using the following equation: Crt mean (target)−Crt mean (reference). The normalized Crt is multiplied by a gene specific coefficient. Exemplary gene specific coefficients are provided in the table below. The sum of the normalized Crt×coefficient=logit value. The logit value is re-calibrated using an intercept and slope. The logit value is converted to a score using the logit equation. Gene specific coefficients, the slope, and the intercept are different for biopsy naïve subjects or subject that had a previous negative prostate biopsy.

TABLE 11

| | Biopsy Naïve | Biopsy Negative |
|---|---|---|
| Target Name | Coefficient | Coefficient |
| APOC1 | −0.06 | −0.0889639 |
| B3GNT6 | 0.05 | 0.07252488 |
| CAMKK2 | −0.35 | −0.2779419 |
| ERG | 0.02 | 0.03008525 |
| HOXC6 | 0.01 | 0 |
| KLK3 | N/A | N/A |
| KLK4 | 0 | 0.21460919 |
| NKAIN1 | −0.08 | −0.0937911 |
| OR51E2 | 0.25 | 0.22879188 |
| PCA3.1 | 0.13 | 0.07420989 |
| PCAT14 | 0.13 | 0.16009867 |
| PCGEM1 | −0.14 | −0.1490843 |
| SCHLAP1 | 0.22 | 0.20526883 |
| SPON2 | 0.11 | 0.1740959 |
| TFF3 | 0.17 | 0.2128103 |
| T2ERG | 0.14 | 0.14858463 |
| (TMPRSS2-ERG) | | |
| TMSB15A | 0.17 | 0.21487077 |
| TRGV9 | 0.13 | 0.17797231 |
| Age | — | 0.02144648 |
| aa | — | 1.23249323 |
| Family history | — | 0.29275737 |
| DRE abnormal | — | 1.09443244 |
| Biopsy prior neg | — | −0.6169421 |
| PSA | — | 0.09255434 |
| Prostate Volume | — | −0.0240516 |
| Intercept | −1.406563436 | −1.412069526 |
| Slope | 1.128771142 | 1.07706078 |

An illustrative calculation for biopsy naïve subjects is:
1. Each Crt mean value for each 17 target normalized to KLK3=(target gene Crt mean value−KLK3 Crt mean value)
2. Normalized value*coefficient for designated target
3. (sum of (2) for all target)+intercept
4. (intercept+(3))*(slope)=logit
5. MPS2 probability=(Exp(4))/Exp(4)+1)
   An illustrative calculation for a subject with a prior negative prostate biopsy is:
1. Each Crt mean value for each 17 target normalized to KLK3=(target gene Crt mean value−KLK3 Crt mean value)
2. Add the following clinical variables and prostate volume to (1):
   a. Age
   b. African American (binary)
   c. Negative biopsy=1
   d. Abnormal DRE (binary)
   e. Family history (binary)
   f. Serum PSA
   g. PSA volume
3. Normalized value*coefficient for designated target+sum of (2a-2g)
4. (sum of (3) for all target)+intercept
5. (intercept+(3))*(slope)=logit
6. MPS2 probability=(Exp(4))/Exp(4)+1)
   The threshold for determining low risk or elevated risk is different for biopsy naïve subjects and subject with a prior negative prostate biopsy:

TABLE 12

| Biopsy Status | MPS2 Score | Risk Category |
|---|---|---|
| Biopsy Naive | 0-7.5% | LOW |
| Biopsy Naive | ≥7.6% | ELEVATED |
| Biopsy Negative | 0-5.4% | LOW |
| Biopsy Negative | ≥5.5% | ELEVATED |

Based on the above results, a report is generated with the score and risk category and provided to the subject's health care provider that requested the test (e.g., subject's urologist).

All publications, patents, patent applications and accession numbers mentioned in the above specification are herein incorporated by reference in their entirety. Although the invention has been described in connection with specific embodiments, it should be understood that the invention as claimed should not be unduly limited to such specific embodiments. Indeed, various modifications and variations of the described compositions and methods of the invention will be apparent to those of ordinary skill in the art and are intended to be within the scope of the following claims.

1.-3. (canceled)

4. A method for sparing a subject from having a prostate biopsy, the method comprising:
a) detecting an amount of expression of genes TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6, wherein the amount of expression is present in the subject's urine;
b) prognosing, from the amount of expression of each of the genes, the subject as having or not having a low likelihood of having Grade Group ≥2 prostate cancer; and
c) 1) recommending to the subject or the subject's healthcare provider that the subject undergo a prostate biopsy if the subject is prognosed as not having a low likelihood of having Grade Group ≥2 prostate cancer, or
2) recommending to the subject or the subject's healthcare provider that the subject not undergo a prostate biopsy if the subject is prognosed as having a low likelihood of having Grade Group ≥2 prostate cancer.

5.-10. (canceled)

11. The method of claim 4, further comprising performing a prostate biopsy of the subject if the subject is prognosed as not having a low likelihood of having Grade Group ≥2 prostate cancer.

12. The method of claim 11, wherein the prostate biopsy indicates that the subject has Grade Group ≥2 prostate cancer.

13. The method of claim 4, further comprising generating a report with the subject's risk category, and forwarding the report to the subject or to a health care provider of the subject.

14.-18. (canceled)

19. The method of claim 4, wherein the subject has not had a prior prostate biopsy.

20. The method of claim 4, wherein the subject has had a prior negative prostate biopsy result.

21.-22. (canceled)

23. The method of claim 4, wherein the urine is obtained within one hour after the subject's digital rectal examination (DRE).

24.-27. (canceled)

28. The method of claim 91, wherein said prostate cancer treatment is one or more of surgery, radiation therapy, hormonal therapy, targeted therapy, chemotherapy, immunotherapy, radiopharmaceuticals, and bone-modifying drugs.

29. The method of claim 4, wherein said amount of expression is the amount of mRNA or protein expressed by said genes.

30. A method for sparing a subject from having a prostate biopsy, the method comprising:
a) detecting an amount of expression of genes TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6, wherein the amount of expression is present in a biological sample from the subject, and wherein said sample is selected from tissue, blood, plasma, serum, urine, prostate secretions, and prostate cancer cells;
b) prognosing, from the amount of expression of each of the genes, the subject as having or not having a low likelihood of having Grade Group ≥2 prostate cancer; and
c) 1) recommending to the subject or the subject's healthcare provider that the subject undergo a prostate biopsy if the subject is prognosed as not having a low likelihood of having Grade Group ≥2 prostate cancer, or
2) recommending to the subject or the subject's healthcare provider that the subject not undergo a prostate biopsy if the subject is prognosed as having a low likelihood of having Grade Group ≥2 prostate cancer.

31. The method of claim 30, wherein the sample is urine, and the urine is obtained within one hour after the subject's digital rectal examination (DRE).

32.-34. (canceled)

35. The method of claim 30, wherein detecting the amount of expression of said genes comprises detecting an amount of mRNA expression of the genes.

36.-37. (canceled)

38. The method of claim 4 or 30, further comprising detecting an amount of expression of a reference gene and normalizing the amount of expression of each of genes TMPRSS2-ERG, SCHLAP1, OR51 E2, APOC1, PCAT14, CAMKK2, PCA3, NKAIN1, B3GNT6, TFF3, SPON2, PCGEM1, TRGV9, TMSB15A, ERG, KLK4, and HOXC6 to the amount of expression of the reference gene.

39. The method of claim 38, wherein detecting the amount of expression of the reference gene comprises detecting an amount of mRNA expressed by the reference gene.

40. The method of claim 38, wherein the reference gene is KLK3, CYPB561A3, EEF1A2, GAPDH, HPN, KLK2, LBH, NUDT8, SPDEF, or TRGV9.

41. The method of claim 40, wherein the reference gene is KLK3.

42.-60. (canceled)

61. A kit comprising:
a container, the container containing a reagent composition useful for detecting an amount of expression of genes; and
instructions for detecting the amount of expression, where the amount of expression is present in a subject's urine and the genes are TMPRSS2-ERG, SCHLAP1, OR51E2, APOC1, PCAT14, CAMKK2, PCA3, NKAIN1, B3GNT6, TFF3, SPON2, PCGEM1, TRGV9, TMSB15A, ERG, KLK4, and HOXC6.

62.-67. (canceled)

68. The kit of claim 61, wherein the subject has not had a prior prostate biopsy.

69. The kit of claim 61, wherein the subject has had a prior negative prostate biopsy result.

70.-79. (canceled)

80. The method of claim 4 or 30, wherein the method does not comprise performing a prostate biopsy of the subject if the subject is prognosed as having a low likelihood of having Grade Group ≥2 prostate cancer.

81. (canceled)

82. The method of claim 30, wherein the method further comprises performing a prostate biopsy of the subject if the subject is prognosed as not having a low likelihood of having Grade Group ≥2 prostate cancer.

83. (canceled)

84. The method of claim 82, wherein the prostate biopsy indicates the subject has Grade Group ≥2 prostate cancer.

85. The method of claim 11 or 82, wherein the prostate biopsy indicates the subject does not have Grade Group ≥2 prostate cancer.

86. (canceled)

87. The method of claim 30, further comprising generating a report with the subject's risk category, and forwarding the report to the subject or to a health care provider of the subject.

88. The method of claim 30, wherein the subject has not had a prior prostate biopsy.

89. The method of claim 30, wherein the subject has had a prior negative prostate biopsy result.

90. The method of claim 12, further comprising administering a prostate cancer treatment to the subject.

91. The method of claim 84, further comprising administering a prostate cancer treatment to the subject.

92. The method of claim 91, wherein said prostate cancer treatment is one or more of surgery, radiation therapy, hormonal therapy, targeted therapy, chemotherapy, immunotherapy, radiopharmaceuticals, and bone-modifying drugs.


SEQUENCE LISTING (17 sequences)

SEQ ID NO: 1 - RNA, 293 nt, Homo sapiens
  source 1..293: mol_type = transcribed RNA
taggcgcgagctaagcaggaggcggaggcggaggcggagggcgaggggcggggagcgccgcctggagcgcggcaggaagccttatcagttgtgagtgaggaccagtcgttgtttgagtgtgcctacggaacgccacacctggctaagacagagatgaccgcgtcctcctccagcgactatggacagacttccaagatgagcccacgcgtccctcagcaggattggctgtctcaacccccagccagggtcaccatcaaaatggaatgtaaccctagccaggtgaatggctcaag

SEQ ID NO: 2 - RNA, 1574 nt, Homo sapiens
  source 1..1574: mol_type = transcribed RNA
gcttttatgagctgtaacactcaccgcgaaggtccgcagcttcactcctgaagccagcgagaccacgagcctactgggaggaacgaacaactcccgacgcgccgccttaagagctgtaacactcaccgcgaaggtctgcagcttcactcctgagccagcgagaccacgaacccaccagaaggaaaaaactccgaacacatctgaacatcagaagcaacaaactccggacacgccgcctttaagaactgtaacactcactgcgagggtccgcggcttcattcttgaagtgagtgagaccaagaacccaccagttctggacacaatttcaagtcctcaggtgccatcaatattctgaaaatggcagtgatttttattcaacctgtataaggcactttcaccatgtacctggaagcaacatctacatctttttcagtttcttctacgccaggtgtgtgcttagctccatgacaaaaggtgacagcttattctgcagcacacacacatcatcaaagtgggaggtggtgagactggcacactgacagtctgtcctagcagatttcagctcacactgcaatctagatgctggggacacaaggtccaccttccaggaatatggccatgacaccagaaatcacaaacatgatgagaatggaatgactggggaagaagtgccagatgcttcacttgtaaatgaagacccagcctctggggatgcagataccacctccctgaagaagctgaatatctgcagataagtggagttcaccaatgatgaggagcgggatggagaaaggaggtagggagagtcatccaaggaacatgagcaacatgttaaaagccaagtggtttaatttctggagatggtgaacccaagaggctctgctgggagacaacaaaaataatgaagaattgaaccagagtccggtgaatatcagcactgggaccagttagcagaggaaaaggaaagaataaaagcgaaaagaatgaagagtcatatgattaccaacttttcctttttcatataaattgagtgtatatgggtctggaacaacctgaatttccatcaagtcctggctaacctcattatgtcctatgaatatttttgactaatcccactttacattaatctgtattgtgaatgtggatattgaattatatttctttgtaatcccattatccaaaatccagttcagagactattagttaccaatgttcactgtgaaggaaaaaaaaaaaaaaaaagctcagaggataaacatgtgatatggtttggctgtgtccccacccaaatatcatcttgaattgtagctcccataattcccacgtgttgtgggagggacccggtgggagataattgtatcatgggggtggttcccccatactattctcatagtagtgaataagtctcacaaaatctgatggttttatgagggaaaacccctttcacctggttctcattctcttctctggtctgtcgtcatgtaagacatgcctttcaccttctccaccatgactgtgaggcctccccagccacgtggaactgtgagcccattaaacctctttcacttataaat

SEQ ID NO: 3 - RNA, 2792 nt, Homo sapiens
  source 1..2792: mol_type = transcribed RNA
cttctgggaatctccacaccctgaagacacagtgagttagcaccaccaccaggaattggcctttcagctctgtgcctgtctccagtcaggctggaataagtctcctcatatttgcaagctcggccctcccctggaatctaaagcctcctcagccttctgagtcagcctgaaaggaacaggccgaactgctgtatgggctctactgccagtgtgacctcaccctctccagtcacccctcctcagttccagctatgagttcctgcaacttcacacatgccacctttgtgcttattggtatcccaggattagagaaagcccatttctgggttggcttccccctcctttccatgtatgtagtggcaatgtttggaaactgcatcgtggtcttcatcgtaaggacggaacgcagcctgcacgctccgatgtacctctttctctgcatgcttgcagccattgacctggccttatccacatccaccatgcctaagatccttgcccttttctggtttgattcccgagagattagctttgaggcctgtcttacccagatgttctttattcatgccctctcagccattgaatccaccatcctgctggccatggcctttgaccgttatgtggccatctgccacccactgcgccatgctgcagtgctcaacaatacagtaacagcccagattggcatcgtggctgtggtccgcggatccctcttttttttcccactgcctctgctgatcaagcggctggccttctgccactccaatgtcctctcgcactcctattgtgtccaccaggatgtaatgaagttggcctatgcagacactttgcccaatgtggtatatggtcttactgccattctgctggtcatgggcgtggacgtaatgttcatctccttgtcctattttctgataatacgaacggttctgcaactgccttccaagtcagagcgggccaaggcctttggaacctgtgtgtcacacattggtgtggtactcgccttctatgtgccacttattggcctctcagtggtacaccgctttggaaacagccttcatcccattgtgcgtgttgtcatgggtgacatctacctgctgctgcctcctgtcatcaatcccatcatctatggtgccaaaaccaaacagatcagaacacgggtgctggctatgttcaagatcagctgtgacaaggacttgcaggctgtgggaggcaagtgacccttaacactacacttctccttatctttattggcttgataaacataattatttctaacactagcttatttccagttgcccataagcacatcagtacttttctctggctggaatagtaaactaaagtatggtacatctacctaaaggactattatgtggaataatacatactaatgaagtattacatgatttaaagactacaataaaaccaaacatgcttataacattaagaaaaacaataaagatacatgattgaaaccaagttgaaaaatagcatatgccttggaggaaatgtgctcaaattactaatgatttagtgttgtccctactttctctctcttttttctttcttttttttttattatggttagctgtcacatacaactttttttttttttgagatggggtctcgctctgtcaccaggctggagtgcagtggcgcgatctcggctcactgcaacctccacatcccatgttgaagtaattcttctgcctcagcctcccgagtagctgggactagaggaacgtgccaccatgactggctaattttctgtattttttagtagagacagagtttcaccatgttggccaggatggtctcgatctcctgaccttgtgatccacccgcctcagcctcccaaagtgttgggattacaggtgtgaaccactgtgcccggcctgtgtacaactttttaaatagggaatatgatagcttcgcatggtggtgtgcacctatagcccccactgcctggaaagctgaggtgggagaatcgcttgagtccaggagtttgaggttacagtgatccacgatcgtaccactacactccagcctgggcaacagagcaagaccctgtctcaaagcataaaatggaataacatatcaaatgaaacagggaaaatgaagctgacaatttatggaagccagggcttgtcacagtctctactgttattatgcattacctgggaatttatataagcccttaataataatgccaatgaacatctcatgtgtgctcacaatgttctggcactattataagtgcttcacaggttttatgtgttcttcgtaactttatggagtaggtaccatttgtgtctctttattataagtgagagaaatgaagtttatattatcaaggggactaaagtcacacggcttgtgggcactgtgccaagatttaaaattaaatttgatggttgaatacagttacttaatgaccatgttatattgcttcctgtgtaacatctgccatttatttcctcagctgtacaaatcctctgttttctctctgttacacactaacatcaatggctttgtacttgtgatgagagataaccttgccctagttgtgggcaacacatgcagaataatcctgttttacagctgcctttcgtgatcttattgcttgcttttttccagattcagggagaatgttgttgtctatttgtctcttacatctccttgatcatgtcttcattttttaatgtgctctgtacctgtcaaaaattttgaatgtacaccacatgctattgtctgaacttgagtataagataaaataaaattttattttaaatttt

SEQ ID NO: 4 - RNA, 514 nt, Homo sapiens
  source 1..514: mol_type = transcribed RNA
aggcggtcaggggaaggctcaggaggagggagatcaacatcaacctgccccgccccctccccagcctgataaaggtcctgcgggcaggacaggacctcccaaccaagccctccagcaaggattcagagtgcccctccggcctcgccatgaggctcttcctgtcgctcccggtcctggtggtggttctgtcgatcgtcttggaaggcccagccccagcccaggggaccccagacgtctccagtgccttggataagctgaaggagtttggaaacacactggaggacaaggctcgggaactcatcagccgcatcaaacagagtgaactttctgccaagatgcgggagtggttttcagagacatttcagaaagtgaaggagaaactcaagattgactcatgaggacctgaagggtgacatcccaggaggggcctctgaaatttcccacaccccagcgcctgtgctgaggactccctccatgtggccccaggtgccaccaataaaaatcctacagaaaa

SEQ ID NO: 5 - RNA, 1201 nt, Homo sapiens
  source 1..1201: mol_type = transcribed RNA
gagatacggcctcgtgggaagggaaagacctgaccgtcccccagcccgacacccgtaaagggtctgtgctgaggaggattagtaaaaggggaaggcctcttgcagttgagataagaggaaggcctccgtctcctgcatgtccttgggaatggaatgtcttggtgtaaaacccgatagtacattccttctattctgagagaagaaaaccaccctgtggctggagggtgaaggtactctacagtgtggtcattgaggacaagttgacgagagagtcccaagtacgtccacggtcagccttgcgacatttaaagttctacaatgaactcactggagatgcaaagaaaagtgtggagatggagacaccccaatcgactcgccagtctacaggtgtatccagcagctccaaagagacagcaaccagcaagaatgggccatagtgacgatggtggttttgtcaaaaagaaaagggggggatatgtaaggaaaagagagatcagactttcactgtgtctatgtagaaaaggaagacataagaaactccattttgatctgtactaagaaaaattgttttgccttgagatgctgttaatctgtaactttagccccaaccctgtgctcacggaaacatgtgctgtaaggtttaagggatctagggctgtgcaggatgtaccttgttaacaatatgtttgcaggcagtatgtttggtaaaagtcatcgccattctccattctcgattaaccaggggctcaatgcactgtggaaagccacaggaacctctgcccaagaaagcctggctgttgtgggaagtcagggaccccgaatggagggaccagctggtgctgcatcaggaaacataaattgtgaagatttcttggacatttatcagtttccaaaattaatacttttataatttcttacacctgtcttactttaatctcttaatcctgttatctttgtaagctgaggatatacgtcacctcaggaccactattgtacaaattgattgtaaaacatgttcacatgtgtttgaacaatatgaaatcagtgcaccttgaaaatgaacagaataacagtgattttagggaacaaaggaagacaaccataaggtctgactgcctgaggggtcgggcaaaaagccatatttttcttcttgcagagagcctataaatggacgtgcaagtaggagagatattgctaaatt

SEQ ID NO: 6 - RNA, 4905 nt, Homo sapiens
  source 1..4905: mol_type = transcribed RNA
agagcaagctgagccgagccgagccgagctgggggcgcagagcgcgggaggcggcggcggcgcggagcccaggtggctccgctgccggatgggagtgccccagtgtgctggatgaagctggcgcatgcaccatgtcatcatgtgtctctagccagcccagcagcaaccgggccgccccccaggatgagctggggggcaggggcagcagcagcagcgaaagccagaagccctgtgaggccctgcggggcctctcatccttgagcatccacctgggcatggagtccttcattgtggtcaccgagtgtgagccgggctgtgctgtggacctcggcttggcgcgggaccggcccctggaggccgatggccaagaggtcccccttgacacctccgggtcccaggcccggccccacctctccggtcgcaagctgtctctgcaagagcggtcccagggtgggctggcagccggtggcagcctggacatgaacggacgctgcatctgcccgtccctgccctactcacccgtcagctccccgcagtcctcgcctcggctgccccggcggccgacagtggagtctcaccacgtctccatcacgggtatgcaggactgtgtgcagctgaatcagtataccctgaaggatgaaattggaaagggctcctatggtgtcgtcaagttggcctacaatgaaaatgacaatacctactatgcaatgaaggtgctgtccaaaaagaagctgatccggcaggccggctttccacgtcgccctccaccccgaggcacccggccagctcctggaggctgcatccagcccaggggccccattgagcaggtgtaccaggaaattgccatcctcaagaagctggaccaccccaatgtggtgaagctggtggaggtcctggatgaccccaatgaggaccatctgtacatggtgttcgaactggtcaaccaagggcccgtgatggaagtgcccaccctcaaaccactctctgaagaccaggcccgtttctacttccaggatctgatcaaaggcatcgagtacttacactaccagaagatcatccaccgtgacatcaaaccttccaacctcctggtcggagaagatgggcacatcaagatcgctgactttggtgtgagcaatgaattcaagggcagtgacgcgctcctctccaacaccgtgggcacgcccgccttcatggcacccgagtcgctctctgagacccgcaagatcttctctgggaaggccttggatgtttgggccatgggtgtgacactatactgctttgtctttggccagtgcccattcatggacgagcggatcatgtgtttacacagtaagatcaagagtcaggccctggaatttccagaccagcccgacatagctgaggacttgaaggacctgatcacccgtatgctggacaagaaccccgagtcgaggatcgtggtgccggaaatcaagctgcacccctgggtcacgaggcatggggcggagccgttgccgtcggaggatgagaactgcacgctggtcgaagtgactgaagaggaggtcgagaactcagtcaaacacattcccagcttggcaaccgtgatcctggtgaagaccatgatacgtaaacgctcctttgggaacccattcgagggcagccggcgggaggaacgctcactgtcagcgcctggaaacttgctcaccaaaaaaccaaccagggaatgtgagtccctgtctgagctcaaggaagcaaggcagcgaagacaacctccagggcaccgacccgccccccgtgggggaggaggaagtgctcttgtgagaggcagtccctgcgtggaaagttgctgggcccccgcccccggctcccccgcacgcatgcatccactgcggccggaggaggccatggagcccgagtagctgcctggatcgctcgacctcgcatgcgcgccgcgtcgcctctggggggctgctgcaccgcgtttccatagcagcatgtcctacggaaacccagcacgtgtgtagagcctcgatcgtcatctctggttatttgttttttcctttgttgttttaaaggggacaaaaaaaaaaaaaggacttgactccatgacgtcgaccgtggccgctggctggctggacaggcgggtgtgaggagttgcagacccaaacccacgtgcattttgggacaattgctttttaaaacgtttttatgccaaaaatccttcattgtgattttcagaaccacgtcagatataccaagtgactgtgtgtggggtttgacaactgtggaaaggcgagcagaaaactccggcggtctgaggccatggaggtggttgctgcatttgagagggagtagggggctagatgtggctcctagtgcaaaccggaaaccatggcaccttccagagccgtggtctcaaggagtcagagcagggctggccctcagtagctgcagggagctttgatgcaacttatttgtaagaaggatttttaaattttttatgggtagaattgtagtcaggaaaacagaaagggcttgaaatttaataagtgctgctggaaggggattttccaagcctggaagggtattcagcagctgtggtggggaaacatttctcctgaaagactgaacgtgtttcttcatgacagctgctcaaagcaggtttctgagatagctgaccgagctctggtaaatctctttgtcaaattacgaaaacttcagggtgaaatcctatgcttccatgtacattacatggcttaagattaaacaaaaacatttttcaagtctctaactagagtgaactctagagcacagtagttcagaaactatttagagcttccaggatatatttcacagcttcaggcatgtgatcagttagagccgatgaaacctatgcccgcctgtatatatattagcagcttagctagttcataacctgtatattctaaagactgctaaggttttgttttcattttaaatcctagctgattgttgtggtcaatgaaatacccagtttctggagggccaggtgggaaatgctttcactggaccaacacacaaatgatcatcctgaggatctgagcttccctagactccacacaataaccttggggcacccttttagagaagactgttgaaacccacagcactcgttggggtatgaggaaaccagggcttggcacaggaagttcccctttgtagctaaaagtccagaaagaaagggttcatctttttgacttccaactgatattgggaagtttggttgaggttcaagtgtgactccttccagagccacaggtaggggagtgtgaagttgagggggaggaaagctggaaggactctgccttgggagattcccagctctgctttccagcgcttggtggaatctgggctggggaaagacggcaccgggaaactctgcttccccattgtttccatctgatcagctgtggtgtgaggacttctcagacaaaggcaaggcctcgtgcccctgcccagcccattcatggagccctgggccttcttggcttccatagatcctaagctcttgactgtagtttagccagacttgttttgctatcttataagcagttcagaattagggaatgctggttttgaagagcaaaggacaggtagtctagagagggtcgtctggcctgcttgctgggtctttgtaacccagcacttcctcttgccctcctggctttatgtttatggggagaggactcaatagctccaccccttctggcaccagatggggcttggttagtttgcaataagcaccttgcagaggttaaagccagcgggtccctagtcttaggcccagcctgcttgtgtgggctctggcctggcctggtggctggcccagggggcagcagtgcttagagcttctgcagggcttctcttgtttacacagctgcatcagacaatgccatttctccccaccacggaaccttccatctaagatttcttccagggaatgccagcaatcaggcagcacccagctgtgggggcagtggggtgggggagacccacattgatgactttttttttttcttttaatgaagaaacaccaaagaaagctgtggaaaggacctgccccacatgaaaaggataagccaagatggctgtaaacacagagcatttgagctgccactcttggagcacattgatttttcaaaagccagctctgtcaggaaaggaggtgctgttatgagcagctcttccagtgggcaaagaggacgcccataatttcttccattgctagctcatctgtgggaccaatttggtgtaagcaacctgtggcctgcacttgtggcctcgaaggaagcacaaaccctccatccacttcccatttcctctgcccttttccacctcccccttccatcccaccagctgccagtggctcccagaaagccttattgagccccttgttgacacttggggctgcggaggcctctccctactggtctggcctttcctgagaggcaggtcttccgtcctcagagcctttctggaacaaggagaatgcctgtgcaggtggacacacaggcctggcctgtcgctctcacttgtcttccagcggggagcttcacgttgccgagtggaagaaccatgacctccacttgcttccaaggtgctagggaagtttcagggtacgctggttcccctctccagctggaggccgagtttctggggactgcagatttttctactctgtgatcgattcaatgcccgatgcttctgtttcattcccgaccctttctactatgcattttccttttatcaggtgtataaagttaaatactgtgtatttatcactaaaaagtacatgaacttaagagacaactaagcctttcgtgtttttccacaggtgtttaagcttctctgtacagttgaaataaacagacagcaaaatggtgccaa

SEQ ID NO: 7 - RNA, 3757 nt, Homo sapiens
  source 1..3757: mol_type = transcribed RNA
acagaagaaatagcaagtgccgagaagctggcatcagaaaaacagaggggagatttgtgtggctgcagccgagggagaccaggaagatctgcatggtgggaaggacctgatgatacagaggtgagaaataagaaaggctgctgactttaccatctgaggccacacatctgctgaaatggagataattaacatcactagaaacagcaagatgacaatataatgtctaagtagtgacatgtttttgcacatttccagcccctttaaatatccacacacacaggaagcacaaaaggaagcacagagatccctgggagaaatgcccggccgccatcttgggtcatcgatgagcctcgccctgtgcctggtcccgcttgtgagggaaggacattagaaaatgaattgatgtgttccttaaaggatgggcaggaaaacagatcctgttgtggatatttatttgaacgggattacagatttgaaatgaagtcacaaagtgagcattaccaatgagaggaaaacagacgagaaaatcttgatggcttcacaagacatgcaacaaacaaaatggaatactgtgatgacatgaggcagccaagctggggaggagataaccacggggcagagggtcaggattctggccctgctgcctaaactgtgcgttcataaccaaatcatttcatatttctaaccctcaaaacaaagctgttgtaatatctgatctctacggttccttctgggcccaacattctccatatatccagccacactcatttttaatatttagttcccagatctgtactgtgacctttctacactgtagaataacattactcattttgttcaaagacccttcgtgttgctgcctaatatgtagctgactgtttttcctaaggagtgttctggcccaggggatctgtgaacaggctgggaagcatctcaagatctttccagggttatacttactagcacacagcatgatcattacggagtgaattatctaatcaacatcatcctcagtgtctttgcccatactgaaattcatttcccacttttgtgcccattctcaagacctcaaaatgtcattccattaatatcacaggattaacttttttttttaacctggaagaattcaatgttacatgcagctatgggaatttaattacatattttgttttccagtgcaaagatgactaagtcctttatccctcccctttgtttgattttttttccagtataaagttaaaatgcttagccttgtactgaggctgtatacagccacagcctctccccatccctccagccttatctgtcatcaccatcaacccctcccatgcacctaaacaaaatctaacttgtaattccttgaacatgtcaggcatacattattccttctgcctgagaagctcttccttgtctcttaaatctagaatgatgtaaagttttgaataagttgactatcttacttcatgcaaagaagggacacatatgagattcatcatcacatgagacagcaaatactaaaagtgtaatttgattataagagtttagataaatatatgaaatgcaagagccacagagggaatgtttatggggcacgtttgtaagcctgggatgtgaagcaaaggcagggaacctcatagtatcttatataatatacttcatttctctatctctatcacaatatccaacaagcttttcacagaattcatgcagtgcaaatccccaaaggtaacctttatccatttcatggtgagtgcgctttagaattttggcaaatcatactggtcacttatctcaactttgagatgtgtttgtccttgtagttaattgaaagaaatagggcactcttgtgagccactttagggttcactcctggcaataaagaatttacaaagagctactcaggaccagttgttaagagctctgtgtgtgtgtgtgtgtgtgtgagtgtacatgccaaagtgtgcctctctctctttgacccattatttcagacttaaaaacaagcatgttttcaaatggcactatgagctgccaatgatgtatcaccaccatatctcattattctccagtaaatgtgataataatgtcatctgttaacataaaaaaagtttgacttcacaaaagcagctggaaatggacaaccacaatatgcataaatctaactcctaccatcagctacacactgcttgacatatattgttagaagcacctcgcatttgtgggttctcttaagcaaaatacttgcattaggtctcagctggggctgtgcatcaggcggtttgagaaatattcaattctcagcagaagccagaatttgaattccctcatcttttaggaatcatttaccaggtttggagaggattcagacagctcaggtgctttcactaatgtctctgaacttctgtccctctttgtgttcatggatagtccaataaataatgttatctttgaactgatgctcataggagagaatataagaactctgagtgatatcaacattagggattcaaagaaatattagatttaagctcacactggtcaaaaggaaccaagatacaaagaactctgagctgtcatcgtccccatctctgtgagccacaaccaacagcaggacccaacgcatgtctgagatccttaaatcaaggaaaccagtgtcatgagttgaattctcctattatggatgctagcttctggccatctctggctctcctcttgacacatattagcttctagcctttgcttccacgacttttatcttttctccaacacatcgcttaccaatcctctctctgctctgttgctttggacttccccacaagaatttcaacgactctcaagtcttttcttccatccccaccactaacctgaatgcctagacccttatttttattaatttccaatagatgctgcctatgggctatattgctttagatgaacattagatatttaaagctcaagaggttcaaaatccaactcattatcttctctttctttcacctccctgctcctctccctatattactgattgcactgaacagcatggtccccaatgtagccatgcaaatgagaaacccagtggctccttgtggtacatgcatgcaagactgctgaagccagaaggatgactgattacgcctcatgggtggaggggaccactcctgggccttcgtgattgtcaggagcaagacctgagatgctccctgccttcagtgtcctctgcatctcccctttctaatgaagatccatagaatttgctacatttgagaattccaattaggaactcacatgttttatctgccctatcaattttttaaacttgctgaaaattaagttttttcaaaatctgtccttgtaaattactttttcttacagtgtcttggcatactatatcaactttgattctttgttacaacttttcttactcttttatcaccaaagtggcttttattctctttattattattattttcttttactactatattacgttgttattattttgttctctatagtatcaatttatttgatttagtttcaatttatttttattgctgacttttaaaataagtgattcggggggtgggagaacaggggagggagagcattaggacaaatacctaatgcatgtgggacttaaaacctagatgatgggttgataggtgcagcaaaccactatggcacacgtatacctgtgtaacaaacctacacattctgcacatgtatcccagaacgtaaagtaaaatttaaaaaaaagtga

SEQ ID NO: 8 - RNA, 2922 nt, Homo sapiens
  source 1..2922: mol_type = transcribed RNA
agtgctgctctgcgctgcgccgcgctcggggctcgctctccttgctccgcgctccccgccagccgccccggggcaggaggcgcgcctgacggacggcccgctagacaaaggaggcgcggctcggcggggccagcgcgcggacggacggaccatggactcggagcgcgggcggccggccccagccttggggaccggacactcccgggcccggccctaggcgcccggccccgccgcccggcgcgcccagcggggaggacgtggagcccgcgcggcgcgagcaggcggcggccgcggagcaagaagggcgccgcggcgtgcggcccgcgcagcccccggagccatgggcaagtgcagcgggcgctgcacgctggtcgccttctgctgcctgcagctggtggctgcgctggagcggcagatctttgacttcctgggctaccagtgggctcccatcctagccaacttcctgcacatcatggcagtcatcctgggcatctttggcaccgtgcagtaccgctcccggtacctcatcctgtatgcagcctggctggtgctctgggttggctggaatgcatttatcatctgcttctacttggaggttggacagctgtcccaggaccgggacttcatcatgaccttcaacacatccctgcaccgctcctggtggatggagaatgggccaggctgcctggtgacacctgttctgaactcccgcctggctctggaggaccaccatgtcatctctgtcactggctgcctgcttgactacccctacattgaagccctcagcagcgccctgcagatcttcctggcactgttcggcttcgtgttcgcctgctacgtgagcaaagtgttcctggaggaggaggacagctttgacttcatcggcggctttgactcctacggataccaggcgccccagaagacgtcgcatttacagctgcagcctctgtacacgtcggggtagcctctgccccgcgcccaccccggcgcctcgccctgggctgaccgcagctgccgcgagctcgggccaaggcgcaggcgtgtccccctggtggcccgcgcgctcactgcagcctgtgcccaaccccgcgtctgcatctggagatgcggacttggacgtggacttggacttggacttggatttgagcttggctcttcgcagcccggacttcggaggagtggggcggggcgggggaggggcaccacgggttttttgttttttgtttgtttgtttttaatctcagccttggcgtgagctggggccttcctctcttctccagcctctccctttcactcttcacccagcatcctgcccccctgtccaaaaacagcaggacatcagacccatcccatcccaccacactcactcaccagctctggggaaagctactgtgaactaggagcaggattcctgggttctaatcgcaggtccatcactgactgtgacgtctagcaaagcccttgccctctctgagcctcggtttccgcacctcaagtaattaatcccttagcaaatggactcttttagacttctcatttaactcaattccctgagctagactgggattaaaattctcattttgcagtacattaaaactgaggcccagagatgtgatttgcttgaggccacacagctagatttttggtggaagtgggccttgaacacagtgtactttctgcagtttctgactgtaaaacccagtgtctgctctctgagttccatttccaagcccccctccatcttggacctatgtggtctccaccatattcacacaccaccaccaccacttgccaatgcctctcttaaagcaatatacccattcgttctcttattgggaactggatggatgaagccccaaattcagccccacccacagagaagccttcctacactcagcctctgtccacccttggcaaatctttcaagctctctcctccaggaaagtggggccccaactcagtcactccacccccttccaggtccctgaggctggttctactgtatccccatcacctccacaactccactcacccctgacggctccatccacctcaccagttggaaggcttgtggtttcagagaggagcaatgctggtcagcgctgcccagactccagtgtttacagatcaccagcatttacaaccaatccaatggccagaagcctcctctaacaagcccagaaggagttctgaaggggcagatgggggtgtgagtagtcggggagtcgggattgccagcaccctcacccttccttgggggcaagtagaggtgagaacactttccccacctccctccacagacactcctgaggacgctgcatcccacgcactgcctggtgcgtccatagagagaggatcaggtctcagcatttcatctgtgaaagaggcatggccctgggttagaaaggagggcaggagacatggaggaactggggggcacccagatggtgcagatggtttgcacacctgagcctgtctgtggtgaccattccgctcctctcccactaccctccaatctatcattccctactctctaaggccaaaatatcctgagcaaggctggcaaccccaccccaccatcccaaatgcaagcagccaggcccaggagttcctctggcccccacaggcatggagctcccagctggtgggtacagcttgagaggggggcagctccctcaggctaagctactgcccttcactgggccagccctgcctccagccctcacctctctcaccccaactctcccccaagcccctttctactcaacgggtgtagccactggtgctttgaagccttttgtttttataagatggtttttgcaaggggaccaggttctcttttcactgggaccttgcaaggaggggagtgctctcctggtttctgtgcaggcgggttgattaaagatggtgttttcttctcta

SEQ ID NO: 9 - RNA, 2503 nt, Homo sapiens
  source 1..2503: mol_type = transcribed RNA
agtgtgtgaagtaaagggattaaaggctagtctcaggctggggatggctcctgtctatttcttctctctcagagactgcagatggcttttccctgccgcaggtccctgactgccaagactctggcctgcctcctggtgggcgtgagtttcttagcactgcagcagtggttcctccaggcgccaaggtccccgcgggaggagaggtccccgcaggaggagacgccagagggtcccaccgacgctcccgcggctgacgagccgccctcggagctcgtccccgggcccccgtgcgtggcgaacgcctcggcgaacgccacggccgacttcgagcagctgcccgcgcgcatccaggacttcctgcggtaccgccactgccgccacttcccgctgctttgggacgcaccggccaagtgcgccggcggccgaggcgtgttcctgctcctggcggtgaagtcggcgcctgagcactacgagcgacgcgagctcatccggcgcacgtgggggcaagagcgcagctacggcgggcggccagtgcgccgcctctttctattgggcaccccgggccccgaggacgaggcgcgcgcggagcggctggcggagctggtggcgctggaggcgcgcgagcacggcgacgtgctgcagtgggccttcgcggacaccttcctcaacctcacgctcaagcacctgcacttgctcgactggctggctgcacgctgcccgcacgcgcgctttctgctcagcggcgacgacgacgtgttcgtgcacaccgccaacgtagtccgcttcctgcaggcgcagccacccggccgccacctgttctccggccagctcatggagggctccgtgcccatccgcgacagctggagcaagtacttcgtgccgccgcagctcttccccgggtccgcttacccggtgtactgcagcggcggcggcttcctcctgtccggccccacggcccgggccctgcgcgcggccgcccgccacaccccgctcttccccatcgacgacgcctacatgggcatgtgtctggagcgcgccggcctggcgcccagcggccacgagggcatccgacccttcggcgtgcagctgcctggcgcacagcagtcctccttcgacccctgcatgtaccgcgagttgctgctagtgcaccgcttcgcgccctacgagatgctgctcatgtggaaggcgctgcacagccccgcgctcagctgtgaccggggacaccgggtctcctgaggccagttgggcggcttcagccccgggcctccaaccatgtccatgctgagaaggcagctttcccgctctgggtaccttacgtcctgcccagctctgtgcacctgaaccccagctgcgcactgaaatcagctggggtggggggtgtggaaaatgcctacatcctggctccatctcccgaagtttcgatttgattagtctggggtggacccagacatgttaagtattttttaagttcctccagtgatgcgaatgtgcagctaggcctgaggaccactcggctagactatctcttcatcctcgcaaagccagctccaccgccctctctgcaagaattccgggcccctcgctcccacactcgggtcctcttgagcagtggagcaagggagacctgggagcgtgggagccaggatcagcgccccctgccatgtgcctacaaatgtcagttgtgatttccactgtttacaagtgagtggagctggagctgggctgacagtatcaggtggatcccgcttccccctcccccaagaagtcagccaacacgcagctgaggcgcatgtggtggccttcttcccaccactaccccagtacaccgtgaggtagaaatcttcaccgtgcaaagtggaaaccagaggcccggtcagacagtgactaatccagggccgtggcattcccagacagcacaccactgtggtcccctccacactcaccccaaccaaagctaatggcctagttgggtcctgcccgccaataatcacccccacgggtcagagacaggctccttgccggggtctgggcctcaggctcagtgggccttggacaacccagcagggagttccggggagtccgaagtggagaaaggctggtgggaacatggaggccagtgttggggagcctgtggaggcaggtgtgtagaattgtgttcgggaggtgggggatctgagaccgaagtggacagtggttaagattgtggggccgggcgaggtggctcacgcctgtaatcccagcactttgggaggctgaggaggtcggatcatgaggtcaagagttcgagaccagcctggccaatatggtgaaaccccgtctctattgggagtacaaaaattagccggccatagtggctcgtgcctgtaatctcagctatttgggaggctgaggcaggagaatcacttgaacctgggaggcggaggttgcagtgagccgagatcgtgccactgcactccagcctgggcgacagagcaagactgcatctcaaaaaaaaaaaaaaaaa

SEQ ID NO: 10 - RNA, 867 nt, Homo sapiens
  source 1..867: mol_type = transcribed RNA
gagtcctgagctgcgtcccggagcccacggtggtcatggctgccagagcgctctgcatgctggggctggtcctggccttgctgtcctccagctctgctgaggagtacgtgggcctgtctgcaaaccagtgtgccgtgccagccaaggacagggtggactgcggctacccccatgtcacccccaaggagtgcaacaaccggggctgctgctttgactccaggatccctggagtgccttggtgtttcaagcccctgcaggaagcagaatgcaccttctgaggcacctccagctgcccccggccgggggatgcgaggctcggagcacccttgcccggctgtgattgctgccaggcactgttcatctcagcttttctgtccctttgctcccggcaagcgcttctgctgaaagttcatatctggagcctgatgtcttaacgaataaaggtcccatgctccacccgaggacagttcttcgtgcctgagactttctgaggttgtgctttatttctgctgcgtcgtgggagagggcgggagggtgtcaggggagagtctgcccaggcctcaagggcaggaaaagactccctaaggagctgcagtgcatgcaaggatattttgaatccagactggcacccacgtcacaggaaagcctaggaacactgtaagtgccgcttcctcgggaaagcagaaaaaatacatttcaggtagaagttttcaaaaatcacaagtctttcttggtgaagacagcaagccaataaaactgtcttccaaagtggtcctttatttcacaaccactctcgctactgttcaatacttgtactattcctgggttttgtttctttgtacagtaaacattatgaacaaacaggca

SEQ ID NO: 11 - RNA, 1579 nt, Homo sapiens
  source 1..1579: mol_type = transcribed RNA
acccgaccgctgccggccgcgctcccgctgctcctgccgggtgatggaaaaccccagcccggccgccgccctgggcaaggccctctgcgctctcctcctggccactctcggcgccgccggccagcctcttgggggagagtccatctgttccgccagagccctggccaaatacagcatcaccttcacgggcaagtggagccagacggccttccccaagcagtaccccctgttccgcccccctgcgcagtggtcttcgctgctgggggccgcgcatagctccgactacagcatgtggaggaagaaccagtacgtcagtaacgggctgcgcgactttgcggagcgcggcgaggcctgggcgctgatgaaggagatcgaggcggcgggggaggcgctgcagagcgtgcacgcggtgttttcggcgcccgccgtccccagcggcaccgggcagacgtcggcggagctggaggtgcagcgcaggcactcgctggtctcgtttgtggtgcgcatcgtgcccagccccgactggttcgtgggcgtggacagcctggacctgtgcgacggggaccgttggcgggaacaggcggcgctggacctgtacccctacgacgccgggacggacagcggcttcaccttctcctcccccaacttcgccaccatcccgcaggacacggtgaccgagataacgtcctcctctcccagccacccggccaactccttctactacccgcggctgaaggccctgcctcccatcgccagggtgacactggtgcggctgcgacagagccccagggccttcatccctcccgccccagtcctgcccagcagggacaatgagattgtagacagcgcctcagttccagaaacgccgctggactgcgaggtctccctgtggtcgtcctggggactgtgcggaggccactgtgggaggctcgggaccaagagcaggactcgctacgtccgggtccagcccgccaacaacgggagcccctgccccgagctcgaagaagaggctgagtgcgtccctgataactgcgtctaagaccagagccccgcagcccctggggccccccggagccatggggtgtcgggggctcctgtgcaggctcatgctgcaggcggccgagggcacagggggtttcgcgctgctcctgaccgcggtgaggccgcgccgaccatctctgcactgaagggccctctggtggccggcacgggcattgggaaacagcctcctcctttcccaaccttgcttcttaggggcccccgtgtcccgtctgctctcagcctcctcctcctgcaggataaagtcatccccaaggctccagctactctaaattatgtctccttataagttattgctgctccaggagattgtccttcatcgtccaggggcctggctcccacgtggttgcagatacctcagacctggtgctctaggctgtgctgagcccactctcccgagggcgcatccaagcgggggccacttgagaagtgaataaatggggcggtttcggaagcgtcagtgtttccatgttatggatctctctgcgtttgaataaagactatctctgttgctcacaaa

SEQ ID NO: 12 - RNA, 1603 nt, Homo sapiens
  source 1..1603: mol_type = transcribed RNA
aaggcactctggcacccagttttggaactgcagttttaaaagtcataaattgaatgaaaatgatagcaaaggtggaggtttttaaagagctatttataggtccctggacagcatcttttttcaattaggcagcaacctttttgccctatgccgtaacctgtgtctgcaacttcctctaattgggaaatagttaagcagattcatagagctgaatgataaaattgtactacgagatgcactgggactcaacgtgaccttatcaagtgagcaggcttggtgcatttgacacttcatgatatcagccaaagtggaactaaaaacagctcctggaagaggactatgacatcatcaggttgggagtctccagggacagcggaccctttggaaaaggactagaaagtgtgaaatctattagtcttcgatatgaaattctctgtctctgtaaaagcatttcatatttacaagacacaggcctactcctagggcagcaaaaagtggcaacaggcaagcagagggaaaagagatcatgaggcatttcagagtgcactgtcttttcatatatttctcaatgccgtatgtttggttttattttggccaagcataacaatctgctcaagaaaaaaaaatctggagaaaacaaaggtgcctttgccaatgttatgtttctttttgacaagccctgagatttctgaggggaattcacataaatgggatcaggtcattcatttacgttgtgtgcaaatatgatttaaagatacaacctttgcagagagcatgctttcctaagggtaggcacgtggaggactaagggtaaagcattcttcaagatcagttaatcaagaaaggtgctctttgcattctgaaatgcccttgttgcaaatattggttatattgattaaatttacacttaatggaaacaacctttaacttacagatgaacaaacccacaaaagcaaaaaatcaaaagccctacctatgatttcatattttctgtgtaactggattaaaggattcctgcttgcttttgggcataaatgataatggaatatttccaggtattgtttaaaatgagggcccatctacaaattcttagcaatactttggataattctaaaattcagctggacattgtctaattgttttttatatacatctttgctagaatttcaaattttaagtatgtgaatttagttaattagctgtgctgatcaattcaaaaacattactttcctaaattttagactatgaaggtcataaattcaacaaatatatctacacatacaattatagattgtttttcattataatgtcttcatcttaacagaattgtctttgtgattgtttttagaaaactgagagttttaattcataattacttgatcaaaaaattgtgggaacaatccagcattaattgtatgtgattgtttttatgtacataaggagtcttaagcttggtgccttgaagtcttttgtacttagtcccatgtttaaaattactactttatatctaaagcatttatgtttttcaattcaatttacatgatgctaattatggcaattataacaaatattaaagatttcgaaatagaaaaaaaaaaaaaaa

SEQ ID NO: 13 - RNA, 1530 nt, Homo sapiens
  source 1..1530: mol_type = transcribed RNA
gtgaggacaccgctttacaacgatgcagggggccccatgtcaccctcacccatgggaagtttgacttggtggactcagccaagccacagaggtctaacgcttctctgcggtgatttcaggctgccctggcagaaagcacagtgcctgcagacatgctgtcactgctccacgcatcaacgctggcagtccttggggctctgtgtgtatatggtgcaggtcacctagagcaacctcaaatttccagtactaaaacgctgtcaaaaacagcccgcctggaatgtgtggtgtctggaataacaatttctgcaacatctgtatattggtatcgagagagacctggtgaagtcatacagttcctggtgtccatttcatatgacggcactgtcagaaaggaatccggcattccgtcaggcaaatttgaggtggataggatacctgaaacgtctacatccactctcaccattcacaatgtagagaaacaggacatagctacctactactgtgccttgttggagggaaattataagaaactctttggcagtggaacaacacttgttgtcacagataaacaacttgatgcagatgtttcccccaagcccactatttttcttccttcaattgctgaaacaaagctccagaaggctggaacatacctttgtcttcttgagaaatttttccctgatgttattaagatacattggcaagaaaagaagagcaacacgattctgggatcccaggaggggaacaccatgaagactaacgacacatacatgaaatttagctggttaacggtgccagaaaagtcactggacaaagaacacagatgtatcgtcagacatgagaataataaaaacggagttgatcaagaaattatctttcctccaataaagacagatgtcatcacaatggatcccaaagacaattgttcaaaagatgcaaatgatacactactgctgcagctcacaaacacctctgcatattacatgtacctcctcctgctcctcaagagtgtggtctattttgccatcatcacctgctgtctgcttagaagaacggctttctgctgcaatggagagaaatcataacagacggtggcacaaggaggccatcttttcctcatcggttattgtccctagaagcgtcttctgaggatctagttgggctttctttctgggtttgggccatttcagttctcatgtgtgtactattctatcattattgtataacggttttcaaaccagtgggcacacagagaacctcactctgtaataacaatgaggaatagccacggcgatctccagcaccaatctctccatgttttccacagctcctccagccaacccaaatagcgcctgctatagtgtagacatcctgcggcttctagccttgtccctctcttagtgttctttaatcagataactgcctggaagcctttcattttacacgccctgaagcagtcttctttgctagttgaattatgtggtgtgtttttccgtaataagcaaaataaatttaaaaaaatgaaaagtt

SEQ ID NO: 14 - RNA, 634 nt, Homo sapiens
  source 1..634: mol_type = transcribed RNA
aacgctaacctggtccggagcgagtctgggtctcagccccgcgaacagcctttcacgagtcttcaagctttcaggctatcttctagtcaagatgagtgataagccagacttgtcggaagtggagaagtttgacaggtcaaaactgaagaaaactaatactgaagaaaaaaatactcttccctcaaaggaaactatccagcaagagaaagagtgtgttcaaacatcataaaatggggatcgcctcccaacagcagatttcgacattacctgagagtcttgattttaggcttgttttttgtaaacccatgtgtttgtagagattttaggcgtcttcggatatcttctcacctatgttccctggctaagaagtcagaggtagccaatgtttccttaaattcatttttaaacttaccattggtgcatatgttccagatggcagatgctgtcaataatctcaccattgatgacctttgtgtatgtagttcttgcatcctatactggataagcctgttttaacctgctatgatgggtgcttccattgcttcataatcttcatgaagttgcatgcttttgcagcttttcacagtttatttgcatttctaatgtagtaataaagtaaccaatataatcatta

SEQ ID NO: 15 - RNA, 4904 nt, Homo sapiens
  source 1..4904: mol_type = transcribed RNA
atccgctctaaacaacctcatcaaaactactttctggtcagagagaagcaataattattattaacatttattaacgatcaataaacttgatcgcattatggccagcactattaaggaagccttatcagttgtgagtgaggaccagtcgttgtttgagtgtgcctacggaacgccacacctggctaagacagagatgaccgcgtcctcctccagcgactatggacagacttccaagatgagcccacgcgtccctcagcaggattggctgtctcaacccccagccagggtcaccatcaaaatggaatgtaaccctagccaggtgaatggctcaaggaactctcctgatgaatgcagtgtggccaaaggcgggaagatggtgggcagcccagacaccgttgggatgaactacggcagctacatggaggagaagcacatgccacccccaaacatgaccacgaacgagcgcagagttatcgtgccagcagatcctacgctatggagtacagaccatgtgcggcagtggctggagtgggcggtgaaagaatatggccttccagacgtcaacatcttgttattccagaacatcgatgggaaggaactgtgcaagatgaccaaggacgacttccagaggctcacccccagctacaacgccgacatccttctctcacatctccactacctcagagagactcctcttccacatttgacttcagatgatgttgataaagccttacaaaactctccacggttaatgcatgctagaaacacagggggtgcagcttttattttcccaaatacttcagtatatcctgaagctacgcaaagaattacaactaggccagatttaccatatgagccccccaggagatcagcctggaccggtcacggccaccccacgccccagtcgaaagctgctcaaccatctccttccacagtgcccaaaactgaagaccagcgtcctcagttagatccttatcagattcttggaccaacaagtagccgccttgcaaatccaggcagtggccagatccagctttggcagttcctcctggagctcctgtcggacagctccaactccagctgcatcacctgggaaggcaccaacggggagttcaagatgacggatcccgacgaggtggcccggcgctggggagagcggaagagcaaacccaacatgaactacgataagctcagccgcgccctccgttactactatgacaagaacatcatgaccaaggtccatgggaagcgctacgcctacaagttcgacttccacgggatcgcccaggccctccagccccaccccccggagtcatctctgtacaagtacccctcagacctcccgtacatgggctcctatcacgcccacccacagaagatgaactttgtggcgccccaccctccagccctccccgtgacatcttccagtttttttgctgccccaaacccatactggaattcaccaactgggggtatataccccaacactaggctccccaccagccatatgccttctcatctgggcacttactactaaagacctggcggaggcttttcccatcagcgtgcattcaccagcccatcgccacaaactctatcggagaacatgaatcaaaagtgcctcaagaggaatgaaaaaagctttactggggctggggaaggaagccggggaagagatccaaagactcttgggagggagttactgaagtcttactacagaaatgaggaggatgctaaaaatgtcacgaatatggacatatcatctgtggactgaccttgtaaaagacagtgtatgtagaagcatgaagtcttaaggacaaagtgccaaagaaagtggtcttaagaaatgtataaactttagagtagagtttggaatcccactaatgcaaactgggatgaaactaaagcaatagaaacaacacagttttgacctaacataccgtttataatgccattttaaggaaaactacctgtatttaaaaatagaaacatatcaaaaacaagagaaaagacacgagagagactgtggcccatcaacagacgttgatatgcaactgcatggcatgtgctgttttggttgaaatcaaatacattccgtttgatggacagctgtcagctttctcaaactgtgaagatgacccaaagtttccaactcctttacagtattaccgggactatgaactaaaaggtgggactgaggatgtgtatagagtgagcgtgtgattgtagacagaggggtgaagaaggaggaggaagaggcagagaaggaggagaccagggctgggaaagaaacttctcaagcaatgaagactggactcaggacatttggggactgtgtacaatgagttatggagactcgagggttcatgcagtcagtgttataccaaacccagtgttaggagaaaggacacagcgtaatggagaaaggggaagtagtagaattcagaaacaaaaatgcgcatctctttctttgtttgtcaaatgaaaattttaactggaattgtctgatatttaagagaaacattcaggacctcatcattatgtgggggctttgttctccacagggtcaggtaagagatggccttcttggctgccacaatcagaaatcacgcaggcattttgggtaggcggcctccagttttcctttgagtcgcgaacgctgtgcgtttgtcagaatgaagtatacaagtcaatgtttttccccctttttatataataattatataacttatgcatttatacactacgagttgatctcggccagccaaagacacacgacaaaagagacaatcgatataatgtggccttgaattttaactctgtatgcttaatgtttacaatatgaagttattagttcttagaatgcagaatgtatgtaataaaataagcttggcctagcatggcaaatcagatttatacaggagtctgcatttgcactttttttagtgactaaagttgcttaatgaaaacatgtgctgaatgttgtggattttgtgttataatttactttgtccaggaacttgtgcaagggagagccaaggaaataggatgtttggcacccaaatggcgtcagcctctccaggtccttcttgcctcccctcctgtcttttatttctagccccttttggaacagaaggaccccgggtttcacattggagcctccatatttatgcctggaatggaaagaggcctatgaagctggggttgtcattgagaaattctagttcagcacctggtcacaaatcacccttaattcctgctatgattaaaatacatttgttgaacagtgaacaagctaccactcgtaaggcaaactgtattattactggcaaataaagcgtcatggatagctgcaatttctcactttacagaaacaagggataacgtctagatttgctgcggggtttctctttcaggagctctcactaggtagacagctttagtcctgctacatcagagttacctgggcactgtggcttgggattcactagccctgagcctgatgttgctggctatcccttgaagacaatgtttatttccataatctagagtcagtttccctgggcatcttttctttgaatcacaaatgctgccaaccttggtccaggtgaaggcaactcaaaaggtgaaaatacaaggtgaccgtgcgaaggcgctagccgaaacatcttagctgaataggtttctgaactggcccttttcatagctgtttcagggcctgtttttttcacgttgcagtccttttgctatgattatgtgaagttgccaaacctctgtgctgtggatgttttggcagtgggctttgaagtcggcaggacacgattaccaatgctcctgacaccccgtgtcatttggattagacggagcccaaccatccatcattttgcagcagcctgggaaggcccacaaagtgcccgtatctccttagggaaaataaataaatacaatcatgaaagctggcagttaggctgacccaaactgtgctaatggaaaagatcagtcatttttattttggaatgcaaagtcaagacacacctacattcttcatagaaatacacatttacttggataatcactcagttctctcttcaagactgtctcatgagcaagatcataaaaacaagacatgattatcatattcaattttaacagatgttttccattagatccctcaaccctccacccccagtccaggttattagcaagtcttatgagcaactgggataattttggataacatgataatactgagttccttcaaatacataattcttaaattgtttcaaaatggcattaactctctgttactgttgtaatctaattccaaagccccctccaggtcatattcataattgcatgaaccttttctctctgtttgtccctgtctcttggcttgccctgatgtatactcagactcctgtacaatcttactcctgctggcaagagatttgtcttcttttcttgtcttcaattggctttcgggccttgtatgtggtaaaatcaccaaatcacagtcaagactgtgtttttgttcctagtttgatgcccttatgtcccggaggggttcacaaagtgctttgtcaggactgctgcagttagaaggctcactgcttctcctaagccttctgcacagatgtggcacctgcaacccaggagcaggagccggaggagctgccctctgacagcaggtgcagcagagatggctacagctcaggagctgggaaggtgatggggcacagggaaagcacagatgttctgcagcgccccaaagtgacccattgcctggagaaagagaagaaaatattttttaaaaagctagtttatttagcttctcattaattcattcaaataaagtcgtgaggtgactaattagagaataaaaattactttggactactcaaaaa

SEQ ID NO: 16 - RNA, 1416 nt, Homo sapiens
  source 1..1416: mol_type = transcribed RNA
aggcagcaggctggagctcagcccagcagtggaatccaggagcccagaggtggccgggtgctgacgtgatggccacagcaggaaatccctggggctggttcctggggtacctcatccttggtgtcgcaggatcgctcgtctctggtagctgcagccaaatcataaacggcgaggactgcagcccgcactcgcagccctggcaggcggcactggtcatggaaaacgaattgttctgctcgggcgtcctggtgcatccgcagtgggtgctgtcagccgcacactgtttccagaactcctacaccatcgggctgggcctgcacagtcttgaggccgaccaagagccagggagccagatggtggaggccagcctctccgtacggcacccagagtacaacagacccttgctcgctaacgacctcatgctcatcaagttggacgaatccgtgtccgagtctgacaccatccggagcatcagcattgcttcgcagtgccctaccgcggggaactcttgcctcgtttctggctggggtctgctggcgaacggcagaatgcctaccgtgctgcagtgcgtgaacgtgtcggtggtgtctgaggaggtctgcagtaagctctatgacccgctgtaccaccccagcatgttctgcgccggcggagggcaagaccagaaggactcctgcaacggtgactctggggggcccctgatctgcaacgggtacttgcagggccttgtgtctttcggaaaagccccgtgtggccaagttggcgtgccaggtgtctacaccaacctctgcaaattcactgagtggatagagaaaaccgtccaggccagttaactctggggactgggaacccatgaaattgacccccaaatacatcctgcggaaggaattcaggaatatctgttcccagcccctcctccctcaggcccaggagtccaggcccccagcccctcctccctcaaaccaagggtacagatccccagcccctcctccctcagacccaggagtccagaccccccagcccctcctccctcagacccaggagtccagcccctcctccctcagacccaggagtccagaccccccagcccctcctccctcagacccaggagtccagcccctcctccctcagacccaggagtccagaccccccagcccctcctccctcagacccaggggtccaggcccccaacccctcctccctcagactcagaggtccaggcccccaacccctccttccccagacccagaggtccaggtcccagcccctcctccctcagacccagcggtccaatgccacctagactctccctgtacacagtgcccccttgtggcacgttgacccaaccttaccagttggtttttcattttttgtccctttcccctagatccagaaataaagtctaagagaagcgca

SEQ ID NO: 17 - RNA, 1651 nt, Homo sapiens
  source 1..1651: mol_type = transcribed RNA
ataaccatctagttccgagtacaaactggagacagaaataaatattaaagaaatcatagaccgaccaggtaaaggcaaagggatgaattcctacttcactaacccttccttatcctgccacctcgccgggggccaggacgtcctccccaacgtcgccctcaattccaccgcctatgatccagtgaggcatttctcgacctatggagcggccgttgcccagaaccggatctactcgactcccttttattcgccacaggagaatgtcgtgttcagttccagccgggggccgtatgactatggatctaattccttttaccaggagaaagacatgctctcaaactgcagacaaaacaccttaggacataacacacagacctcaatcgctcaggattttagttctgagcagggcaggactgcgccccaggaccagaaagccagtatccagatttacccctggatgcagcgaatgaattcgcacagtggggtcggctacggagcggaccggaggcgcggccgccagatctactcgcggtaccagaccctggaactggagaaggaatttcacttcaatcgctacctaacgcggcgccggcgcatcgagatcgccaacgcgctttgcctgaccgagcgacagatcaaaatctggttccagaaccgccggatgaagtggaaaaaagaatctaatctcacatccactctctcggggggcggcggaggggccaccgccgacagcctgggcggaaaagaggaaaagcgggaagagacagaagaggagaagcagaaagagtgaccaggactgtccctgccacccctctctccctttctccctcgctccccaccaactctcccctaatcacacactctgtatttatcactggcacaattgatgtgttttgattccctaaaacaaaattagggagtcaaacgtggacctgaaagtcagctctggaccccctccctcaccgcacaactctctttcaccacgcgcctcctcctcctcgctcccttgctagctcgttctcggcttgtctacaggcccttttccccgtccaggccttgggggctcggaccctgaactcagactctacagattgccctccaagtgaggacttggctcccccactccttcgacgcccccacccccgccccccgtgcagagagccggctcctgggcctgctggggcctctgctccagggcctcagggcccggcctggcagccggggagggccggaggcccaaggagggcgcgccttggccccacaccaacccccagggcctccccgcagtccctgcctagcccctctgccccagcaaatgcccagcccaggcaaattgtatttaaagaatcctgggggtcattatggcattttacaaactgtgaccgtttctgtgtgaagatttttagctgtatttgtggtctctgtatttatatttatgtttagcaccgtcagtgttcctatccaatttcaaaaaaggaaaaaaaagagggaaaattacaaaaagagagaaaaaaagtgaatgacgtttgtttagccagtaggagaaaataaataaataaataaatcccttcgtgttaccctcctgtataaatccaacctctgggtccgttctcgaatatttaataaaactgatattatttttaaaacttta